(19) United States
(12) Patent Application Publication
(10) Pub. No.: US 2012/0302622 A1
(43) Pub. Date: Nov. 29, 2012

(54) CONJUGATES, PARTICLES, COMPOSITIONS, AND RELATED METHODS

(75) Inventors: Scott Eliasof, Lexington, MA (US); Oliver S. Fetzer, Needham, MA (US); Jungyeon Hwang, Lexington, MA (US); Patrick Lim Soo, Somerville, MA (US); Pei-Sze Ng, Cambridge, MA (US); Sonke Svenson, Arlington, MA (US); Donald E. Bergstrom, West Lafayette, IN (US)

(73) Assignee: CERULEAN PHARMA INC., Cambridge, MA (US)

(21) Appl. No.: 13/443,765

(22) Filed: Apr. 10, 2012

Related U.S. Application Data
(63) Continuation of application No. PCT/US2011/048305, filed on Aug. 18, 2011.
(60) Provisional application No. 61/375,783, filed on Aug. 20, 2010.
(60) Provisional application No. 61/387,882, filed on Sep. 29, 2010.
(60) Provisional application No. 61/443,972, filed on Feb. 17, 2011.
(60) Provisional application No. 61/475,923, filed on Apr. 15, 2011.

(51) Int. Cl. A61K 31/713; C07H 1/00; C08F 16/06; C07H 21/02

(52) U.S. Cl. 514/44 A; 536/24.5; 536/25.3; 525/54.2

(57) ABSTRACT

Particles and conjugates for delivering nucleic acid agents. Compositions containing the particles, the conjugates, or both. Methods of using the particles, the conjugates, and the compositions.

### CLAIM OF PRIORITY

This application is a continuation of International Serial No. PCT/US2011/048305 filed Aug. 11, 2011 which claims priority to U.S. Ser. No. 61/375,783, filed Aug. 20, 2010; U.S. Ser. No. 61/387,882, filed Sep. 29, 2010; U.S. Ser. No. 61/443,972, filed Feb. 17, 2011; and U.S. Ser. No. 61/475,923, filed Apr. 15, 2011, the contents of each of which are incorporated herein by reference.

### BACKGROUND OF INVENTION

Effective delivery of a nucleic acid agent to a therapeutic target is desirable to provide optimal use and effectiveness of that nucleic acid agent. Particle delivery systems may increase the efficacy or tolerability of the nucleic acid agent.

### SUMMARY OF INVENTION

Described herein are particles, which can be used, for example, in the delivery of a nucleic acid agent. Typically, the particles include a nucleic acid agent, and at least one of a cationic moiety, a hydrophobic moiety, such as a polymer, or a hydrophilic-hydrophobic polymer. In some embodiments, the particles include a nucleic acid agent and a cationic moiety, and at least one of a hydrophobic moiety, such as a polymer, or a hydrophilic-hydrophobic polymer. In some embodiments, the particle includes a nucleic acid agent, a cationic moiety, and both a hydrophobic moiety, such as a polymer, and a hydrophilic-hydrophobic polymer. In other embodiments the particle includes a nucleic acid agent, a cationic moiety, and either i) a hydrophobic moiety, such as a polymer, or ii) a hydrophilic-hydrophobic polymer is present, and when one is present, the other is substantially absent, or one of the two is present at less than 5, 2 or 1% by weight of the other, for example, as determined by amount in the particle or as determined by the amounts of material used to make the particle. In an embodiment one or more of a hydrophobic moiety (e.g., a hydrophobic polymer), hydrophilic-hydrophobic polymer, cationic moiety, or nucleic acid agent can be attached to another moiety, e.g., another moiety recited just above or elsewhere herein. For example, in an embodiment, the cationic moiety and/or nucleic acid agent can be attached to the hydrophobic moiety (e.g., hydrophobic polymer) and/or the hydrophilic-hydrophobic polymer. The particle can also include other components such as a surfactant or a hydrophilic polymer (e.g., a hydrophilic polymer such as PEG, which can be further attached to a lipid). Also described herein are conjugates, such as nucleic acid agent-polymer conjugates, mixtures, compositions and dosage forms containing the particles or conjugates, methods of using the particles (e.g., to treat a disorder), kits including the nucleic acid agent-polymer conjugates and particles, methods of making the nucleic acid agent-polymer conjugates and particles, methods of storing the particles and methods of analyzing the particles.

Particles disclosed herein provide for the delivery of nucleic acid agents, e.g., siRNA or an agent that promotes RNAi.

Accordingly, in one aspect, the disclosure features, a particle comprising:

a) a plurality of hydrophobic moieties, e.g., hydrophobic polymers;

b) a plurality of hydrophilic-hydrophobic polymers;

c) optionally, a plurality of cationic moieties; and

d) a plurality of nucleic acid agents, wherein at least a portion of the plurality of nucleic acid agents are

(i) covalently attached to either of

a hydrophobic moiety, e.g., a hydrophobic polymer of a) or

a hydrophilic-hydrophobic polymer of b), or

(ii) form a duplex (e.g., a heteroduplex) with a nucleic acid which is covalently attached to either of a hydrophobic moiety, e.g., hydrophobic polymer, of a) or the hydrophilic-hydrophobic polymer b).

In some embodiments, the particle comprises a cationic moiety.

In an embodiment, the particle is a nanoparticle.

In some embodiments, the hydrophobic moiety is a hydrophobic polymer. In some embodiments, the hydrophobic moiety is not a polymer.

In some embodiments, at least a portion of the hydrophobic moieties, e.g., hydrophobic polymers, of a) are not covalently attached to a nucleic acid agent. In some embodiments, at least a portion of the hydrophobic polymers of a) are not covalently attached to a cationic moiety.

In some embodiments, substantially all of the cationic moieties of c) are not covalently attached to a hydrophobic moiety, e.g., a hydrophobic polymer, and are free of covalent attachment to a polymer of b).

In some embodiments, at least a portion of plurality of hydrophobic polymers are free of covalent attachment one or both of a cationic moiety of c) or a nucleic acid agent of d).

In some embodiments, at least a portion of the hydrophobic moieties, e.g., hydrophobic polymers, of a) are each covalently attached to a nucleic acid agent of d).

In some embodiments, at least a portion of the hydrophobic moieties, e.g., hydrophobic polymers, of a) are each covalently attached to a single nucleic acid agent of d). In some embodiments, at least a portion of the hydrophobic polymers of a) are, each, covalently attached to a plurality of nucleic acid agents of d).

In some embodiments, at least a portion of the hydrophobic moieties, e.g., hydrophobic polymers of a) are each directly covalently attached (e.g., without the presence of atoms from an intervening spacer moiety), to a nucleic acid agent of d) (e.g., at the carboxy terminal or hydroxyl terminal of the hydrophobic polymers).

In some embodiments, at least a portion of the nucleic acid agents of d) are covalently attached to the hydrophobic polymer via a linker. Exemplary linkers include a linker that comprises a bond formed using click chemistry (e.g., as described in WO 2006/115547) and a linker that comprises an amide, an ester, a disulfide, a sulfide, a ketal, a succinate, an oxime, a carbamate, a carbonate, a silyl ether, or a triazole (e.g., an amide, an ester, a disulfide, a sulfide, a ketal, a succinate, or a triazole). In some embodiments, the linker comprises a functional group such as a bond that is cleavable under physiological conditions. In some embodiments, the linker comprises a plurality of functional groups such as bonds that are cleavable under physiological conditions. In some embodiments, the linker includes a functional group such as a bond or functional group described herein that is not directly attached either to a first or second moiety linked through the linker at the terminal ends of the linker, but is interior to the linker. In some embodiments, the linker is hydrolysable under physiologic conditions, the linker is enzymatically cleavable under physiological conditions, or the linker comprises a disulfide which can be reduced under physiological conditions. In some embodiments, the linker is not cleaved under physiological conditions, for example, the linker is of a sufficient length that the nucleic acid agent does not need to be cleaved to be active, e.g., the length of the linker is at least about 20 angstroms (e.g., at least about 24 angstroms).

In some embodiments, the nucleic acid agent forms a duplex with a nucleic acid that is attached to the hydrophobic polymer. For example, the nucleic acid agent (e.g., an siRNA or an agent that promotes RNAi) can form a duplex (e.g., a heteroduplex) with a DNA attached to the hydrophobic polymer.

In some embodiments, at least a portion of the hydrophobic moieties, e.g., hydrophobic polymers, of a) are each covalently attached to a nucleic acid agent of d) through the 3′ and/or 5′ position of the nucleic acid agent. In some embodiments, at least a portion of the hydrophobic moieties, e.g., hydrophobic polymers, of a) are each covalently attached to a nucleic acid agent of d) through the 2′ position of the nucleic acid agent.

In some embodiments, at least a portion of the hydrophilic-hydrophobic polymers of b) are each covalently attached to a nucleic acid agent of d) (e.g., at the carboxy terminal or hydroxyl terminal of the hydrophobic polymers or at a terminal end of the hydrophilic polymers). In some embodiments, at least a portion of the hydrophilic-hydrophobic polymers of b) are each covalently attached to a single nucleic acid agent of d). In some embodiments, at least a portion of the hydrophilic-hydrophobic polymers of b) are each covalently attached to a plurality of nucleic acid agents of d).

In some embodiments, at least a portion of the hydrophilic-hydrophobic polymers of b) are each directly covalently attached (e.g., without the presence of atoms from an intervening spacer moiety) to a nucleic acid agent of d) (e.g., at the carboxy terminal or hydroxyl terminal of the hydrophobic polymers or at a terminal end of the hydrophilic polymers). In some embodiments, at least a portion of the nucleic acid agents are each covalently attached to the hydrophilic-hydrophobic polymer via a linker.

Exemplary linkers include a linker that comprises a bond formed using click chemistry (e.g., as described in WO 2006/115547) and a linker that comprises an amide, an ester, a disulfide, a sulfide, a ketal, a succinate, an oxime, a carbamate, a carbonate, a silyl ether, or a triazole (e.g., an amide, an ester, a disulfide, a sulfide, a ketal, a succinate, or a triazole). In some embodiments, the linker comprises a functional group such as a bond that is cleavable under physiological conditions. In some embodiments, the linker comprises a plurality of functional groups such as bonds that are cleavable under physiological conditions. In some embodiments, the linker includes a functional group such as a bond or functional group described herein that is not directly attached either to a first or second moiety linked through the linker at the terminal ends of the linker, but is interior to the linker. In some embodiments, the linker is hydrolysable under physiologic conditions, the linker is enzymatically cleavable under physiological conditions, or the linker comprises a disulfide which can be reduced under physiological conditions. In some embodiments, the linker is not cleaved under physiological conditions, for example, the linker is of a sufficient length that the nucleic acid agent does not need to be cleaved to be active, e.g., the length of the linker is at least about 20 angstroms (e.g., at least about 24 angstroms).

In some embodiments, a nucleic acid agent forms a duplex with a nucleic acid that is attached to a hydrophobic polymer. For example, a nucleic acid agent (e.g., an RNAi) can form a duplex (e.g., a heteroduplex) with a DNA attached to a hydrophobic moiety, e.g., a hydrophobic polymer. In some embodiments, a nucleic acid agent forms a duplex with a nucleic acid that is attached to a hydrophilic-hydrophobic polymer. For example, a nucleic acid agent (e.g., an RNAi) can form a duplex (e.g., a heteroduplex) with a DNA attached to a hydrophobic moiety, e.g., a hydrophobic polymer.

In some embodiments, at least a portion of the plurality of hydrophilic-hydrophobic polymers of b) are each covalently attached to a nucleic acid agent through the 3′ and/or 5′ position of the nucleic acid agent. In some embodiments, at least a portion of the plurality of hydrophilic-hydrophobic polymers of b) is each covalently attached to the nucleic acid agent through the 2′ position of the nucleic acid agent.

In some embodiments, at least a portion of the hydrophobic moieties, e.g., hydrophobic polymers, of a) are each covalently attached to a cationic moiety of c), e.g., at least a portion of the plurality of hydrophobic moieties, e.g., hydrophobic polymers of a) are each directly covalently attached (e.g., without the presence of atoms from an intervening spacer moiety), to a cationic moiety of c). In some embodiments, at least a portion of the plurality of hydrophobic moieties, e.g., hydrophobic, polymers of a) are each covalently attached to a cationic moiety of c) through an amide, ester, thioether, or ether (e.g., at the carboxy terminal of the hydrophobic polymers).

In some embodiments, at least a portion of the plurality of hydrophobic moieties, e.g., hydrophobic, polymers of a) are each covalently attached to a cationic moiety of c) at a terminal end of the hydrophobic polymer. In some embodiments, a single cationic moiety of c) is covalently attached to a single hydrophobic polymer of a) (e.g., at the terminal end of the hydrophobic polymer). In some embodiments, a single hydrophobic polymer of a) is covalently attached to a plurality of cationic moieties of c).

In some embodiments, at least a portion of the plurality of cationic moieties of c) is each attached to the backbone of a hydrophobic polymer, of a).

In some embodiments, at least a portion of the plurality of hydrophobic moieties, e.g., hydrophobic polymers, of a) are each covalently attached to a cationic moiety of c), and at least a portion of the plurality of hydrophobic moieties, e.g., hydrophobic, polymers of a) are each attached to a nucleic acid agent of d).

In some embodiments, the particle comprises the cationic moieties of c), and further comprises a plurality of additional cationic moieties, wherein the additional cationic moieties differ from the cationic moieties of c). The additional cationic moiety can be, e.g., a cationic polymer (e.g., PEI, cationic PVA, poly(histidine), poly(lysine), or poly(2-dimethylamino)ethyl methacrylate). In some embodiments, at least a portion of the plurality of the additional cationic moieties are each attached to at least a portion of the plurality of hydrophobic moieties, e.g., hydrophobic, polymers of a) and/or the plurality of hydrophilic-hydrophobic polymers of b). In some embodiments, at least a portion of the plurality of the additional cationic moieties are attached to at least a portion of the plurality of hydrophobic moieties, e.g., hydrophobic, polymers of a).

In some embodiments, the particle further comprises a plurality of additional nucleic acid agents, wherein the additional nucleic agents differ, e.g., in structure, e.g., sequence, length, length of overhang, or derivitization (e.g., modification of the sugar or base) of the nucleic acid agents, from the plurality of nucleic acid agents of d). In some embodiments, at least a portion of the plurality of the additional nucleic acid agents are attached to at least a portion of either the plurality of hydrophobic moieties, e.g., hydrophobic polymers, of a) and/or the plurality of hydrophilic-hydrophobic polymers of b). In some embodiments, at least a portion of the plurality of the additional nucleic acid agents are attached to at least a portion of the plurality of hydrophobic moieties, e.g., hydrophobic, polymers of a).

Particles disclosed herein provide for delivery of nucleic acid agents, e.g., an agent that promotes RNAi such as siRNA, wherein the nucleic acid agents are attached to a hydrophobic polymer, or duplexed with a nucleic acid that is attached to a hydrophobic polymer.

Accordingly, in another aspect, the disclosure features, a particle comprising:

a) a plurality of nucleic acid agent-polymer conjugates, each of which comprises a nucleic acid agent which

(i) is attached to a hydrophobic polymer or

(ii) forms a duplex (e.g., a heteroduplex) with a nucleic acid which is covalently attached to a hydrophobic polymer;

b) a plurality of hydrophilic-hydrophobic polymers; and

c) optionally, a plurality of cationic moieties.

In some embodiments, particle comprises a cationic moiety.

In an embodiment, the particle is a nanoparticle.

In some embodiments, the particle further comprises a hydrophobic polymer, for example, wherein the hydrophobic polymer is not attached to a nucleic acid such as a nucleic acid agent. In some embodiments, the particle comprises the plurality of cationic moieties of c), at least a portion of which are each covalently attached to a hydrophobic polymer (e.g., a hydrophobic polymer that is not attached to a nucleid acid such as a nucleic acid agent) Exemplary cationic moiety-hydrophobic polymer conjugates include N1-PLGA-N5,N10,N14-tetramethylated-spermine.

In some embodiments, the particle comprises the plurality of cationic moieties of c), and at least a portion of the plurality of hydrophilic-hydrophobic polymers of b) is each covalently attached to a cationic moiety of c). In some embodiments, at least a portion of the plurality of cationic moieties of c) are each covalently attached to the hydrophobic portion of a hydrophilic-hydrophobic polymer of b) (e.g., through a linker described herein such as an amide, ester or ether). In some embodiments, at least a portion of the plurality of cationic moieties of c) are each covalently attached to the hydrophilic portion of the hydrophilic-hydrophobic polymer of b).

In some embodiments, a nucleic acid agent is covalently attached to a hydrophobic polymer via a linker. Exemplary linkers include a linker that comprises a bond formed using click chemistry (e.g., as described in WO 2006/115547) and a linker that comprises an amide, an ester, a disulfide, a sulfide, a ketal, a succinate, an oxime, a carbamate, a carbonate, a silyl ether, or a triazole (e.g., an amide, an ester, a disulfide, a sulfide, a ketal, a succinate, or a triazole). In some embodiments, the linker comprises a functional group such as a bond that is cleavable under physiological conditions. In some embodiments, the linker comprises a plurality of functional groups such as bonds that are cleavable under physiological conditions. In some embodiments, the linker includes a functional group such as a bond or functional group described herein that is not directly attached either to a first or second moiety linked through the linker at the terminal ends of the linker, but is interior to the linker. In some embodiments, the linker is hydrolysable under physiologic conditions, the linker is enzymatically cleavable under physiological conditions, or the linker comprises a disulfide which can be reduced under physiological conditions. In some embodiments, the linker is not cleaved under physiological conditions, for example, the linker is of a sufficient length that the nucleic acid agent does not need to be cleaved to be active, e.g., the length of the linker is at least about 20 angstroms (e.g., at least about 24 angstroms).

In some embodiments, a nucleic acid agent forms a duplex with a nucleic acid that is attached to the hydrophobic polymer. For example, the nucleic acid agent (e.g., an siRNA or an agent that promotes RNAi) can form a duplex (e.g., a homo or heteroduplex) with a nucleic acid (for example and RNA or DNA) attached to the hydrophobic polymer.

In some embodiments, the particle comprises the cationic moieties of c), and further comprises a plurality of additional cationic moieties, wherein the additional cationic moieties differ, e.g., in molecular weight, viscosity, charge, or structure, from the plurality of cationic moieties of c). In some embodiments, at least a portion of the plurality of the additional cationic moieties is attached to hydrophobic polymers and/or at least a portion of the hydrophilic-hydrophobic polymers of b). In some embodiments, at least a portion of the plurality of the additional cationic moieties is attached to a hydrophobic polymer.

In some embodiments, the particle further comprises a plurality of additional nucleic acid agents, wherein the additional nucleic agents differ, e.g., in structure, e.g., sequence, length, length of overhang, or derivitization (e.g., modification of the sugar or base) of the nucleic acid agents, from the plurality of nucleic acid agents of a). In some embodiments, at least a portion of the plurality of the additional nucleic acid agents are attached to hydrophobic polymers and/or at least a portion of the plurality of hydrophilic-hydrophobic polymers of b). In some embodiments, at least a portion of the plurality of the additional nucleic acid agents is attached to a hydrophobic polymer.

Particles of the invention provide for the attachment of a nucleic acid agent, e.g., an siRNA or an agent that promotes RNAi, to a hydrophilic-hydrophobic polymer. Hydrophobic moieties and cationic moieties are also included, e.g., as described below.

Accordingly, in another aspect, the invention features a particle comprising:

a) a plurality of hydrophobic moieties, e.g., hydrophobic polymers;

b) a plurality of nucleic acid agent-hydrophilic-hydrophobic polymer conjugates wherein the nucleic acid agent of each nucleic acid agent-hydrophilic-hydrophobic polymer conjugate of the plurality
(i) is covalently attached to the hydrophilic-hydrophobic polymer or
(ii) forms a duplex (e.g., a heteroduplex) with a nucleic acid which is covalently attached the hydrophilic-hydrophobic polymer; and
   c) optionally, a plurality of cationic moieties.

In some embodiments, the particle comprises a plurality of cationic moieties.

In an embodiment, the particle is a nanoparticle.

In some embodiments, the particle also includes a plurality of hydrophilic-hydrophobic polymers, wherein the hydrophilic-hydrophobic polymers are not covalently attached to a nucleic acid such as a nucleic acid agent.

In some embodiments, the particle comprises the plurality of cationic moieties of c), and at least a portion of the plurality of cationic moieties of c) is covalently attached to a hydrophilic-hydrophobic polymer, for example, the cationic moieties of c) is covalently attached to a hydrophilic-hydrophobic polymer that is not attached to a nucleic acid agent.

In some embodiments, the particle comprises the plurality of cationic moieties of c), and at least a portion of the plurality of hydrophilic-hydrophobic polymers are covalently attached to a cationic moiety of c) through the hydrophobic portion of the hydrophobic-hydrophilic polymer (e.g., through an amide, ester or ether). In some embodiments, at least a portion of the plurality of hydrophobic polymers of a) is covalently attached to a cationic moiety of c) (e.g., through an amide, ester or ether). In some embodiments, the hydrophobic-hydrophilic polymer of the conjugate of b) is covalently attached to the nucleic acid agent via a linker. Exemplary linkers include a linker that comprises a bond formed using click chemistry (e.g., as described in WO 2006/115547) and a linker that comprises an amide, an ester, a disulfide, a sulfide, a ketal, a succinate, an oxime, a carbamate, a carbonate, a silyl ether, or a triazole (e.g., an amide, an ester, a disulfide, a sulfide, a ketal, a succinate, or a triazole). In some embodiments, the linker comprises a functional group such as a bond that is cleavable under physiological conditions. In some embodiments, the linker comprises a plurality of functional groups such as bonds that are cleavable under physiological conditions. In some embodiments, the linker includes a functional group such as a bond or functional group described herein that is not directly attached either to a first or second moiety linked through the linker at the terminal ends of the linker, but is interior to the linker. In some embodiments, the linker is hydrolysable under physiologic conditions, the linker is enzymatically cleavable under physiological conditions, or the linker comprises a disulfide which can be reduced under physiological conditions. In some embodiments, the linker is not cleaved under physiological conditions, for example, the linker is of a sufficient length that the nucleic acid agent does not need to be cleaved to be active, e.g., the length of the linker is at least about 20 angstroms (e.g., at least about 24 angstroms).

In some embodiments, the particle comprises the cationic moieties of c), and further comprises a plurality of additional cationic moieties, wherein the additional cationic moieties differ, e.g., in molecular weight, viscosity, charge, or structure, from the cationic moieties of c). In some embodiments, at least a portion of the plurality of the additional cationic moieties are attached to at least a portion of the plurality of hydrophobic polymers of a) and/or plurality of hydrophilic-hydrophobic polymers. In some embodiments, at least a portion of the plurality of the additional cationic moieties is attached to at least a portion of the plurality of hydrophobic polymers of a).

In some embodiments, the particle further comprises a plurality of additional nucleic acid agents, wherein the additional nucleic agents differ, e.g., in structure, e.g., sequence, length, length of overhang, or derivitization (e.g., modification of the sugar or base) of the nucleic acid agents, from the plurality of nucleic acid agents of b). In some embodiments, at least a portion of the plurality of the additional nucleic acid agents are attached to at least a portion of either the plurality of hydrophobic polymers of a) and/or plurality of hydrophilic-hydrophobic polymers. In some embodiments, at least a portion of the plurality of the additional nucleic acid agents is attached to at least a portion of the plurality of hydrophobic polymers of a).

In some embodiments, the nucleic acid agent forms a duplex with a nucleic acid that is attached to at least a portion of the plurality of hydrophobic polymers of a). For example, the nucleic acid agent (e.g., an siRNA or an agent that promotes RNAi) can form a duplex (e.g., a homo or heteroduplex) with a nucleic acid (for example an RNA or DNA) attached to the hydrophobic polymer.

Particles of the invention provide for delivery of nucleic acid agents, e.g., siRNA or an agent that promotes RNAi, in particles that comprise cationic moieties attached to a polymer, as described herein.

Accordingly, in another aspect, the invention features a particle comprising:

a) a plurality of hydrophobic moieties, e.g., hydrophobic polymers;

b) a plurality of hydrophilic-hydrophobic polymers;

c) a plurality of cationic moieties, wherein at least a portion of the plurality of cationic moieties is attached to either a hydrophobic polymer of a) or a hydrophilic-hydrophobic polymer of b); and

d) a plurality of nucleic acid agents.

In some embodiments, at least a portion of the plurality of hydrophobic moieties, e.g., polymers, of a) is not covalently attached to a cationic moiety of c). In some embodiments, at least a portion of the plurality of hydrophobic polymers of a) is not covalently attached to a nucleic acid agent of d).

In an embodiment, the particle is a nanoparticle.

In some embodiments, substantially all of the plurality of nucleic acid agents of d) is not covalently attached to a polymer (e.g., a polymer of a) or b)). In some embodiments, at least a portion of plurality of hydrophobic polymers of a) is not covalently attached to a cationic moiety of c) or a nucleic acid agent of d).

In some embodiments, the nucleic acid agent is covalently attached to a hydrophilic polymer such as a PEG polymer. In some embodiments, the PEG is attached to a lipid and or modified at a terminal end with a methyl group.

In some embodiments, at least a portion of the plurality of hydrophobic polymers of a) are each covalently attached to a cationic moiety of c), for example, a plurality of hydrophobic polymers are covalently attached to tetramethylated spermine (e.g., N1-PLGA-N5, N10, N14 tetramethylated-spermine). In some embodiments, at least a portion of the plurality of hydrophobic polymers of a) are each covalently attached to a cationic moiety of c) through an amide, ester or ether (e.g., at the carboxy terminal of the hydrophobic polymers). In some embodiments, at least a portion of the plurality of hydrophobic polymers of a) are each covalently attached to a cationic moiety of c) at a terminal end of the hydrophobic polymer. In some embodiments, at least a portion of the plurality of cationic moieties of c) are directly covalently attached (e.g., without the presence of atoms from an intervening spacer moiety), to the hydrophobic polymer of a) (e.g., at the carboxy terminal or hydroxyl terminal of the hydrophobic polymers). In some embodiments, at least a portion of the plurality of cationic moieties of c) are covalently attached to the hydrophobic polymer of a) via a linker (e.g., at the carboxy terminal or hydroxyl terminal of the hydrophobic polymers). In some embodiments, the linker comprises a bond formed using click chemistry (e.g., as described in WO 2006/115547). In some embodiments, the linker comprises an amide, an ester, a disulfide, a sulfide (i.e., a thioether bond), a ketal, a succinate, an oxime, a carbonate, a carbamate, a silyl ether, or a triazole. In some embodiments, a single cationic moiety of c) is covalently attached to a single hydrophobic polymer of a) (e.g., at the terminal end of the hydrophobic polymer). In some embodiments, at least a portion of the plurality of cationic moieties of c) is covalently attached to the hydrophilic-hydrophobic polymer of b) through the hydrophobic portion via an amide, ester, thioether, or ether bond. In some embodiments, a single hydrophobic polymer of a) is covalently attached to a plurality of cationic moieties of c). In some embodiments, at least a portion of the plurality of cationic moieties of c) is attached to the backbone of at least a portion of the hydrophobic polymers of a).

In some embodiments, at least a portion of the plurality of hydrophilic-hydrophobic polymers of b) is covalently attached to a cationic moiety of c). In some embodiments, at least a portion of the plurality of cationic moieties of c) are directly covalently attached (e.g., without the presence of atoms from an intervening spacer moiety), to a hydrophilic-hydrophobic polymer of b) (e.g., at the carboxy terminal or hydroxyl terminal of the hydrophobic polymers). In some embodiments, at least a portion of the plurality of cationic moieties of c) are covalently attached to the hydrophilic-hydrophobic polymer of a) via a linker (e.g., at the carboxy terminal or hydroxyl terminal of the hydrophobic polymers). In some embodiments, the linker comprises a bond formed using click chemistry (e.g., as described in WO 2006/115547). In some embodiments, the linker comprises an amide, an ester, a disulfide, a sulfide, a ketal, a succinate, an oxime, a carbonate, a carbamate, a silyl ether, or a triazole. In some embodiments, a single cationic moiety of c) is covalently attached to a single hydrophilic-hydrophobic polymer of b) (e.g., at the terminal end of the hydrophilic-hydrophobic polymer). In some embodiments, at least a portion of the plurality of cationic moieties of c) is covalently attached to the hydrophilic-hydrophobic polymer of b) through the hydrophobic portion. In some embodiments, at least a portion of the plurality of cationic moieties of c) is covalently attached to the hydrophilic-hydrophobic polymer of b) through the hydrophobic portion. In some embodiments, at least a portion of the plurality of cationic moieties of c) is covalently attached to the hydrophilic-hydrophobic polymer of b) through the hydrophobic portion via an amide, ester or ether bond. In some embodiments, a single hydrophilic-hydrophobic polymer of b) is covalently attached to a plurality of cationic moieties of c). In some embodiments, at least a portion of the plurality of cationic moieties of c) is attached to the backbone of at least a portion of the hydrophilic-hydrophobic polymers of b).

In some embodiments, at least a portion of the plurality of hydrophobic polymers of a) is covalently attached to a nucleic acid agent of d). In some embodiments, at least a portion of the hydrophobic polymers of a) is covalently attached to a single nucleic acid agent of d). In some embodiments, at least a portion of the hydrophobic polymers of a) is covalently attached to a plurality of nucleic acid agents of d). In some embodiments, the nucleic acid agent of d) is directly covalently attached (e.g., without the presence of atoms from an intervening spacer moiety), to the hydrophobic polymer of a) (e.g., at the hydroxyl terminal of the hydrophilic-hydrophobic polymer). In some embodiments, the nucleic acid agent is covalently attached to the hydrophobic polymer of a) via a linker (e.g., at the hydroxyl terminal of the hydrophilic-hydrophobic polymer). Exemplary linkers include a linker that comprises a bond formed using click chemistry (e.g., as described in WO 2006/115547) and a linker that comprises an amide, an ester, a disulfide, a sulfide, a ketal, a succinate, an oxime, a carbamate, a carbonate, a silyl ether, or a triazole (e.g., an amide, an ester, a disulfide, a sulfide, a ketal, a succinate, or a triazole). In some embodiments, the linker comprises a functional group such as a bond that is cleavable under physiological conditions. In some embodiments, the linker comprises a plurality of functional groups such as bonds that are cleavable under physiological conditions. In some embodiments, the linker includes a functional group such as a bond or functional group described herein that is not directly attached either to a first or second moiety linked through the linker at the terminal ends of the linker, but is interior to the linker. In some embodiments, the linker is hydrolysable under physiologic conditions, the linker is enzymatically cleavable under physiological conditions, or the linker comprises a disulfide which can be reduced under physiological conditions. In some embodiments, the linker is not cleaved under physiological conditions, for example, the linker is of a sufficient length that the nucleic acid agent does not need to be cleaved to be active, e.g., the length of the linker is at least about 20 angstroms (e.g., at least about 24 angstroms).

In some embodiments, at least a portion of the hydrophobic polymers of a) is covalently attached to a nucleic acid agent of d) through the 3′ and/or 5′ position of the nucleic acid agent. In some embodiments, at least a portion of the hydrophobic polymers of a) is covalently attached to a nucleic acid agent of d) through the 2′ position of the nucleic acid agent.

In some embodiments, a nucleic acid agent forms a duplex with a nucleic acid that is attached to at least a portion of the plurality of hydrophobic polymers of a). For example, the nucleic acid agent (e.g., an siRNA or an agent that promotes RNAi) can form a duplex (e.g., a homo or heteroduplex) with a nucleic acid (for example an RNA or DNA) attached to the hydrophobic polymer.

In some embodiments, at least a portion of the hydrophilic-hydrophobic polymers of b) are covalently attached to a nucleic acid agent of d). In some embodiments, at least a portion of the hydrophilic-hydrophobic polymers of b) are each covalently attached to a single nucleic acid agent of d). In some embodiments, at least a portion of the hydrophilic-hydrophobic polymers of b) are each covalently attached to a plurality of nucleic acid agents of d). In some embodiments, at least a portion of the nucleic acid agents of d) are directly covalently attached (e.g., without the presence of atoms from an intervening spacer moiety), to the hydrophilic-hydrophobic polymer of b) (e.g., at the hydroxyl terminal of the hydrophilic-hydrophobic polymer). In some embodiments, at least a portion of the nucleic acid agents of d) are each covalently attached to the hydrophilic-hydrophobic polymer of b) via a linker (e.g., at the hydroxyl terminal of the hydrophilic-hydrophobic polymer). Exemplary linkers include a linker that comprises a bond formed using click chemistry (e.g., as described in WO 2006/115547) and a linker that comprises an amide, an ester, a disulfide, a sulfide, a ketal, a succinate, an oxime, a carbamate, a carbonate, a silyl ether, or a triazole (e.g., an amide, an ester, a disulfide, a sulfide, a ketal, a succinate, or a triazole). In some embodiments, the linker comprises a functional group such as a bond that is cleavable under physiological conditions. In some embodiments, the linker comprises a plurality of functional groups such as bonds that are cleavable under physiological conditions. In some embodiments, the linker includes a functional group such as a bond or functional group described herein that is not directly attached either to a first or second moiety linked through the linker at the terminal ends of the linker, but is interior to the linker. In some embodiments, the linker is hydrolysable under physiologic conditions, the linker is enzymatically cleavable under physiological conditions, or the linker comprises a disulfide which can be reduced under physiological conditions. In some embodiments, the linker is not cleaved under physiological conditions, for example, the linker is of a sufficient length that the nucleic acid agent does not need to be cleaved to be active, e.g., the length of the linker is at least about 20 angstroms (e.g., at least about 24 angstroms).

In some embodiments, at least a portion of the hydrophilic-hydrophobic polymers of b) are each covalently attached to the nucleic acid agent of d) through the 3′ and/or 5′ position of the nucleic acid agent. In some embodiments, at least a portion of the hydrophilic-hydrophobic polymers of b) are covalently attached to the nucleic acid agent of d) through the 2′ position of the nucleic acid agent.

In some embodiments, at least a portion of the hydrophobic polymers of a) are covalently attached to a cationic moiety of c), and at least a portion of the hydrophobic polymers of a) are attached to a nucleic acid agent of d).

In some embodiments, the particle further comprises a plurality of additional cationic moieties, wherein the additional cationic moieties differ, e.g., in molecular weight, viscosity, charge, or structure, from the cationic moieties of c). In some embodiments, at least a portion of the plurality of the additional cationic moieties is attached to at least a portion of the hydrophobic polymers of a) and/or the hydrophilic-hydrophobic polymers of b). In some embodiments, at least a portion of the plurality of the additional cationic moieties is attached to at least a portion of the hydrophobic polymers of a).

In some embodiments, the particle further comprises a plurality of additional nucleic acid agents, wherein the additional nucleic agents differ, e.g., in structure, e.g., sequence, length, length of overhang, or derivitization (e.g., modification of the sugar or base) of the nucleic acid agents, from the nucleic acid agents of d). In some embodiments, at least a portion of the plurality of the additional nucleic acid agents are attached to at least a portion of either the hydrophobic polymers of a) and/or the hydrophilic-hydrophobic polymers of b). In some embodiments, at least a portion of the plurality of the additional nucleic acid agents is attached to at least a portion of the hydrophobic polymers of a).

Particles of the invention provide for delivery of nucleic acid agents, e.g., siRNA or an agent that promotes RNAi, wherein the nucleic acid agent is covalently attached to a hydrophilic polymer, or forms a duplex with a nucleic acid covalently attached to a hydrophilic polymer.

Accordingly, in another aspect, the invention features a particle comprising:

a) a plurality of hydrophobic moieties (e.g., hydrophobic polymers);

b) optionally a plurality of hydrophilic-hydrophobic polymers;

c) a plurality of cationic moieties; and

d) a plurality of nucleic acid agents, wherein at least a portion of the plurality of nucleic acid agents are covalently attached to a hydrophilic polymer or form a duplex (e.g., a heteroduplex) with a nucleic acid that is covalently attached to a hydrophilic polymer.

In an embodiment, the particle is a nanoparticle.

In some embodiments, the nucleic acid agent is covalently attached to a hydrophilic polymer (e.g., comprising PEG). In some embodiments, the PEG has a molecular weight of about 2 kDa. In some embodiments, the polymer (e.g., hydrophilic polymer) is covalently attached to a lipid (e.g., 1,2-distearoyl-sn-glycero-3-phosphoethanolamine-N-[PDP(polyethylene glycol)-2k]). Exemplary lipids are described herein such as DSPE. In one embodiment, the polymer is PEG covalently attached to a lipid, e.g., PEG covalently attached to 1,2-distearoyl-sn-glycero-3-phosphoethanolamine-N-[PDP(polyethylene glycol)-2kDa].

In an embodiment, the particle is substantially free of a hydrophobic-hydrophilic polymer. In an embodiment, a hydrophobic-hydrophilic polymer, if present amounts to less than 5, 2, or 1%, by weight, of the components, e.g., polymers, in, or used as starting materials to make, the particles.

In some embodiments, the hydrophobic moiety is a hydrophobic polymer such as PLGA. In some embodiments, the hydrophilic-hydrophobic polymer is a PEG-PLGA polymer.

Particles of the invention provide for delivery of nucleic acid agents, e.g., siRNA or an agent that promotes RNAi, wherein the nucleic acid agent is not attached (e.g., covalently attached) to a hydrophobic moiety such as a polymer or a hydrophilic-hydrophobic polymer and does not form a duplex with a nucleic acid that is attached (e.g., covalently attached) to a hydrophobic moiety such as a polymer or a hydrophilic-hydrophobic polymer. In the alternative, in some particles, less than 5, 2, or 1%, by weight, of the nucleic acid agent in, or used as starting materials to make, the particles, are attached to such polymers.

Accordingly, in another aspect, the invention features, a particle comprising:

a) a plurality of hydrophobic moieties (e.g., hydrophobic polymers);

b) a plurality of hydrophilic-hydrophobic polymers; and

c) a plurality of nucleic acid agent-cationic polymer conjugates.

In an embodiment, the particle is a nanoparticle.

In an embodiment the nucleic acid agent is not attached, e.g., covalently attached, to a hydrophobic polymer or hydrophilic-hydrophobic polymer. In an embodiment, less than 5, 2, or 1%, by weight, of the nucleic acid agent in, or used as starting materials to make, the particle, are attached to hydrophobic polymers or hydrophilic-hydrophobic polymers.

In some embodiments, the cationic polymer is PVA, e.g., the nucleic acid agent-cationic polymer conjugate is an siRNA-cationic PVA conjugate. In some embodiments, the hydrophobic moiety is a hydrophobic polymer such as PLGA. In some embodiments, the hydrophilic-hydrophobic polymer is a PEG-PLGA polymer

Particles of the invention provide for delivery of nucleic acid agents, e.g., siRNA or an agent that promotes RNAi, wherein the neither the nucleic acid agent nor the cationic polymer is attached, e.g., covalently attached, to hydrophobic polymer or hydrophilic-hydrophobic polymer or wherein, independently, less than 5, 2, or 1%, by weight, of the nucleic acid agents and cationic moieties in, or used as starting materials to make, the particles, are attached to such polymers. Thus nucleic acid agents and cationic moieties of the particle, e.g., substantially all of the nucleic acid agents and cationic moieties of the particle are embedded within the particle, as opposed to being covalently linked to a polymer component.

Accordingly, in another aspect, the invention features a particle comprising:

a) a plurality of hydrophobic moieties (e.g., hydrophobic polymers);

b) a plurality of hydrophilic-hydrophobic polymers;

c) optionally, a plurality of cationic moieties; and

d) a plurality of nucleic acid agents;

wherein a substantial portion of the cationic moieties of c) and a substantial portion of the nucleic acid agents of d) is not covalently attached to a hydrophobic polymer or a hydrophilic-hydrophobic polymer. For example, the nucleic acid agents or cationic moieties are embedded in the particle.

In some embodiments, the particle comprises a plurality of cationic moieties.

In an embodiment, the particle is a nanoparticle.

In an embodiment, independently, less than 5, 2, or 1%, by weight, of the nucleic acid agent in, or used as starting materials to make, the particles, are attached to such polymers and, less than 5, 2, or 1%, by weight of the cationic moieties in, or used as starting materials to make, the particle, are attached to such polymers.

In some embodiments, the cationic moiety is a cationic polymer. Exemplary cationic polymers include cationic PVA such as a cationic PVA described herein or spermine, including modified spermine (e.g., tetramethylated spermine). The nucleic acid agent can form complex with the cationic moiety such as a cationic polymer described herein. The nucleic acid agent complexed with the cationic moiety can be embedded in the particle. In some embodiments, the ratio of the charge of the cationic moiety to the charge of the backbone of the nucleic acid agent is from about 2:1 to about 1:1 (e.g., about 1.5:1 to about 1:1).

In some embodiments, the hydrophobic moiety is a hydrophobic polymer such as PLGA. In some embodiments, the hydrophilic-hydrophobic polymer is a PEG-PLGA polymer.

A particle described herein can have one or more of the following properties. In one embodiment, at least a portion of the hydrophobic polymers of a) has a carboxy terminal end. In one embodiment, a terminal end such as the carboxy terminal end is modified (e.g., with a reactive group including a reactive group described herein). In one embodiment, at least a portion of the hydrophobic polymers of a) has a hydroxyl terminal end. In one embodiment, the hydroxyl terminal end is modified (e.g., with a reactive group). In one embodiment, at least a portion of the hydrophobic polymers of a) having a hydroxyl terminal end have the hydroxyl terminal end capped (e.g., capped with an acyl moiety). In one embodiment, at least a portion of the hydrophobic polymers of a) have both a carboxy terminal end and a hydroxyl terminal end. In one embodiment, at least a portion of the hydrophobic polymers of a) comprise monomers of lactic and/or glycolic acid. In one embodiment, at least a portion of the hydrophobic polymers of a) comprise PLA or PGA. In one embodiment, at least a portion of the hydrophobic polymers of a) comprises copolymers of lactic and glycolic acid (i.e., PLGA). In one embodiment, the polymer polydispersity index is less than about 2.5 (e.g., less than about 1.5). In one embodiment, a portion of the hydrophobic polymers of a) comprises PLGA having a ratio of from about 25:75 to about 75:25 of lactic acid to glycolic acid. In one embodiment, a portion of the hydrophobic polymers of a) comprises PLGA having a ratio of about 50:50 of lactic acid to glycolic acid. In one embodiment, the hydrophobic polymers of a) have a Mw of from about 4 to about 66 kDa, for example from about 4 to about 12 kDa from about 8 to about 12 kDa. In one embodiment, the hydrophobic polymers of a) have a weight average molecular weight of from about 4 to about 12 kDa (e.g., from about 4 to about 8 kDa). In one embodiment, the hydrophobic polymers of a) comprise from about 35 to about 90% by weight in, or used as starting materials to make, the particle (e.g., from about 35 to about 80% by weight). In one embodiment, at least a portion of the hydrophobic polymers of a) are each covalently attached to a single cationic moiety and a portion of the hydrophobic polymers of a) are attached to a plurality of cationic moieties. In one embodiment, at least a portion of the hydrophobic polymers of a) are each covalently attached to a single nucleic acid agent and a portion of the hydrophobic polymers of a) are attached to a plurality of nucleic acid agents.

Additional properties of the particles described herein include the following. In some embodiments, the hydrophilic-hydrophobic polymers of b) are block copolymers. In some embodiments, the hydrophilic-hydrophobic polymers of b) are diblock copolymers. In some embodiments, the hydrophobic portion of at least a portion of the hydrophilic-hydrophobic polymers of b) has a hydroxyl terminal end. In some embodiments, the hydrophobic portion of at least a portion of the hydrophilic-hydrophobic polymers of b) having a hydroxyl terminal end have the hydroxyl terminal end capped (e.g., capped with an acyl moiety). In some embodiments, the hydrophobic portion of at least a portion of the hydrophilic-hydrophobic polymers of b) having a hydroxyl terminal end have the hydroxyl terminal end capped with an acyl moiety.

Additional properties of the particles described herein include the following. In some embodiments, the hydrophobic portion of at least a portion of the hydrophilic-hydrophobic polymers of b) comprises copolymers of lactic and glycolic acid (i.e., PLGA). In some embodiments, the hydrophobic portion of at least a portion of the hydrophilic-hydrophobic polymers of b) comprises PLGA having a ratio of from about 25:75 to about 75:25 of lactic acid to glycolic acid. In some embodiments, the hydrophobic portion of at least a portion of the hydrophilic-hydrophobic polymers of b) comprises PLGA having a ratio of about 50:50 of lactic acid to glycolic acid.

Additional properties of the particles described herein include the following. In some embodiments, the hydrophobic portion of at least a portion of the hydrophilic-hydrophobic polymers of b) has a weight average molecular weight of from about 4 to about 20 kDa (e.g., from about 4 to about 12 kDa, from about 6 to about 20 kDa or from about 8 to about 15 kDa). In some embodiments, hydrophilic portion of at least a portion of the hydrophilic-hydrophobic polymers of b) has a weight average molecular weight of from about 1 to about 8 kDa (e.g., from about 2 to about 6 kDa). In some embodiments, at least a portion of the plurality of hydrophilic-hydrophobic polymers of b) is from about 2 to about 30 by weight % in, or used as starting materials to make, the particle (e.g., from about 4 to about 25 by weight %). In some embodiments, at least a portion of the hydrophilic portion of the hydrophilic-hydrophobic polymers of b) comprises PEG, polyoxazoline, polyvinylpyrrolidine, polyhydroxylpropylmethacrylamide, or polysialic acid (e.g., PEG). In some embodiments, at least a portion of the hydrophilic portion of the hydrophilic-hydrophobic polymers of b) terminates in a methoxy. In some embodiments, at least a portion of the hydrophilic-hydrophobic polymers of b) are each covalently attached to a single cationic moiety and a portion of the hydrophilic-hydrophobic polymers of b) are attached to a plurality of cationic moieties. In some embodiments, at least a portion of the hydrophilic-hydrophobic polymers of b) are each covalently attached to a single nucleic acid agent and a portion of the hydrophilic-hydrophobic polymers of b) are attached to a plurality of nucleic acid agents.

Additional properties of the particles described herein include the following. In some embodiments, at least a portion of the cationic moieties of c) comprise at least one amine (e.g., a primary, secondary, tertiary or quaternary amine). In some embodiments, at least a portion of the cationic moieties of c) comprise a plurality of amines (e.g., a primary, secondary, tertiary or quaternary amines). In some embodiments, at least one amine in the cationic moiety is a secondary or tertiary amine. In some embodiments, at least a portion of the cationic moieties of c) comprise a polymer, for example, polyethylene imine or polylysine Polymeric cationic moieties have a variety of molecular weights (e.g., ranging from about 500 to about 5000 Da, for example, from about 1 to about 2 kDa or about 2.5 kDa). In some embodiments, at least a portion of the cationic moieties of c) comprise a cationic PVA (e.g., as provided by Kuraray, such as CM-318 or C-506). Other exemplary cationic moieties include polyamino acids, poly(histidine) and poly(2-dimethylamino)ethyl methacrylate. In some embodiments, the cationic moiety has a pKa of 5 or greater. In some embodiments, the amine is positively charged at acidic pH. In some embodiments, the amine is positively charged at physiological pH. In some embodiments, at least a portion of the cationic moieties of c) is selected from the group consisting of protamine sulfate, hexademethrine bromide, cetyl trimethylammonium bromide, spermine (e.g., tetramethylated spermine), and spermidine. In some embodiments, at least a portion of the cationic moieties of c) are selected from the group consisting of tetraalkyl ammonium moieties, trialkyl ammonium moieties, imidazolium moieties, aryl ammonium moieties, iminium moieties, amidinium moieties, guanadinium moieties, thiazolium moieties, pyrazolylium moieties, pyrazinium moieties, pyridinium moieties, and phosphonium moieties. In some embodiments, at least a portion of the cationic moieties of c) are a cationic lipid. In some embodiments, at least a portion of the cationic moieties of c) are conjugated to a non-polymeric hydrophobic moiety (e.g., cholesterol or Vitamin E TPGS). In some embodiments, the plurality of cationic moieties of c) is from about 0.1 to about 60 weight by % in, or used as starting materials to make, the particle, e.g., from about 1 to about 60 by weight % of the particle. In some embodiments, the ratio of the charge of the plurality of cationic moieties to the charge from the plurality of nucleic acid agents is from about 1:1 to about 50:1 (e.g., 1:1 to about 10:1 or 1:1 to 5:1, about 1.5:1 or about 1:1). In embodiments where the cationic moiety is a nitrogen containing moiety this ratio can be referred to as the N/P ratio.

Additional properties of the particles described herein include the following. In some embodiments, at least a portion of the nucleic acid agents are DNA agents. In some embodiments, at least a portion of the nucleic acid agents are RNA agents (e.g., siRNA or microRNA or an agent that promotes RNAi). In some embodiments, at least a portion of the nucleic acid agents are selected from the group consisting of siRNA, an antisense oligonucleotide, a microRNA (miRNA), shRNA, an antagomir, an aptamer, genomic DNA, cDNA, mRNA, and a plasmid. In some embodiments, at least a portion of the plurality of nucleic acid agents are chemically modified (e.g., include one or more backbone modifications, base modifications, and or modifications to the sugar) to increase the stability of the nucleic acid agent. In some embodiments, the plurality of nucleic acid agents are from about 1 to about 50 weight % in, or used as starting materials to make, the particle (e.g., from about 1 to about 20%, from about 2 to about 15%, from about 3 to about 12%).

Additional properties of the particles described herein include the following. In some embodiments, the particle also includes a surfactant. In some embodiments, the surfactant is a polymer such as PVA. In some embodiments, the PVA has a viscosity of from about 2 to about 27 cP. In some embodiments, the surfactant is from about 0 to about 40 weight % in, or used as starting materials to make, the particle (e.g., from about 15 to about 35 weight %). In some embodiments, the diameter of the particle is less than about 200 nm (e.g., from about 200 to about 20 nm, from about 150 to about 50 nm, or less than about 150 nm). In some embodiments, the surface of the particle is substantially coated with PEG, PVA, polyoxazoline, polyvinylpyrrolidine, polyhydroxylpropylmethacrylamide, or polysialic acid (e.g., PEG). In some embodiments, the particle comprises a targeting agent. In some embodiments, the surface of the particle is substantially free of nucleic acid agent.

Additional properties of the particles described herein include the following. In some embodiments, the plurality of nucleic acid agents of d) is substantially intact. In some embodiments, the zeta potential of the particle is from about −20 to about 50 mV (e.g., from about −20 to about 20 mV, from about −10 to about 10 mV, or neutral). In some embodiments, the particle is chemically stable under conditions, comprising a temperature of 23 degrees Celsius and 60% percent humidity for at least 1 day (e.g., at least 7 days, at least 14 days, at least 21 days, at least 30 days). In some embodiments, the particle is a lyophilized particle. In some embodiments, the particle is formulated into a pharmaceutical composition. In some embodiments, the surface of the particle is substantially free of a targeting agent.

In some embodiments, the particles described herein can deliver an effective amount of the nucleic acid agent such that expression of the targeted gene in the subject is reduced by at least 20%, 25%, 30%, 35%, 40%, 45%, 50%, 55%, 60%, 65%, 70%, 75%, 80%, 85%, 90%, 95% or more at approximately 72 hours, 96 hours, 120 hours, 144 hours, 168 hours, 192 hours, 216 hours, 240 hours, 264 hours after administration of the particles to the subject. In one embodiment, the particles described herein can deliver an effective amount of the nucleic acid agent such that expression of the targeted gene in the subject is reduced by at least 50%, 55%, 60%, 65%, 70%, 75% or 80%, approximately 120 hours after administration of the particles to the subject. In some embodiments, the level of target gene expression in a subject administered a particle or composition described herein is compared to the level of expression of the target gene seen when the nucleic acid agent is administered in a formulation other than a particle or a conjugate (i.e., not in a particle, e.g., not embedded in a particle or conjugated to a polymer, for example, a particle deascibed herein) or than expression of the target gene seen in the absence of the administration of the nucleic acid agent or other therapeutic agent).

In some embodiments, the particle includes a hydrophobic polymer, e.g., wherein a nucleic acid agent is attached to a hydrophobic polymer of a) and wherein the hydrophobic polymer, or nucleic acid agent-hydrophobic polymer conjugate, has one or more of the following properties:

i) the hydrophobic polymer attached to the nucleic acid agent can be a homopolymer or a polymer made up of more than one kind of monomeric subunit;

ii) the hydrophobic polymer attached to the nucleic acid agent has a weight average molecular weight of from about 4 to about 20 kDa;

iii) the hydrophobic polymer is made up of a first and a second type of monomeric subunit, and the ratio of the first to second type of monomeric subunit in the hydrophobic polymer attached to the agent is from about 25:75 to about 75:25, e.g., about 50:50;

iv) the hydrophobic polymer is PLGA;

v) the nucleic acid agent is about 1 to about 20 weight % of the particle;

vi) the plurality of nucleic acid agent-hydrophobic polymer conjugates is about 10 weight % of the particle.

In some embodiments, hydrophobic polymer attached to the nucleic acid agent has a weight average molecular weight of from about 4 to about 12 kDa, e.g., from about 6 to about 12 kDa or from about 8 to about 12 kDa.

In some embodiments, the hydrophilic-hydrophobic polymers of b) have one or more of the following properties:

i) the hydrophilic portion has a weight average molecular weight of from about 1 to about 6 kDa (e.g., from about 2 to about 6 kDa),

ii) the hydrophobic polymer has a weight average molecular weight of from about 4 to about 15 kDa;

iii) the plurality of hydrophilic-hydrophobic polymers is about 25 weight % of the particle;

iv) the hydrophilic polymer is PEG;

v) the hydrophobic polymer is made up of a first and a second type of monomeric subunit, and the ratio of the first to second type of monomeric subunit in the hydrophobic polymer attached to the agent is from about 25:75 to about 75:25, e.g., about 50:50; and

vi) the hydrophobic polymer is PLGA.

In some embodiments, if the weight average molecular weight of the hydrophilic portion is from about 1 to about 3 kDa, e.g., about 2 kDa, the ratio of the weight average molecular weight of the hydrophilic portion to the weight average molecular weight of the hydrophobic portion is between 1:3-1:7, and if the weight average molecular weight of the hydrophilic portion is from about 4 to about 6 kDa, e.g., about 5 kDa, the ratio of the weight average molecular weight of the hydrophilic portion to the weight average molecular weight of the hydrophobic portion is between 1:1-1:4.

In some embodiments, the hydrophilic portion has a weight average molecular weight of from about 2 to about 6 kDa and the hydrophobic portion has a weight average molecular weight of from about 8 to about 13 kDa. In some embodiments, the hydrophilic portion of the hydrophilic-hydrophobic polymer terminates in a methoxy.

In some embodiments, a nucleic acid agent is attached to a hydrophobic polymer of and wherein the nucleic acid agent-hydrophobic polymer conjugate has one or more of the following properties:

i) the hydrophobic polymer attached to the nucleic acid agent can be a homopolymer or a polymer made up of more than one kind of monomeric subunit;

ii) the hydrophobic polymer attached to the nucleic acid agent has a weight average molecular weight of from about 4 to about 15 kDa;

iii) the hydrophobic polymer is made up of a first and a second type of monomeric subunit, and the ratio of the first to second type of monomeric subunit in the hydrophobic polymer attached to the agent is from about 25:75 to about 75:25, e.g., about 50:50;

iv) the hydrophobic polymer is PLGA;

v) the charge ratio of cationic moiety to nucleic acid agent is about 1:1 to about 4:1;

vi) the plurality of nucleic acid agent-hydrophobic polymer conjugates is about 10 weight % of the particle. In some embodiments, the particle also includes a surfactant (e.g. PVA).

In another aspect, the invention features a composition comprising a plurality of particles described herein. In some embodiments, the composition is a pharmaceutical composition.

In some embodiments, at least 50%, 60%. 70%, 80%, 90%, 95%, 99% or all of the particles in the composition have a diameter of less than about 200 nm. In some embodiments, the particles have a Dᵥ90 of less than 200 nm (e.g., from about 200 to about 20 nm, from about 150 to about 50 nm, or less than about 150 nm).

In some embodiments, the composition is substantially free of polymers having a molecular weight of less than about 1 kDa (e.g., less than about 500 Da). In some embodiments, the composition is substantially free of free nucleic acid agents (i.e., nucleic acid agent that is not embedded in or attached to the particles). In some embodiments, the composition further comprises a targeting agent. In some embodiments, the composition is substantially free of cationic moieties (i.e., cationic moieties that are not embedded in or attached to a component in the particles).

In some embodiments, the composition is chemically stable under conditions, comprising a temperature of 23 degrees Celsius and 60% percent humidity for at least 1 day (e.g., at least 7 days, at least 14 days, at least 21 days, at least 30 days). In some embodiments, the composition is a lyophilized composition.

In some embodiments, the particle is formulated into a pharmaceutical composition.

In another aspect, the invention features a kit comprising a plurality of particles described herein or a composition described herein.

In another aspect, the invention features a single dosage unit comprising a plurality of particles described herein or a composition described herein.

In another aspect, the invention features a method of treating a subject having a disorder comprising administering to the subject an effective amount of particles described herein or a composition described herein, to thereby treat a subject.

In one embodiment, the disorder is a proliferative disorder, e.g., a slow-growing proliferative disorder. In one embodiment, the proliferative disorder is cancer, e.g., a cancer described herein. In one embodiment, the cancer is a slow-growing cancer, e.g., a solid tumor or leukemia. For example, the slow-growing cancer can be a stage I or stage II solid tumor. Exemplary cancers include, but are not limited to, a cancer of the bladder (including accelerated and metastatic bladder cancer), breast (e.g., estrogen receptor positive breast cancer; estrogen receptor negative breast cancer; HER-2 positive breast cancer; HER-2 negative breast cancer; progesterone receptor positive breast cancer; progesterone receptor negative breast cancer; estrogen receptor negative, HER-2 negative and progesterone receptor negative breast cancer (i.e., triple negative breast cancer); inflammatory breast cancer), colon (including colorectal cancer), kidney, liver, lung (including small and non-small cell lung cancer, lung adenocarcinoma and squamous cell cancer), genitourinary tract, e.g., ovary (including fallopian tube and peritoneal cancers), cervix, prostate and testes, lymphatic system, rectum, larynx, pancreas (including exocrine pancreatic carcinoma), esophagus, stomach, gall bladder, thyroid, skin (including squamous cell carcinoma), brain (including glioblastoma multiforme), and head and neck. Preferred cancers include breast cancer (e.g., metastatic or locally advanced breast cancer), prostate cancer (e.g., hormone refractory prostate cancer), renal cell carcinoma, lung cancer (e.g., non-small cell lung cancer, small cell lung cancer, lung adenocarcinoma, and squamous cell cancer, e.g., advanced non-small cell lung cancer, small cell lung cancer, lung adenocarcinoma, and squamous cell cancer), pancreatic cancer, gastric cancer (e.g., metastatic gastric adenocarcinoma), colorectal cancer, rectal cancer, squamous cell cancer of the head and neck, lymphoma (Hodgkin's lymphoma or non-Hodgkin's lymphoma), renal cell carcinoma, carcinoma of the urothelium, soft tissue sarcoma, gliomas, melanoma (e.g., advanced or metastatic melanoma), germ cell tumors, ovarian cancer (e.g., advanced ovarian cancer, e.g., advanced fallopian tube or peritoneal cancer) and gastrointestinal cancer.

In another aspect, the invention features a method of reducing target gene expression in a subject, e.g., a subject having a disorder that can be treated by reducing expression of the targeted gene. The method comprises administering an effective amount of particles described herein or a composition described herein, wherein the nucleic acid agent delivered by the particle reduces expression of the targeted gene in the subject by at least 20%, 25%, 30%, 35%, 40%, 45%, 50%, 55%, 60%, 65%, 70%, 75%, 80%, 85%, 90%, 95% or more approximately 72 hours, 96 hours, 120 hours, 144 hours, 168 hours, 192 hours, 216 hours, 240 hours, 264 hours after administration of the particles. In one embodiment, the nucleic acid agent delivered by the particle reduces expression of the targeted gene in the subject by at least 50%, 55%, 60%, 65%, 70%, 75% or 80%, approximately 120 hours after administration of the particles. In some embodiments, the level of target gene expression in a subject administered a particle or composition described herein is compared to the level of expression of the target gene seen when the nucleic acid agent is administered in a formulation other than a particle or a conjugate (i.e., not in a particle, e.g., not embedded in a particle or conjugated to a polymer, for example, a particle deascibed herein) or than expression of the target gene seen in the absence of the administration of the nucleic acid agent or other therapeutic agent).

In another aspect, the invention features a nucleic acid agent-hydrophobic polymer conjugate comprising a nucleic acid agent covalently attached to a hydrophobic polymer or a nucleic acid agent that forms a duplex (e.g., a heteroduplex) with a nucleic acid which is covalently attached to the hydrophobic polymer.

In some embodiments, the nucleic acid agent is covalently attached to the hydrophobic polymer via the 2′, 3′, and/or 5′ end of the nucleic acid agent. In some embodiments, the nucleic acid agent is covalently attached to the hydrophobic polymer at a terminal end of the polymer. In some embodiments, the nucleic acid agent is covalently attached to the polymer on the backbone of the hydrophobic polymer. In some embodiments, a single nucleic acid agent is covalently attached to a single hydrophobic polymer. In some embodiments, a plurality of nucleic acid agents are each covalently attached to a single hydrophobic polymer.

In some embodiments, the nucleic acid agent is directly covalently attached (e.g., without the presence of atoms from an intervening spacer moiety), to the hydrophobic polymer (e.g., via an ester). In some embodiments, the nucleic acid agent is covalently attached to the hydrophobic polymer via a linker. Exemplary linkers include a linker that comprises a bond formed using click chemistry (e.g., as described in WO 2006/115547) and a linker that comprises an amide, an ester, a disulfide, a sulfide, a ketal, a succinate, an oxime, a carbamate, a carbonate, a silyl ether, or a triazole (e.g., an amide, an ester, a disulfide, a sulfide, a ketal, a succinate, or a triazole). In some embodiments, the linker comprises a functional group such as a bond that is cleavable under physiological conditions. In some embodiments, the linker comprises a plurality of functional groups such as bonds that are cleavable under physiological conditions. In some embodiments, the linker includes a functional group such as a bond or functional group described herein that is not directly attached either to a first or second moiety linked through the linker at the terminal ends of the linker, but is interior to the linker. In some embodiments, the linker is hydrolysable under physiologic conditions, the linker is enzymatically cleavable under physiological conditions, or the linker comprises a disulfide which can be reduced under physiological conditions. In some embodiments, the linker is not cleaved under physiological conditions, for example, the linker is of a sufficient length such that the nucleic acid agent does not need to be cleaved to be active, e.g., the length of the linker is at least about 20 angstroms (e.g., at least about 24 angstroms).

In some embodiments, the hydrophobic polymer has a terminal hydroxyl moiety. In some embodiments, the hydrophobic polymer has a terminal hydroxyl moiety is capped (e.g., with an acyl moiety).

In some embodiments, the hydrophobic polymer has one or more of the following properties:

i) the hydrophobic polymer attached to the nucleic acid agent is a homopolymer or a polymer made up of more than one kind of monomeric subunit;

ii) the hydrophobic polymer attached to the nucleic acid agent has a weight average molecular weight of from about 4 to about 15 kDa (e.g., from about 4 to about 12 kDa, from about 6 to about 12 kDa, or from about 8 to about 12 kDa);

iii) the hydrophobic polymer is made up of a first and a second type of monomeric subunit, and the ratio of the first to second type of monomeric subunit in the hydrophobic polymer attached to the agent is from about 25:75 to about 75:25, e.g., about 50:50; and

iv) the hydrophobic polymer is PLGA.

In an embodiment the nucleic acid agent is an RNA, a DNA or a mixed polymer of RNA and DNA. In an embodiment an RNA is an mRNA or a siRNA. In an embodiment a DNA is a cDNA or genomic DNA. In an embodiment the nucleic acid agent is single stranded and in another embodiment it comprises two strands. In an embodiment the nucleic acid agent can have a duplexed region, comprised of strands from one or two molecules. In an embodiment the nucleic acid agent is an agent that inhibits gene expression, e.g., an agent that promotes RNAi. In some embodiments, the nucleic acid agent is selected from the group consisting of siRNA, shRNA, an antisense oligonucleotide, or a microRNA (miRNA). In an embodiment the nucleic acid agent is an antagomir or an aptamer.

In another aspect, the invention features a composition comprising a plurality of nucleic acid agent-hydrophobic polymer conjugates described herein. In some embodiments, the composition is a pharmaceutical composition. In some embodiments, the composition is a reaction mixture. In some embodiments, the composition is substantially free of un-conjugated nucleic acid agent. In some embodiments, at least about 50% of the nucleic acid agents on the nucleic acid agent-polymer conjugates are intact.

In some embodiments, the composition is substantially free of hydrophobic polymer having a molecular weight of less than about 1 kDa (e.g., less than about 500 Da).

In another aspect, the invention features a method of making a nucleic acid agent-hydrophobic polymer conjugate, the method comprising:

providing a nucleic acid agent and a polymer; and

subjecting the nucleic acid agent and polymer to conditions that effect the covalent attachment of the nucleic acid agent to the polymer.

In some embodiments, the method is performed in a reaction mixture. In some embodiments, the reaction mixture comprises a single solvent. In some embodiments, the reaction mixture comprises a solvent system comprising a plurality of solvents. In some embodiments, the plurality of solvents is miscible. In some embodiments, the solvent system comprises water and a polar solvent such as a solvent described herein (e.g., DMF, DMSO, acetone, benzyl alcohol, dioxane, tetrahydrofuran, or acetonitrile). In some embodiments, the solvent system comprises an aqueous buffer (e.g., phosphate buffer solution (PBS),4-(2-hydroxyethyl)-1-piperazineethanesulfonice acid (HEPES), TE buffer, or 2-(N-morpholino)ethanesulfonic acid buffer (MES)). In some embodiments, the solvent system is bi-phasic (e.g., comprises an organic and aqueous phase).

In some embodiments, at least one of the nucleic acid agent or polymer is attached to an insoluble substrate. In some embodiments, the polymer is attached to an insoluble substrate.

In some embodiments, the method results in the formation of a bond formed using click chemistry (e.g., as described in WO 2006/115547). In some embodiments, the method results in the formation of an amide, a disulfide, a sulfide, an ester, a ketal, a succinate, oxime, carbonate, carbamate, silyl ether, and/or a triazole.

In some embodiments, the hydrophobic polymer has an aqueous solubility of less than about 1 mg/ml.

In some embodiments, the nucleic acid agent is covalently attached to the hydrophobic polymer via the 2′, 3′, and/or 5′ end of the nucleic acid agent. In some embodiments, the nucleic acid agent is covalently attached to the polymer at a terminal end of the hydrophobic polymer. In some embodiments, the hydrophobic polymer has a hydroxyl and/or a carboxylic acid terminal end. In some embodiments, the nucleic acid agent is covalently attached to the polymer on the backbone of the hydrophobic polymer. In some embodiments, a single nucleic acid agent is covalently attached to a single hydrophobic polymer. In some embodiments, a plurality of nucleic acid agents are each covalently attached to a single hydrophobic polymer.

In some embodiments, the method results in a nucleic acid agent-hydrophobic polymer conjugate having a purity of at least about 80% (e.g., at least about 85%, at least about 90%, at least about 95%, at least about 99%). In some embodiments, the method produces at least about 100 mg of the nucleic acid agent-hydrophobic polymer conjugate (e.g., at least about 1 g).

In another aspect, the invention features a nucleic acid agent-hydrophobic polymer conjugate made by a method described herein.

In another aspect, the invention features, a nucleic acid agent-hydrophilic-hydrophobic polymer conjugate comprising a nucleic acid agent covalently attached to a hydrophilic-hydrophobic polymer or a nucleic acid agent that forms a duplex (e.g., a heteroduplex) with a nucleic acid which is covalently attached to a hydrophilic-hydrophobic polymer, wherein the hydrophilic-hydrophobic polymer comprises a hydrophilic portion attached to a hydrophobic portion.

In some embodiments, the nucleic acid agent is attached to the hydrophilic portion of the hydrophilic-hydrophobic polymer. In some embodiments, the nucleic acid agent is attached to the hydrophobic portion of the hydrophilic-hydrophobic polymer. In some embodiments, the nucleic acid agent is covalently attached to the hydrophilic-hydrophobic polymer via the 2′, 3′, and/or 5′ end of the nucleic acid agent. In some embodiments, the nucleic acid agent is covalently attached to the hydrophilic-hydrophobic polymer at a terminal end of the polymer. In some embodiments, the nucleic acid agent is covalently attached to the polymer on the backbone of the hydrophilic-hydrophobic polymer. In some embodiments, a single nucleic acid agent is covalently attached to a single hydrophilic-hydrophobic polymer. In some embodiments, a plurality of nucleic acid agents are each covalently attached to a single hydrophilic-hydrophobic polymer.

In some embodiments, the nucleic acid agent is directly covalently attached (e.g., without the presence of atoms from an intervening spacer moiety), to the hydrophobic portion of the hydrophobic-hydrophobic polymer (e.g., via an ester). In some embodiments, the nucleic acid agent is directly covalently attached (e.g., without the presence of atoms from an intervening spacer moiety), to the hydrophilic portion of the hydrophilic-hydrophobic polymer (e.g., via an ester). In some embodiments, the nucleic acid agent is attached to the hydrophilic-hydrophobic polymer via a linker (e.g., the hydrophilic portion of the polymer or the hydrophobic portion of the polymer).

Exemplary linkers include a linker that comprises a bond formed using click chemistry (e.g., as described in WO 2006/115547) and a linker that comprises an amide, an ester, a disulfide, a sulfide, a ketal, a succinate, an oxime, a carbamate, a carbonate, a silyl ether, or a triazole (e.g., an amide, an ester, a disulfide, a sulfide, a ketal, a succinate, or a triazole). In some embodiments, the linker comprises a functional group such as a bond that is cleavable under physiological conditions. In some embodiments, the linker comprises a plurality of functional groups such as bonds that are cleavable under physiological conditions. In some embodiments, the linker includes a functional group such as a bond or functional group described herein that is not directly attached either to a first or second moiety linked through the linker at the terminal ends of the linker, but is interior to the linker. In some embodiments, the linker is hydrolysable under physiologic conditions, the linker is enzymatically cleavable under physiological conditions, or the linker comprises a disulfide which can be reduced under physiological conditions. In some embodiments, the linker is not cleaved under physiological conditions, for example, the linker is of a sufficient length that the nucleic acid agent does not need to be cleaved to be active, e.g., the length of the linker is at least about 20 angstroms (e.g., at least about 24 angstroms).

In some embodiments, the hydrophilic-hydrophobic polymers have one or more of the following properties:

i) the hydrophilic portion has a weight average molecular weight of from about 1 to about 6 kDa (e.g., from about 2 to about 6 kDa),

ii) the hydrophobic polymer has a weight average molecular weight of from about 4 to about 15 kDa (e.g., from about 4 to about 12 kDa, from about 6 to about 12 kDa, or from about 8 to about 12 kDa);

iii) the hydrophilic polymer is PEG;

iv) the hydrophobic polymer is made up of a first and a second type of monomeric subunit, and the ratio of the first to second type of monomeric subunit in the hydrophobic polymer attached to the nucleic acid agent is from about 25:75 to about 75:25, e.g., about 50:50; and

v) the hydrophobic polymer is PLGA.

In some embodiments, if the weight average molecular weight of the hydrophilic portion of the hydrophilic-hydrophobic polymer is from about 1 to about 3 kDa, e.g., about 2 kDa, the ratio of the weight average molecular weight of the hydrophilic portion to the weight average molecular weight of the hydrophobic portion is between 1:3-1:7, and if the weight average molecular weight of the hydrophilic portion is from about 4 to about 6 kDa, e.g., about 5 kDa, the ratio of the weight average molecular weight of the hydrophilic portion to the weight average molecular weight of the hydrophobic portion is between 1:1-1:4. In some embodiments, the hydrophilic portion has a weight average molecular weight of from about 2 to about 6 kDa and the hydrophobic portion has a weight average molecular weight of from about 8 to about 13 kDa.

In some embodiments, the hydrophilic portion of the hydrophilic-hydrophobic polymer terminates in a methoxy.

In an embodiment the nucleic acid agent is an RNA, a DNA or a mixed polymer of RNA and DNA. In an embodiment an RNA is an mRNA or a siRNA. In an embodiment a DNA is a cDNA or genomic DNA. In an embodiment the nucleic acid agent is single stranded and in another embodiment it comprises two strands. In an embodiment the nucleic acid agent can have a duplexed region, comprised of strands from one or two molecules. In an embodiment the nucleic acid agent is an agent that inhibits gene expression, e.g., an agent that promotes RNAi. In some embodiments, the nucleic acid agent is selected from the group consisting of siRNA, shRNA, an antisense oligonucleotide, or a microRNA (miRNA). In an embodiment the nucleic acid agent is an antagomir or an aptamer.

In another aspect, the invention features a composition comprising a plurality of nucleic acid agent-hydrophilic-hydrophobic polymer conjugates described herein.

In some embodiments, the composition is a reaction mixture. In some embodiments, the composition is a pharmaceutical composition. In some embodiments, the composition is substantially free of un-conjugated nucleic acid agent. In some embodiments, at least about 50% of the nucleic acid agent on the nucleic acid agent-polymer conjugates are intact. In some embodiments, the composition is substantially free of hydrophilic-hydrophobic polymer having a molecular weight of less than about 1 kDa.

In another aspect, the invention features a method of making a nucleic acid agent-hydrophilic-hydrophobic polymer conjugate described herein; the method including:

providing a nucleic acid agent and a polymer; and

subjecting the nucleic acid agent and polymer to conditions that effect the covalent attachment of the nucleic acid agent to the polymer.

In some embodiments, the method is performed in a reaction mixture. In some embodiments, the reaction mixture comprises a single solvent. In some embodiments, the reaction mixture comprises a solvent system comprising a plurality of solvents. In some embodiments, the plurality of solvents are miscible. In some embodiments, the solvent system comprises water and a polar solvent (e.g., DMF, DMSO, acetone, benzyl alcohol, dioxane, tetrahydrofuran, or acetonitrile). In some embodiments, the solvent system comprises an aqueous buffer (e.g., phosphate buffer solution (PBS),4-(2-hydroxyethyl)-1-piperazineethanesulfonice acid (HEPES), TE buffer, or 2-(N-morpholino)ethanesulfonic acid buffer (MES)). In some embodiments, the solvent system is bi-phasic (e.g., comprises an organic and aqueous phase).

In some embodiments, at least one of the nucleic acid agent or polymer is attached to an insoluble substrate. In some embodiments, the polymer is attached to an insoluble substrate.

In some embodiments, the method comprises forming a bond through click chemistry (e.g., as described in WO 2006/115547). In some embodiments, the method results in the formation of an amide, a disulfide, a sulfide, an ester, oxime, carbonate, carbamate, silyl ether, and/or a triazole.

In some embodiments, the hydrophilic-hydrophobic polymer has an aqueous solubility of less than about 50 mg/ml.

In some embodiments, the nucleic acid agent is covalently attached to the hydrophobic-hydrophilic polymer via the 2′, 3′, and/or 5′ end of the nucleic acid agent. In some embodiments, the nucleic acid agent is covalently attached to the hydrophobic-hydrophilic polymer at a terminal end of the polymer. In some embodiments, the nucleic acid agent is covalently attached to the hydrophobic-hydrophilic polymer on the hydrophilic portion of the polymer. In some embodiments, the nucleic acid agent is covalently attached to the hydrophobic-hydrophilic polymer on the hydrophobic portion of the polymer. In some embodiments, the nucleic acid agent is covalently attached to the hydrophobic-hydrophilic polymer on the backbone of the polymer. In some embodiments, a single nucleic acid agent is covalently attached to a single hydrophobic-hydrophilic polymer (e.g., to the hydrophilic portion or the hydrophobic portion). In some embodiments, a plurality of nucleic acid agents are each covalently attached to a single hydrophobic-hydrophilic polymer.

In some embodiments, the method results in a nucleic acid agent-hydrophilic-hydrophobic polymer conjugate having a purity of at least about 80% (e.g., at least about 85%, at least about 90%, at least about 95%, at least about 99%). In some embodiments, the method produces at least about 100 mg of the nucleic acid agent-hydrophobic polymer conjugate (e.g., at least about 1 g).

In another aspect, the invention features a nucleic acid agent-hydrophilic-hydrophobic polymer conjugate made by a method described herein.

In another aspect, the invention features a particle, the particle including

a plurality of nucleic acid agent-polymer conjugates;

a plurality of cationic polymers or lipids; and

a plurality of polymers or lipids, wherein the polymers or lipids substantially surround the plurality of nucleic acid agent-polymer conjugates. In some embodiments, the particle is self-assembled.

In another aspect, the invention features a method of making a particle, the method comprising:
a) forming a particle comprising a plurality of nucleic acid agent-polymer conjugates;
b) contacting the particle with a plurality of cationic polyvalent polymers or lipids; and
c) contacting the product of b) with a plurality of polymers or lipids, wherein the a plurality of polymers or lipids substantially surround the product of b) forming the particle.

In another aspect, the invention features a method of making a particle, e.g., a nanoparticle, comprising an a nucleic acid agent, e.g., an siRNA moiety, combining, in a polar solvent (e.g., DMF, DMSO, acetone, benzyl alcohol, dioxane, tetrahydrofuran, or acetonitrile) under conditions that allow formation of a particle, e.g., by precipitation,
(a) nucleic acid agent-hydrophobic polymer conjugates, each nucleic acid agent-hydrophobic polymer conjugate comprising a nucleic acid agent, e.g., an siRNA moiety, covalently attached to a hydrophobic polymer, wherein the nucleic acid agent-hydrophobic polymer conjugates are associated with a cationic moiety,
(b) a plurality of hydrophilic-hydrophobic polymers, e.g., PEG-PLGA, and
(c) a plurality of hydrophobic polymers (not covalently attached to a nucleic acid agent)

to thereby form a particle.

In some embodiments, the combining is performed in a solvent system comprising acetone. In some embodiments, the solvent is a mixed solvent system (e.g., a combination aqueous/organic solvent system such as acetonitrile and an aqueous buffer system).

In some embodiments, the method comprises:

combining,

(i) a plurality of nucleic acid agents, each nucleic acid agent, e.g., an siRNA or other nucleic acid agent, coupled to a hydrophobic polymer and associated with a cationic moiety, in acetonitrile/TE buffer (e.g., from about 90/10 to about 50/50 wt %, e.g., from about 90/10 to about 70/30 wt %, e.g., about 80/20 wt %); with

(ii) a plurality of hydrophilic-hydrophobic polymers, e.g., PEG-PLGA, and a plurality of hydrophobic polymers (not coupled to a nucleic acid agent), in acetonitrile/TE buffer (e.g., from about 90/10 to about 50/50 wt %, e.g., from about 90/10 to about 70/30 wt %, e.g., about 80/20 wt %).

In another aspect, the invention features a reaction mixture of step a), or composition or pharmaceutical preparation thereof.

In another aspect, the invention features a reaction mixture of step (i) or composition or pharmaceutical preparation thereof.

In another aspect, the invention features a reaction mixture of step (ii) or composition or pharmaceutical preparation thereof.

In another aspect, the invention features a particle made by the process above.

In another aspect, the invention features a composition (e.g., a pharmaceutical composition) comprising a particle made by the process above.

In another aspect, the invention features a method of making a particle, e.g., a nanoparticle, which comprises a water soluble nucleic acid agent, e.g., an siRNA moiety, an hydrophobic-hydrophilic polymer and a hydrophobic polymer comprising
a) contacting, e.g., in an aqueous solvent

i) a first plurality of hydrophobic-hydrophilic polymers, e.g., PEG-PLGA, with

ii) a first plurality of hydrophobic polymers, e.g., PLGA, each having a first reactive moiety, e.g., a sulfhydryl moiety;

to form a water soluble intermediate particle;
b) contacting, e.g., in aqueous solvent the intermediate particle with a plurality of water soluble nucleic acid agent, e.g., siRNA moieties, each having a second reactive moiety, e.g., an SH moiety, under conditions which allow formation of an intermediate complex (e.g. having a diameter of less than about 100 nm), e.g., an intermediate structure comprising hydrophilic-hydrophobic polymers and hydrophobic polymers coupled to the nucleic acid agent and,
c) contacting, e.g., in a non-aqueous solvent, e.g., DMF, DMSO, acetone, benzyl alcohol, dioxane, tetrahydrofuran, or acetonitrile, the intermediate complex with a second plurality of hydrophilic-hydrophobic polymers, e.g., PEG-PLGA, and a second plurality of hydrophobic polymers, e.g., PLGA, under conditions that allow the formation of a particle,

thereby forming a particle.

In another aspect, the invention features a method of forming a particle, e.g., a nanoparticle, comprising
a) contacting, e.g., in acetonitrile/TE buffer (e.g., from about 90/10 to about 50/50 wt %, e.g., from about 90/10 to about 70/30 wt %, e.g., about 80/20 wt %)

i) a first plurality of hydrophilic-hydrophobic polymers, e.g., PEG-PLGA, with

ii) a first plurality of hydrophobic polymers, e.g., PLGA, each having a first reactive moiety, e.g., a sulfhydryl moiety;

to form an intermediate particle (e.g. having a diameter of less than about 100 nm), wherein, In some embodiments, the intermediate particle is functionally soluble in aqueous solution, e.g., by virtue of having sufficient hydrophilic portion such that it is soluble in aqueous solution;
b) contacting, e.g., in acetonitrile/TE buffer (e.g., from about 90/10 to about 50/50 wt %, e.g., from about 90/10 to about 70/30 wt %, e.g., about 80/20 wt %), the intermediate particle with a plurality of drug moieties, e.g., siRNA or other nucleic acid drug moieties, each having a second reactive moiety, e.g., an SH moiety, under conditions which allow formation of an intermediate complex, e.g., an intermediate structure comprising hydrophilic-hydrophobic polymers and hydrophobic polymers coupled to the drug moiety and,
c) contacting, e.g., in acetonitrile/TE buffer (e.g., from about 90/10 to about 50/50 wt %, e.g., from about 90/10 to about 70/30 wt %, e.g., about 80/20 wt %), the intermediate complex with a second plurality of hydrophilic-hydrophobic polymers, e.g., PEG-PLGA, and a second plurality of hydrophobic polymers, e.g., PLGA, under conditions that allow the formation of a particle,

thereby forming a particle.

In some embodiments, the diameter of the intermediate particle a) is less than 100 nm. In some embodiments, the diameter of the particle is less than 150 nm. In some embodiments, a plurality of cationic moieties covalently attached to hydrophobic polymers are added in step b).

In another aspect, the invention features a reaction mixture of step a), or composition or pharmaceutical preparation thereof.

In another aspect, the invention features a reaction mixture of step b), or composition or pharmaceutical preparation thereof.

In another aspect, the invention features a particle made by the process above.

In another aspect, the invention features a composition (e.g., a pharmaceutical composition) comprising a particle made by the process above.

In another aspect, the invention features a composition described herein (e.g., a pharmaceutical composition), which, when administered to a subject, results in a reduction in the expression of a target gene that is at least 10, 20, 50, 75, 80, 90, 100, 200, or 500%, greater than the reduction in the expression of the target gene seen with the nucleic acid agent administered in a formulation other than a particle or a conjugate (i.e., not in a particle, for example, not embedded in a particle or conjugated to a polymer, for example, in a particle described herein) to the subject or than expression of the target gene seen in the absence of the administration of the nucleic acid agent or other therapeutic agent.

In an embodiment the nucleic acid agent is an RNA, a DNA or a mixed polymer of RNA and DNA. In an embodiment an RNA is an mRNA or a siRNA. In an embodiment a DNA is a cDNA or genomic DNA. In an embodiment the nucleic acid agent is single stranded and in another embodiment it comprises two strands. In an embodiment the nucleic acid agent can have a duplexed region, comprised of strands from one or two molecules. In an embodiment the nucleic acid agent is an agent that inhibits gene expression, e.g., an agent that promotes RNAi. In some embodiments, the nucleic acid agent is selected from the group consisting of siRNA, shRNA, an antisense oligonucleotide, or a microRNA (miRNA). In an embodiment the nucleic acid agent is an antagomir or an aptamer.

In some embodiments, the reduction is a reduction compared to a control sample not treated with the composition or the free nucleic acid agent. In some embodiments, the composition and nucleic acid agent administered free are administered under similar conditions. In some embodiments, the amount of nucleic acid agent in the particle composition administered to the subject is the same, e.g., in terms of weight or number of molecules, as the amount of nucleic acid agent administered free. In some embodiments, the target gene is a fluorescent protein, e.g., GFP or RFP. In some embodiments, the target gene is a fusion gene which encodes a fusion protein which comprises a label, e.g., a fluorescent moiety, e.g., GFP or RFP. In some embodiments, the reduction is measured at 1 minute, 10 minutes, 60 minutes, 2 hours, 12 hours, 24 hours, 2 days or 7 days after, administration of a dose of the composition or free nucleic acid agent. In some embodiments, the subject is any of a mouse, rat, dog, or human. In some embodiments, the subject is a mouse, the target gene is GFP, and the GFP is expressed in HeLa cells implanted in the mouse. In some embodiments, the target gene is expressed in MDA-MB-231 GFP or MDA-MB-468 GFP cells implanted in the mouse.

In another aspect, the invention features a composition described herein (e.g., a pharmaceutical composition), which, when contacted with cultured cells, results in: a reduction in the expression of a target gene that is at least 10, 20, 25, 30, 40, 50, 60, 60, 80, 90, 100, 200, 300, 400 or 500% greater than the reduction seen for the nucleic acid agent (which can be a DNA agent, an RNA agent, e.g., an agent that promotes RNAi or a microRNA, an siRNA, an shRNA, an antisense oligonucleotide, an antagomir, an aptamer, genomic DNA, cDNA, mRNA, or a plasmid) administered free to the subject.

In some embodiments, the reduction is a reduction compared to a control sample not treated with the composition or the free nucleic acid agent. In some embodiments, the composition and nucleic acid agent administered free are contacted with the cells under similar conditions. In some embodiments, the amount of nucleic acid agent in the particle composition contacted with the cultured cells is the same, e.g., in terms of weight or number of molecules, as the amount contacted free. In some embodiments, the target gene is a fluorescent protein, e.g., GFP or RFP. In some embodiments, the target gene is a fusion gene which encodes a fusion protein which comprises a label, e.g., a fluorescent moiety, e.g., GFP or RFP. In some embodiments, the reduction is measured 10 minutes, 60 minutes, 2 hours, 12 hours, 24 hours, 2 days or 7 days after, contact with the cultured cells. In some embodiments, the cultured cells are HeLa cells. In some embodiments, the cultured cells are MDA-MB-231 GFP or MDA-MB-468 GFP cells. In some embodiments, the target gene is GFP and the reduction in target gene expression is determined by contacting an aliquot of the composition and with cultured HeLA cells transfected with GFP, contacting an aliquot of the free nucleic acid agent with cultured HeLA cells transfected with GFP, and evaluating the level of GFP activity in each.

In another aspect, the invention features a composition described herein (e.g., a pharmaceutical composition), which, when incubated in serum, or cell lysate, and then contacted with cultured cells, retains at least 10, 20, 25, 30, 40, 50, 60, 60, 80, 90, or 100% of the ability of a control composition of the particles, e.g., one that has not been incubated with serum or cell lysate, e.g., has been incubated under otherwise similar conditions in a buffer of physiological pH, to reduce the expression of a target gene when contacted with cultured cells.

In some embodiments, the reduction is a reduction compared to a control sample not treated with the composition or the free nucleic acid agent. In some embodiments, incubation in serum or cell lysate is for 10 minutes, 20 minutes, 30 minutes, 1 hour, 2 hours, 5 hours, 24 hours, 2 days, 3, days, 5 days, or 10 days. In some embodiments, the target gene is a fluorescent protein, e.g., GFP or RFP. In some embodiments, the target gene is a fusion gene which encodes a fusion protein which comprises a label, e.g., a fluorescent moiety, e.g., GFP or RFP. In some embodiments, the target gene is GFP and the reduction in target gene expression is determined by contacting an aliquot of the composition and with cultured HeLA cells transfected with GFP, contacting an aliquot of the free nucleic acid agent with cultured HeLA cells transfected with GFP, and evaluating the level of GFP activity in each. In some embodiments, the composition and nucleic acid agent (which can be a DNA agent, an RNA agent, e.g., an agent that promotes RNAi, a microRNA, an siRNA, an shRNA, an antisense oligonucleotide, an antagomir, an aptamer, genomic DNA, cDNA, mRNA, or a plasmid) administered free are contacted with the cells under similar conditions. In some embodiments, the amount of nucleic acid agent in the particle composition contacted with the cultured cells is the same, e.g., in terms of weight or number of molecules, as the amount contacted free. In some embodiments, the cultured cells are HeLa cells. In some embodiments, the cultured cells are MDA-MB-231 GFP or MDA-MB-468 GFP cells.

In another aspect, the invention features a composition described herein (e.g., a pharmaceutical composition), which, when incubated in serum and then contacted with cultured cells, has at least one of the following properties:

a) retains at least 10, 20, 25, 30, 40, 50, 60, 60, 80, 90, or 100% of the ability of a control composition of the particles, e.g., one that has not been incubated with serum, e.g., has been incubated under otherwise similar conditions in a buffer of physiological pH, to reduce the expression of a target gene when contacted with cultured cells; or

b) retains at least 10, 20, 25, 30, 40, 50, 60, 60, 80, 90, or 100% of the ability of a control composition of the particles, e.g., one that has not been incubated with serum, e.g., has been incubated under otherwise similar conditions in a buffer of physiological pH, to release intact nucleic acid agent.

In some embodiments, incubation in serum is for 10 minutes, 20 minutes, 30 minutes, 1 hour, 2 hours, 5 hours, 24 hours, 2 days, 3, days, 5, days or 10 days. In some embodiments, the composition and nucleic acid agent administered in a formulation other than a particle or a conjugate (i.e., not in a particle, for example, not embedded in a particle or conjugated to a polymer in a particle described herein) are contacted with the cells under similar conditions. In some embodiments, the amount of nucleic acid agent in the particle composition contacted with the cultured cells is the same, e.g., in terms of weight or number of molecules, as the amount contacted free. In an embodiment the nucleic acid agent is an RNA, a DNA or a mixed polymer of RNA and DNA. In an embodiment an RNA is an mRNA or a siRNA. In an embodiment a DNA is a cDNA or genomic DNA. In an embodiment the nucleic acid agent is single stranded and in another embodiment it comprises two strands. In an embodiment the nucleic acid agent can have a duplexed region, comprised of strands from one or two molecules. In an embodiment the nucleic acid agent is an agent that inhibits gene expression, e.g., an agent that promotes RNAi. In some embodiments, the nucleic acid agent is selected from the group consisting of siRNA, shRNA, an antisense oligonucleotide, or a microRNA (miRNA). In an embodiment the nucleic acid agent is an antagomir or an aptamer.

In another aspect, the invention features, a method of storing a conjugate, particle or composition, the method comprising:

providing said conjugate, particle or composition disposed in a container, e.g., an air or liquid tight container, e.g., a container described herein, e.g., a container having an inert gas, e.g., argon or nitrogen, filled headspace;

storing said conjugate, particle or composition, e.g., under preselected conditions, e.g., temperature, e.g., a temperature described herein;

and, moving said container to a second location or removing all or an aliquot of said conjugate, particle or composition, from said container.

In an embodiment the conjugate, particle or composition is evaluated, e.g., for stability or activity of the nucleic acid agent, a physical property, e.g., color, clumping, ability to flow or be poured, or particle size or charge. The evaluation can be compared to a standard, and optionally, responsive to said standard, the conjugate, particle or composition, is classified.

In an embodiment, a conjugate, particle or composition is stored as a re-constituted formulation (e.g., in a liquid as a solution or suspension).

### BRIEF DESCRIPTION OF THE FIGURES

FIGS. 1A-C describe exemplary linkers which may be used to attach moieties described herein.

FIG. 2 is a gel showing the results of a digestion assay wherein particles containing siRNA embedded (non-conjugated) therein were treated with RNAse.

FIG. 3 is a gel showing the results of a digestion assay wherein particles containing siRNA conjugated to a polymer were treated with RNAse.

FIG. 4 is a gel showing the specific cleavage of target (EGFP) mRNA in human breast tumor cells engineered to express EGFP, in xeno-mice, when the xeno-mice were treated in vivo with siEGFP particles. The gel shows the level of cleavage-specific amplification products generated by 5′ RLM RACE-PCR in RNA extracts of tumor from treated xeno-mice.

FIG. 5 shows C3a and Bb concentrations in human whole blood samples exposed to particles prepared according to Example 61a and Example 32a.

### DETAILED DESCRIPTION

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Particles, conjugates (e.g., nucleic acid agent-polymer conjugates), and compositions are described herein. Also disclosed are dosage forms containing the conjugates, particles and compositions; methods of using the conjugates, particles and compositions (e.g., to treat a disorder); kits including the conjugates, particles and compositions; methods of making the conjugates, particles and compositions; methods of storing the conjugates, particles and compositions; and methods of analyzing the particles and compositions comprising the particles.

Headings, and other identifiers, e.g., (a), (b), (i) etc, are presented merely for ease of reading the specification and claims. The use of headings or other identifiers in the specification or claims does not require the steps or elements be performed in alphabetical or numerical order or the order in which they are presented.

### DEFINITIONS

The term “ambient conditions,” as used herein, refers to surrounding conditions at about one atmosphere of pressure, 50% relative humidity and about 25° C., unless specified as otherwise.

The term “attach,” as used herein with respect to the relationship of a first moiety to a second moiety, e.g., the attachment of an agent to a polymer, refers to the formation of a covalent bond between a first moiety and a second moiety. In the same context, the noun “attachment” refers to a covalent bond between the first and second moiety. For example, a nucleic acid agent agent attached to a polymer is a therapeutic agent, in this case a nucleic acid agent, covalently bonded to the polymer (e.g., a hydrophobic polymer described herein). The attachment can be a direct attachment, e.g., through a direct bond of the first moiety to the second moiety, or can be through a linker (e.g., through a covalently linked chain of one or more atoms disposed between the first and second moiety). For example, where an attachment is through a linker, a first moiety (e.g., a drug) is covalently bonded to a linker, which in turn is covalently bonded to a second moiety (e.g., a hydrophobic polymer described herein).

The term “biodegradable” includes polymers, compositions and formulations, such as those described herein, that are intended to degrade during use. Biodegradable polymers typically differ from non-biodegradable polymers in that the former may be degraded during use. In certain embodiments, such use involves in vivo use, such as in vivo therapy, and in other certain embodiments, such use involves in vitro use. In general, degradation attributable to biodegradability involves the degradation of a biodegradable polymer into its component subunits, or digestion, e.g., by a biochemical process, of the polymer into smaller, non-polymeric subunits. In certain embodiments, two different types of biodegradation may generally be identified. For example, one type of biodegradation may involve cleavage of bonds (whether covalent or otherwise) in the polymer backbone. In such biodegradation, monomers and oligomers typically result, and even more typically, such biodegradation occurs by cleavage of a bond connecting one or more of subunits of a polymer. In contrast, another type of biodegradation may involve cleavage of a bond (whether covalent or otherwise) internal to a side chain or that connects a side chain to the polymer backbone. In certain embodiments, one or the other or both general types of biodegradation may occur during use of a polymer.

The term “biodegradation,” as used herein, encompasses both general types of biodegradation described above. The degradation rate of a biodegradable polymer often depends in part on a variety of factors, including the chemical identity of the linkage responsible for any degradation, the molecular weight, crystallinity, biostability, and degree of cross-linking of such polymer, the physical characteristics (e.g., shape and size) of a polymer, assembly of polymers or particle, and the mode and location of administration. For example, a greater molecular weight, a higher degree of crystallinity, and/or a greater biostability, usually lead to slower biodegradation.

The term “cationic moiety” refers to a moiety, which has a pKa 5 or greater (e.g., a lewis base having a pKa of 5 or greater) and/or a positive charge in at least one of the following conditions: during the production of a particle described herein, when formulated into a particle described herein, or subsequent to administration of a particle described herein to a subject, for example, while circulating in the subject and/or while in the endosome. Exemplary cationic moieties include amine containing moieties (e.g., charged amine moieties such as a quaternary amine), guanidine containing moieties (e.g., a charged guanidine such as a quanadinium moiety), and heterocyclic and/or heteroaromatic moieties (e.g., charged moieties such as a pyridinium or a histidine moiety). Cationic moieties include polymeric species, such as moieties having more than one charge, e.g., contributed by repeated presence of a moiety, (e.g., a cationic PVA and/or a polyamine). Cationic moieties also include zwitterions, meaning a compound that has both a positive charge and a negative charge (e.g., an amino acid such as arginine, lysine, or histidine).

The term “cationic polymer,” for example, a polyamine, refers to a polymer (the term polymer is described herein below) that has a plurality of positive charges (i.e., at least 2) when formulated into a particle described herein. In some embodiments, the cationic polymer, for example, a polyamine, has at least 3, 4, 5, 10, 15, or 20 positive charges.

The phrase “cleavable under physiological conditions” refers to a bond having a half life of less than about 50 or 100 hours, when subjected to physiological conditions. For example, enzymatic degradation can occur over a period of less than about five years, one year, six months, three months, one month, fifteen days, five days, three days, or one day upon exposure to physiological conditions (e.g., an aqueous solution having a pH from about 4 to about 8, and a temperature from about 25° C. to about 37° C.

An “effective amount” or “an amount effective” refers to an amount of the polymer-agent conjugate, particle, or composition which is effective, upon single or multiple dose administrations to a subject, in treating a cell, or curing, alleviating, relieving or improving a symptom of a disorder. An effective amount of the composition may vary according to factors such as the disease state, age, sex, and weight of the individual, and the ability of the compound to elicit a desired response in the individual. An effective amount is also one in which any toxic or detrimental effects of the composition are outweighed by the therapeutically beneficial effects.

The term “embed” as used herein, refers to disposing a first moiety with, or within, a second moiety by the formation of a non-covalent interaction between the first moiety and a second moiety, e.g., a nucleic acid agent or a cationic moiety and a polymer. In some embodiments, when referring to a moiety embedded in a particle, that moiety (e.g., a nucleic acid agent or a cationic moiety) is associated with a polymer or other component of the particle through one or more non-covalent interactions such as van der Waals interactions, hydrophobic interactions, hydrogen bonding, dipole-dipole interactions, ionic interactions, and pi-stacking, and covalent bonds between the moieties and polymer or other components of the particle are absent. An embedded moiety may be completely or partially surrounded by the polymer or particle in which it is embedded.

The term “hydrophobic,” as used herein, describes a moiety that can be dissolved in an aqueous solution at physiological ionic strength only to the extent of less than about 0.05 mg/mL (e.g., about 0.01 mg/mL or less).

The term “hydrophilic,” as used herein, describes a moiety that has a solubility, in aqueous solution at physiological ionic strength, of at least about 0.05 mg/mL or greater.

The term “hydrophilic-hydrophobic polymer” as used herein, describes a polymer comprising a hydrophilic portion attached to a hydrophobic portion. Exemplary hydrophilic-hydrophobic polymers include block-copolymers, e.g., of hydrophilic and hydrophobic polymers.

A “hydroxy protecting group” as used herein, is well known in the art and includes those described in detail in Protecting Groups in Organic Synthesis, T. W. Greene and P. G. M. Wuts, 3^{rd} edition, John Wiley & Sons, 1999, the entirety of which is incorporated herein by reference. Suitable hydroxy protecting groups include, for example, acyl (e.g., acetyl), triethylsilyl (TES), t-butyldimethylsilyl (TBDMS), 2,2,2-trichloroethoxycarbonyl (Troc), and carbobenzyloxy (Cbz).

The term “intact,” as used herein to describe a nucleic acid agent, means that the nucleic acid agent retains a sufficient amount of structure required to effectively silence its target gene. A target gene is “effectively silenced” if its expression is decreased by at least 90%, 80%, 70%, 60%, 50%, 40%, 30%, 20% or at least 10% when contacted with the intact nucleic acid agent. Typically, in an intact preparation of nucleic acid agents, e.g., siRNA, at least 60%, 70%, 80%, 90%, or all of the nucleic acid agent molecules have the same molecular weight or length of an intact nucleic acid agent molecule.

“Inert atmosphere,” as used herein, refers to an atmosphere composed primarily of an inert gas, which does not chemically react with the polymer-agent conjugates, particles, compositions or mixtures described herein. Examples of inert gases are nitrogen (N₂), helium, and argon.

“Linker,” as used herein, is a moiety that connects two or more moieties together (e.g., a nucleic acid agent or cationic moiety and a polymer such as a hydrophobic or hydrophilic-hydrophobic, or hydrophilic polymer). Linkers have at least two functional groups. For example, a linker having two functional groups may have a first functional group capable of reacting with a functional group on a moiety such as a nucleic acid agent, a cationic moiety, a hydrophobic moiety such as a polymer, or a hydrophilic-hydrophobic polymer described herein, and a second functional group capable of reacting with a functional group on a second moiety such as a nucleic acid agent described herein.

A linker may have more than two functional groups (e.g., 3, 4, 5, 6, 7, 8, 9, 10 or more functional groups), which may be used, e.g., to link multiple agents to a polymer or to provide a biocleavable moiety within the linker. In some embodiments, for example, when a linker has more than two functional groups, e.g., and the linker comprises a functional group in addition to the two functional groups connecting a first moiety to a second moiety, the additional functional group (e.g., a third functional group) can be positioned in between the first and second group, and in some embodiments, can be cleaved, for example, under physiological conditions. For example, a linker may be of the form

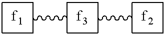

wherein f₁ is a first functional group, e.g., a functional group capable of reacting with a functional group on a moiety such as a nucleic acid agent, a cationic moiety, a hydrophobic moiety such as a polymer, or a hydrophilic-hydrophobic polymer described herein; f₂ is a second functional group, e.g., a functional group capable of reacting with a functional group on a second moiety such as a nucleic acid agent described herein; f₃ is a biocleavable functional group, e.g., a biocleaveable bond described herein; and

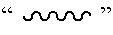

represents a spacer connecting the functional groups, e.g., an alkylene (divalent alkyl) group wherein, optionally, one or more carbon atoms of the alkylene linker is replaced with one or more heteroatoms (e.g., resulting in one of the following groups: thioether, amino, ester, ether, keto, amide, silyl ether, oxime, carbamate, carbonate, disulfide, heterocyclic, or heteroaromatic). Depending on the context, linker can refer to a linker moiety before attachment to either of a first or second moiety (e.g., nucleic acid agent or polymer), after attachment to one moiety but before attachment to a second moiety, or the residue of the linker present after attachment to both the first and second moiety.

The term “lyoprotectant,” as used herein refers to a substance present in a lyophilized preparation. Typically it is present prior to the lyophilization process and persists in the resulting lyophilized preparation. Typically a lyoprotectant is added after the formation of the particles. If a concentration step is present, e.g., between formation of the particles and lyophilization, a lyoprotectant can be added before or after the concentration step. A lyoprotectant can be used to protect particles, during lyophilization, for example to reduce or prevent aggregation, particle collapse and/or other types of damage. In an embodiment the lyoprotectant is a cryoprotectant.

In an embodiment the lyoprotectant is a carbohydrate. The term “carbohydrate,” as used herein refers to and encompasses monosaccharides, disaccharides, oligosaccharides and polysaccharides.

In an embodiment, the lyoprotectant is a monosaccharide. The term “monosaccharide,” as used herein refers to a single carbohydrate unit (e.g., a simple sugar) that can not be hydrolyzed to simpler carbohydrate units. Exemplary monosaccharide lyoprotectants include glucose, fructose, galactose, xylose, ribose and the like.

In an embodiment, the lyoprotectant is a disaccharide. The term “disaccharide,” as used herein refers to a compound or a chemical moiety formed by 2 monosaccharide units that are bonded together through a glycosidic linkage, for example through 1-4 linkages or 1-6 linkages. A disaccharide may be hydrolyzed into two monosaccharides. Exemplary disaccharide lyoprotectants include sucrose, trehalose, lactose, maltose and the like.

In an embodiment, the lyoprotectant is an oligosaccharide. The term “oligosaccharide,” as used herein refers to a compound or a chemical moiety formed by 3 to about 15, preferably 3 to about 10 monosaccharide units that are bonded together through glycosidic linkages, for example through 1-4 linkages or 1-6 linkages, to form a linear, branched or cyclic structure. Exemplary oligosaccharide lyoprotectants include cyclodextrins, raffinose, melezitose, maltotriose, stachyose acarbose, and the like. An oligosaccharide can be oxidized or reduced.

In an embodiment, the lyoprotectant is a cyclic oligosaccharide. The term “cyclic oligosaccharide,” as used herein refers to a compound or a chemical moiety formed by 3 to about 15, preferably 6, 7, 8, 9, or 10 monosaccharide units that are bonded together through glycosidic linkages, for example through 1-4 linkages or 1-6 linkages, to form a cyclic structure. Exemplary cyclic oligosaccharide lyoprotectants include cyclic oligosaccharides that are discrete compounds, such as α cyclodextrin, β cyclodextrin, or γ cyclodextrin.

Other exemplary cyclic oligosaccharide lyoprotectants include compounds which include a cyclodextrin moiety in a larger molecular structure, such as a polymer that contains a cyclic oligosaccharide moiety. A cyclic oligosaccharide can be oxidized or reduced, for example, oxidized to dicarbonyl forms. The term “cyclodextrin moiety,” as used herein refers to cyclodextrin (e.g., an α, β, or γ cyclodextrin) radical that is incorporated into, or a part of, a larger molecular structure, such as a polymer. A cyclodextrin moiety can be bonded to one or more other moieties directly, or through an optional linker. A cyclodextrin moiety can be oxidized or reduced, for example, oxidized to dicarbonyl forms.

Carbohydrate lyoprotectants, e.g., cyclic oligosaccharide lyoprotectants, can be derivatized carbohydrates. For example, in an embodiment, the lyoprotectant is a derivatized cyclic oligosaccharide, e.g., a derivatized cyclodextrin, e.g., 2 hydroxy propyl-beta cyclodextrin, e.g., partially etherified cyclodextrins (e.g., partially etherified β cyclodextrins) disclosed in U.S. Pat. No. 6,407,079, the contents of which are incorporated herein by this reference. Another example of a derivatized cyclodextrin is β-cyclodextrin sulfobutylether sodium.

An exemplary lyoprotectant is a polysaccharide. The term “polysaccharide,” as used herein refers to a compound or a chemical moiety formed by at least 16 monosaccharide units that are bonded together through glycosidic linkages, for example through 1-4 linkages or 1-6 linkages, to form a linear, branched or cyclic structure, and includes polymers that comprise polysaccharides as part of their backbone structure. In backbones, the polysaccharide can be linear or cyclic. Exemplary polysaccharide lyoprotectants include glycogen, amylase, cellulose, dextran, maltodextrin and the like.

The term “derivatized carbohydrate,” refers to an entity which differs from the subject non-derivatized carbohydrate by at least one atom. For example, instead of the —OH present on a non-derivatized carbohydrate the derivatized carbohydrate can have —OX, wherein X is other than H. Derivatives may be obtained through chemical functionalization and/or substitution or through de novo synthesis—the term “derivative” implies no process-based limitation.

The term “nanoparticle” is used herein to refer to a material structure whose size in at least any one dimension (e.g., x, y, and z Cartesian dimensions) is less than about 1 micrometer (micron), e.g., less than about 500 nm or less than about 200 nm or less than about 100 nm, and greater than about 5 nm. In embodiments the size is less than about 70 nm but greater than about 20 nm. A nanoparticle can have a variety of geometrical shapes, e.g., spherical, ellipsoidal, etc. The term “nanoparticles” is used as the plural of the term “nanoparticle.”

The term “nucleic acid agent” refers to any synthetic or naturally occurring therapeutic agent including two or more nucleotide residues. In an embodiment the nucleic acid agent is an RNA, a DNA or a mixed polymer of RNA and DNA. In an embodiment an RNA is an mRNA or a siRNA. In an embodiment a DNA is a cDNA or genomic DNA. In an embodiment the nucleic acid agent is single stranded and in another embodiment it comprises two strands. In an embodiment the nucleic acid agent can have a duplexed region, comprised of strands from one or two molecules. In an embodiment the nucleic acid agent is an agent that inhibits gene expression, e.g., an agent that promotes RNAi. In some embodiments, the nucleic acid agent is siRNA, shRNA, an antisense oligonucleotide, or a microRNA (miRNA). In an embodiment the nucleic acid agent is an antagomir or an aptamer.

As used herein, “particle polydispersity index (PDI)” or “particle polydispersity” refers to the width of the particle size distribution. Particle PDI can be calculated from the equation PDI=2a₂/a₁² where a₁ is the 1^{st} Cumulant or moment used to calculate the intensity weighted Z average mean size and a₂ is the 2^{nd} moment used to calculate a parameter defined as the polydispersity index (PdI). A particle PDI of 1 is the theoretical maximum and would be a completely flat size distribution plot. Compositions of particles described herein may have particle PDIs of less than 0.5, less than 0.4, less than 0.3, less than 0.2, or less than 0.1.

“Pharmaceutically acceptable carrier or adjuvant,” as used herein, refers to a carrier or adjuvant that may be administered to a patient, together with a polymer-agent conjugate, particle or composition described herein, and which does not destroy the pharmacological activity thereof and is nontoxic when administered in doses sufficient to deliver a therapeutic amount of the particle. Some examples of materials which can serve as pharmaceutically acceptable carriers include: (1) sugars, such as lactose, glucose, mannitol and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical compositions.

The term “polymer,” as used herein, is given its ordinary meaning as used in the art, i.e., a molecular structure featuring one or more repeat units (monomers), connected by covalent bonds. The repeat units may all be identical, or in some cases, there may be more than one type of repeat unit present within the polymer. Polymers may be natural or unnatural (synthetic) polymers. Polymers may be homopolymers or copolymers containing two or more monomers. Polymers may be linear or branched.

If more than one type of repeat unit is present within the polymer, then the polymer is to be a “copolymer.” It is to be understood that in any embodiment employing a polymer, the polymer being employed may be a copolymer. The repeat units forming the copolymer may be arranged in any fashion. For example, the repeat units may be arranged in a random order, in an alternating order, or as a “block” copolymer, i.e., containing one or more regions each containing a first repeat unit (e.g., a first block), and one or more regions each containing a second repeat unit (e.g., a second block), etc. Block copolymers may have two (a diblock copolymer), three (a triblock copolymer), or more numbers of distinct blocks. In terms of sequence, copolymers may be random, block, or contain a combination of random and block sequences.

In some cases, the polymer is biologically derived, i.e., a biopolymer. Non-limiting examples of biopolymers include peptides or proteins (i.e., polymers of various amino acids), or nucleic acids such as DNA or RNA.

As used herein, “polymer polydispersity index (PDI)” or “polymer polydispersity” refers to the distribution of molecular mass in a given polymer sample. The polymer PDI calculated is the weight average molecular weight divided by the number average molecular weight. It indicates the distribution of individual molecular masses in a batch of polymers. The polymer PDI has a value typically greater than 1, but as the polymer chains approach uniform chain length, the PDI approaches unity (1).

As used herein, the term “prevent” or “preventing” as used in the context of the administration of an agent to a subject, refers to subjecting the subject to a regimen, e.g., the administration of a polymer-agent conjugate, particle or composition, such that the onset of at least one symptom of the disorder is delayed as compared to what would be seen in the absence of the regimen.

As used herein, the term “subject” is intended to include human and non-human animals. Exemplary human subjects include a human patient having a disorder, e.g., a disorder described herein, or a normal subject. The term “non-human animals” includes all vertebrates, e.g., non-mammals (such as chickens, amphibians, reptiles) and mammals, such as non-human primates, domesticated and/or agriculturally useful animals, e.g., sheep, dog, cat, cow, pig, etc.

As used herein, the term “treat” or “treating” a subject having a disorder refers to subjecting the subject to a regimen, e.g., the administration of a polymer-agent conjugate, particle or composition, such that at least one symptom of the disorder is cured, healed, alleviated, relieved, altered, remedied, ameliorated, or improved. Treating includes administering an amount effective to alleviate, relieve, alter, remedy, ameliorate, improve or affect the disorder or the symptoms of the disorder. The treatment may inhibit deterioration or worsening of a symptom of a disorder.

The term “acyl” refers to an alkylcarbonyl, cycloalkylcarbonyl, arylcarbonyl, heterocyclylcarbonyl, or heteroarylcarbonyl substituent, any of which may be further substituted (e.g., by one or more substituents). Exemplary acyl groups include acetyl (CH₃C(O)—), benzoyl (C₆H₅C(O)—), and acetylamino acids (e.g., acetylglycine, CH₃C(O)NHCH₂C(O)—.

The term “alkoxy” refers to an alkyl group, as defined below, having an oxygen radical attached thereto. Representative alkoxy groups include methoxy, ethoxy, propyloxy, tert-butoxy and the like.

The term “carboxy” refers to a —C(O)OH or salt thereof.

The term “hydroxy” and “hydroxyl” are used interchangeably and refer to —OH.

The term “substituents” refers to a group “substituted” on an alkyl, cycloalkyl, alkenyl, alkynyl, heterocyclyl, heterocycloalkenyl, cycloalkenyl, aryl, or heteroaryl group at any atom of that group. Any atom can be substituted. Suitable substituents include, without limitation, alkyl (e.g., C1, C2, C3, C4, C5, C6, C7, C8, C9, C10, C11, C12 straight or branched chain alkyl), cycloalkyl, haloalkyl (e.g., perfluoroalkyl such as CF₃), aryl, heteroaryl, aralkyl, heteroaralkyl, heterocyclyl, alkenyl, alkynyl, cycloalkenyl, heterocycloalkenyl, alkoxy, haloalkoxy (e.g., perfluoroalkoxy such as OCF₃), halo, hydroxy, carboxy, carboxylate, cyano, nitro, amino, alkyl amino, SO₃H, sulfate, phosphate, methylenedioxy (—O—CH₂—O— wherein oxygens are attached to vicinal atoms), ethylenedioxy, oxo, thioxo (e.g., C═S), imino (alkyl, aryl, aralkyl), S(O)ₙalkyl (where n is 0-2), S(O)ₙaryl (where n is 0-2), S(O)ₙheteroaryl (where n is 0-2), S(O)ₙheterocyclyl (where n is 0-2), amine (mono-, di-, alkyl, cycloalkyl, aralkyl, heteroaralkyl, aryl, heteroaryl, and combinations thereof), ester (alkyl, aralkyl, heteroaralkyl, aryl, heteroaryl), amide (mono-, di-, alkyl, aralkyl, heteroaralkyl, aryl, heteroaryl, and combinations thereof), sulfonamide (mono-, di-, alkyl, aralkyl, heteroaralkyl, and combinations thereof). In one aspect, the substituents on a group are independently any one single, or any subset of the aforementioned substituents. In another aspect, a substituent may itself be substituted with any one of the above substituents.

### Particles

The particles, in general, include a nucleic acid agent, and at least one of a cationic moiety, a hydrophobic moiety, such as a polymer, or a hydrophilic-hydrophobic polymer. In some embodiments, the particles include a nucleic acid agent and a cationic moiety, and at least one of a hydrophobic moiety, such as a polymer, or a hydrophilic-hydrophobic polymer. In some embodiments, a particle described herein includes a hydrophobic moiety such as a hydrophobic polymer or lipid (e.g., hydrophobic polymer), a polymer containing a hydrophilic portion and a hydrophobic portion, a nucleic acid agent, and a cationic moiety. In some embodiments, the nucleic acid agent and/or cationic moiety is attached to a moiety. For example, the nucleic acid agent and/or cationic moiety can be attached to a polymer (e.g., the hydrophobic polymer or the polymer containing a hydrophilic portion and a hydrophobic portion) or the nucleic acid agent forms a duplex with a nucleic acid that is attached to a polymer. In some embodiments, the nucleic acid agent is attached to a polymer (e.g., a hydrophobic polymer or a polymer containing a hydrophilic and a hydrophobic portion), and the cationic moiety is not attached to a polymer (e.g., the cationic moiety is embedded in the particle). In some embodiments, the nucleic acid agent and the cationic moiety are both attached to a polymer (e.g., a hydrophobic polymer or a polymer containing a hydrophilic and a hydrophobic portion) or the nucleic acid agent forms a duplex with a nucleic acid that is attached to a polymer and the cationic moiety is attached to a polymer. In some embodiments, the cationic moiety is attached to a polymer (e.g., a hydrophobic polymer or a polymer containing a hydrophilic and a hydrophobic portion), and the nucleic acid agent is not attached to a polymer (e.g., the nucleic acid agent is embedded in the particle). In some embodiments, neither the nucleic acid agent nor cationic moiety is attached to a polymer. The nucleic acid agent and/or cationic moiety can also be attached to other moieties. For example, the nucleic acid agent can be attached to the cationic moiety or to a hydrophilic polymer such as PEG.

In addition to a hydrophobic moiety such as a hydrophobic polymer or lipid (e.g., hydrophobic polymer), a polymer containing a hydrophilic portion and a hydrophobic portion, a nucleic acid agent, and a cationic moiety, the particles described herein may include one or more additional components such as an additional nucleic acid agent or an additional cationic moiety. A particle described herein may also include a compound having at least one acidic moiety, such as a carboxylic acid group. The compound may be a small molecule or a polymer having at least one acidic moiety. In some embodiments, the compound is a polymer such as PLGA.

In some embodiments, the particle is configured such that when administered to a subject there is preferential release of the nucleic acid agent, e.g., siRNA, in a preselected compartment. The preselected compartment can be a target site, location, tissue type, cell type, e.g., a disease specific cell type, e.g., a cancer cell, or subcellular compartment, e.g., the cytosol. In an embodiment a particle provides preferential release in a tumor, as opposed to other compartments, e.g., non-tumor compartments, e.g., the peripheral blood. In embodiments, where the nucleic acid agent, e.g., an siRNA, is attached to a polymer or a cationic moiety, the nucleic acid agent is released (e.g., through reductive cleavage of a linker) to a greater degree in a tumor than in non-tumor compartments, e.g., the peripheral blood, of a subject. In some embodiments, the particle is configured such that when administered to a subject, it delivers more nucleic acid agent, e.g, siRNA, to a compartment of the subject, e.g., a tumor, than if the nucleic acid agent were administered free.

In some embodiments, the particle is associated with an excipient, e.g., a carbohydrate component, or a stabilizer or lyoprotectant, e.g., a carbohydrate component, stabilizer or lyoprotectant described herein. While not wishing to be bound be theory the carbohydrate component may act as a stabilizer or lyoprotectant. In some embodiments, the carbohydrate component, stabilizer or lyoprotectant, comprises one or more carbohydrates (e.g., one or more carbohydrates described herein, such as, e.g., sucrose, cyclodextrin or a derivative of cyclodextrin (e.g. 2-hydroxypropyl-β-cyclodextrin, sometimes referred to herein as HP-β-CD)), salt, PEG, PVP or crown ether. In some embodiments, the carbohydrate component, stabilizer or lyoprotectant comprises two or more carbohydrates, e.g., two or more carbohydrates described herein. In one embodiment, the carbohydrate component, stabilizer or lyoprotectant includes a cyclic carbohydrate (e.g., cyclodextrin or a derivative of cyclodextrin, e.g., an α-, β-, or γ-, cyclodextrin (e.g. 2-hydroxypropyl-β-cyclodextrin)) and a non-cyclic carbohydrate. Exemplary non-cyclic oligosaccharides include those of less than 10, 8, 6 or 4 monosaccharide subunits (e.g., a monosaccharide or a disaccharide (e.g., sucrose, trehalose, lactose, maltose) or combinations thereof).

In an embodiment the carbohydrate component, stabilizer or lyoprotectant comprises a first and a second component, e.g., a cyclic carbohydrate and a non-cyclic carbohydrate, e.g., a mono-, di, or tetra saccharide.

In one embodiment, the weight ratio of cyclic carbohydrate to non-cyclic carbohydrate associated with the particle is a weight ratio described herein, e.g., 0.5:1.5 to 1.5:0.5.

In an embodiment the carbohydrate component, stabilizer or lyoprotectant comprises a first and a second component (designated here as A and B) as follows:
(A) comprises a cyclic carbohydrate and (B) comprises a disaccharide;
(A) comprises more than one cyclic carbohydrate, e.g., a β-cyclodextrin (sometimes referred to herein as β-CD) or a β-CD derivative, e.g., HP-β-CD, and (B) comprises a disaccharide;
(A) comprises a cyclic carbohydrate, e.g., a β-CD or a β-CD derivative, e.g., HP-β-CD, and
(B) comprises more than one disaccharide;
(A) comprises more than one cyclic carbohydrate, and (B) comprises more than one disaccharide; (A) comprises α cyclodextrin, e.g., a β-CD or a β-CD derivative, e.g., HP-β-CD, and (B) comprises a disaccharide;
(A) comprises a β-cyclodextrin, e.g a β-CD derivative, e.g., HP-β-CD, and (B) comprises a disaccharide;
(A) comprises a β-cyclodextrin, e.g., a β-CD derivative, e.g., HP-β-CD, and (B) comprises sucrose;
(A) comprises a β-CD derivative, e.g., HP-β-CD, and (B) comprises sucrose;
(A) comprises a β-cyclodextrin, e.g., a β-CD derivative, e.g., HP-β-CD, and (B) comprises trehalose;
(A) comprises a β-cyclodextrin, e.g., a β-CD derivative, e.g., HP-β-CD, and (B) comprises sucrose and trehalose.
(A) comprises HP-β-CD, and (B) comprises sucrose and trehalose.

In an embodiment components A and B are present in the following ratio: 0.5:1.5 to 1.5:0.5. In an embodiment, components A and B are present in the following ratio: 3-1:0.4-2; 3-1:0.4-2.5; 3-1:0.4-2; 3-1:0.5-1.5; 3-1:0.5-1; 3-1:1; 3-1:0.6-0.9; and 3:1:0.7. In an embodiment, components A and B are present in the following ratio: 2-1:0.4-2; 3-1:0.4-2.5; 2-1:0.4-2; 2-1:0.5-1.5; 2-1:0.5-1; 2-1:1; 2-1:0.6-0.9; and 2:1:0.7. In an embodiment components A and B are present in the following ratio: 2-1.5:0.4-2; 2-1.5:0.4-2.5; 2-1.5:0.4-2; 2-1.5:0.5-1.5; 2-1.5:0.5-1; 2-1.5:1; 2-1.5:0.6-0.9; 2:1.5:0.7. In an embodiment components A and B are present in the following ratio: 2.5-1.5:0.5-1.5; 2.2-1.6:0.7-1.3; 2.0-1.7:0.8-1.2; 1.8:1; 1.85:1 and 1.9:1.

In an embodiment component A comprises a cyclodextin, e.g., a β-cyclodextrin, e.g., a β-CD derivative, e.g., HP-β-CD, and (B) comprises sucrose, and they are present in the following ratio: 2.5-1.5:0.5-1.5; 2.2-1.6:0.7-1.3; 2.0-1.7:0.8-1.2; 1.8:1; 1.85:1 and 1.9:1.

In some embodiments, the particle is a nanoparticle. In some embodiments, the nanoparticle has a diameter of less than or equal to about 220 nm (e.g., less than or equal to about 215 nm, 210 nm, 205 nm, 200 nm, 195 nm, 190 nm, 185 nm, 180 nm, 175 nm, 170 nm, 165 nm, 160 nm, 155 nm, 150 nm, 145 nm, 140 nm, 135 nm, 130 nm, 125 nm, 120 nm, 115 nm, 110 nm, 105 nm, 100 nm, 95 nm, 90 nm, 85 nm, 80 nm, 75 nm, 70 nm, 65 nm, 60 nm, 55 nm or 50 nm). In an embodiment, the nanoparticle has a diameter of at least 10 nm (e.g., at least about 20 nm).

A particle described herein may also include a targeting agent or a lipid (e.g., on the surface of the particle).

A composition of a plurality of particles described herein may have an average diameter of about 50 nm to about 500 nm (e.g., from about 50 nm to about 200 nm). A composition of a plurality of particles particle may have a median particle size (Dv50 (particle size below which 50% of the volume of particles exists) of about 50 nm to about 500 nm (e.g., about 75 nm to about 220 nm)) from about 50 nm to about 220 nm (e.g., from about 75 nm to about 200 nm). A composition of a plurality of particles may have a Dv90 (particle size below which 90% of the volume of particles exists) of about 50 nm to about 500 nm (e.g., about 75 nm to about 220 nm). In some embodiments, a composition of a plurality of particles has a Dv90 of less than about 150 nm. A composition of a plurality of particles may have a particle PDI of less than 0.5, less than 0.4, less than 0.3, less than 0.2, or less than 0.1.

A particle described herein may have a surface zeta potential ranging from about −20 mV to about 50 mV, when measured in water. Zeta potential is a measurement of surface potential of a particle. In some embodiments, a particle may have a surface zeta potential, when measured in water, ranging between about −20 mV to about 20 mV, about −10 mV to about 10 mV, or neutral.

In an embodiment, a particle, or a composition comprising a plurality of particles, described herein, has a sufficient amount of nucleic acid agent (e.g., an siRNA), to observe an effect (e.g., knock-down) when administered, for example, in an in vivo model system, (e.g., a mouse model such as any of those described herein).

In an embodiment, a particle, or a composition comprising a plurality of particles described herein, is one in which at least 30, 40, 50, 60, 70, 80, or 90% of its nucleic acid agent, e.g., siRNA, by number or weight, is intact (e.g., as measured by functionality of physical properties, e.g., molecular weight).

In an embodiment, a particle, or a composition comprising a plurality of particles, described herein, is one in which at least 30, 40, 50, 60, 70, 80, or 90% of its nucleic acid agent, e.g., siRNA, by number or weight, is inside, as opposed to exposed at the surface of, the particle.

In an embodiment, a particle, or a composition comprising a plurality of particles, described herein, when incubated in 50/50 mouse/human serum, exhibits little or no aggregation. E.g., when incubated less than 30, 20, or 10%, by number or weight, of the particles will aggregate.

In an embodiment, a particle, or a composition comprising a plurality of particles, described herein may, when stored at 25° C.±2° C./60% relative humidity ±5% relative humidity in an open, or closed, container, for 20, 30, 40, 50 or 60 days, retains at least 30, 40, 50, 60, 70, 80, 90, or 95% of its activity, e.g., as determined in an in vivo model system, (e.g., a mouse model such any of those described herein).

In an embodiment, a particle, or a composition comprising a plurality of particles, described herein may, results in at least 20, 30, 40, 50, or 60% reduction in protein and/or mRNA knockdown when administered as a single dose of 1 or 3 mg/kg in an in vivo model system, (e.g., a mouse model such as any of those described herein).

In an embodiment, a particle or a composition comprising a plurality of particles described herein results in less than 20, 10, 5%, or no knockdown for off target genes, as measured by protein or mRNA, when administered (e.g., as a single dose of 1 or 3 mg/kg) in an in vivo model system, (e.g., a mouse model such as any of those described herein).

In some embodiments, the particles described herein can deliver an effective amount of the nucleic acid agent such that expression of the targeted gene in the subject is reduced by at least 20%, 25%, 30%, 35%, 40%, 45%, 50%, 55%, 60%, 65%, 70%, 75%, 80%, 85%, 90%, 95% or more at approximately 72 hours, 96 hours, 120 hours, 144 hours, 168 hours, 192 hours, 216 hours, 240 hours, 264 hours after administration of the particles to the subject. In one embodiment, the particles described herein can deliver an effective amount of the nucleic acid agent such that expression of the targeted gene in the subject is reduced by at least 50%, 55%, 60%, 65%, 70%, 75% or 80%, approximately 120 hours after administration of the particles to the subject. In some embodiments, the level of target gene expression in a subject administered a particle or composition described herein is compared to the level of expression of the target gene seen when the nucleic acid agent is administered in a formulation other than a particle or a conjugate (i.e., not in a particle, e.g., not embedded in a particle or conjugated to a polymer, for example, a particle deascibed herein) or than expression of the target gene seen in the absence of the administration of the nucleic acid agent or other therapeutic agent).

In an embodiment, a particle or a composition comprising a plurality of particles, described herein, when contacted with target gene mRNA, results in cleavage of the mRNA.

In an embodiment, a particle or a composition comprising a plurality of particles, described herein, results in less than 2, 5, or 10 fold cytokine induction, when administered (e.g., as a single dose of 1 or 3 mg/kg) in an in vivo model system, (e.g., a mouse model such as any of those described herein). E.g., the administration results in less than 2, 5, or 10 fold induction of one, or more, e.g., two, three, four, five, six, or seven, or all, of: tumor necrosis factor-alpha, interleukin-1alpha, interleukin-1beta, interleukin-6, interleukin-10, interleukin-12, keratinocyte-derived cytokine and interferon-gamma.

In an embodiment, a particle, or a composition comprising a plurality of particles, described herein, results in less than 2, 5, or 10 fold increase in alanine aminotransferase (ALT) and aspartate aminotransferase (AST), when administered (e.g., as a single dose of 1 or 3 mg/kg) in an in vivo model system (e.g., a mouse model such as any of those described herein). In an embodiment, a particle, or a composition comprising a plurality of particles, described herein, results in no significant changes in blood count 48 hours after 2 doses of 3 mg/kg in an in vivo model system, (e.g., a mouse model such as one described herein).

In an embodiment a particle is stable in non-polar organic solvent (e.g., any of hexane, chloroform, or dichloromethane). By way of example, the particle does not substantially invert, e.g., if present, an outer layer does not internalize, or a substantial amount of surface components do internalize, relative to their configuration in aqueous solvent. In embodiments the distribution of components is substantially the same in a non-polar organic solvent and in an aqueous solvent.

In an embodiment a particle lacks at least one component of a micelle, e.g., it lacks a core which is substantially free of hydrophilic components.

In an embodiment the core of the particle comprises a substantial amount of a hydrophilic component.

In an embodiment the core of the particle comprises a substantial amount e.g., at least 10, 20, 30, 40, 50, 60 or 70% (by weight or number) of the nucleic acid agent, e.g., siRNA, of the particle.

In an embodiment the core of the particle comprises a substantial amount e.g., at least 10, 20, 30, 40, 50, 60 or 70% (by weight or number) of the cationic, e.g., polycationic moiety, of the particle.

A particle described herein may include a small amount of a residual solvent, e.g., a solvent used in preparing the particles such as acetone, tert-butylmethyl ether, benzyl alcohol, dioxane, heptane, dichloromethane, dimethylformamide, dimethylsulfoxide, ethyl acetate, acetonitrile, tetrahydrofuran, ethanol, methanol, isopropyl alcohol, methyl ethyl ketone, butyl acetate, or propyl acetate (e.g., isopropylacetate). In some embodiments, the particle may include less than 5000 ppm of a solvent (e.g., less than 4500 ppm, less than 4000 ppm, less than 3500 ppm, less than 3000 ppm, less than 2500 ppm, less than 2000 ppm, less than 1500 ppm, less than 1000 ppm, less than 500 ppm, less than 250 ppm, less than 100 ppm, less than 50 ppm, less than 25 ppm, less than 10 ppm, less than 5 ppm, less than 2 ppm, or less than 1 ppm).

In some embodiments, the particle is substantially free of a class II or class III solvent as defined by the United States Department of Health and Human Services Food and Drug Administration “Q3c—Tables and List.” In some embodiments, the particle comprises less than 5000 ppm of acetone. In some embodiments, the particle comprises less than 5000 ppm of tert-butylmethyl ether. In some embodiments, the particle comprises less than 5000 ppm of heptane. In some embodiments, the particle comprises less than 600 ppm of dichloromethane. In some embodiments, the particle comprises less than 880 ppm of dimethylformamide. In some embodiments, the particle comprises less than 5000 ppm of ethyl acetate. In some embodiments, the particle comprises less than 410 ppm of acetonitrile. In some embodiments, the particle comprises less than 720 ppm of tetrahydrofuran. In some embodiments, the particle comprises less than 5000 ppm of ethanol. In some embodiments, the particle comprises less than 3000 ppm of methanol. In some embodiments, the particle comprises less than 5000 ppm of isopropyl alcohol. In some embodiments, the particle comprises less than 5000 ppm of methyl ethyl ketone. In some embodiments, the particle comprises less than 5000 ppm of butyl acetate. In some embodiments, the particle comprises less than 5000 ppm of propyl acetate.

A particle described herein may include varying amounts of a hydrophobic moiety such as a hydrophobic polymer, e.g., from about 20% to about 90% by weight of, or used as starting materials to make, the particle (e.g., from about 20% to about 80%, from about 25% to about 75%, or from about 30% to about 70% by weight).

A particle described herein may include varying amounts of a polymer containing a hydrophilic portion and a hydrophobic portion, e.g., up to about 50% by weight of, or used as starting materials to make, the particle (e.g., from about 4 to any of about 50%, about 5%, about 8%, about 10%, about 15%, about 20%, about 23%, about 25%, about 30%, about 35%, about 40%, about 45% or about 50% by weight). For example, the percent by weight of the hydrophobic-hydrophilic polymer of the particle is from about 3% to 30%, from about 5% to 25% or from about 8% to 23%.

In a particle described herein, the ratio of the hydrophobic polymer to the hydrophobic-hydrophilic polymer is such that the particle comprises at least 5%, 8%, 10%, 12%, 15%, 18%, 20%, 23%, 25%, or 30% by weight of a polymer of, or used as starting materials to make, the particle having a hydrophobic portion and a hydrophilic portion.

A particle described herein may include varying amounts of a cationic moiety, e.g., from about 0.1% to about 60% by weight of, or used as starting materials to make, the particle (e.g., from about 1% to about 60%, from about 2% to about 20%, from about 3% to about 30%, from about 5% to about 40%, from about or from about 10% to about 30%). When the cationic moiety is a nitrogen containing moiety, the ratio of nitrogen moieties in the particle to phosphates from the nucleic acid agent backbone in the particle (i.e., N/P ratio) can be from about 1:1 to about 50:1 (e.g., from about 1:1 to about 25:1, from about 1:1 to about 10:1, from about 1:1 to about 5:1, or from about 1:1 to about 1.5 to 1:1).

A particle described herein may include varying amounts of a nucleic acid agent, e.g., from about 0.1% to about 50% by weight of, or used as starting materials to make, the particle (e.g., from about 1% to about 50%, from about 0.5% to about 20%, from about 2% to about 20%, from about or from about 5% to about 15%).

When the particle includes a surfactant, the particle may include varying amounts of the surfactant, e.g., up to about 40% by weight of, or used as starting materials to make, the particle, or from about 15% to about 35% or from about 3% to about 10%. In some embodiments, the surfactant is PVA and the cationic moiety is cationic PVA. In some embodiments, the particle may include about 2% to about 5% of PVA (e.g., about 4%) and from about 0.1% to about 3% cationic PVA (e.g., about 1%).

A particle described herein may be substantially free of a targeting agent (e.g., of a targeting agent covalently linked to a component in the particle, e.g., a targeting agent able to bind to or otherwise associate with a target biological entity, e.g., a membrane component, a cell surface receptor, prostate specific membrane antigen, or the like). A particle described herein may be substantially free of a targeting agent selected from nucleic acid aptamers, growth factors, hormones, cytokines, interleukins, antibodies, integrins, fibronectin receptors, p-glycoprotein receptors, peptides and cell binding sequences. In some embodiments, no polymer within the particle is conjugated to a targeting moiety. A particle described herein may be free of moieties added for the purpose of selectively targeting the particle to a site in a subject, e.g., by the use of a moiety on the particle having a high and specific affinity for a target in the subject.

In some embodiments the particle is free of a lipid, e.g., free of a phospholipid. A particle described herein may be substantially free of an amphiphilic layer that reduces water penetration into the nanoparticle. A particle described herein may comprise less than 5 or 10% (e.g., as determined as w/w, v/v) of a lipid, e.g., a phospholipid. A particle described herein may be substantially free of a lipid layer, e.g., a phospholipid layer, e.g., that reduces water penetration into the nanoparticle. A particle described herein may be substantially free of lipid, e.g., is substantially free of phospholipid.

A particle described herein may be substantially free of a radiopharmaceutical agent, e.g., a radiotherapeutic agent, radiodiagnostic agent, prophylactic agent, or other radioisotope. A particle described herein may be substantially free of an immunomodulatory agent, e.g., an immunostimulatory agent or immunosuppressive agent. A particle described herein may be substantially free of a vaccine or immunogen, e.g., a peptide, sugar, lipid-based immunogen, B cell antigen or T cell antigen.

A particle described herein may be substantially free of a water-soluble hydrophobic polymer such as PLGA, e.g., PLGA having a molecular weight of less than about 1 kDa (e.g., less than about 500 Da).

Exemplary Particles

One exemplary particle includes a particle comprising:

a) a plurality of hydrophobic moieties, e.g., hydrophobic polymers;

b) a plurality of hydrophilic-hydrophobic polymers;

c) optionally, a plurality of cationic moieties; and

d) a plurality of nucleic acid agents wherein at least a portion of the plurality of nucleic acid agents are

(i) covalently attached to either of

a hydrophobic moiety, e.g., a hydrophobic polymer of a) or

a hydrophilic-hydrophobic polymer of b), or

(ii) form a duplex (e.g., a heteroduplex) with a nucleic acid which is covalently attached to either of a hydrophobic moiety, e.g., hydrophobic polymer, of a) or the hydrophilic-hydrophobic polymer b).

Another exemplary particle includes a particle comprising:

a) a plurality of nucleic acid agent-polymer conjugates, each of which

comprises a nucleic acid agent which

(i) is attached to a hydrophobic polymer or

(ii) forms a duplex (e.g., a heteroduplex) with a nucleic acid which is covalently attached to a hydrophobic polymer;

b) a plurality of hydrophilic-hydrophobic polymers; and

c) optionally, a plurality of cationic moieties.

Another exemplary particle includes a particle comprising:

a) a plurality of hydrophobic moieties, e.g., hydrophobic polymers;

b) a plurality of nucleic acid agent-hydrophilic-hydrophobic polymer conjugates wherein the nucleic acid agent of each nucleic acid agent-hydrophilic-hydrophobic polymer conjugate of the plurality
(i) is covalently attached to the hydrophilic-hydrophobic polymer or
(ii) forms a duplex (e.g., a heteroduplex) with a nucleic acid which is covalently attached the hydrophilic-hydrophobic polymer; and

c) optionally, a plurality of cationic moieties.

Another exemplary particle includes a particle comprising:

a) a plurality of hydrophobic moieties, e.g., hydrophobic polymers;

b) a plurality of hydrophilic-hydrophobic polymers;

c) a plurality of cationic moieties, wherein at least a portion of the plurality of cationic moieties is attached to either a hydrophobic polymer of a) or a hydrophilic-hydrophobic polymer of b); and

d) a plurality of nucleic acid agents.

Another exemplary particle includes a particle comprising:

a) a plurality of hydrophobic moieties (e.g., hydrophobic polymers);

b) a plurality of hydrophilic-hydrophobic polymers;

c) optionally, a plurality of cationic moieties; and

d) a plurality of nucleic acid agents;

wherein a substantial portion of the cationic moieties of c) and a substantial portion of the nucleic acid agents of d) is not covalently attached to a hydrophobic polymer or a hydrophilic-hydrophobic polymer. For example, the nucleic acid agents or cationic moieties are embedded in the particle.

Another exemplary particle includes a particle comprising:

a) a plurality of hydrophobic moieties, e.g., hydrophobic polymers;

b) optionally a plurality of hydrophilic-hydrophobic polymers;

c) a plurality of cationic moieties; and

d) a plurality of nucleic acid agents, wherein at least a portion of the plurality of nucleic acid agents are covalently attached to a hydrophilic polymer or form a duplex (e.g., a heteroduplex) with a nucleic acid that is covalently attached to a hydrophilic polymer.

Another exemplary particle includes a particle comprising:

a) a plurality of hydrophobic moieties, e.g., hydrophobic polymers;

b) a plurality of hydrophilic-hydrophobic polymers; and

c) a plurality of nucleic acid agent-cationic polymer conjugates.

In an embodiment the nucleic acid agent is not attached, e.g., covalently attached, to hydrophobic polymer or hydrophilic-hydrophobic polymer. In an embodiment, less than 5, 2, or 1%, by weight, of the nucleic acid agent in, or used as starting materials to make, the particles, are attached to hydrophobic polymers or hydrophilic-hydrophobic polymers.

Another exemplary particle includes a plurality of nucleic acid agent-polymer conjugates; a plurality of cationic polymers or lipids; and a plurality of polymers or lipids, wherein the polymers or lipids substantially surround the plurality of nucleic acid agent-polymer conjugates, for example, such the nucleic acid agent is substantially inside the particle, absent from the surface of the particle.

### Hydrophobic Moieties

Hydrophobic Polymers

A particle described herein may include a hydrophobic polymer. The hydrophobic polymer may be attached to a nucleic acid agent and/or cationic moiety to form a conjugate (e.g., a nucleic acid agent-hydrophobic polymer conjugate or cationic moiety-hydrophobic polymer conjugate). In some embodiments, the nucleic acid agent forms a duplex with a nucleic acid that is attached to the hydrophobic polymer.

In some embodiments, the hydrophobic polymer is not attached to another moiety. A particle can include a plurality of hydrophobic polymers, for example where some are attached to another moiety such as a nucleic acid agent and/or cationic moiety and some are free.

Exemplary hydrophobic polymers include the following: acrylates including methyl acrylate, ethyl acrylate, propyl acrylate, n-butyl acrylate (BA), isobutyl acrylate, 2-ethyl acrylate, and t-butyl acrylate; methacrylates including ethyl methacrylate, n-butyl methacrylate, and isobutyl methacrylate; acrylonitriles; methacrylonitrile; vinyls including vinyl acetate, vinylversatate, vinylpropionate, vinylformamide, vinylacetamide, vinylpyridines, and vinylimidazole; aminoalkyls including aminoalkylacrylates, aminoalkylmethacrylates, and aminoalkyl(meth)acrylamides; styrenes; cellulose acetate phthalate; cellulose acetate succinate; hydroxypropylmethylcellulose phthalate; poly(D,L-lactide); poly(D,L-lactide-co-glycolide); poly(glycolide); poly(hydroxybutyrate); poly(alkylcarbonate); poly(orthoesters); polyesters; poly(hydroxyvaleric acid); polydioxanone; poly(ethylene terephthalate); poly(malic acid); poly(tartronic acid); polyanhydrides; polyphosphazenes; poly(amino acids) and their copolymers (see generally, Svenson, S (ed.)., Polymeric Drug Delivery: Volume I: Particulate Drug Carriers. 2006; ACS Symposium Series; Amiji, M. M (ed.)., Nanotechnology for Cancer Therapy. 2007; Taylor & Francis Group, LLP; Nair et al. Prog. Polym. Sci. (2007) 32: 762-798); hydrophobic peptide-based polymers and copolymers based on poly(L-amino acids) (Lavasanifar, A., et al., Advanced Drug Delivery Reviews (2002) 54:169-190); poly(ethylene-vinyl acetate) (“EVA”) copolymers; silicone rubber; polyethylene; polypropylene; polydienes (polybutadiene, polyisoprene and hydrogenated forms of these polymers); maleic anhydride copolymers of vinyl methylether and other vinyl ethers; polyamides (nylon 6,6); polyurethane; poly(ester urethanes); poly(ether urethanes); and poly(ester-urea).

Hydrophobic polymers useful in preparing the polymer-agent conjugates or particles described herein also include biodegradable polymers. Examples of biodegradable polymers include polylactides, polyglycolides, caprolactone-based polymers, poly(caprolactone), polydioxanone, polyanhydrides, polyamines, polyesteramides, polyorthoesters, polydioxanones, polyacetals, polyketals, polycarbonates, polyphosphoesters, polyesters, polybutylene terephthalate, polyorthocarbonates, polyphosphazenes, succinates, poly(malic acid), poly(amino acids), poly(vinylpyrrolidone), polyethylene glycol, polyhydroxycellulose, polysaccharides, chitin, chitosan and hyaluronic acid, and copolymers, terpolymers and mixtures thereof. Biodegradable polymers also include copolymers, including caprolactone-based polymers, polycaprolactones and copolymers that include polybutylene terephthalate.

In some embodiments, the polymer is a polyester synthesized from monomers selected from the group consisting of D,L-lactide, D-lactide, L-lactide, D,L-lactic acid, D-lactic acid, L-lactic acid, glycolide, glycolic acid, ε-caprolactone, ε-hydroxy hexanoic acid, γ-butyrolactone, γ-hydroxy butyric acid, δ-valerolactone, δ-hydroxy valeric acid, hydroxybutyric acids, and malic acid.

A copolymer may also be used in a polymer-agent conjugate or particle described herein. In some embodiments, a polymer may be PLGA, which is a biodegradable random copolymer of lactic acid and glycolic acid. A PLGA polymer may have varying ratios of lactic acid:glycolic acid, e.g., ranging from about 0.1:99.9 to about 99.9:0.1 (e.g., from about 75:25 to about 25:75, from about 60:40 to 40:60, or about 55:45 to 45:55). In some embodiments, e.g., in PLGA, the ratio of lactic acid monomers to glycolic acid monomers is 50:50, 60:40 or 75:25.

In particular embodiments, by optimizing the ratio of lactic acid to glycolic acid monomers in the PLGA polymer of the polymer-agent conjugate or particle, parameters such as water uptake, agent release (e.g., “controlled release”) and polymer degradation kinetics may be optimized. Furthermore, tuning the ratio will also affect the hydrophobicity of the copolymer, which may in turn affect drug loading.

In certain embodiments wherein the biodegradable polymer also has a nucleic acid agent or other material such as a cationic moiety attached to it or a nucleic acid agent that forms a duplex with a nucleic acid attached to it, the biodegradation rate of such polymer may be characterized by a release rate of such materials. In such circumstances, the biodegradation rate may depend on not only the chemical identity and physical characteristics of the polymer, but also on the identity of material(s) attached thereto. Degradation of the subject compositions includes not only the cleavage of intramolecular bonds, e.g., by oxidation and/or hydrolysis, but also the disruption of intermolecular bonds, such as dissociation of host/guest complexes by competitive complex formation with foreign inclusion hosts. In some embodiments, the release can be affected by an additional component in the particle, e.g., a compound having at least one acidic moiety (e.g., free-acid PLGA).

In certain embodiments, particles comprising one or more polymers, such as a hydrophobic polymer, biodegrade within a period that is acceptable in the desired application. In certain embodiments, such as in vivo therapy, such degradation occurs in a period usually less than about five years, one year, six months, three months, one month, fifteen days, five days, three days, or even one day on exposure to a physiological solution with a pH between 4 and 8 having a temperature of between 25° C. and 37° C. In other embodiments, the polymer degrades in a period of between about one hour and several weeks, depending on the desired application.

When polymers are used for delivery of nucleic acid agents in vivo, it is important that the polymers themselves be nontoxic and that they degrade into non-toxic degradation products as the polymer is eroded by the body fluids. Many synthetic biodegradable polymers, however, yield oligomers and monomers upon erosion in vivo that adversely interact with the surrounding tissue (D. F. Williams, J. Mater. Sci. 1233 (1982)). To minimize the toxicity of the intact polymer carrier and its degradation products, polymers have been designed based on naturally occurring metabolites. Exemplary polymers include polyesters derived from lactic and/or glycolic acid and polyamides derived from amino acids.

A number of biodegradable polymers are known and used for controlled release of pharmaceuticals. Such polymers are described in, for example, U.S. Pat. Nos. 4,291,013; 4,347,234; 4,525,495; 4,570,629; 4,572,832; 4,587,268; 4,638,045; 4,675,381; 4,745,160; and 5,219,980; and PCT publication WO2006/014626, each of which is hereby incorporated by reference in its entirety.

A hydrophobic polymer described herein may have a variety of end groups. In some embodiments, the end group of the polymer is not further modified, e.g., when the end group is a carboxylic acid, a hydroxy group or an amino group. In some embodiments, the end group may be further modified. For example, a polymer with a hydroxyl end group may be derivatized with an acyl group to yield an acyl-capped polymer (e.g., an acetyl-capped polymer or a benzoyl capped polymer), an alkyl group to yield an alkoxy-capped polymer (e.g., a methoxy-capped polymer), or a benzyl group to yield a benzyl-capped polymer. The end group can also be further reacted with a functional group, for example to provide a linkage to another moiety such as a nucleic acid agent, a cationic moiety, or an insoluble substrate. In some embodiments a particle comprises a functionalized hydrophobic polymer, e.g., a hydrophobic polymer, such as PLGA (e.g., 50:50 PLGA), functionalized with a moiety, e.g., N-(2-aminoethyl)maleimide, 2-(2-(pyridine-2-yl)disulfanyl)ethylamino, or a succinimidyl-N-methyl ester, that has not reacted with another moiety, e.g., a nucleic acid agent.

A hydrophobic polymer may have a weight average molecular weight ranging from about 1 kDa to about 70 kDa (e.g., from about 4 kDa to about 66 kDa, from about 2 kDa to about 12 kDa, from about 6 kDa to about 20 kDa, from about 5 kDa to about 15 kDa, from about 6 kDa to about 13 kDa, from about 7 kDa to about 11 kDa, from about 5 kDa to about 10 kDa, from about 7 kDa to about 10 kDa, from about 5 kDa to about 7 kDa, from about 6 kDa to about 8 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa or about 17 kDa).

A hydrophobic polymer described herein may have a polymer polydispersity index (PDI) of less than or equal to about 2.5 (e.g., less than or equal to about 2.2, less than or equal to about 2.0, or less than or equal to about 1.5). In some embodiments, a hydrophobic polymer described herein may have a polymer PDI of about 1.0 to about 2.5, about 1.0 to about 2.0, about 1.0 to about 1.7, or from about 1.0 to about 1.6.

A particle described herein may include varying amounts of a hydrophobic polymer, e.g., from about 10% to about 90% by weight of the particle (e.g., from about 20% to about 80%, from about 25% to about 75%, or from about 30% to about 70%).

A hydrophobic polymer described herein may be commercially available, e.g., from a commercial supplier such as BASF, Boehringer Ingelheim, Durcet Corporation, Purac America and SurModics Pharmaceuticals. A polymer described herein may also be synthesized. Methods of synthesizing polymers are known in the art (see, for example, Polymer Synthesis: Theory and Practice Fundamentals, Methods, Experiments. D. Braun et al., 4th edition, Springer, Berlin, 2005). Such methods include, for example, polycondensation, radical polymerization, ionic polymerization (e.g., cationic or anionic polymerization), or ring-opening metathesis polymerization.

A commercially available or synthesized polymer sample may be further purified prior to formation of a polymer-agent conjugate or incorporation into a particle or composition described herein. In some embodiments, purification may reduce the polydispersity of the polymer sample. A polymer may be purified by precipitation from solution, or precipitation onto a solid such as Celite. A polymer may also be further purified by size exclusion chromatography (SEC).

Other Hydrophobic Moieties

Other suitable hydrophobic moieties for the particles described herein include lipids e.g., a phospholipid. Exemplary lipids include lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dioleoylphosphatidylethanolamine 4-(N-maleimidomethyl)-cyclohexane-1-carboxylate (DOPE-mal), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), lysophosphatidylcholine, and dilinoleoylphosphatidylcholine.

Other exemplary hydrophobic moieties include cholesterol and Vitamin E TPGS.

In an embodiment, the hydrophobic moiety is not a lipid (e.g., not a phospholipid) or does not comprise a lipid.

### Hydrophobic-Hydrophilic Polymers

A particle described herein may include a polymer containing a hydrophilic portion and a hydrophobic portion, e.g., a hydrophobic-hydrophilic polymer. The hydrophobic-hydrophilic polymer may be attached to another moiety such as a nucleic acid agent (e.g., through the hydrophilic or hydrophobic portion) and/or a cationic moiety or a nucleic acid agent can form a duplex with a nucleic acid attached to the hydrophobic-hydrophilic polymer. In some embodiments, the hydrophobic-hydrophilic polymer is free (i.e., not attached to another moiety). A particle can include a plurality of hydrophobic-hydrophilic polymers, for example where some are attached to another moiety such as a nucleic acid agent and/or cationic moiety and some are free.

A polymer containing a hydrophilic portion and a hydrophobic portion may be a copolymer of a hydrophilic block coupled with a hydrophobic block. These copolymers may have a weight average molecular weight between about 5 kDa and about 30 kDa (e.g., from about 5 kDa to about 25 kDa, from about 10 kDa to about 22 kDa, from about 10 kDa to about 15 kDa, from about 12 kDa to about 22 kDa, from about 7 kDa to about 15 kDa, from about 15 kDa to about 19 kDa, or from about 11 kDa to about 13 kDa, e.g., about 9 kDa, about 10 kDa, about 11 kDa, about 12 kDa, about 13 kDa, about 14 kDa about 15 kDa, about 16 kDa, about 17 kDa, about 18 kDa or about 19 kDa). The polymer containing a hydrophilic portion and a hydrophobic portion may be attached to an agent.

Examples of suitable hydrophobic portions of the polymers include those described above. The hydrophobic portion of the copolymer may have a weight average molecular weight of from about 1 kDa to about 20 kDa (e.g., from about 8 kDa to about 15, kDa from about 1 kDa to about 18 kDa, 17 kDa, 16 kDa, 15 kDa, 14 kDa or 13 kDa, from about 2 kDa to about 12 kDa, from about 6 kDa to about 20 kDa, from about 5 kDa to about 18 kDa, from about 7 kDa to about 17 kDa, from about 8 kDa to about 13 kDa, from about 9 kDa to about 11 kDa, from about 10 kDa to about 14 kDa, from about 6 kDa to about 8 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa or about 17 kDa).

Examples of suitable hydrophilic portions of the polymers include the following: carboxylic acids including acrylic acid, methacrylic acid, itaconic acid, and maleic acid; polyoxyethylenes or polyethylene oxide (PEG); polyacrylamides (e.g. polyhydroxylpropylmethacrylamide), and copolymers thereof with dimethylaminoethylmethacrylate, diallyldimethylammonium chloride, vinylbenzylthrimethylammonium chloride, acrylic acid, methacrylic acid, 2-acrylamido-2-methylpropane sulfonic acid and styrene sulfonate, poly(vinylpyrrolidone), polyoxazoline, polysialic acid, starches and starch derivatives, dextran and dextran derivatives; polypeptides, such as polylysines, polyarginines, polyglutamic acids; polyhyaluronic acids, alginic acids, polylactides, polyethyleneimines, polyionenes, polyacrylic acids, and polyiminocarboxylates, gelatin, and unsaturated ethylenic mono or dicarboxylic acids. A listing of suitable hydrophilic polymers can be found in Handbook of Water-Soluble Gums and Resins, R. Davidson, McGraw-Hill (1980). The hydrophilic portion of the copolymer may have a weight average molecular weight of from about 1 kDa to about 21 kDa (e.g., from about 1 kDa to about 8 kDa, from about 1 kDa to about 3 kDa, e.g., about 2 kDa, or from about 2 kDa to about 6 kDa, e.g., about 3.5 kDa, or from about 4 kDa to about 6 kDa, e.g., about 5 kDa). In one embodiment, the hydrophilic portion is PEG, and the weight average molecular weight is from about 1 kDa to about 21 kDa (e.g., from about 1 kDa to about 8 kDa, from about 1 kDa to about 3 kDa, e.g., about 2 kDa, or from about 2 kDa to about 6 kDa, e.g., about 3.5 kDa, or from about 4 kDa to about 6 kDa, e.g., about 5 kDa). In one embodiment, the hydrophilic portion is PVA, and the weight average molecular weight is from about 1 kDa to about 21 kDa (e.g., from about 1 kDa to about 8 kDa, from about 1 kDa to about 3 kDa, e.g., about 2 kDa, or from about 2 kDa to about 6 kDa, e.g., about 3.5 kDa, or from about 4 kDa to about 6 kDa, e.g., about 5 kDa). In one embodiment, the hydrophilic portion is polyoxazoline, and the weight average molecular weight is from about 1 kDa to about 21 kDa (e.g., from about 1 kDa to about 8 kDa, from about 1 kDa to about 3 kDa, e.g., about 2 kDa, or from about 2 kDa to about 6 kDa, e.g., about 3.5 kDa, or from about 4 kDa to about 6 kDa, e.g., about 5 kDa). In one embodiment, the hydrophilic portion is polyvinylpyrrolidine, and the weight average molecular weight is from about 1 kDa to about 21 kDa (e.g., from about 1 kDa to about 8 kDa, from about 1 kDa to about 3 kDa, e.g., about 2 kDa, or from about 2 kDa to about 6 kDa, e.g., about 3.5 kDa, or from about 4 kDa to about 6 kDa, e.g., about 5 kDa). In one embodiment, the hydrophilic portion is polyhydroxylpropylmethacrylamide, and the weight average molecular weight is from about 1 kDa to about 21 kDa (e.g., from about 1 kDa to about 8 kDa, from about 1 kDa to about 3 kDa, e.g., about 2 kDa, or from about 2 kDa to about 6 kDa, e.g., about 3.5 kDa, or from about 4 kDa to about 6 kDa, e.g., about 5 kDa). In one embodiment, the hydrophilic portion is polysialic acid, and the weight average molecular weight is from about 1 kDa to about 21 kDa (e.g., from about 1 kDa to about 8 kDa, from about 1 kDa to about 3 kDa, e.g., about 2 kDa, or from about 2 kDa to about 6 kDa, e.g., about 3.5 kDa, or from about 4 kDa to about 6 kDa, e.g., about 5 kDa).

A polymer containing a hydrophilic portion and a hydrophobic portion may be a block copolymer, e.g., a diblock or triblock copolymer. In some embodiments, the polymer may be a diblock copolymer containing a hydrophilic block and a hydrophobic block. In some embodiments, the polymer may be a triblock copolymer containing a hydrophobic block, a hydrophilic block and another hydrophobic block. The two hydrophobic blocks may be the same hydrophobic polymer or different hydrophobic polymers. The block copolymers used herein may have varying ratios of the hydrophilic portion to the hydrophobic portion, e.g., ranging from 1:1 to 1:40 by weight (e.g., about 1:1 to about 1:10 by weight, about 1:1 to about 1:2 by weight, or about 1:3 to about 1:6 by weight).

A polymer containing a hydrophilic portion and a hydrophobic portion may have a variety of end groups. In some embodiments, the end group may be a hydroxy group or an alkoxy group (e.g., methoxy). In some embodiments, the end group of the polymer is not further modified. In some embodiments, the end group may be further modified. For example, the end group may be capped with an alkyl group, to yield an alkoxy-capped polymer (e.g., a methoxy-capped polymer), may be derivatized with a targeting agent (e.g., folate) or a dye (e.g., rhodamine), or may be reacted with a functional group.

A polymer containing a hydrophilic portion and a hydrophobic portion may include a linker between the two blocks of the copolymer. Such a linker may be an amide, ester, ether, amino, carbamate or carbonate linkage, for example.

A polymer containing a hydrophilic portion and a hydrophobic portion described herein may have a polymer polydispersity index (PDI) of less than or equal to about 2.5 (e.g., less than or equal to about 2.2, or less than or equal to about 2.0, or less than or equal to about 1.5). In some embodiments, the polymer PDI is from about 1.0 to about 2.5, e.g., from about 1.0 to about 2.0, from about 1.0 to about 1.8, from about 1.0 to about 1.7, or from about 1.0 to about 1.6.

A particle described herein may include varying amounts of a polymer containing a hydrophilic portion and a hydrophobic portion, e.g., up to about 50% by weight of the particle (e.g., from about 4 to about 50%, about 5%, about 10%, about 15%, about 20%, about 25%, about 30%, about 35%, about 40%, about 45% or about 50% by weight). For example, the percent by weight of the second polymer within the particle is from about 3% to 30%, from about 5% to 25% or from about 8% to 23%.

A polymer containing a hydrophilic portion and a hydrophobic portion described herein may be commercially available, or may be synthesized. Methods of synthesizing polymers are known in the art (see, for example, Polymer Synthesis: Theory and Practice Fundamentals, Methods, Experiments. D. Braun et al., 4th edition, Springer, Berlin, 2005). Such methods include, for example, polycondensation, radical polymerization, ionic polymerization (e.g., cationic or anionic polymerization), or ring-opening metathesis polymerization. A block copolymer may be prepared by synthesizing the two polymer units separately and then conjugating the two portions using established methods. For example, the blocks may be linked using a coupling agent such as EDC (1-ethyl-3-(3-dimethylaminopropyl) carbodiimide hydrochloride). Following conjugation, the two blocks may be linked via an amide, ester, ether, amino, carbamate or carbonate linkage.

A commercially available or synthesized polymer sample may be further purified prior to formation of a polymer-agent conjugate or incorporation into a particle or composition described herein. In some embodiments, purification may remove lower molecular weight polymers that may lead to unfilterable polymer samples. A polymer may be purified by precipitation from solution, or precipitation onto a solid such as Celite. A polymer may also be further purified by size exclusion chromatography (SEC).

### Cationic Moieties

Exemplary cationic moieties for use in the particles and conjugates described herein include amines, including for example, primary, secondary, tertiary, and quaternary amines, and polyamines (e.g., branched and linear polyethylene imine (PEI) or derivatives thereof such as polyethyleneimine-PLGA, polyethylene imine-polyethylene glycol —N-acetylgalactosamine (PEI-PEG-GAL) or polyethylene imine-polyethylene glycol-tri-N-acetylgalactosamine (PEI-PEG-triGAL) derivatives). In some embodiments, the cationic moiety comprises a cationic lipid (e.g., 1-[2-(oleoyloxy)ethyl]-2-oleyl-3-(2-hydroxyethyl)imidazolinium chloride (DOTIM), dimethyldioctadecyl ammonium bromide, 1,2 dioleyloxypropyl-3-trimethyl ammonium bromide, DOTAP, 1,2-dimyristyloxypropyl-3-dimethyl-hydroxyethyl ammonium bromide, 1,2-dimyristoyl-sn-glycero-3-ethylphosphocholine (EDMPC), ethyl-PC, 1,2-dioleoyl-3-dimethylammonium-propane (DODAP), DC-cholesterol, and MBOP, CLinDMA, 1,2-dilinoleyloxy-3-dimethylaminopropane (DLinDMA), pCLinDMA, eCLinDMA, DMOBA, and DMLBA). In some embodiments, for example, where the cationic moiety is a polyamine, the polyamine comprises, polyamino acids (e.g., poly(lysine), poly(histidine), and poly(arginine)) and derivatives (e.g. poly(lysine)-PLGA, imidazole modified poly(lysine)) or polyvinyl pyrrolidone (PVP). In some embodiments, for example, where the cationic moiety is a cationic polymer comprising a plurality of amines, the amines can be positioned along the polymer such that the amines are from about 4 to about 10 angstroms apart (e.g., from about 5 to about 8 or from about 6 to about 7). In some embodiments, the amines can be positioned along the polymer so as to be in register with phosphates on a nucleic acid agent.

The cationic moiety can have a pKa of 5 or greater and/or be positively charged at physiological pH.

In some embodiments, the cationic moiety includes at least one amine (e.g., a primary, secondary, tertiary or quaternary amine), or a plurality of amines, each independently a primary, secondary, tertiary or quaternary amine). In some embodiments the cationic moiety is a polymer, for example, having one or more secondary or tertiary amines, for example cationic polyvinyl alcohol (PVA) (e.g., as provided by Kuraray, such as CM-318 or C-506), chitosan, polyamine-branched and star PEG and polyethylene imine. Cationic PVA can be made, for example, by polymerizing a vinyl acetate/N-vinaylformamide co-polymer, e.g., as described in US 2002/0189774, the contents of which are incorporated herein by reference. Other examples of cationic PVA include those described in U.S. Pat. No. 6,368,456 and Fatehi (Carbohydrate Polymers 79 (2010) 423-428), the contents of which are incorporated herein by reference.

In some embodiments, the cationic moiety includes a nitrogen containing heterocyclic or heteroaromatic moiety (e.g, pyridinium, immidazolium, morpholinium, piperizinium, etc.). In some embodiments, the cationic polymer comprises a nitrogen containing heterocyclic or heteroaromatic moiety such as polyvinyl pyrrolidine or polyvinylpyrrolidinone.

In some embodiments, the cationic moiety includes a guanadinium moiety (e.g., an arginine moiety).

In some embodiments, the cationic moiety is a surfactant, for example, a cationic PVA such as a cationic PVA described herein.

Additional exemplary cationic moieties include agamatine, protamine sulfate, hexademethrine bromide, cetyl trimethylammonium bromide, 1-hexyltriethyl-ammonium phosphate, 1-dodecyltriethyl-ammonium phosphate, spermine (e.g., spermine tetrahydrochloride), spermidine, and derivatives thereof (e.g. N1-PLGA-spermine, N1-PLGA-N5,N10,N14-trimethylated-spermine, (N1-PLGA-N5,N10,N14, N14-tetramethylated-spermine), PLGA-glu-di-triCbz-spermine, triCbz-spermine, amiphipole, PMAL-C8, and acetyl-PLGA5050-glu-di(N-1-amino-N5,N10,N14-spermine), poly(2-dimethylamino)ethyl methacrylate), hexyldecyltrimethylammonium chloride, hexadimethrine bromide, and atelocollagen and those described for example in WO2005007854, U.S. Pat. No. 7,641,915, and WO2009055445, the contents of each of which are incorporated herein by reference.

In an embodiment, a cationic moiety is one, the presence of which, in a particle described herein, is accompanied by the presence of less than 50, 40, 30, 20, or 10% (by weight or number) of the nucleic acid agent, e.g., siRNA, on the outside of the particle.

In an embodiment, the cationic moiety is not a lipid (e.g., not a phospholipid) or does not comprise a lipid.

### Nucleic Acid Agents

A nucleic acid agent can be delivered using a particle, conjugate, or composition described herein. Examples of suitable nucleic acid agents include, but are not limited to polynucleotides, such as siRNA, antisense oligonucleotides, microRNAs (miRNAs), antagomirs, aptamers, genomic DNA, cDNA, mRNA, and plasmids. The nucleic acid agent agents can target a variety of genes of interest, such as a gene whose overexpression is associated with a disease or disorder.

The nucleic acid agents delivered using a polymer-nucleic acid agent conjugate, particle or composition described herein can be administered alone, or in combination, (e.g., in the same or separate formulations). In one embodiment, multiple agents, such as, siRNAs, are administered to target different sites on the same gene for treatment of a disease or disorder. In another embodiment, multiple agents, e.g., siRNAs, are administered to target two or more different genes for treatment of a disease or disorder.

siRNA

A therapeutic nucleic acid suitable for delivery by a polymer-nucleic acid agent conjugate, particle or composition described herein can be a “short interfering RNA” or “siRNA.” As used herein, an siRNA refers to any nucleic acid molecule capable of inhibiting or down regulating gene expression or viral replication by mediating RNA interference “RNAi” or gene silencing in a sequence-specific manner. For example the siRNA can be a double-stranded nucleic acid molecule comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof.

In one embodiment, the therapeutic siRNA molecule suitable for delivery with a conjugate, particle or composition described herein interacts with a nucleotide sequence of a target gene in a manner that causes inhibition of expression of the target gene.

siRNA comprises a double stranded structure typically containing 15-50 base pairs, e.g., 19-25, 19-23, 21-25, 21-23, or 24-29 base pairs, and having a nucleotide sequence identical or nearly identical to an expressed target gene or RNA within the cell. An siRNA may be composed of two annealed polynucleotides or a single polynucleotide that forms a hairpin structure. In one embodiment, the therapeutic siRNA is provided in the form of an expression vector, which is packaged in a conjugate, particle or composition described herein, where the vector has a coding sequence that is transcribed to produce one or more transcriptional products that produce siRNA after administration to a subject.

The siRNA can be assembled from two separate oligonucleotides, where one strand is the sense strand and the other is the antisense strand, where the antisense and sense strands are self-complementary (i.e., each strand comprises nucleotide sequence that is complementary to nucleotide sequence in the other strand); such as where the antisense strand and sense strand form a duplex or double stranded structure, for example where the double stranded region is about 15 to about 30 basepairs, e.g., about 15, 16, 17, 18, 19, 20, 21, 22, 23, 24, 25, 26, 27, 28, 29 or 30 base pairs; the antisense strand includes nucleotide sequence that is complementary to nucleotide sequence in a target nucleic acid molecule or a portion thereof and the sense strand comprises nucleotide sequence corresponding to the target nucleic acid sequence or a portion thereof (e.g., about 15 to about 25 or more nucleotides of the siRNA molecule are complementary to the target nucleic acid or a portion thereof). Alternatively, the siRNA is assembled from a single oligonucleotide, where the self-complementary sense and antisense regions of the siRNA are linked by means of a nucleic acid based or non-nucleic acid-based linker(s).

In certain embodiments, at least one strand of the siRNA molecule has a 3′ overhang from about 1 to about 6 nucleotides in length, though may be from 2 to 4 nucleotides in length. Typically, the 3′ overhangs are 1-3 nucleotides in length. In some embodiments, one strand has a 3′ overhang and the other strand is blunt-ended or also has an overhang. The length of the overhangs may be the same or different for each strand. To further enhance the stability of the siRNA, the 3′ overhangs can be stabilized against degradation.

The siRNAs have significant sequence similarity to a target RNA so that the siRNAs can pair to the target RNA and result in sequence-specific degradation of the target RNA through an RNA interference mechanism. Optionally, the siRNA molecules include a 3′ hydroxyl group. In one embodiment, the RNA is stabilized by including purine nucleotides, such as adenosine or guanosine nucleotides. Alternatively, substitution of pyrimidine nucleotides by modified analogues, e.g., substitution of uridine nucleotide 3′ overhangs by 2′-deoxythymidine is tolerated and does not affect the efficiency of RNAi. The absence of a 2′-hydroxyl significantly enhances the nuclease resistance of the overhang in tissue culture medium and may be beneficial in vivo.

The siRNA can be a polynucleotide with a duplex, asymmetric duplex, hairpin or asymmetric hairpin secondary structure, having self-complementary sense and antisense regions, wherein the antisense region comprises nucleotide sequence that is complementary to nucleotide sequence in a separate target nucleic acid molecule or a portion thereof and the sense region having nucleotide sequence corresponding to the target nucleic acid sequence or a portion thereof. The siRNA can be a circular single-stranded polynucleotide having two or more loop structures and a stem comprising self-complementary sense and antisense regions, where the antisense region includes nucleotide sequence that is complementary to nucleotide sequence in a target nucleic acid molecule or a portion thereof and the sense region having nucleotide sequence corresponding to the target nucleic acid sequence or a portion thereof, and where the circular polynucleotide can be processed either in vivo or in vitro to generate an active siRNA molecule capable of mediating RNAi.

The siRNA can also include a single stranded polynucleotide having nucleotide sequence complementary to nucleotide sequence in a target nucleic acid molecule or a portion thereof (for example, where such siRNA molecule does not require the presence within the siRNA molecule of nucleotide sequence corresponding to the target nucleic acid sequence or a portion thereof), where the single stranded polynucleotide can further include a terminal phosphate group, such as a 5′-phosphate (see for example Martinez et al., 2002, Cell., 110, 563-574 and Schwarz et al., 2002, Molecular Cell, 10, 537-568), or 5′,3′-diphosphate. In certain embodiments, the siRNA molecule of the invention comprises separate sense and antisense sequences or regions, where the sense and antisense regions are covalently linked by nucleotide or non-nucleotide linkers molecules as is known in the art, or are alternately non-covalently linked by ionic interactions, hydrogen bonding, van der Waals interactions, hydrophobic interactions, and/or stacking interactions.

The siRNA need only be sufficiently similar to natural RNA that it has the ability to mediate RNAi. Thus, an siRNA can tolerate sequence variations that might be expected due to genetic mutation, strain polymorphism or evolutionary divergence. The number of tolerated nucleotide mismatches between the target sequence and the RNAi construct sequence is no more than 1 in 5 basepairs, or 1 in 10 basepairs, or 1 in 20 basepairs, or 1 in 50 basepairs. In some embodiments, the agent comprises a strand that has at least about 70%, e.g., at least about 80%, 84%, 89%, 90%, 91%, 92%, 93%, 94%, 95%, 96%, 97%, 98%, 99%, or 100% precise sequence complementarity with the target transcript over a window of evaluation between 15-29 nucleotides in length, such a sequence of at least 15 nucleotides, at least about 17 nucleotide, or at least about 18 or 19 to about 21-23 or 24-29 nucleotides in length. Alternatively worded, in an siRNA of about 19-25 nucleotides in length, siRNAs having no greater than about 4 mismatches are generally tolerated, as are siRNAs having no greater than 3 mismatches, 2 mismatches, and or 1 mismatch.

Mismatches in the center of the siRNA duplex are less tolerated, and may essentially abolish cleavage of the target RNA. In contrast, the 3′ nucleotides of the siRNA (e.g., the 3′ nucleotides of the siRNA antisense strand) typically do not contribute significantly to specificity of the target recognition. In particular, 3′ residues of the siRNA sequence which are complementary to the target RNA (e.g., the guide sequence) generally are not as critical for target RNA cleavage.

An siRNA suitable for delivery by a conjugate, particle or composition described herein may be defined functionally as including a nucleotide sequence (or oligonucleotide sequence) that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing). Additional preferred hybridization conditions include hybridization at 70° C. in 1×SSC or 50° C. in 1×SSC, 50% formamide followed by washing at 70° C. in 0.3×SSC or hybridization at 70° C. in 4×SSC or 50° C. in 4×SSC, 50% formamide followed by washing at 67° C. in 1×SSC. The hybridization temperature for hybrids anticipated to be less than 50 base pairs in length should be 5-10° C. less than the melting temperature (Tm) of the hybrid, where Tm is determined according to the following equations. For hybrids less than 18 base pairs in length, Tm(° C.)=2(# of A+T bases)+4(# of G+C bases). For hybrids between 18 and 49 base pairs in length, Tm(° C.)=81.5+16.6 (log 10[Na+])+0.41 (% G+C) (600/N), where N is the number of bases in the hybrid, and [Na+] is the concentration of sodium ions in the hybridization buffer ([Na+] for 1×SSC=0.165 M). Additional examples of stringency conditions for polynucleotide hybridization are provided in Sambrook, J., et al., 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M. Ausubel, et al., eds., John Wiley & Sons, Inc., sections 2.10 and 6.3-6.4, incorporated herein by reference. The length of the identical nucleotide sequences may be at least about 10, 12, 15, 17, 20, 22, 25, 27, 30, 32, 35, 37, 40, 42, 45, 47 or 50 bases.

As used herein, siRNA molecules need not be limited to those molecules containing only RNA, but may further encompass chemically-modified nucleotides and non-nucleotides. In certain embodiments, a therapeutic siRNA lacks 2′-hydroxy (2′-OH) containing nucleotides. In certain embodiments, a therapeutic siRNA does not require the presence of nucleotides having a 2′-hydroxy group for mediating RNAi and as such, an siRNA will not include any ribonucleotides (e.g., nucleotides having a 2′-OH group). Such siRNA molecules that do not require the presence of ribonucleotides to support RNAi can however have an attached linker or linkers or other attached or associated groups, moieties, or chains containing one or more nucleotides with 2′-OH groups. Optionally, an siRNA molecule can include ribonucleotides at about 5, 10, 20, 30, 40, or 50% of the nucleotide positions.

Other useful therapeutic siRNA oligonucleotides can have phosphorothioate backbones and oligonucleosides with heteroatom backbones, and in particular CH₂NHOCH₂, CH₂N(CH₃)OCH₂, CH₂ON(CH₃)CH₂, CH₂N(CH₃)N(CH₃)CH₂, and ON(CH₃)CH₂CH₂ (wherein the native phosphodiester backbone is represented as OPOCH₂) as taught in U.S. Pat. No. 5,489,677, and the amide backbones disclosed in U.S. Pat. No. 5,602,240.

Substituted sugar moieties also can be included in modified oligonucleotides. Therapeutic antisense oligonucleotides for delivery by a conjugate, particle or composition described herein can include one or more of the following at the 2′ position: OH; F; O-, S-, or N-alkyl; O-, S-, or N-alkenyl; O-, S-, or N-alkynyl; or O-alkyl-O-alkyl, wherein the alkyl, alkenyl and alkynyl can be substituted or unsubstituted C₁ to C₁₀ alkyl or C₂ to C₁₀ alkenyl and alkynyl. Useful modifications also can include O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(C₂)ₙCH₃]₂, where n and m are from 1 to about 10. In addition, oligonucleotides can include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, groups for improving the pharmacokinetic or pharmacodynamic properties of an oligonucleotide, and other substituents having similar properties. Other useful modifications include an alkoxyalkoxy group, e.g., 2′-methoxyethoxy (2′-OCH₂CH₂OCH₃), a dimethylaminooxyethoxy group (2′—O(CH₂)₂ON(CH₃)₂), or a dimethylamino-ethoxyethoxy group (2′-OCH₂OCH₂N(CH₂)₂). Other modifications can include 2′-methoxy (2′-OCH₃), 2′-aminopropoxy (2′-OCH₂CH₂CH₂NH₂), or 2′-fluoro (2′-F). Similar modifications also can be made at other positions within the oligonucleotide, such as the 3′ position of the sugar on the 3′ terminal nucleotide or in 2′-5′ linked oligonucleotides, and the 5′ position of the 5′ terminal nucleotide. Oligonucleotides also can have sugar mimetics such as cyclobutyl moieties in place of the pentofuranosyl group. References that teach the preparation of such substituted sugar moieties include U.S. Pat. Nos. 4,981,957 and 5,359,044.

An siRNA formulated with a polymer-nucleic acid agent conjugate, particle or composition described herein may include naturally occurring nucleosides (e.g., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose). Suitable modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Other useful nucleobases include those disclosed, for example, in U.S. Pat. No. 3,687,808.

A therapeutic siRNA for incorporation into a polymer-nucleic acid agent conjugate, particle or composition described herein may be chemically synthesized, or derived from a longer double-stranded RNA or a hairpin RNA. The siRNA can be produced enzymatically or by partial/total organic synthesis, and any modified ribonucleotide can be introduced by in vitro enzymatic or organic synthesis. A single-stranded species comprised at least in part of RNA may function as an siRNA antisense strand or may be expressed from a plasmid vector.

By “RNA interference” or “RNAi” is meant a process of inhibiting or down regulating gene expression in a cell as is generally known in the art and which is mediated by short interfering nucleic acid molecules. In addition, as used herein, the term RNAi is meant to be equivalent to other terms used to describe sequence specific RNA interference, such as post transcriptional gene silencing, translational inhibition, transcriptional inhibition, or epigenetics. For example, therapeutic siRNA molecules suitable for delivery by conjugate, particle or composition described herein can epigenetically silence genes at both the post-transcriptional level or the pre-transcriptional level. In a non-limiting example, epigenetic modulation of gene expression by siRNA molecules of the invention can result from siRNA mediated modification of chromatin structure or methylation patterns to alter gene expression. In another non-limiting example, modulation of gene expression by an siRNA molecule can result from siRNA mediated cleavage of RNA (either coding or non-coding RNA) via RISC, or alternately, translational inhibition as is known in the art. In another embodiment, modulation of gene expression by siRNA molecules of the invention can result from transcriptional inhibition. RNAi also includes translational repression by microRNAs or siRNAs acting like microRNAs. RNAi can be initiated by introduction of small interfering RNAs (siRNAs) or production of siRNAs intracellularly (e.g., from a plasmid or transgene), to silence the expression of one or more target genes. Alternatively, RNAi occurs in cells naturally to remove foreign RNAs (e.g., viral RNAs). Natural RNAi proceeds via dicer-directed fragmentation of precursor dsRNA which direct the degradation mechanism to other cognate RNA sequences.

As used herein, the term siRNA is meant to be equivalent to other terms used to describe nucleic acid molecules that are capable of mediating sequence specific RNAi, and includes, for example, short interfering RNA (siRNA), double-stranded RNA (dsRNA), short hairpin RNA (shRNA), short interfering oligonucleotide, short interfering nucleic acid, short interfering modified oligonucleotide, chemically-modified siRNA, post-transcriptional gene silencing RNA (ptgsRNA), and others.

miRNAs

In one embodiment, a therapeutic nucleic acid suitable for delivery by a polymer-nucleic acid agent conjugate, particle or composition described herein is a microRNA (miRNA). By “microRNA” or “miRNA” is meant a small double stranded RNA that regulates the expression of target messenger RNAs either by mRNA cleavage, translational repression/inhibition or heterochromatic silencing (see for example Ambros, 2004, Nature, 431, 350-355; Bartel, 2004, Cell, 116, 281-297; Cullen, 2004, Virus Research., 102, 3-9; He et al., 2004, Nat. Rev. Genet., 5, 522-531; and Ying et al., 2004, Gene, 342, 25-28). MicroRNAs (miRNAs) are small noncoding polynucleotides, about 22 nucleotides long, which direct destruction or translational repression of their mRNA targets.

In one embodiment, the therapeutic microRNA, has partial complementarity (i.e., less than 100% complementarity) between the sense strand or sense region and the antisense strand or antisense region of the miRNA molecule, or between the antisense strand or antisense region of the miRNA and a corresponding target nucleic acid molecule. For example, partial complementarity can include various mismatches or non-base paired nucleotides (e.g., 1, 2, 3, 4, 5 or more mismatches or non-based paired nucleotides, such as nucleotide bulges) within the double stranded nucleic acid molecule, structure which can result in bulges, loops, or overhangs that result between the sense strand or sense region and the antisense strand or antisense region of the miRNA or between the antisense strand or antisense region of the miRNA and a corresponding target nucleic acid molecule. Agents that act via the microRNA translational repression pathway contain at least one bulge and/or mismatch in the duplex formed with the target. In certain embodiments, a GU or UG base pair in a duplex formed by a guide strand and a target transcript is not considered a mismatch for purposes of determining whether an RNAi agent is targeted to a transcript.

In one embodiment, a therapeutic nucleic acid suitable for delivery by a polymer-nucleic acid agent conjugate, particle or composition described herein is an antagomir, which is a chemically modified oligonucleotide capable of inhibition of complementary miRNA, e.g., by promoting their degradation (see, e.g., Krutzfeldt et al., Nature, 438:685-689, 2005).

Antisense Oligonucleotides

Therapeutic “antisense oligonucleotides” are suitable for delivery via a polymer-nucleic acid agent conjugate, particle or composition described herein. The term “oligonucleotide” refers to an oligomer or polymer of ribonucleic acid (RNA) or deoxyribonucleic acid (DNA) or analogs thereof. This term includes oligonucleotides composed of naturally occurring nucleobases, sugars and covalent internucleoside (backbone) linkages, as well as oligonucleotides having non-naturally occurring portions which function similarly. Such modified or substituted oligonucleotides are often preferred over native forms because of desirable properties such as, for example, enhanced cellular uptake, enhanced affinity for a nucleic acid target, and increased stability in the presence of nucleases.

A therapeutic antisense oligonucleotide is typically from about 10 to about 50 nucleotides in length (e.g., 12 to 40, 14 to 30, or 15 to 25 nucleotides in length). Antisense oligonucleotides that are 15 to 23 nucleotides in length are particularly useful. However, an antisense oligonucleotide containing even fewer than 10 nucleotides (for example, a portion of one of the preferred antisense oligonucleotides) is understood to be included within the present invention so long as it demonstrates the desired activity of inhibiting expression of a target gene.

An antisense oligonucleotide may consist essentially of a nucleotide sequence that specifically hybridizes with an accessible region in the target nucleic acid. Such antisense oligonucleotides, however, may contain additional flanking sequences of 5 to 10 nucleotides at either end. Flanking sequences can include, for example, additional sequences of the target nucleic acid, sequences complementary to an amplification primer, or sequences corresponding to a restriction enzyme site.

For maximal effectiveness, further criteria can be applied to the design of antisense oligonucleotides. Such criteria are well known in the art, and are widely used, for example, in the design of oligonucleotide primers. These criteria include the lack of predicted secondary structure of a potential antisense oligonucleotide, an appropriate G and C nucleotide content (e.g., approximately 50%), and the absence of sequence motifs such as single nucleotide repeats (e.g., GGGG runs).

While antisense oligonucleotides are a preferred form of antisense compounds, the present invention includes other oligomeric antisense compounds, including but not limited to, oligonucleotide analogs such as those described below. As is known in the art, a nucleoside is a base-sugar combination, wherein the base portion is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric molecule. The respective ends of this linear polymeric molecule can be further joined to form a circular molecule, although linear molecules are generally preferred. Within the oligonucleotide molecule, the phosphate groups are commonly referred to as forming the internucleoside backbone of the oligonucleotide. The normal linkage or backbone of RNA and DNA is a 3′ to 5′ phosphodiester linkage.

The therapeutic antisense oligonucleotides suitable for delivery by a polymer-nucleic acid agent conjugate, particle or composition described herein include oligonucleotides containing modified backbones or non-natural internucleoside linkages. As defined herein, oligonucleotides having modified backbones include those that have a phosphorus atom in the backbone and those that do not have a phosphorus atom in the backbone. For the purposes of this specification, and as sometimes referenced in the art, modified oligonucleotides that do not have a phosphorus atom in their internucleoside backbone also can be considered to be oligonucleotides.

Modified oligonucleotide backbones can include, for example, phosphorothioates, chiral phosphorothioates, phosphorodithioates, phosphotriesters, aminoalkylphosphotriesters, methyl and other alkyl phosphonates (e.g., 3′-alkylene phosphonates and chiral phosphonates), phosphinates, phosphoramidates (e.g., 3′-amino phosphoramidate and aminoalkylphosphoramidates), thionophosphoramidates, thionoalkylphosphonates, thionoalkyl phosphotriesters, and boranophosphates having normal 3′-5′ linkages, as well as 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. References that teach the preparation of such modified backbone oligonucleotides are provided, for example, in U.S. Pat. Nos. 4,469,863 and 5,750,666.

Therapeutic antisense molecules with modified oligonucleotide backbones that do not include a phosphorus atom therein can have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. References that teach the preparation of such modified backbone oligonucleotides are provided, for example, in U.S. Pat. Nos. 5,235,033 and 5,596,086.

In another embodiment, a therapeutic antisense compound is an oligonucleotide analog, in which both the sugar and the internucleoside linkage (i.e., the backbone) of the nucleotide units are replaced with novel groups, while the base units are maintained for hybridization with an appropriate nucleic acid target. One such oligonucleotide analog that has been shown to have excellent hybridization properties is referred to as a peptide nucleic acid (PNA). In PNA compounds, the sugar-backbone of an oligonucleotide is replaced with an amide containing backbone (e.g., an aminoethylglycine backbone). The nucleobases are retained and are bound directly or indirectly to aza nitrogen atoms of the amide portion of the backbone. References that teach the preparation of such modified backbone oligonucleotides are provided, for example, in Nielsen et al., Science 254:1497-1500 (1991), and in U.S. Pat. No. 5,539,082.

Other useful therapeutic antisense oligonucleotides can have phosphorothioate backbones and oligonucleosides with heteroatom backbones, and in particular CH₂NHOCH₂, CH₂N(CH₃)OCH₂, CH₂ON(CH₃)CH₂, CH₂N(CH₃)N(CH₃)CH₂, and ON(CH₃)CH₂CH₂ (wherein the native phosphodiester backbone is represented as OPOCH₂) as taught in U.S. Pat. No. 5,489,677, and the amide backbones disclosed in U.S. Pat. No. 5,602,240.

Substituted sugar moieties also can be included in modified oligonucleotides. Therapeutic antisense oligonucleotides for delivery by a polymer-nucleic acid agent conjugate, particle or composition described herein can include one or more of the following at the 2′ position: OH; F; O-, S-, or N-alkyl; O-, S-, or N-alkenyl; O-, S-, or N-alkynyl; or O-alkyl-O-alkyl, wherein the alkyl, alkenyl and alkynyl can be substituted or unsubstituted C₁ to C₁₀ alkyl or C₂ to C₁₀ alkenyl and alkynyl. Useful modifications also can include O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(C₂)ₙCH₃]₂, where n and m are from 1 to about 10. In addition, oligonucleotides can include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, groups for improving the pharmacokinetic or pharmacodynamic properties of an oligonucleotide, and other substituents having similar properties. Other useful modifications include an alkoxyalkoxy group, e.g., 2′-methoxyethoxy (2′-OCH₂CH₂OCH₃), a dimethylaminooxyethoxy group (2′-O(CH₂)₂ON(CH₃)₂), or a dimethylamino-ethoxyethoxy group (2′-OCH₂OCH₂N(CH₂)₂). Other modifications can include 2′-methoxy (2′-OCH₃), 2′-aminopropoxy (2′-OCH₂CH₂CH₂NH₂), or 2′-fluoro (2′-F). Similar modifications also can be made at other positions within the oligonucleotide, such as the 3′ position of the sugar on the 3′ terminal nucleotide or in 2′-5′ linked oligonucleotides, and the 5′ position of the 5′ terminal nucleotide. Oligonucleotides also can have sugar mimetics such as cyclobutyl moieties in place of the pentofuranosyl group. References that teach the preparation of such substituted sugar moieties include U.S. Pat. Nos. 4,981,957 and 5,359,044.

Therapeutic antisense oligonucleotides can also include nucleobase modifications or substitutions. As used herein, “unmodified” or “natural” nucleobases include the purine bases adenine (A) and guanine (G), and the pyrimidine bases thymine (T), cytosine (C), and uracil (U). Modified nucleobases can include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Other useful nucleobases include those disclosed, for example, in U.S. Pat. No. 3,687,808.

Certain nucleobase substitutions can be particularly useful for increasing the binding affinity of the antisense oligonucleotides of the invention. For example, 5-methylcytosine substitutions have been shown to increase nucleic acid duplex stability by 0.6 to 1.2° C. (Sanghvi et al., eds., Antisense Research and Applications, pp. 276-278, CRC Press, Boca Raton, Fla. (1993)). Other useful nucleobase substitutions include 5-substituted pyrimidines, 6-azapyrimidines and N-2, N-6 and O-6 substituted purines such as 2-aminopropyladenine, 5-propynyluracil and 5-propynylcytosine.

It is not necessary for all nucleobase positions in a given antisense oligonucleotide be uniformly modified. More than one of the aforementioned modifications can be incorporated into a single oligonucleotide or even at a single nucleoside within an oligonucleotide. The therapeutic nucleic acids suitable for delivery by a conjugate, particle or compositions described herein also include antisense oligonucleotides that are chimeric oligonucleotides. “Chimeric” antisense oligonucleotides can contain two or more chemically distinct regions, each made up of at least one monomer unit (e.g., a nucleotide in the case of an oligonucleotide). Chimeric oligonucleotides typically contain at least one region wherein the oligonucleotide is modified so as to confer, for example, increased resistance to nuclease degradation, increased cellular uptake, and/or increased affinity for the target nucleic acid. For example, a region of a chimeric oligonucleotide can serve as a substrate for an enzyme such as RNase H, which is capable of cleaving the RNA strand of an RNA:DNA duplex such as that formed between a target mRNA and an antisense oligonucleotide. Cleavage of such a duplex by RNase H, therefore, can greatly enhance the effectiveness of an antisense oligonucleotide.

The therapeutic antisense oligonucleotides can be synthesized in vitro. Antisense oligonucleotides used in accordance with this invention can be conveniently produced through known methods, e.g., by solid phase synthesis. Similar techniques also can be used to prepare modified oligonucleotides such as phosphorothioates or alkylated derivatives.

Antisense polynucleotides include sequences that are complementary to a genes or mRNA. Antisense polynucleotides include, but are not limited to: morpholinos, 2′-O-methyl polynucleotides, DNA, RNA and the like. The polynucleotide-based expression inhibitor may be polymerized in vitro, recombinant, contain chimeric sequences, or derivatives of these groups. The polynucleotide-based expression inhibitor may contain ribonucleotides, deoxyribonucleotides, synthetic nucleotides, or any suitable combination such that the target RNA and/or gene is inhibited.

The term “hybridization,” as used herein, means hydrogen bonding, which can be Watson-Crick, Hoogsteen, or reversed Hoogsteen hydrogen bonding, between complementary nucleoside or nucleotide bases. For example, adenine and thymine, and guanine and cytosine, respectively, are complementary nucleobases (often referred to in the art simply as “bases”) that pair through the formation of hydrogen bonds. “Complementary,” as used herein, refers to the capacity for precise pairing between two nucleotides. For example, if a nucleotide at a certain position of an oligonucleotide is capable of hydrogen bonding with a nucleotide in a target nucleic acid molecule, then the oligonucleotide and the target nucleic acid are considered to be complementary to each other at that position. The oligonucleotide and the target nucleic acid are complementary to each other when a sufficient number of corresponding positions in each molecule are occupied by nucleotides that can hydrogen bond with each other. Thus, “specifically hybridizable” is used to indicate a sufficient degree of complementarity or precise pairing such that stable and specific binding occurs between the oligonucleotide and the target nucleic acid.

It is understood in the art that the sequence of an antisense oligonucleotide need not be 100% complementary to that of its target nucleic acid to be specifically hybridizable. An antisense oligonucleotide is specifically hybridizable when (a) binding of the oligonucleotide to the target nucleic acid interferes with the normal function of the target nucleic acid, and (b) there is sufficient complementarity to avoid non-specific binding of the antisense oligonucleotide to non-target sequences under conditions in which specific binding is desired, i.e., under conditions in which in vitro assays are performed or under physiological conditions for in vivo assays or therapeutic uses.

Stringency conditions in vitro are dependent on temperature, time, and salt concentration (see e.g., Sambrook et al., Molecular Cloning: A Laboratory Manual, Cold Spring Harbor Laboratory Press, NY (1989)). Typically, conditions of high to moderate stringency are used for specific hybridization in vitro, such that hybridization occurs between substantially similar nucleic acids, but not between dissimilar nucleic acids. Specific hybridization conditions are hybridization in 5×SSC (0.75 M sodium chloride/0.075 M sodium citrate) for 1 hour at 40° C., followed by washing 10 times in 1×SSC at 40° C. and 5× in 1×SSC at room temperature.

In vivo hybridization conditions consist of intracellular conditions (e.g., physiological pH and intracellular ionic conditions) that govern the hybridization of antisense oligonucleotides with target sequences. In vivo conditions can be mimicked in vitro by relatively low stringency conditions. For example, hybridization can be carried out in vitro in 2×SSC (0.3 M sodium chloride/0.03 M sodium citrate), 0.1% SDS at 37° C. A wash solution containing 4×SSC, 0.1% SDS can be used at 37° C., with a final wash in 1×SSC at 45° C.

The specific hybridization of an antisense molecule with its target nucleic acid can interfere with the normal function of the target nucleic acid. For a target DNA nucleic acid, antisense technology can disrupt replication and transcription. For a target RNA nucleic acid, antisense technology can disrupt, for example, translocation of the RNA to the site of protein translation, translation of protein from the RNA, splicing of the RNA to yield one or more mRNA species, and catalytic activity of the RNA. The overall effect of such interference with target nucleic acid function is, in the case of a nucleic acid encoding a target gene, inhibition of the expression of target gene. In the context of the present invention, “inhibiting expression of a target gene” means to disrupt the transcription and/or translation of the target nucleic acid sequences resulting in a reduction in the level of target polypeptide or a complete absence of target polypeptide.

An antisense oligonucleotide, e.g., an antisense strand of an siRNA may preferably be directed at specific targets within a target nucleic acid molecule. The targeting process includes the identification of a site or sites within the target nucleic acid molecule where an antisense interaction can occur such that a desired effect, e.g., inhibition of target gene expression, will result. Traditionally, preferred target sites for antisense oligonucleotides have included the regions encompassing the translation initiation or termination codon of the open reading frame (ORF) of the gene. In addition, the ORF has been targeted effectively in antisense technology, as have the 5′ and 3′ untranslated regions. Furthermore, antisense oligonucleotides have been successfully directed at intron regions and intron-exon junction regions.

Simple knowledge of the sequence and domain structure (e.g., the location of translation initiation codons, exons, or introns) of a target nucleic acid, however, is generally not sufficient to ensure that an antisense oligonucleotide directed to a specific region will effectively bind to and inhibit transcription and/or translation of the target nucleic acid. In its native state, an mRNA molecule is folded into complex secondary and tertiary structures, and sequences that are on the interior of such structures are inaccessible to antisense oligonucleotides. For maximal effectiveness, antisense oligonucleotides can be directed to regions of a target mRNA that are most accessible, i.e., regions at or near the surface of a folded mRNA molecule. Accessible regions of an mRNA molecule can be identified by methods known in the art, including the use of RiboTAG™, or mRNA Accessible Site Tagging (MAST), technology. RiboTAG™ technology is disclosed in PCT Application Number SE01/02054.

Once one or more target sites have been identified, antisense oligonucleotides can be synthesized that are sufficiently complementary to the target (i.e., that hybridize with sufficient strength and specificity to give the desired effect). The effectiveness of an antisense oligonucleotide to inhibit expression of a target nucleic acid can be evaluated by measuring levels of target mRNA or protein using, for example, Northern blotting, RT-PCR, Western blotting, ELISA, or immunohistochemical staining.

In some embodiments, it may be useful to target multiple accessible regions of a target nucleic acid. In such embodiments, multiple antisense oligonucleotides can be used that each specifically hybridize to a different accessible region. Multiple antisense oligonucleotides can be used together or sequentially. In some embodiments, it may be useful to target multiple accessible regions of multiple target nucleic acids

Aptamers

A therapeutic nucleic acid suitable for delivery by a polymer-nucleic acid agent conjugate, particle or composition described herein can be an aptamer (also called a nucleic acid ligand or nucleic acid aptamer), which is a polynucleotide that binds specifically to a target molecule where the nucleic acid molecule has a sequence that is distinct from a sequence recognized by the target molecule in its natural setting. Alternately, an aptamer can be a nucleic acid molecule that binds to a target molecule where the target molecule does not naturally bind to a nucleic acid. The target molecule can be any molecule of interest. The target molecule can be, for example, a polypeptide, a carbohydrate, a nucleic acid molecule or a cell. The target of an aptamer is a three dimensional chemical structure that binds to the aptamer. For example, an aptamer that targets a nucleic acid (e.g., an RNA or a DNA) may include regions that bind via complementary Watson-Crick base pairing to a nucleic acid target interrupted by other structures such as hairpin loops. In another embodiment, the aptamer binds a target protein at a ligand-binding domain, thereby preventing interaction of the naturally occurring ligand with the target protein.

In one embodiment, the aptamer binds to a cell or tissue in a specific developmental stage or a specific disease state. A target is an antigen on the surface of a cell, such as a cell surface receptor, an integrin, a transmembrane protein, an ion channel or a membrane transport protein. In one embodiment, the target is a tumor-marker. A tumor-marker can be an antigen that is present in a tumor that is not present in normal tissue or an antigen that is more prevalent in a tumor than in normal tissue.

The nucleic acid that forms the nucleic acid ligand may be composed of naturally occurring nucleosides, modified nucleosides, naturally occurring nucleosides with hydrocarbon linkers (e.g., an alkylene) or a polyether linker (e.g., a PEG linker) inserted between one or more nucleosides, modified nucleosides with hydrocarbon or PEG linkers inserted between one or more nucleosides, or a combination of thereof. In one embodiment, nucleotides or modified nucleotides of the nucleic acid ligand can be replaced with a hydrocarbon linker or a polyether linker provided that the binding affinity and selectivity of the nucleic acid ligand is not substantially reduced by the substitution (e.g., the dissociation constant of the aptamer for the target is typically not greater than about 1×10⁻⁶ M).

An aptamer may be prepared by any method, such as by Systemic Evolution of Ligands by Exponential Enrichment (SELEX). The SELEX process for obtaining nucleic acid ligands is described in U.S. Pat. No. 5,567,588, the entire teachings of which are incorporated herein by reference.

Within the particles described herein, the nucleic acid agent can be attached to another moiety such as a polymer described above, a cationic moiety described herein, or a hydrophilic polymer such as PEG. The nucleic acid agent can also be “free,” meaning not attached to another moiety. Where a particle includes a plurality of nucleic acid agents, some of the nucleic acid agents can be attached to another moiety and some can be free. For example, in certain embodiments, the nucleic acid agent in the particle is attached to a polymer of the particle. The nucleic acid agent may be attached to any polymer in the particle, e.g., a hydrophobic polymer or a polymer containing a hydrophilic and a hydrophobic portion.

In certain embodiments, a nucleic acid is “free” in the particle. The nucleic acid agent may be associated with a polymer or other component of the particle through one or more non-covalent interactions such as van der Waals interactions, hydrophobic interactions, hydrogen bonding, dipole-dipole interactions, ionic interactions, and pi stacking.

A nucleic acid agent may be present in varying amounts of a polymer-nucleic acid agent conjugate, particle or composition described herein. When present in a particle, the nucleic acid agent may be present in an amount, e.g., from about 0.1 to about 50% by weight of the particle (e.g., from about 1% to about 50%, from about 1 to about 30% by weight of the particle, from about 1 to about 20% by weight of the particle, from about 4 to about 25% by weight of the particle, or from about 5 to about 13%, 14%, 15%, 16%, 17%, 18%, 19% or 20% by weight of the particle).

### Additional Components

In some embodiments, the particle further comprises a surfactant or a mixture of surfactants. In some embodiments, the surfactant is PEG, poly(vinyl alcohol) (PVA), poly(vinylpyrrolidone) (PVP), poloxamer, hexyldecyltrimethylammonium chloride, a polysorbate, a polyoxyethylene ester, a PEG-lipid (e.g., PEG-ceramide, d-alpha-tocopheryl polyethylene glycol 1000 succinate), 1,2-Distearoyl-sn-Glycero-3-[Phospho-rac-(1-glycerol)], lecithin, or a mixture thereof. In some embodiments, the surfactant is PVA and the PVA is from about 3 kDa to about 50 kDa (e.g., from about 5 kDa to about 45 kDa, about 7 kDa to about 42 kDa, from about 9 kDa to about 30 kDa, or from about 11 to about 28 kDa) and up to about 98% hydrolyzed (e.g., about 75-95%, about 80-90% hydrolyzed, or about 85% hydrolyzed) In some embodiments, the PVA has a viscosity of from about 2 to about 27 cP. In some embodiments, the PVA is a cationic PVA, for example, as described above, for example, a cationic moiety such as a cationic PVA can also serve as a surfactant. In some embodiments, the surfactant is polysorbate 80. In some embodiments, the surfactant is Solutol® HS 15. In some embodiments, the surfactant is not a lipid (e.g., a phospholipid) or does not comprise a lipid. In some embodiments, the surfactant is present in an amount of up to about 35% by weight of the particle (e.g., up to about 20% by weight or up to about 25% by weight, from about 15% to about 35% by weight, from about 20% to about 30% by weight, or from about 23% to about 26% by weight).

In some embodiments, the particle is associated with an excipient, e.g., a carbohydrate component, or a stabilizer or lyoprotectant, e.g., a carbohydrate component, stabilizer or lyoprotectant described herein. While not wishing to be bound be theory the carbohydrate component may act as a stabilizer or lyoprotectant. In some embodiments, the carbohydrate component, stabilizer or lyoprotectant, comprises one or more sugars, sugar alcohols, carbohydrates (e.g., sucrose, mannitol, cyclodextrin or a derivative of cyclodextrin (e.g. 2-hydroxypropyl-β-cyclodextrin, sometimes referred to herein as HP-β-CD, or sulfobutyl-CD, sometimes referred to herein as CYTOSOL.)), salt, PEG, PVP or crown ether. In some embodiments, the carbohydrate component, stabilizer or lyoprotectant comprises two or more carbohydrates, e.g., two or more carbohydrates described herein. In one embodiment, the carbohydrate component, stabilizer or lyoprotectant includes a cyclic carbohydrate (e.g., cyclodextrin or a derivative of cyclodextrin, e.g., an α-, β-, or γ-, cyclodextrin (e.g. 2-hydroxypropyl-β-cyclodextrin)) and a non-cyclic carbohydrate. Exemplary non-cyclic oligosaccharides include those of less than 10, 8, 6 or 4 monosaccharide subunits (e.g., a monosaccharide or a disaccharide (e.g., sucrose, trehalose, lactose, maltose) or combinations thereof). In some embodiments, the lyoprotectant is a monosaccharide such as a sugar alcohol (e.g., mannitol).

In an embodiment the carbohydrate component, stabilizer or lyoprotectant comprises a first and a second component, e.g., a cyclic carbohydrate and a non-cyclic carbohydrate, e.g., a mono-, di, or tetra saccharide.

In one embodiment, the weight ratio of cyclic carbohydrate to non-cyclic carbohydrate associated with the particle is a weight ratio described herein, e.g., 0.5:1.5 to 1.5:0.5.

In an embodiment the carbohydrate component, stabilizer or lyoprotectant comprises a first and a second component (designated here as A and B) as follows:
(A) comprises a cyclic carbohydrate and (B) comprises a disaccharide;
(A) comprises more than one cyclic carbohydrate, e.g., a β-cyclodextrin (sometimes referred to herein as β-CD) or a β-CD derivative, e.g., HP-β-CD, and (B) comprises a disaccharide;
(A) comprises a cyclic carbohydrate, e.g., a β-CD or a β-CD derivative, e.g., HP-β-CD, and
(B) comprises more than one disaccharide;
(A) comprises more than one cyclic carbohydrate, and (B) comprises more than one disaccharide;
(A) comprises a cyclodextrin, e.g., a β-CD or a β-CD derivative, e.g., HP-β-CD, and (B) comprises a disaccharide;
(A) comprises a β-cyclodextrin, e.g a β-CD derivative, e.g., HP-β-CD, and (B) comprises a disaccharide;
(A) comprises a β-cyclodextrin, e.g., a β-CD derivative, e.g., HP-β-CD, and (B) comprises sucrose;
(A) comprises a β-CD derivative, e.g., HP-β-CD, and (B) comprises sucrose;
(A) comprises a β-cyclodextrin, e.g., a β-CD derivative, e.g., HP-β-CD, and (B) comprises trehalose;
(A) comprises a β-cyclodextrin, e.g., a β-CD derivative, e.g., HP-β-CD, and (B) comprises sucrose and trehalose.
(A) comprises HP-β-CD, and (B) comprises sucrose and trehalose.

In an embodiment components A and B are present in the following ratio: 0.5:1.5 to 1.5:0.5. In an embodiment, components A and B are present in the following ratio: 3-1:0.4-2; 3-1:0.4-2.5; 3-1:0.4-2; 3-1:0.5-1.5; 3-1:0.5-1; 3-1:1; 3-1:0.6-0.9; and 3:1:0.7. In an embodiment, components A and B are present in the following ratio: 2-1:0.4-2; 3-1:0.4-2.5; 2-1:0.4-2; 2-1:0.5-1.5; 2-1:0.5-1; 2-1:1; 2-1:0.6-0.9; and 2:1:0.7. In an embodiment components A and B are present in the following ratio: 2-1.5:0.4-2; 2-1.5:0.4-2.5; 2-1.5:0.4-2; 2-1.5:0.5-1.5; 2-1.5:0.5-1; 2-1.5:1; 2-1.5:0.6-0.9; 2:1.5:0.7. In an embodiment components A and B are present in the following ratio: 2.5-1.5:0.5-1.5; 2.2-1.6:0.7-1.3; 2.0-1.7:0.8-1.2; 1.8:1; 1.85:1 and 1.9:1.

In an embodiment component A comprises a cyclodextin, e.g., a β-cyclodextrin, e.g., a β-CD derivative, e.g., HP-β-CD, and (B) comprises sucrose, and they are present in the following ratio: 2.5-1.5:0.5-1.5; 2.2-1.6:0.7-1.3; 2.0-1.7:0.8-1.2; 1.8:1; 1.85:1 and 1.9:1.

In some embodiments, the surface of the particle can be substantially coated with a surfactant or polymer, for example, PVA, polyoxazoline, polyvinylpyrrolidine, polyhydroxylpropylmethacrylamide, polysialic acid, or PEG.

### Conjugates

One or more of the components of the particle can be in the form of a conjugate, i.e., attached to another moiety. Exemplary conjugates include nucleic acid agent-polymer conjugates (e.g., a nucleic acid agent-hydrophobic polymer conjugate, a nucleic acid agent-hydrophobic-hydrophilic polymer conjugate, or a nucleic acid agent-hydrophilic polymer conjugate), cationic moiety-polymer conjugates (e.g., a cationic moiety-hydrophobic polymer conjugate or a cationic moiety-hydrophobic-hydrophilic polymer conjugate), nucleic acid agent-cationic polymer conjugates, and nucleic acid agent-hydrophobic moiety conjugates.

A nucleic acid agent-polymer conjugate described herein includes a polymer (e.g., a hydrophobic polymer, a hydrophilic polymer, or a hydrophilic-hydrophobic polymer) and a nucleic acid agent. A nucleic acid agent described herein may be attached to a polymer described herein, e.g., directly (e.g., without the presence of atoms from an intervening spacer moiety), or through a linker. A nucleic acid agent may be attached to a hydrophobic polymer (e.g., PLGA), a hydrophilic polymer (e.g., PEG) or a hydrophilic-hydrophobic polymer (e.g., PEG-PLGA). A nucleic acid agent may be attached to a terminal end of a polymer, to both terminal ends of a polymer, or to a point along a polymer chain. In some embodiments, multiple nucleic acid agents may be attached to points along a polymer chain, or multiple nucleic acid agents may be attached to a terminal end of a polymer via a multifunctional linker. A nucleic acid agent may be attached to a polymer described herein through the 2′, 3′, or 5′ position of the nucleic acid agent. In embodiments where the nucleic acid agent is double stranded (e.g., an siRNA), the nucleic acid agent can be attached through the sense or antisense strand.

A cationic moiety-polymer conjugate described herein includes a polymer (e.g., a hydrophobic polymer or a polymer containing a hydrophilic portion and a hydrophobic portion) and a cationic moiety. A cationic moiety described herein may be attached to a polymer described herein, e.g., directly (e.g., without the presence of atoms from an intervening spacer moiety), or through a linker. A cationic moiety may be attached to a hydrophobic polymer (e.g., PLGA) or a polymer having a hydrophobic portion and a hydrophilic portion (e.g., PEG-PLGA). A cationic moiety may be attached to a terminal end of a polymer, to both terminal ends of a polymer, or to a point along a polymer chain. In some embodiments, multiple cationic moieties may be attached to points along a polymer chain, or multiple cationic moieties may be attached to a terminal end of a polymer via a multifunctional linker.

A nucleic acid agent-cationic polymer conjugate described herein includes a cationic polymer (e.g., PEI, cationic PVA, poly(histidine), poly(lysine), or poly(2-dimethylamino)ethyl methacrylate) and a nucleic acid agent. A nucleic acid agent described herein may be attached to a polymer described herein, e.g., directly (e.g., without the presence of atoms from an intervening spacer moiety), or through a linker. A nucleic acid agent may be attached to a hydrophobic polymer (e.g., PLGA), a hydrophilic polymer (e.g., PEG) or a polymer having a hydrophobic portion and a hydrophilic portion (e.g., PEG-PLGA). A nucleic acid agent may be attached to a terminal end of a polymer, to both terminal ends of a polymer, or to a point along a polymer chain. In some embodiments, multiple nucleic acid agents may be attached to points along a polymer chain, or multiple nucleic acid agents may be attached to a terminal end of a polymer via a multifunctional linker.

In some embodiment a conjugate can include a nucleic acid that forms a duplex with a nucleic acid agent attached to a polymer described herein. For example, a polymer described herein can be attached to a nucleic acid oligomer (e.g., a single stranded DNA), which hybridizes with a nucleic acid agent to form a duplex. The duplex can be cleaved, releasing the nucleic acid agent in vivo, for example with a cellular nuclease.

Modes of Attachment

A nucleic acid agent or cationic moiety described herein may be directly (e.g., without the presence of atoms from an intervening spacer moiety), attached to a polymer or hydrophobic moiety described herein (e.g., a polymer). The attachment may be at a terminus of the polymer or along the backbone of the polymer. The nucleic acid agent, for example, when the nucleic acid agent is double stranded, can be attached to a polymer or a cationic moiety through the sense strand or the antisense strand. In some embodiments, the nucleic acid agent is modified at the point of attachment to the polymer; for example, a terminal hydroxy moiety of the nucleic acid agent (e.g., a 5′ or 3′ terminal hydroxyl moiety) is converted to a functional group that is reacted with the polymer (e.g., the hydroxyl moiety is converted to a thiol moiety). A reactive functional group of a nucleic acid agent or cationic moiety may be directly attached (e.g., without the presence of atoms from an intervening spacer moiety), to a functional group on a polymer. A nucleic acid agent or cationic moiety may be attached to a polymer via a variety of linkages, e.g., an amide, ester, sulfide (e.g., a maleimide sulfide), disulfide, succinimide, oxime, silyl ether, carbonate or carbamate linkage. For example, in one embodiment, a hydroxy group of a nucleic acid agent or cationic moiety may be reacted with a carboxylic acid group of a polymer, forming a direct ester linkage between the nucleic acid agent or cationic moiety and the polymer. In another embodiment, an amino group of a nucleic acid agent or cationic moiety may be linked to a carboxylic acid group of a polymer, forming an amide bond. In an embodiment a thiol modified nucleic acid agent may be reacted with a reactive moiety on the terminal end of the polymer (e.g., an acrylate PLGA, or a pyridinyl-SS-activated PLGA, or a maleimide activated PLGA) to form a sulfide or disulfide or thioether bond (i.e., sulfide bond). Exemplary modes of attachment include those resulting from click chemistry (e.g., an amide bond, an ester bond, a ketal, a succinate, or a triazole and those described in WO 2006/115547).

In some embodiments, a nucleic acid agent or cationic moiety may be directly attached (e.g., without the presence of atoms from an intervening spacer moiety), to a terminal end of a polymer. For example, a polymer having a carboxylic acid moiety at its terminus may be covalently attached to a hydroxy, thiol, or amino moiety of a nucleic acid agent or cationic moiety, forming an ester, thioester, or amide bond. In another embodiment, a nucleic acid agent or cationic moiety may be directly attached (e.g., without the presence of atoms from an intervening spacer moiety), along the backbone of a polymer. The nucleic acid agent, for example, when the nucleic acid agent is double stranded, can be attached to a polymer or a cationic moiety through the sense strand or the antisense strand.

In certain embodiments, suitable protecting groups may be required on the other polymer terminus or on other reactive substituents on the agent, to facilitate formation of the specific desired conjugate. For example, a polymer having a hydroxy terminus may be protected, e.g., with a silyl group (e.g., trimethylsilyl) or an acyl group (e.g., acetyl). A nucleic acid agent or cationic moiety may be protected, e.g., with an acetyl group or other protecting group.

In some embodiments, the process of attaching a nucleic acid agent or cationic moiety to a polymer may result in a composition comprising a mixture of conjugates having the same polymer and the same nucleic acid agent or cationic moiety, but which differ in the nature of the linkage between the nucleic acid agent or cationic moiety and the polymer. For example, when a nucleic acid agent or cationic moiety has a plurality of reactive moieties that may react with a polymer, the product of a reaction of the nucleic acid agent or cationic moiety and the polymer may include a conjugate wherein the nucleic acid agent or cationic moiety is attached to the polymer via one reactive moiety, and a conjugate wherein the nucleic acid agent or cationic moiety is attached to the polymer via another reactive moiety. For example, when a nucleic acid agent is attached to a polymer, the product of the reaction may include a conjugate where some of the nucleic acid agent is attached to the polymer through the 3′ end of the nucleic acid agent and some of the nucleic acid is attached to the polymer through the 5′ end of the nucleic acid agent. For example, when a nucleic acid agent having a double-stranded region is attached to a polymer, the product of the reaction may include a conjugate where some of the nucleic acid agent having a double-stranded region is attached to the polymer through the sense end and some of the nucleic acid agent having a double-stranded region is attached to the anti-sense end. Likewise, where a cationic moiety has multiple reactive groups such as a plurality of amines, the product of the reaction may include a conjugate where some of cationic moiety is attached to the polymer through a first reactive group and some of the cationic moiety is attached to the polymer through a second reactive group.

In some embodiments, the process of attaching a nucleic acid agent or cationic moiety to a polymer may involve the use of protecting groups. For example, when a nucleic acid agent or cationic moiety has a plurality of reactive moieties that may react with a polymer, the nucleic acid agent or cationic moiety may be protected at certain reactive positions such that a polymer will be attached via a specified position. In one embodiment, a nucleic acid or nucleic acid agent may be protected on the 3′ or 5′ end of the nucleic acid agent when attaching to a polymer. In one embodiment, a nucleic acid agent having a double-stranded region may be protected on the sense or anti-sense end when attaching to a polymer.

In some embodiments, selectively-coupled products such as those described above may be combined to form mixtures of polymer-agent conjugates. For example, PLGA attached to a nucleic acid agent through the 3′ end of the nucleic acid agent, and PLGA attached to a nucleic acid agent through the 5′ end of the nucleic acid agent, may be combined to form a mixture of the two conjugates, and the mixture may be used in the preparation of a particle. In another embodiment, PLGA attached to an siRNA through the sense end (e.g., the 5′ end of the sense strand), and PLGA attached to an siRNA through the anti-sense end, may be combined to form a mixture of the two conjugates, and the mixture may be used in the preparation of a particle.

A polymer-agent conjugate may comprise a single nucleic acid agent or cationic moiety attached to a polymer. The nucleic acid agent or cationic moiety may be attached to a terminal end of a polymer, or to a point along a polymer chain.

In some embodiments, the conjugate may comprise a plurality of nucleic acid agents or cationic moieties attached to a polymer (e.g., 2, 3, 4, 5, 6 or more agents may be attached to a polymer). The nucleic acid agents or cationic moieties may be the same or different. In some embodiments, a plurality of nucleic acid agents or cationic moieties may be attached to a multifunctional linker (e.g., a polyglutamic acid linker). In some embodiments, a plurality of nucleic acid agents or cationic moieties may be attached to points along the polymer chain.

Linkers

A nucleic acid agent or cationic moiety may be attached to a moiety such as a polymer or a hydrophobic moiety such as a lipid, or to each other, via a linker, such as a linker described herein. For example: a hydrophobic polymer may be attached to a cationic moiety; a hydrophobic polymer may be attached to a nucleic acid agent; a hydrophilic-hydrophobic polymer may be attached to a nucleic acid agent; a hydrophilic polymer may be attached to a nucleic acid agent; a hydrophilic polymer may be attached to a cationic moiety; or a hydrophobic moiety may be attached to a cationic moiety, or a nucleic acid agent may be attached to a cationic moiety. A nucleic acid agent may be attached to a moiety such as a polymer described herein through the 2′, 3′, or 5′ position of the nucleic acid agent, such as a terminal 2′, 3′, or 5′ position of the nucleic acid agent (e.g., through a linker described herein). In embodiments where the nucleic acid agent is double stranded (e.g., an siRNA), the nucleic acid agent can be attached through the sense or antisense strand. In some embodiments, the nucleic acid agent is attached through a terminal end of a polymer (e.g., a PLGA polymer, where the attachment is at the hydroxyl terminal or carboxy terminal).

In certain embodiments, a plurality of the linker moieties is attached to a polymer, allowing attachment of a plurality of nucleic acid agents or cationic moieties to the polymer through linkers, for example, where the linkers are attached at multiple places on the polymer such as along the polymer backbone. In some embodiments, a linker is configured to allow for a plurality of a first moiety to be linked to a second moiety through the linker, for example, a plurality of nucleic acid agents can be linked to a single polymer such as a PLGA polymer via a branched linker, wherein the branched linker comprises a plurality of functional groups through which the nucleic acid can be attached. In some embodiments, the nucleic acid agent or cationic moiety is released from the linker under biological conditions (i.e., cleavable under physiological conditions). In another embodiment a single linker is attached to a polymer, e.g., at a terminus of the polymer.

The linker may comprise, for example, an alkylene (divalent alkyl) group. In some embodiments, one or more carbon atoms of the alkylene linker may be replaced with one or more heteroatoms or functional groups (e.g., thioether, amino, ether, keto, amide, silyl ether, oxime, carbamate, carbonate, disulfide, or heterocyclic or heteroaromatic moieties). For example, an acrylate polymer (e.g., an acrylate PLGA) can be reacted with a thiol modified nucleic acid agent (e.g., a thiol modified siRNA) to form a nucleic acid agent-polymer conjugate attached through a sulfide bond (e.g., a thiopropionate linkage). The acrylate can be attached to a terminal end of the polymer (e.g., a hydroxyl terminal end of a PLGA polymer such as a 50:50 PLGA polymer) by reacting an acrylacyl chloride with the hydroxyl terminal end of the polymer.

In some embodiments, a linker, in addition to the functional groups that allow for attachment of a first moiety to a second moiety, has an additional functional group. In some embodiments, the additional functional group can be cleaved under physiological conditions. Such a linker can be formed, for example, by reacting a first activated moiety such as a nucleic acid agent or cationic moiety, e.g., a nucleic acid agent or cationic moiety described herein, with a second activated moiety such as a polymer, e.g., a polymer described herein, to produce a linker that includes a functional group that is formed by joining the nucleic acid agent or cationic moiety to the polymer. Optionally, the additional functional group can provide a site for additional attachments or allow for cleavage under physiological conditions. For example, the additional functional group may include a disulfide, ester, oxime, carbonate, carbamate, or amide bonds that are cleavable under physiological conditions. In some embodiments, one or both of the functional groups that attach the linker to the first or second moiety may be cleavable under physiological conditions such as esters, amides, or disulfides.

In some embodiments, the additional functional group is a heterocyclic or heteroaromatic moiety.

A nucleic acid agent may be attached through a linker (e.g., a linker comprising two or three functional groups such as a linker described herein) to a moiety such as a polymer described herein through the 2′, 3′, or 5′ position of the nucleic acid agent, such as a terminal 2′, 3′, or 5′ position of the nucleic acid agent. In embodiments where the nucleic acid agent is double stranded (e.g., an siRNA), the nucleic acid agent can be attached through the sense or antisense strand. In some embodiments, the nucleic acid agent is attached through a terminal end of a polymer (e.g., a PLGA polymer, where the attachment is at the hydroxyl terminal or carboxy terminal).

In some embodiments, the linker includes a moiety that can modulate the reactivity of a functional group in the linker (e.g., another functional group or atom that can increase or decrease the reactivity of a functional group, for example, under biological conditions).

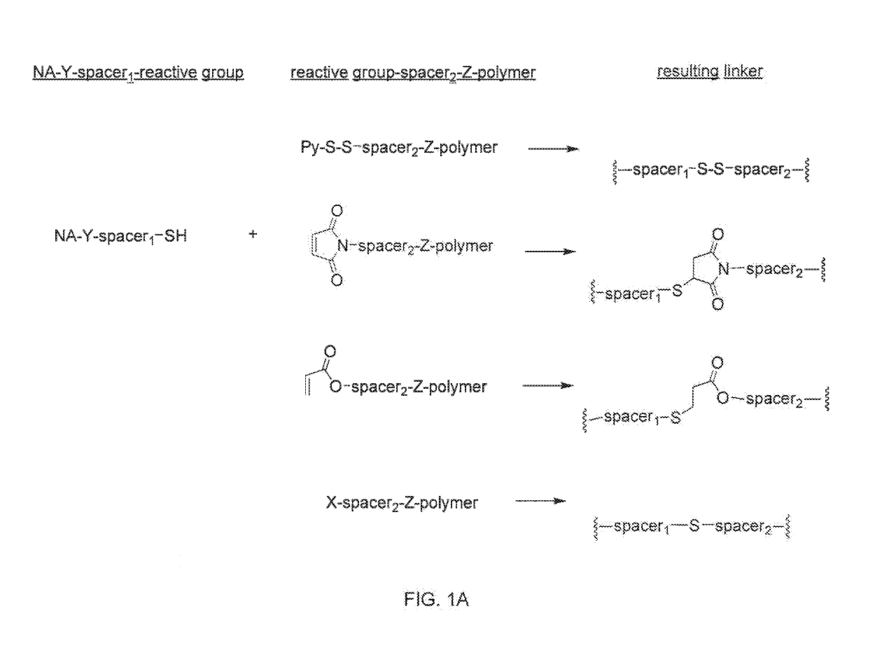

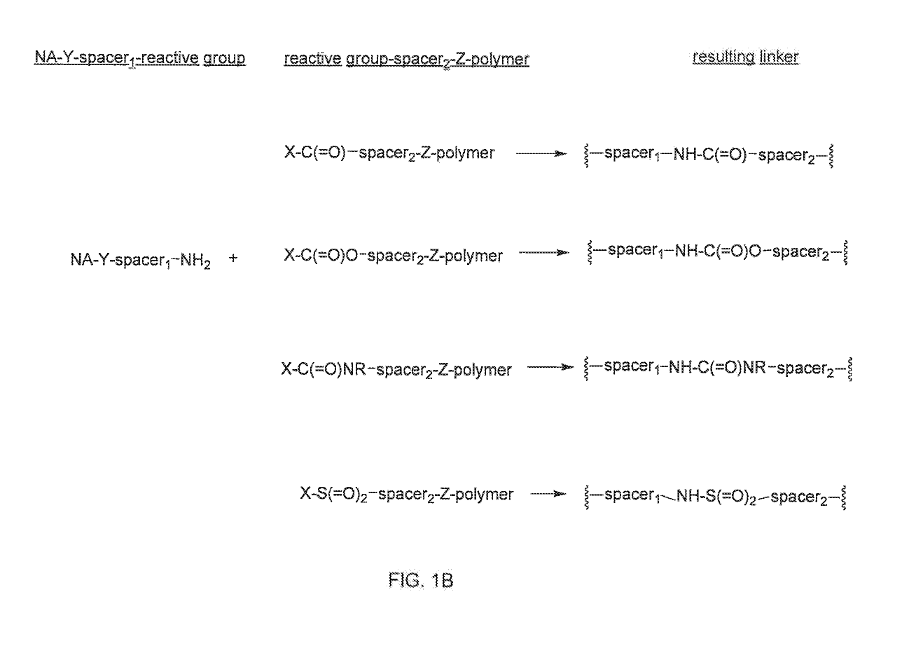

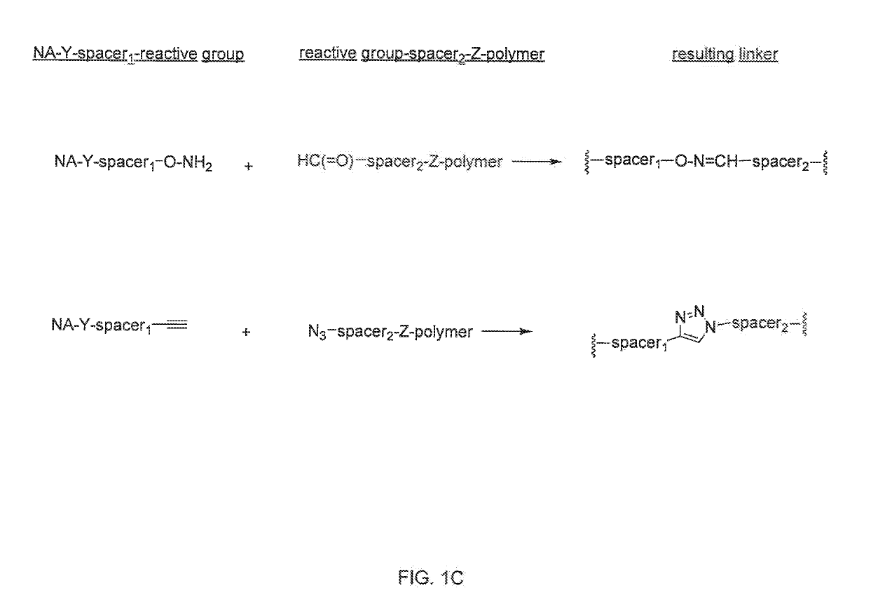

For example, as shown in FIGS. 1A-C, a nucleic acid agent (NA), e.g., RNA, having a first reactive group may be reacted with a polymer having a second reactive group to attach the nucleic acid agent to the polymer while providing a biocleavable functional group. The resulting linker includes a first spacer such as an alkylene spacer that attaches the nucleic acid agent to the functional group resulting from the attachment (i.e., by way of formation of a covalent bond), and a second spacer such as an alkylene spacer (e.g., from about C₁ to about C₆) that attaches the polymer to the functional group resulting from the attachment.

As shown in FIGS. 1A-C, the nucleic acid agent (NA) may be attached to the first spacer via a moiety Y, which also biocleavable. Y may be, for example, —O—, —S—, or —NH—. In some embodiments, the second spacer may be attached to a leaving group X—, for example halo (e.g., chloro) or N-hydroxysuccinimidyl (NHS). The second spacer may be attached to the polymer via an additional functional group (Z) that links with the polymer terminus, e.g., a terminal —OH, —CO₂H, —NH₂, or —SH, of a polymer, e.g., a terminal —OH or —CO₂H of PLGA. The additional functional group (Z) may be, for example, —O—, —OC(═O)—, —OC(═O)0-, —OC(═O)NR—, —NR—, —NRC(═O)—, —NRC(═O)O—, —NRC(═O)NR′—, —NRS(═O)₂—, —S—, —S(═O)—, —S(═O)₂—, —C(═O)O—, or —C(═O)NR—, and provides an additional site for reactivity, e.g., attachment or cleavage.

The nucleic acid agent may be attached through the 2′, 3′, or 5′ position of the nucleic acid agent, such as a terminal 2′, 3′, or 5′ position of the nucleic acid agent. In embodiments where the nucleic acid agent is double stranded (e.g., an siRNA), the nucleic acid agent can be attached through the sense or antisense strand. In some embodiments, the nucleic acid agent is attached through a spacer to the terminal end of a polymer (e.g., a PLGA polymer, where the attachment is at the hydroxyl terminal or carboxy terminal).

In an embodiment, e.g., as shown in FIG. 1A, a thiol modified nucleic acid agent (e.g., a thiol modified siRNA) can be reacted with a pyridynyl-SS-activated polymer (e.g., a pyridynyl-SS-activated PLGA, e.g., pyridynyl-SS-activated 5050 PLGA) to form a nucleic acid agent-polymer conjugate attached through a disulfide bond. In an embodiment, a thiol modified nucleic acid agent (e.g., a thiol modified siRNA) can be reacted with a maleimide-activated polymer (e.g., a maleimide-activated PLGA, e.g., maleimide-activated 5050 PLGA) to form a nucleic acid agent-polymer conjugate attached through a maleimide sulfide bond. In an embodiment, a thiol modified nucleic acid agent (e.g., a thiol modified siRNA) can be reacted with an acrylate-activated polymer (e.g., an acrylate-activated PLGA, e.g., acrylate-activated 5050 PLGA) to form a nucleic acid agent-polymer conjugate through a mercaptoproponate bond. The nucleic acid agent may be attached through the 2′, 3′, or 5′ position of the nucleic acid agent, such as a terminal 2′, 3′, or 5′ of the nucleic acid agent. In embodiments where the nucleic acid agent is double stranded (e.g., an siRNA), the nucleic acid agent can be attached through the sense or antisense strand. In some embodiments, the nucleic acid agent is attached through a spacer to the terminal end of a polymer (e.g., a PLGA polymer, where the attachment is at the hydroxyl terminal or carboxy terminal). In an embodiment, e.g., as shown in FIG. 1B, an amine modified nucleic acid agent (e.g., an amine modified siRNA) can be reacted with an polymer having an activated carboxylic acid or ester (e.g., an activated carboxylic acid PLGA, e.g., activated carboxylic acid 5050 PLGA, e.g., an SPA activated carboxylic acid PLGA, e.g., an SPA activated carboxylic acid 5050 PLGA) to form a nucleic acid agent-polymer conjugate attached through an amide bond. In an embodiment, an amine modified nucleic acid agent (e.g., an amine modified siRNA) can be reacted with an activated polymer (e.g., an activated PLGA, e.g., -activated 5050 PLGA) to form a nucleic acid agent-polymer conjugate attached through a carbamate bond. In an embodiment, an amine modified nucleic acid agent (e.g., an amine modified siRNA) can be reacted with an activated polymer (e.g., an activated PLGA, e.g., activated 5050 PLGA) to form a nucleic acid agent-polymer conjugate attached through a carbamide bond (urea). In an embodiment, an amine modified nucleic acid agent (e.g., an amine modified siRNA) can be reacted with an activated polymer (e.g., an activated PLGA, e.g., activated 5050 PLGA,) to form a nucleic acid agent-polymer conjugate attached through an aminoalkylsulfonamide bond. The nucleic acid agent may be attached through the 2′, 3′, or 5′ position of the nucleic acid agent, such as a terminal 2′, 3′, or 5′ of the nucleic acid agent. In embodiments where the nucleic acid agent is double stranded (e.g., an siRNA), the nucleic acid agent can be attached through the sense or antisense strand. In some embodiments, the nucleic acid agent is attached through a spacer to the terminal end of a polymer (e.g., a PLGA polymer, where the attachment is at the hydroxyl terminal or carboxy terminal).

In an embodiment, e.g., as shown in FIG. 1C, a hydroxylamine modified nucleic acid agent (e.g., a hydroxylamine modified siRNA) can be reacted with an aldehyde-activated polymer (e.g., an aldehyde-activated PLGA, e.g., aldehyde-activated 5050 PLGA, e.g., a formaldehyde-activated PLGA, e.g., formaldehyde-activated 5050 PLGA) to form a nucleic acid agent-polymer conjugate attached through an aldoxime bond. The nucleic acid agent may be attached through the 2′, 3′, or 5′ position of the nucleic acid agent, such as a terminal 2′, 3′, or 5′ of the nucleic acid agent. In embodiments where the nucleic acid agent is double stranded (e.g., an siRNA), the nucleic acid agent can be attached through the sense or antisense strand. In some embodiments, the nucleic acid agent is attached through a spacer to the terminal end of a polymer (e.g., a PLGA polymer, where the attachment is at the hydroxyl terminal or carboxy terminal).

In an embodiment, e.g., as shown in FIG. 1C, an alkylyne modified nucleic acid agent (e.g., an alkylyne modified siRNA, e.g., an acetylene modified siRNA) can be reacted with an azide-activated polymer (e.g., an azide-activated PLGA, e.g., azide-activated 5050 PLGA) to form a nucleic acid agent-polymer conjugate attached through a triazole bond. The nucleic acid agent may be attached through the 2′, 3′, or 5′ position of the nucleic acid agent, such as a terminal 2′, 3′, or 5′ of the nucleic acid agent. In embodiments where the nucleic acid agent is double stranded (e.g., an siRNA), the nucleic acid agent can be attached through the sense or antisense strand. In some embodiments, the nucleic acid agent is attached through a spacer to the terminal end of a polymer (e.g., a PLGA polymer, where the attachment is at the hydroxyl terminal or carboxy terminal).

In some embodiments, the linker, prior to attachment to the agent and the polymer, may have one or more of the following functional groups: amine, amide, hydroxyl, carboxylic acid, ester, halogen, thiol, maleimide, carbonate, or carbamate. In some embodiments, the functional group remains in the linker subsequent to the attachment of the first and second moiety through the linker. In some embodiments, the linker includes one or more atoms or groups that modulate the reactivity of the functional group (e.g., such that the functional group cleaves such as by hydrolysis or reduction under physiological conditions).

In some embodiments, the linker may comprise an amino acid or a peptide within the linker. Frequently, in such embodiments, the peptide linker is cleavable by hydrolysis, under reducing conditions, or by a specific enzyme (e.g., under physiological conditions).

When the linker is the residue of a divalent organic molecule, the cleavage of the linker may be either within the linker itself, or it may be at one of the bonds that couples the linker to the remainder of the conjugate, e.g. either to the nucleic acid agent or the polymer.

In some embodiments, a linker may be selected from one of the following or a linker may comprise one of the following:

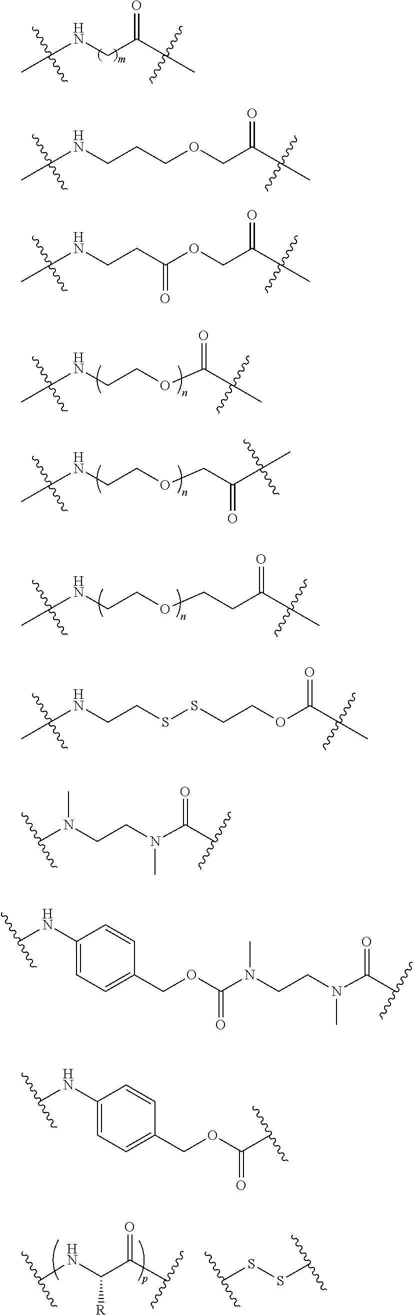

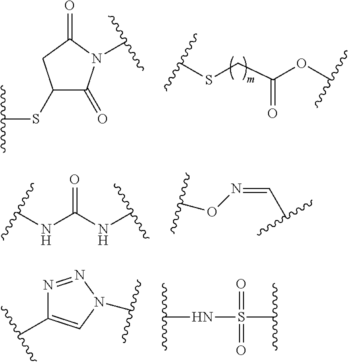

wherein m is 1-10, n is 1-10, p is 1-10, and R is an amino acid side chain.

A linker may include a bond resulting from click chemistry (e.g., an amide bond, an ester bond, a ketal, a succinate, or a triazole and those described in WO 2006/115547). A linker may be, for example, cleaved by hydrolysis, reduction reactions, oxidative reactions, pH shifts, photolysis, or combinations thereof; or by an enzyme reaction. The linker may also comprise a bond that is cleavable under oxidative or reducing conditions, or may be sensitive to acids.

In some embodiments, the linker is not cleaved under physiological conditions, for example, the linker is of a sufficient length that the nucleic acid agent does not need to be cleaved to be active, e.g., the length of the linker is at least about 20 angstroms (e.g., at least about 24 angstroms).

Methods of Making Conjugates

The conjugates may be prepared using a variety of methods, including those described herein. In some embodiments, to covalently link the nucleic acid agent or cationic moiety to a polymer, the polymer or agent may be chemically activated using a technique known in the art. The activated polymer is then mixed with the agent, or the activated agent is mixed with the polymer, under suitable conditions to allow a covalent bond to form between the polymer and the agent. In some embodiments, a nucleophile, such as a thiol, hydroxyl group, or amino group, on the agent attacks an electrophile (e.g., activated carbonyl group) to create a covalent bond. A nucleic acid agent or cationic moiety may be attached to a polymer via a variety of linkages, e.g., an amide, ester, succinimide, carbonate or carbamate linkage.

In some embodiments, a nucleic acid agent or cationic moiety may be attached to a polymer via a linker. In such embodiments, a linker may be first covalently attached to a polymer, and then attached to a nucleic acid agent or cationic moiety. In other embodiments, a linker may be first attached to a nucleic acid agent or cationic moiety, and then attached to a polymer.

In some embodiments, where the method includes forming a nucleic acid agent-polymer conjugate such as a nucleic acid agent-hydrophobic polymer conjugate or a nucleic acid agent-hydrophobic-hydrophilic-polymer conjugate, the solubility of the nucleic acid agent and the polymer are significantly different. For example, the nucleic acid agent can be highly water soluble and the polymer (e.g., a hydrophobic polymer) can have low solubility (e.g., less than about 1 mg/mL). Such reactions can be done in a single solvent, or a solvent system comprising a plurality of solvents (e.g., miscible solvents). The solvent system can include water (e.g., an aqueous buffer system) and a polar solvent such as dimethylformamide (DMF), dimethylsulfoxide (DMSO), dimethylacetamine (DMA), N-methylpyrolydine (NMP), hexamethylphosphoramide (HMPA), fluoroisopropanol, trifluoroethanol, propylene carbonate, acetone, benzyl alcohol, dioxane, tetrahydrofuran (THF), or acetonitrile (e.g., ACN). Exemplary aqueous buffers include phosphate buffer solution (PBS),4-(2-hydroxyethyl)-1-piperazineethanesulfonice acid (HEPES), TE buffer, or 2-(N-morpholino)ethanesulfonic acid buffer (MES)). The solvent system can be bi-phasic (e.g., having an organic and aqueous phase). In some embodiments, the ratio of polar solvent (e.g., “org”) to water (e.g., an aqueous buffer system) is from about 90/10 to about 40/60 (e.g., from about 80/10 to about 50/50, from about 80/10 to about 60/40, about 80/20, about 60/40 or about 50/50).

Exemplary solvent systems that can be used to attach a nucleic acid agent to a hydrophobic polymer include those in Table 1 below.

TABLE 1

| | 50/50 | 60/40 | 60/40 | 80/20 | 80/20 |
|---|---|---|---|---|---|
| Solvent | Org*/PBS** | Org/TE*** | Org/PBS | Org/TE | Org/PBS |
| DMSO | Translucent | Translucent | Turbid | Translucent | Translucent |
| | Some ppt. | Some ppt. | | | |
| Acetonitrile | Translucent | Milky | Translucent | Clear | Clear |
| | oil droplets | | Some tiny oil | | |
| | | | droplets | | |
| Acetone | Translucent | Milky | Translucent | Milky | Translucent |
| | Some tiny oil | | Some tiny oil | | |
| | droplets | | droplets | | |
| THF | Translucent | Milky | Translucent | Translucent | Translucent |
| | Some tiny oil | | Some tiny oil | | |
| | droplets | | droplets | | |
| DMF | Milky | Milky | Milky | Milky | Translucent |
| | | | | | w/ppt |

The above table is for a concentration of 10 mg/mL polymer.
*Org refers to an organic solvent.
**TE refers to an aqueous buffer solution having TE as the buffer (i.e., 1 mM Tris, brrought to pH 8.0 with HCl, and 1 mM EDTA)
***PBS refers to an aqueous buffer solution having PBS as the buffer (i.e., phosphate buffered saline.

Exemplary solvent systems that can be used to attach a nucleic acid agent to a hydrophobic-hydrophilic polymer include those in Table 2 below.

TABLE 2

| | 50/50 | 60/40 | 60/40 | 80/20 | 80/20 |
|---|---|---|---|---|---|
| Solvent | Org*/PBS*** | Org/TE** | Org/PBS | Org/TE | Org/PBS |
| DMSO | Translucent | Translucent | Turbid | Translucent | Translucent |
| | | Some ppt | | | |
| Acetonitrile | Clear | Clear | Clear | Clear | Clear |
| Acetone | Clear | Clear | Clear | Milky | Clear |
| THF | Clear | Clear | Translucent | Translucent | Clear |
| DMF | Slight | Translucent | Translucent | Milky | Translucent |
| | translucent | | | | w/oil droplet |

The above table is for a concentration of 10 mg/mL polymer.
*Org refers to an organic solvent.
**TE refers to an aqueous buffer solution having TE as the buffer (i.e., 1 mM Tris, brrought to pH 8.0 with HCl, and 1 mM EDTA)
***PBS refers to an aqueous buffer solution having PBS as the buffer (i.e., phosphate buffered saline.

The methods described herein can be done using an excess of one or more reagents. For example, when forming a nucleic acid agent polymer conjugate, the reaction can be performed using an excess of either the polymer or the nucleic acid agent.

The methods described herein can be performed where at least one of the nucleic acid agent or polymer is attached to an insoluble substrate (e.g., the polymer).

The methods described herein can result in a nucleic acid agent-polymer conjugate having a purity of at least about 80% (e.g., at least about 85%, at least about 90%, at least about 95%, at least about 99%). In some embodiments, method produces at least about 100 mg of the nucleic acid agent-polymer conjugate (e.g., at least about 1 g).

### Compositions of Conjugates

Compositions of conjugates described above (e.g., nucleic acid agent-polymer conjugates or cationic moiety-polymer conjugates) may include mixtures of products. For example, the conjugation of a nucleic acid agent or cationic moiety to a polymer may proceed in less than 100% yield, and the composition comprising the conjugate may thus also include unconjugated polymer, unconjugated nucleic acid agent, and/or unconjugated cationic moiety.

Compositions of conjugates (nucleic acid agent-polymer conjugates or cationic moiety-polymer conjugates) may also include conjugates that have the same polymer and the same nucleic acid agent and/or cationic moiety, and differ in the nature of the linkage between the nucleic acid agent and/or cationic moiety and the polymer. For example, in some embodiments, when the conjugate is a nucleic acid agent-polymer conjugate, the composition may include polymers attached to the nucleic acid agent via different hydroxyl groups present on the nucleic acid agent (e.g., the 2′, 3′, or 5′ hydroxyl groups such as the 3′ or 5′). When the conjugate is a cationic moiety-polymer conjugate and the cationic moiety includes multiple reactive groups, the composition may include polymers attached to the cationic moiety via different reactive groups present on the cationic moiety (e.g., different reactive amines).

The conjugates may be present in the composition in varying amounts. For example, when a nucleic acid agent and/or cationic moiety having a plurality of available attachment points is reacted with a polymer, the resulting composition may include more of a product conjugated via a more reactive group (e.g., a first hydroxyl or amino group), and less of a product attached via a less reactive group (e.g., a second hydroxyl or amino group).

Additionally, compositions of conjugates may include nucleic acid agents and/or cationic moieties that are attached to more than one polymer chain. For example, in the case of a nucleic acid agent-polymer conjugate, the nucleic acid agent may be attached to a first polymer chain through a 3′ hydroxyl and a second polymer chain through a 5′ hydroxyl. For example, in the case of a cationic moiety-polymer conjugate wherein cationic moiety includes multiple reactive groups, the cationic moiety may be attached to a first polymer chain through a first reactive group (e.g., a first amine) and a second polymer chain through a second reactive group (e.g., a second amine).

### Methods of Making Particles and Compositions

A particle described herein may be prepared using any method known in the art for preparing particles, e.g., nanoparticles. Exemplary methods include spray drying, emulsion (e.g., emulsion-solvent evaporation or double emulsion), precipitation (e.g., nanoprecipitation) and phase inversion.

In one embodiment, a particle described herein can be prepared by precipitation (e.g., nanoprecipitation). This method involves dissolving the components of the particle (i.e., one or more polymers, an optional additional component or components, a cationic moiety and a nucleic acid agent), individually or combined, in one or more solvents to form one or more solutions. For example, a first solution containing one or more of the components may be poured into a second solution containing one or more of the components (at a suitable rate or speed). The solutions may be combined, for example, using a syringe pump, a MicroMixer, or any device that allows for vigorous, controlled mixing. In some cases, nanoparticles can be formed as the first solution contacts the second solution, e.g., precipitation of the polymer upon contact causes the polymer to form nanoparticles. The control of such particle formation can be readily optimized.

In one set of embodiments, the particles are formed by providing one or more solutions containing one or more polymers and additional components, and contacting the solutions with certain solvents to produce the particle. In a non-limiting example, a hydrophobic polymer (e.g., PLGA), is conjugated to a nucleic acid agent or cationic moiety to form a conjugate. This polymer-conjugate, a polymer containing a hydrophilic portion and a hydrophobic portion (e.g., PEG-PLGA), nucleic acid agent and/or cationic moiety, and optionally a third polymer (e.g., a biodegradable polymer, e.g., PLGA) are dissolved in a partially water miscible organic solvent (e.g., acetone). This solution is added to an aqueous solution containing a surfactant, forming the desired particles. These two solutions may be individually sterile filtered prior to mixing/precipitation.

The formed nanoparticles can be exposed to further processing techniques to remove the solvents or purify the nanoparticles (e.g., dialysis). For purposes of the aforementioned process, water miscible solvents include acetone, ethanol, methanol, and isopropyl alcohol; and partially water miscible organic solvents include acetonitrile, tetrahydrofuran, ethyl acetate, isopropyl alcohol, isopropyl acetate or dimethylformamide.

Another method that can be used to generate a particle described herein is a process termed “flash nanoprecipitation” as described by Johnson, B. K., et al, AlChE Journal (2003) 49:2264-2282 and U.S. 2004/0091546, each of which is incorporated herein by reference in its entirety. This process is capable of producing controlled size, polymer-stabilized and protected nanoparticles of hydrophobic organics at high loadings and yields. The flash nanoprecipitation technique is based on amphiphilic diblock copolymer arrested nucleation and growth of hydrophobic organics. Amphiphilic diblock copolymers dissolved in a suitable solvent can form micelles when the solvent quality for one block is decreased. In order to achieve such a solvent quality change, a tangential flow mixing cell (vortex mixer) is used. The vortex mixer consists of a confined volume chamber where one jet stream containing the diblock copolymer and nucleic acid agent dissolved in a water-miscible solvent is mixed at high velocity with another jet stream containing water, an anti-solvent for the nucleic acid agent and the hydrophobic block of the copolymer. The fast mixing and high energy dissipation involved in this process provide timescales that are shorter than the timescale for nucleation and growth of particles, which leads to the formation of nanoparticles with nucleic acid agent loading contents and size distributions not provided by other technologies. When forming the nanoparticles via flash nanoprecipitation, mixing occurs fast enough to allow high supersaturation levels of all components to be reached prior to the onset of aggregation. Therefore, the nucleic acid agent(s) and polymers precipitate simultaneously, and overcome the limitations of low active agent incorporations and aggregation found with the widely used techniques based on slow solvent exchange (e.g., dialysis). The flash nanoprecipitation process is insensitive to the chemical specificity of the components, making it a universal nanoparticle formation technique.

A particle described herein may also be prepared using a mixer technology, such as a static mixer or a micro-mixer (e.g., a split-recombine micro-mixer, a slit-interdigital micro-mixer, a star laminator interdigital micro-mixer, a superfocus interdigital micro-mixer, a liquid-liquid micro-mixer, or an impinging jet micro-mixer).

A split-recombine micromixer uses a mixing principle involving dividing the streams, folding/guiding over each other and recombining them per each mixing step, consisting of 8 to 12 such steps. Mixing finally occurs via diffusion within milliseconds, exclusive of residence time for the multi-step flow passage. Additionally, at higher-flow rates, turbulences add to this mixing effect, improving the total mixing quality further.

A slit interdigital micromixer combines the regular flow pattern created by multi-lamination with geometric focusing, which speeds up liquid mixing. Due to this double-step mixing, a slit mixer is amenable to a wide variety of processes.

A particle described herein may also be prepared using Microfluidics Reaction Technology (MRT). At the core of MRT is a continuous, impinging jet microreactor scalable to at least 50 lit/min. In the reactor, high-velocity liquid reactants are forced to interact inside a microliter scale volume. The reactants mix at the nanometer level as they are exposed to high shear stresses and turbulence. MRT provides precise control of the feed rate and the mixing location of the reactants. This ensures control of the nucleation and growth processes, resulting in uniform crystal growth and stabilization rates.

A particle described herein may also be prepared by emulsion. An exemplary emulsification method is disclosed in U.S. Pat. No. 5,407,609, which is incorporated herein by reference. This method involves dissolving or otherwise dispersing agents, liquids or solids, in a solvent containing dissolved wall-forming materials, dispersing the nucleic acid agent/polymer-solvent mixture into a processing medium to form an emulsion and transferring all of the emulsion immediately to a large volume of processing medium or other suitable extraction medium, to immediately extract the solvent from the microdroplets in the emulsion to form a microencapsulated product, such as microcapsules or microspheres. The most common method used for preparing polymer delivery vehicle formulations is the solvent emulsification-evaporation method. This method involves dissolving the polymer and drug in an organic solvent that is completely immiscible with water (for example, dichloromethane). The organic mixture is added to water containing a stabilizer, most often poly(vinyl alcohol) (PVA) and then typically sonicated.

After the particles are prepared, they may be fractionated by filtering, sieving, extrusion, or ultracentrifugation to recover particles within a specific size range. One sizing method involves extruding an aqueous suspension of the particles through a series of polycarbonate membranes having a selected uniform pore size; the pore size of the membrane will correspond roughly with the largest size of particles produced by extrusion through that membrane. See e.g., U.S. Pat. No. 4,737,323, incorporated herein by reference. Another method is serial ultracentrifugation at defined speeds (e.g., 8,000, 10,000, 12,000, 15,000, 20,000, 22,000, and 25,000 rpm) to isolate fractions of defined sizes. Another method is tangential flow filtration, wherein a solution containing the particles is pumped tangentially along the surface of a membrane. An applied pressure serves to force a portion of the fluid through the membrane to the filtrate side. Particles that are too large to pass through the membrane pores are retained on the upstream side. The retained components do not build up at the surface of the membrane as in normal flow filtration, but instead are swept along by the tangential flow. Tangential flow filtration may thus be used to remove excess surfactant present in the aqueous solution or to concentrate the solution via diafiltration.

An exemplary method of making a particle described herein includes combining, in polar solvent (e.g., DMF, DMSO, acetone, benzyl alcohol, dioxane, tetrahydrofuran, or acetonitrile) under conditions that allow formation of a particle, e.g., by precipitation, (a) nucleic acid agent-hydrophobic polymer conjugates, each nucleic acid agent-hydrophobic polymer conjugate comprising a nucleic acid agent, e.g., an siRNA moiety, covalently attached to a hydrophobic polymer, wherein the nucleic acid agent-hydrophobic polymer conjugates are associated with a cationic moiety, (b) a plurality of hydrophilic-hydrophobic polymers, e.g., PEG-PLGA, and (c) a plurality of hydrophobic polymers (not covalently attached to a nucleic acid agent) to thereby form a particle. The combining can be done in a polar solvent, for example, acetone, or in a mixed solvent system (e.g., a combination aqueous/organic solvent system such as acetonitrile and an aqueous buffer system). The method can also include: (i) a plurality of nucleic acid agents, each nucleic acid agent comprising a nucleic acid agent, e.g., an siRNA or other nucleic acid agent, coupled to a hydrophobic polymer and associated with a cationic moiety, in acetonitrile/TE buffer (e.g., 80/20 wt %); with (ii) a plurality of hydrophilic-hydrophobic polymers, e.g., PEG-PLGA, and a plurality of hydrophobic polymers (not coupled to a nucleic acid agent), in acetonitrile/TE buffer (e.g., 80/20 wt %).

Another exemplary method of making a particle described herein includes: a) contacting, e.g., in an aqueous solvent i) a first plurality of hydrophobic-hydrophilic polymers, e.g., PEG-PLGA, with ii) a first plurality of hydrophobic polymers, e.g., PLGA, each having a first reactive moiety, e.g., a sulfhydryl moiety; to form a water soluble intermediate particle (e.g., having a diameter of less than about 100 nm); b) contacting, e.g., in aqueous solvent the intermediate particle with a plurality of water soluble nucleic acid agent, e.g., siRNA moieties, each having a second reactive moiety, e.g., an SH moiety, under conditions which allow formation of an intermediate complex, e.g., an intermediate structure comprising hydrophilic-hydrophobic polymers and hydrophobic polymers coupled to the drug moiety; and c) contacting, e.g., in a non-aqueous solvent, e.g., DMF, DMSO, acetone, benzyl alcohol, dioxane, tetrahydrofuran, or acetonitrile, the intermediate complex with a second plurality of hydrophilic-hydrophobic polymers, e.g., PEG-PLGA, and a second plurality of hydrophobic polymers, e.g., PLGA, under conditions that allow the formation of a particle, thereby forming a particle (wherein the formed particle is larger than the intermediate particle).

Another exemplary method of making a particle described herein includes a) contacting, e.g., in acetonitrile/TE buffer (e.g., 80/20 wt %) i) a first plurality of hydrophilic-hydrophobic polymers, e.g., PEG-PLGA, with ii) a first plurality of hydrophobic polymers, e.g., PLGA, each having a first reactive moiety, e.g., a sulfhydryl moiety; to form an intermediate particle (e.g., having a diameter of less than about 100 nm), wherein, In some embodiments, the intermediate particle is functionally soluble in aqueous solution, e.g., by virtue of having sufficient hydrophilic portion such that it is soluble in aqueous solution; b) contacting the intermediate particle with a plurality of nucleic acid agents, e.g., siRNA or other nucleic acid agents, each having a second reactive moiety, e.g., an SH moiety, under conditions which allow formation of an intermediate complex, e.g., an intermediate structure comprising hydrophilic-hydrophobic polymers and hydrophobic polymers coupled to the nucleic acid agent and, c) contacting the intermediate complex with a second plurality of hydrophilic-hydrophobic polymers, e.g., PEG-PLGA, and a second plurality of hydrophobic polymers, e.g., PLGA, under conditions that allow the formation of a particle, thereby forming a particle (e.g., wherein the diameter of the particle is less than 150 nm). A plurality of cationic moieties can be covalently attached to the hydrophobic polymers from b.

Another exemplary method of making a particle described herein includes dissolving cationic-PLGA and nucleic acid-conjugated 5050-O-acetyl-PLGA into a solution. The resulting solution will be added to water to form a nanoparticle suspension. A lipid mixture, e.g., including DOTAP, cholesterol and DOPE-PEG₂ₖ would be added to the particle suspension under conditions to allow the lipid mixture to coat the particle.

Another exemplary method of making a particle described herein includes dissolving nucleic acid-conjugated 5050-O-acetyl-PLGA (Mw ˜23.7 kDa) into a solution. The resulting solution will be added to water to form a nanoparticle suspension. A cationic polymer (e.g., polyhistidine, polylysine, polyarginine, polyethylene imine, and chitosan 60 wt. %) would be dissolved in acetone to form a 1% polymer solution and would be added to the particle suspension under conditions to allow the polymer mixture to coat the particle.

Another exemplary method of making a particle described herein includes forming a particle comprising a plurality of nucleic acid agent-polymer conjugates; contacting the particle with a plurality of cationic polyvalent polymers or lipids; and

contacting the product of b) with a plurality of polymers or lipids, wherein the a plurality of polymers or lipids substantially surround the product of b) forming the particle.

In some embodiments, the particle is further processed, for example, purified. Exemplary methods of purification include gel electrophoresis, capillary electrophoresis, gel permeation chromatography, dialysis, tangential flow filtration (e.g., using a 300 kDa filter), and size exclusion chromatography.

After purification of the particles, they may be sterile filtered (e.g., using a 0.22 micron filter) while in solution.

In certain embodiments, the particles are prepared to be substantially homogeneous in size within a selected size range. The particles are preferably in the range from 30 nm to 300 nm in their greatest diameter, (e.g., from about 30 nm to about 250 nm). The particles may be analyzed by techniques known in the art such as dynamic light scattering and/or electron microscopy, (e.g., transmission electron microscopy or scanning electron microscopy) to determine the size of the particles. The particles may also be tested for nucleic acid agent loading and/or the presence or absence of impurities (such as residual solvent).

Lyophilization

A particle described herein may be prepared for dry storage via lyophilization, commonly known as freeze-drying. Lyophilization is a process which extracts water from a solution to form a granular solid or powder. The process is carried out by freezing the solution and subsequently extracting any water or moisture by sublimation under vacuum. Advantages of lyophilization include maintenance of substance quality and minimization of therapeutic compound degradation. Lyophilization may be particularly useful for developing pharmaceutical drug products that are reconstituted and administered to a patient by injection, for example parenteral drug products. Alternatively, lyophilization is useful for developing oral drug products, especially fast melts or flash dissolve formulations.

Lyophilization may take place in the presence of a lyoprotectant, e.g., a lyoprotectant described herein. In some embodiments, the lyoprotectant is a carbohydrate (e.g., a carbohydrate described herein, such as, e.g., sucrose, cyclodextrin or a derivative of cyclodextrin (e.g. 2-hydroxypropyl-β-cyclodextrin)), salt, PEG, PVP or crown ether.

In some embodiments, aggregation of PEGylated particles during lyophilization may be reduced or minimized by the use of lyoprotectants comprising a cyclic oligosaccharide. Using suitable lyoprotectants provides lyophilized preparations that have extended shelf-lives.

The present disclosure features liquid formulations and lyophilized preparations that comprise a cyclic oligosaccharide. In some embodiments, the liquid formulation or lyophilized preparation can comprise at least two carbohydrates, e.g., a cyclic oligosaccharide (e.g., a cyclodextran or derivative thereof) and a non-cyclic oligosaccharide (e.g., a non-cyclic oligosaccharide less than about 10, 8, 6, 4 monosaccharides in length, e.g., a monosaccharide or disaccharide). In some embodiments, the liquid formulations also comprise a reconstitution reagent.

Examples of suitable cyclic oligosaccharides, include, but are not limited to, a-cyclodextrins, β-cyclodextrins, such as 2-hydroxypropyl-β-cyclodextrins, β-cyclodextrin sulfobutylethers sodiums, γ-cyclodextrins, any derivative thereof, and any combination thereof.

In certain embodiments, the cyclic carbohydrate, e.g., cyclic oligosaccharide, may be included in a larger molecular structure such as a polymer. Suitable polymers are disclosed herein with respect to the polymer composition of the particle. In such embodiments, the cyclic oligosaccharide may be incorporated within a backbone of the polymer. See, e.g., U.S. Pat. No. 7,270,808 and U.S. Pat. No. 7,091,192, which disclose exemplary polymers that contain cyclodextrin moieties in the polymer backbone that can be used in accordance with the invention. The entire teachings of U.S. Pat. No. 7,270,808 and U.S. Pat. No. 7,091,192 are incorporated herein by reference. In some embodiments, the cyclic oligosaccharide may contain at least one oxidized occurrence.

A lyoprotectant comprising a cyclic oligosaccharide, may inhibit the rate of intermolecular aggregation of particles that include hydrophilic polymers such as PEG during their lyophilization and/or storage, and therefore, provide for extended shelf-life. Without wishing to be limited by theory, the mechanism for the cyclic oligosaccharide to prevent particle aggregation may be due to the cyclic oligosaccharide reducing or preventing the crystallization of the hydrophilic polymer such as PEG present in the particles during lyophilization. This may occur through the formation of an inclusion complex between a cyclic oligosaccharide and the hydrophilic polymer (e.g., PEG). Such a complex may be formed between a cyclodextrin and, for example, the chain of polyethylene glycol. The inside cavity of cyclodextrin is lipophilic, while the outside of the cyclodextrin is hydrophilic. These properties may allow for the formation of inclusion complexes with other components of the particles described herein. For the purpose of stabilizing the formulations during lyophilization, the poly(ethyleneglycol) chain may fit into the cavity of the cyclodextrins. An additional mechanism that may allow the cyclic oligosaccharide to reduced or minimized or prevent particle degradation relates to the formation of hydrogen bonds between the cyclic oligosaccharide and the hydrophilic polymer (PEG) during lyophilization. For example, hydrogen bonding between cyclodextrin and poly(ethyleneglycol) chains may prevent ordered polyethylene glycol structures such as crystals.

The cyclic oligosaccharide may be present in varying amounts in the formulations described herein. In certain embodiments, the cyclic oligosaccharide to liquid formulation ratio is in the range of from about 0.75:1 to about 3:1 by weight. In preferred embodiments, the cyclic oligosaccharide to total polymer ratio is in the range of from about 0.75:1 to about 3:1 by weight.

In preferred aspects, the formulation contains two or more carbohydrates, e.g., a cyclic oligosaccharide and a non-cyclic carbohydrate, e.g., a non-cyclic oligosaccharide, e.g., a non-cyclic oligosaccharide having 10, 8, 6, 4 or less monosaccharide units. As described herein, including a non-cyclic carbohydrate, e.g., a non-cyclic oligosaccharide, into a liquid formulation that is to be lyophilized can promote uptake of water by the resulting lyophilized preparation, and promote disintegration of the lyophilized preparation.

In preferred aspects, the lyophilized or liquid formulation comprises a cyclic oligosaccharide, such as an α-cyclodextrin, β-cyclodextrin, γ-cyclodextrin, any derivative thereof, and any combination thereof, and a non-cyclic oligosaccharide, e.g., a non-cyclic oligosaccharide described herein. In some preferred embodiments, the lyoprotectant comprises a cyclic oligosaccharide, such as an α-cyclodextrin, β-cyclodextrin, γ-cyclodextrin, any derivative thereof, and any combination thereof, and the non-cyclic oligosaccharide is a disaccharide, such as sucrose, lactose, maltose, trehalose, and derivatives thereof, and a monosaccharide, such as glucose. In one preferred embodiment, the lyoprotectant comprises a β-cyclodextrin or derivative thereof, such as 2-hydroxypropyl-β-cyclodextrin or β-cyclodextrin sulfobutylether; and the non-cyclic oligosaccharide is a disaccharide, such as sucrose. The β-cyclodextrin or derivative thereof and the non-cyclic oligosaccharide can be present in any suitable relative amounts. Preferably, the ratio of cyclic oligosaccharide to non-cyclic oligosaccharide (w/w) is from about 0.5:1.5 to about 1.5:0.5, and more preferably from 0.7:1.3 to 1.3:0.7. In some examples, the ratio of cyclic oligosaccharide to non-cyclic oligosaccharide (w/w) is 0.7:1.3, 1:0.7, 1:1, 1.3:1 or 1.3:0.7. When the liquid or lyophilized formulation comprises a particle described herein, the ratio of cyclic oligosaccharide plus non-cyclic oligosaccharide to polymer (w/w) is from about 1:1 to about 10:1, and preferably, from about 1.1 to about 3:1.

In certain embodiments, the lyophilized preparations may be reconstituted with a reconstitution reagent. In some embodiments, a suitable reconstitution reagent may be any physiologically acceptable liquid. Suitable reconstitution reagents include, but are not limited to, water, 5% Dextrose Injection, Lactated Ringer's and Dextrose Injection, or a mixture of equal parts by volume of Dehydrated Alcohol, USP and a nonionic surfactant, such as a polyoxyethylated castor oil surfactant available from GAF Corporation, Mount Olive, N.J., under the trademark, Cremophor EL. To minimize the amount of surfactant in the reconstituted solution, only a sufficient amount of the vehicle may be provided to form a solution of the lyophilized preparation. Once dissolution of the lyophilized preparation is achieved, the resulting solution may be further diluted prior to injection with a suitable parenteral diluent. Such diluents are well known to those of ordinary skill in the art. These diluents are generally available in clinical facilities. Examples of typical diluents include, but are not limited to, Lactated Ringer's Injection, 5% Dextrose Injection, Sterile Water for Injection, and the like. However, because of its narrow pH range, pH 6.0 to 7.5, Lactated Ringer's Injection is most typical. Per 100 mL, lactated ringer's injection contains sodium chloride USP 0.6 g, sodium lactate 0.31 g, potassium chloride USP 0.03 g and calcium chloride₂H₂O USP 0.02 g. The osmolarity is 275 mOsmol/L, which is very close to isotonicity.

Accordingly, a liquid formulation can be a resuspended or rehydrated lyophilized preparation in a suitable reconstitution reagent. Suitable reconstitution reagents include physiologically acceptable carriers, e.g., a physiologically acceptable liquid as described herein. Preferably, resuspension or rehydration of the lyophilized preparations forms a solution or suspension of particles which have substantially the same properties (e.g., average particle diameter (Zave), size distribution (Dv₉₀, Dv₅₀), polydispersity, drug concentration) and morphology of the original particles in the liquid formulation of the present invention before lyophilization, and further maintains the therapeutic agent to polymer ratio of the original liquid formulation before lyophilization. In certain embodiments, about 50% to about 100%, preferably about 80% to about 100%, of the particles in the resuspended or rehydrated lyophilized preparation maintain the size distribution and/or drug to polymer ratio of the particles in the original liquid formulation. Preferably, the Zave, Dv₉₀, and polydispersity of the particles in the formulation produced by resuspending a lyophilized preparation do not differ from the Zave, Dv₉₀, and polydispersity of the particles in the original solution or suspension prior to lyophilization by more than about 5%, more than about 10%, more than about 15%, more than about 20%, more than about 15%, more than about 30%, more than about 35%, more than about 40%, more than about 45%, or more than about 50%.

Preferably liquid formulations of this aspect contain particles, and are characterized by a higher polymer concentration (the concentration of polymer(s) that form the particle) than can be lyophilized and resuspended using either a lyoprotectant that comprises one or more carbohydrates (e.g., a cyclic oligosaccharide and/or a non-cyclic oligosaccharide). For example, the polymer concentration can be at least about 20 mg/mL, at least about 25 mg/mL, at least about 30 mg/mL, at least about 31 mg/mL, at least about 32 mg/mL, at least about 33 mg/mL, at least about 34 mg/mL, at least about 35 mg/mL, at least about 36 mg/mL, at least about 37 mg/mL, at least about 38 mg/mL, at least about 39 mg/mL, at least about 40 mg/mL, at least about 45 mg/mL, at least about 50 mg/mL, at least about 55 mg/mL, at least about 60 mg/mL, at least about 65 mg/mL, at least about 70 mg/mL, at least about 75 mg/mL, at least about 80 mg/mL, at least about 85 mg/mL, at least about 90 mg/mL, at least about 95 mg/mL, are at least about 100 mg/mL. For example, the liquid formulation can be a reconstituted lyophilized preparation.

### Methods of Storing Particles and Compositions

In another aspect, the invention features, a method of storing a conjugate, particle or composition, e.g., a pharmaceutical composition.

In an embodiment, methods of storing a conjugate, particle, or composition described herein include, e.g., the steps of: (a) providing said conjugate, particle or composition disposed in a container; (b) storing said conjugate, particle or composition; and, optionally, (c) moving said container to a second location or removing all or an aliquot of said conjugate, particle or composition, from said container.

The conjugate, particle or composition can be in liquid, dry, lyophilized, or re-constituted (e.g., in a liquid as a solution or suspension) formulation or form. The conjugate, particle or composition can be stored in single, or multi-dose amounts, e.g., it can be stored in amounts sufficient for at least 2, 5, 10, or 100 dosages. In an embodiment, the method comprises dialyzing, diluting, concentrating, drying, lyophilizing, or packaging (e.g., disposing the material in a container) the conjugate, particle or composition. In an embodiment the method comprises combining the conjugate, particle or composition with another component, e.g., an excipient, lyoprotectant, or inert substance, e.g., an insert gas. In an embodiment the method comprises dividing a preparation of the conjugate, particle or composition into aliquots, and optionally disposing a plurality of aliquots in a plurality of containers. In embodiments conjugate, particle or composition, e.g., pharmaceutical composition, is stored for a period disclosed herein. In embodiments, after a period of storage, the stored conjugate, particle or composition, is evaluated, e.g., for aggregation, color, or other parameter.

In embodiments a conjugate, particle or composition described herein may be stored, e.g., in a container, for at least about 1 hour (e.g., at least about 2 hours, 4 hours, 8 hours, 12 hours, 24 hours, 2 days, 1 week, 1 month, 2 months, 3 months, 4 months, 5 months, 6 months, 1 year, 2 years or 3 years). Accordingly, described herein are containers including a conjugate, particle or composition described herein.

In embodiments, a conjugate, particle or composition may be stored under a variety of conditions, including ambient conditions, or other conditions described herein. In an embodiment a conjugate, particle or composition is stored at low temperature, e.g., at a temperature less than or equal to about 5° C. (e.g., less than or equal to about 4° C. or less than or equal to about 0° C.). A conjugate, particle or composition may also be frozen and stored at a temperature of less than about 0° C. (e.g., between −80° C. and −20° C.). A conjugate, particle or composition may also be stored under an inert atmosphere, e.g., an atmosphere containing an inert gas such as nitrogen or argon. Such an atmosphere may be substantially free of atmospheric oxygen and/or other reactive gases, and/or substantially free of moisture.

In some embodiments, a conjugate, particle or composition can be stored as a re-constituted formulation (e.g., in a liquid as a solution or suspension).

In an embodiment a conjugate, particle or composition described herein can be stored in a variety of containers, including a light-blocking container such as an amber vial. A container can be a vial, e.g., a sealed vial having a rubber or silicone enclosure (e.g., an enclosure made of polybutadiene or polyisoprene). A container can be substantially free of atmospheric oxygen and/or other reactive gases, and/or substantially free of moisture.

In another aspect, the invention features, a conjugate, particle or composition, disposed in a container, e.g., a container described herein, e.g., in an amount, form or formulation described herein.

### Methods of Evaluating Particles and Compositions

In another aspect, the invention features, a method of evaluating a particle or preparation of particles, e.g., for a property described herein. In an embodiment the property is a physical property, e.g., average diameter. In another embodiment the property is a functional property, e.g., the ability to mediate knockdown of a target gene, e.g., as measured in an assay described herein. The method comprises:

providing a sample comprising one or a plurality of said particles, e.g., as a composition, e.g., a pharmaceutical composition;

evaluating, e.g., by a physical test, a property described herein, to provide a determined value for the property,

thereby evaluating a particle or preparation of particles.

In an embodiment the method comprises one or both of:
a) comparing the determined value with a reference or standard value, e.g., a range of values (e.g., value disclosed herein, or set by a regulatory agency, manufacturer, or compendia authority), or
b) responsive to said determination or comparison, classifying said particles.

In an embodiment, responsive to said determination or comparison, a decision or step is taken, e.g., a production parameter in a process for making a particle is altered, the sample is classified, selected, accepted or discarded, released or withheld, processed into a drug product, shipped, moved to a different location, formulated, e.g., formulated with another substance, e.g., an excipient, labeled, packaged, released into commerce, or sold or offered for sale.

In an embodiment, the determined value for a property is compared with a reference, and responsive to said comparison said particle or preparation of particles is classified, e.g., as suitable for use in human subjects, not suitable for use in human subjects, suitable for sale, meeting a release specification, or not meeting a release specification.

In an embodiment a particle or preparation of particles is subjected to a measurement to determine whether an impurity or residual solvent is present (e.g., via gas chromatography (GC)), to determine relative amounts of one or more components (e.g., via high performance liquid chromatography (HPLC)), to measure particle size (e.g., via dynamic light scattering and/or scanning electron microscopy), or determine the presence or absence of surface components.

In an embodiment a particle or preparation of particles is evaluated for the average diameter of the particles in the composition. In an embodiment experiments including physical measurements are performed to determine average value. The average diameter of the composition can then be compared with a reference value. In an embodiment the average diameter for the particles is about 50 nm to about 500 nm (e.g., from about 50 nm to about 200 nm). A composition of a plurality of particles particle may have a median particle size (Dv50 (particle size below which 50% of the volume of particles exists) of about 50 nm to about 500 nm (e.g., about 75 nm to about 220 nm)) from about 50 nm to about 220 nm (e.g., from about 75 nm to about 200 nm). A composition of a plurality of particles may have a Dv90 (particle size below which 90% of the volume of particles exists) of about 50 nm to about 500 nm (e.g., about 75 nm to about 220 nm). In some embodiments, a composition of a plurality of particles has a Dv90 of less than about 150 nm. A composition of a plurality of particles may have a particle PDI of less than 0.5, less than 0.4, less than 0.3, less than 0.2, or less than 0.1.

In an embodiment a particle or preparation of particles is subjected to dynamic light scattering, e.g., to determine size or diameter. Particles may be illuminated with a laser, and the intensity of the scattered light fluctuates at a rate that is dependent upon the size of the particles as smaller particles are “kicked” further by the solvent molecules and move more rapidly. Analysis of these intensity fluctuations yields the velocity of the Brownian motion and hence the particle size using the Stokes-Einstein relationship. The diameter that is measured in dynamic light scattering is called the hydrodynamic diameter and refers to how a particle diffuses within a fluid. The diameter obtained by this technique is that of a sphere that has the same translational diffusion coefficient as the particle being measured.

In an embodiment a particle or preparation of particles is evaluated using cryo scanning electron microscopy (Cryo-SEM), e.g., to determine structure or composition. SEM is a type of electron microscopy in which the sample surface is imaged by scanning it with a high-energy beam of electrons in a raster scan pattern. The electrons interact with the atoms that make up the sample producing signals that contain information about the sample's surface topography, composition and other properties such as electrical conductivity. For Cryo-SEM, the SEM is equipped with a cold stage for cryo-microscopy. Cryofixation may be used and low-temperature scanning electron microscopy performed on the cryogenically fixed specimens. Cryo-fixed specimens may be cryo-fractured under vacuum in a special apparatus to reveal internal structure, sputter coated and transferred onto the SEM cryo-stage while still frozen.

In an embodiment a particle or preparation of particles is evaluated using transmission electron microscopy (TEM), e.g., to determine structure or composition. In this technique, a beam of electrons is transmitted through an ultra thin specimen, interacting with the specimen as it passes through. An image is formed from the interaction of the electrons transmitted through the specimen; the image is magnified and focused onto an imaging device, such as a fluorescent screen, on a layer of photographic film, or to be detected by a sensor such as a charge-coupled device (CCD) camera.

In an embodiment a particle or preparation of particles is evaluated for a surface zeta potential. In an embodiment experiments including physical measurements are performed to determine average value a surface zeta potential. The surface zeta potential can then be compared with a reference value. In an embodiment the surface zeta potential is between about −20 mV to about 50 mV, when measured in water. Zeta potential is a measurement of surface potential of a particle. In some embodiments, a particle may have a surface zeta potential, when measured in water, ranging between about −20 mV to about 20 mV, about −10 mV to about 10 mV, or neutral.

In an embodiment a particle or preparation of particles is evaluated for the effective amount of nucleic acid agent (e.g., an siRNA) it contains. In embodiment particles are administered, for example, in an in vivo model system, (e.g., a mouse model such as any of those described herein), and the level of effect (e.g., knock-down) observed. In embodiments the level is compared with a reference standard.

In an embodiment a particle or preparation of particles is evaluated for the presence of nucleic acid agent on its surface. For example, an intercalating agent such as RIBOGREEN, or HPLC, can be used to determine the presence or amount of a double stranded nucleic acid agent on the surface of the particle (e.g., the presence or amount of siRNA).

In an embodiment a particle or preparation of particles is evaluated for the amount of nucleic acid agent, e.g., siRNA, inside, as opposed to exposed at the surface, of the particle. In embodiments the level is compared with a reference standard. In embodiments at least 30, 40, 50, 60, 70, 80, or 90% of the nucleic acid agent, e.g., siRNA, by number or weight, in a particle is inside the particle.

In an embodiment a particle or preparation of particles is evaluated using an assay that provides information about the structure or function of the nucleic acid agent (e.g., a digestion assay). For example, the particle can be evaluated in an experiment that evaluates the ability of the nucleic acid agent to modulate expression of a target (e.g., knockdown). The particle can also be evaluated for its ability to treat a disorder, e.g, modulate tumor growth. In some embodiments, the evaluation is in an in vitro or in vivo assay (e.g., a xenograph model). The evaluation can be compared to a standard, and optionally, responsive to said standard, the particle is classified.

In an embodiment a particle or preparation of particles is evaluated for the ability to deliver a nucleic acid agent, e.g., an siRNA, that knocks down a target gene, in vivo, e.g., in an experimental animal, e.g., a mouse. The activity of the composition can be compared to that of an equal amount of free nucleic acid agent. In some embodiments the target gene is GFP the GFP is expressed in HeLA cells. E.g., the assay can use the anti-GFP siRNA, the GFP plasmid, the HeLA-GFP cells, the mice, and the GFP expression assays described in Bertrand et al., 2002, BBRC 296:1000-1004, hereby incorporated by reference. Other exemplary cells for evaluating conjugates, particles, and compositions include MDA-MB-435 and MDA-MB-468 GFP cells.

In an embodiment a particle or preparation of particles is evaluated for the ability to deliver a nucleic acid agent, e.g., an siRNA, that knocks down a target gene in vitro, e.g., in cultured cells. The activity of the composition can be compared to that of an equal amount of free nucleic acid agent. In some embodiments the target gene is GFP and the cultured cells are HeLA cells transfected with GFP. E.g., the assay can use the anti-GFP siRNA, the GFP plasmid, the HeLA-GFP cells, the cell culture conditions, and the GFP expression assay described in Bertrand et al., 2002, BBRC 296:1000-1004, hereby incorporated by reference. Other exemplary cells for evaluating particles and compositions described herein include MDA-MB-435 and MDA-MB-468 GFP cells.

In an embodiment a particle or preparation of particles is evaluated for the ability to deliver a nucleic acid agent, e.g., an siRNA, that knocks down a target gene in vitro, e.g., in cultured cells, after incubation in serum or a cell lysate. The activity of the treated composition can be compared to that of an equal amount of free nucleic acid agent. In some embodiments the target gene is GFP and the cultured cells are HeLA cells transfected with GFP. E.g., the assay can use the anti-GFP siRNA, the GFP plasmid, the HeLA-GFP cells, the cell culture conditions, the GFP expression assay, and, in the case of an assay that uses a cell lysate, the HeLa cell lysate, described in Bertrand et al., 2002, BBRC 296:1000-1004, hereby incorporated by reference. Alternatively, the mouse expression system described in Hu-Lieskovan et al., 2005, Cancer Res. 65: 8984-8992, hereby incorporated by reference, can be used to evaluate the performance of a composition. The target gene and constructs of Hu-Lieskovan et al., or other target genes and constructs can be used with the mouse system described in Hu-lieskovan et al. Other exemplary cells for evaluating particles and compositions described herein include MDA-MB-435 and MDA-MB-468 GFP cells.

In an embodiment a particle or preparation of particles is evaluated for the ability to protect a nucleic acid agent from a degradant such as an RNase (e.g., RNase A). In some embodiments, a composition described herein can confer protection on a nucleic acid agent such as an siRNA relative to untreated nucleic acid agent (e.g., free siRNA). The evaluation can include an assay where the composition and/or free nucleic acid agent is incubated with a degradant such as an RNase, and, e.g., wherein the composition and free nucleic acid are evaluated over various time points, e.g., using gel chromatography.

In an embodiment a particle or preparation of particles is evaluated for the level of intact nucleic acid agent (e.g., an siRNA) it contains. In embodiment the intactness can be determined by presence of a physical property, e.g., molecular weight, or by functionality for example, in an in vivo model system, (e.g., a mouse model such as any one of those described herein). In embodiments the level is compared with a reference standard. In embodiments at least 30, 40, 50, 60, 70, 80, or 90% of the nucleic acid agent, e.g., siRNA, by number or weight, in a particle may be intact.

In an embodiment a particle or preparation of particles is evaluated for its tendency to aggregate. E.g., aggregation can be measured in a preselected medium, e.g., 50/50 mouse/human serum. In embodiment, when incubated 50/50 mouse human serum, the particles exhibit little or no aggregation. E.g., less than 30, 20, or 10%, by number or weight, of the particles will aggregate. In embodiments the level is compared with a reference standard.

In an embodiment a particle or preparation of particles is evaluated for stability, e.g., stability at a preselected condition, e.g., at 25° C.±2° C./60% relative humidity ±5% relative humidity, e.g., in an open, or closed, container. In embodiments, when stored at 25° C.±2° C./60% relative humidity ±5% relative humidity in an open, or closed, container, for 20, 30, 40, 50 or 60 days, the particle retains at least 30, 40, 50, 60, 70, 80, 90, or 95% of its activity, e.g., as determined in an in vivo model system, (e.g., a mouse model such as one described herein). In embodiments the level of retained activity is compared with a reference standard.

In an embodiment a particle or preparation of particles is evaluated its ability to reduce protein and or mRNA, e.g., at a preselected dosage. E.g., particles can be evaluated by administration as a single dose of 1 or 3 mg/kg in an in vivo model system, (e.g., a mouse model such one of those described herein). A particle described herein may result in at least 20, 30, 40, 50, or 60% reduction in protein and or mRNA knockdown. In embodiments the level is compared with a reference standard.

In an embodiment a particle or preparation of particles is evaluated its ability to reduce protein and or mRNA, of a target gene, e.g., at a preselected dosage. E.g., particles can be evaluated by administration as a single dose of 1 or 3 mg/kg in an in vivo model system, (e.g., a mouse model such as any of those described herein). A particle described herein may result in at least 20, 30, 40, 50, or 60% reduction in protein and or mRNA knockdown. In embodiments the level is compared with a reference standard.

In an embodiment a particle or preparation of particles is evaluated for reduction of protein and or mRNA, of an off-target gene, e.g., at a preselected dosage. E.g., particles can be evaluated by administration, e.g., as a single dose of 1 or 3 mg/kg in an in vivo model system, (e.g., a mouse model such as any of those described herein). A particle or preparation described herein may result in less than 20, 10, 5%, or no knockdown, as measured by protein or mRNA, when administered (e.g., as a single dose of 1 or 3 mg/kg) in an in vivo model system, (e.g., a mouse model such as any of those described herein).

In an embodiment a particle or preparation of particles is evaluated for the ability to cleave mRNA.

In an embodiment a particle or preparation of particles is evaluated for the ability to induce cytokines. A particle or preparation described herein may result in less than 2, 5, or 10 fold cytokine induction, when administered (e.g., as a single dose of 1 or 3 mg/kg) in an in vivo model system, (e.g., a mouse model such as any of those described herein). E.g., the administration results in less than 2, 5, or 10 fold induction of one, or more, e.g., two, three, four, five, six, or seven, or all, of: tumor necrosis factor-alpha, interleukin-1alpha, interleukin-1beta, interleukin-6, interleukin-10, interleukin-12, keratinocyte-derived cytokine and interferon-gamma.

In an embodiment a particle or preparation of particles is evaluated for the ability to increase in alanine aminotransferase (ALT) and or aspartate aminotransferase (AST), when administered (e.g., as a single dose of 1 or 3 mg/kg) in an in vivo model system, (e.g., a mouse model such as any of those described herein). In an embodiment a particle or preparation results in less than 2, 5, or 10 fold increase.

In an embodiment a particle or preparation of particles is evaluated for the ability to alter blood count. In an embodiment a particle or preparation results in no changes in blood count, e.g., no change 48 hours after 2 doses of 3 mg/kg in an in vivo model system, (e.g., a mouse model such as any of those described herein).

A particle described herein may be subjected to a number of analytical methods. For example, a particle described herein may be subjected to a measurement to determine whether an impurity or residual solvent is present (e.g., via gas chromatography (GC)), to determine relative amounts of one or more components (e.g., via high performance liquid chromatography (HPLC)), to measure particle size (e.g., via dynamic light scattering and/or scanning electron microscopy), or determine the presence or absence of surface components.

Compositions disclosed herein can be evaluated, for example, for the ability to deliver a nucleic acid agent, e.g., an siRNA, that knocks down a target gene, in vivo, e.g., in an experimental animal, e.g., a mouse. The activity of the composition can be compared to that of an equal amount of free nucleic acid agent. In some embodiments the target gene is GFP (e.g., an EGFP) the GFP is expressed in HeLA cells. E.g., the assay can use the anti-GFP siRNA, the GFP plasmid, the HeLA-GFP cells, the mice, and the GFP expression assays described in Bertrand et al., 2002, BBRC 296:1000-1004, hereby incorporated by reference. Other exemplary cells for evaluating particles and compositions described herein include MDA-MB-435 and M4A4 GFP cells.

Compositions disclosed herein can be evaluated for the ability to deliver a nucleic acid agent, e.g., an siRNA, that knocks down a target gene in vitro, e.g., in cultured cells. The activity of the composition can be compared to that of an equal amount of free nucleic acid agent. In some embodiments the target gene is GFP and the cultured cells are HeLA cells transfected with GFP. E.g., the assay can use the anti-GFP siRNA, the GFP plasmid, the HeLA-GFP cells, the cell culture conditions, and the GFP expression assay described in Bertrand et al., 2002, BBRC 296:1000-1004, hereby incorporated by reference. Other exemplary cells for evaluating particles and compositions described herein include MDA-MB-435 and M4A4 GFP cells.

Compositions disclosed herein can be evaluated for the ability to deliver a nucleic acid agent, e.g., an siRNA, that knocks down a target gene in vitro, e.g., in cultured cells, after incubation in serum or a cell lysate. The activity of the treated composition can be compared to that of an equal amount of free nucleic acid agent. In some embodiments the target gene is GFP and the cultured cells are HeLA cells transfected with GFP. E.g., the assay can use the anti-GFP siRNA, the GFP plasmid, the HeLA-GFP cells, the cell culture conditions, the GFP expression assay, and, in the case of an assay that uses a cell lysate, the HeLa cell lysate, described in Bertrand et al., 2002, BBRC 296:1000-1004, hereby incorporated by reference. Alternatively, the mouse expression system described in Hu-Lieskovan et al., 2005, Cancer Res. 65: 8984-8992, hereby incorporated by reference, can be used to evaluate the performance of a composition. The target gene and constructs of Hu-Lieskovan et al., or other target genes and constructs can be used with the mouse system described in Hu-lieskovan et al. Other exemplary cells for evaluating particles and compositions described herein include MDA-MB-435 and M4A4 GFP cells.

Compositions disclosed herein can be evaluated for the ability to protect a nucleic acid agent from a degradant such as an RNase (e.g., RNase A). In some embodiments, a composition described herein can confer protection on a nucleic acid agent such as an siRNA relative to untreated nucleic acid agent (e.g., free siRNA). The evaluation can include an assay where the composition and/or free nucleic acid agent is incubated with a degradant such as an RNase, and wherein the composition and free nucleic acid are evaluated over various time points, e.g., using gel chromatography.

### Pharmaceutical Compositions

Provided herein is a composition, e.g., a pharmaceutical composition, comprising a plurality of particles described herein and a pharmaceutically acceptable carrier or adjuvant.

In some embodiments, a pharmaceutical composition may include a pharmaceutically acceptable salt of a compound described herein, e.g., a conjugate. Pharmaceutically acceptable salts of the compounds described herein include those derived from pharmaceutically acceptable inorganic and organic acids and bases. Examples of suitable acid salts include acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, methanesulfonate, 2-naphthalenesulfonate, nicotinate, nitrate, palmoate, phosphate, picrate, pivalate, propionate, salicylate, succinate, sulfate, tartrate, tosylate and undecanoate. Salts derived from appropriate bases include alkali metal (e.g., sodium), alkaline earth metal (e.g., magnesium), ammonium and N-(alkyl)₄⁺ salts. This invention also envisions the quaternization of any basic nitrogen-containing groups of the compounds described herein. Water or oil-soluble or dispersible products may be obtained by such quaternization.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gailate, alpha-tocopherol, and the like; and (3) metal chelating agents, such as citric acid, ethylenediamine tetraacetic acid (EDTA), sorbitol, tartaric acid, phosphoric acid, and the like.

A composition may include a liquid used for suspending a conjugate, particle or composition, which may be any liquid solution compatible with the conjugate, particle or composition, which is also suitable to be used in pharmaceutical compositions, such as a pharmaceutically acceptable nontoxic liquid. Suitable suspending liquids including but are not limited to suspending liquids selected from the group consisting of water, aqueous sucrose syrups, corn syrups, sorbitol, polyethylene glycol, propylene glycol, D5W and mixtures thereof.

A composition described herein may also include another component, such as an antioxidant, antibacterial, buffer, bulking agent, chelating agent, an inert gas, a tonicity agent and/or a viscosity agent.

In one embodiment, the polymer-agent conjugate, particle or composition is provided in lyophilized form and is reconstituted prior to administration to a subject. The lyophilized polymer-agent conjugate, particle or composition can be reconstituted by a diluent solution, such as a salt or saline solution, e.g., a sodium chloride solution having a pH between 6 and 9, lactated Ringer's injection solution, or a commercially available diluent, such as PLASMA-LYTE A Injection pH 7.4® (Baxter, Deerfield, Ill.).

In one embodiment, a lyophilized formulation includes a lyoprotectant or stabilizer to maintain physical and chemical stability by protecting the particle and active from damage from crystal formation and the fusion process during freeze-drying. The lyoprotectant or stabilizer can be one or more of polyethylene glycol (PEG), a PEG lipid conjugate (e.g., PEG-ceramide or D-alpha-tocopheryl polyethylene glycol 1000 succinate), poly(vinyl alcohol) (PVA), poly(vinylpyrrolidone) (PVP), polyoxyethylene esters, poloxamers, polysorbates, polyoxyethylene esters, lecithins, saccharides, oligosaccharides, polysaccharides, carbohydrates, cyclodextrins (e.g. 2-hydroxypropyl-β-cyclodextrin) and polyols (e.g., trehalose, mannitol, sorbitol, lactose, sucrose, glucose and dextran), salts and crown ethers.

In some embodiments, the lyophilized polymer-agent conjugate, particle or composition is reconstituted with water, 5% Dextrose Injection, Lactated Ringer's and Dextrose Injection, or a mixture of equal parts by volume of Dehydrated Alcohol, USP and a nonionic surfactant, such as a polyoxyethylated castor oil surfactant available from GAF Corporation, Mount Olive, N.J., under the trademark, Cremophor EL. The lyophilized product and vehicle for reconstitution can be packaged separately in appropriately light-protected vials. To minimize the amount of surfactant in the reconstituted solution, only a sufficient amount of the vehicle may be provided to form a solution of the polymer-agent conjugate, particle or composition. Once dissolution of the drug is achieved, the resulting solution is further diluted prior to injection with a suitable parenteral diluent. Such diluents are well known to those of ordinary skill in the art. These diluents are generally available in clinical facilities. It is, however, within the scope of the present invention to package the subject polymer-agent conjugate, particle or composition with a third vial containing sufficient parenteral diluent to prepare the final concentration for administration. A typical diluent is Lactated Ringer's Injection.

The final dilution of the reconstituted polymer-agent conjugate, particle or composition may be carried out with other preparations having similar utility, for example, 5% dextrose injection, lactated ringer's and dextrose injection, sterile water for injection, and the like. However, because of its narrow pH range, pH 6.0 to 7.5, lactated ringer's injection is most typical. Per 100 mL, Lactated Ringer's Injection contains sodium chloride USP 0.6 g, Sodium Lactate 0.31 g, potassium chloride USP 0.03 g and calcium chloride2H2O USP 0.02 g. The osmolarity is 275 mOsmol/L, which is very close to isotonicity.

The compositions may conveniently be presented in unit dosage form and may be prepared by any methods well known in the art of pharmacy. The amount of nucleic acid agent which can be combined with a pharmaceutically acceptable carrier to produce a single dosage form will vary depending upon the host being treated, the particular mode of administration. The amount of nucleic acid agent which can be combined with a pharmaceutically acceptable carrier to produce a single dosage form will generally be that amount of the compound which produces a therapeutic effect.

### Routes of Administration

The pharmaceutical compositions described herein may be administered orally, parenterally (e.g., via intravenous, subcutaneous, intracutaneous, intramuscular, intraarticular, intraarterial, intrasynovial, intrasternal, intrathecal, intralesional, intraocular, or intracranial injection), topically, mucosally (e.g., rectally or vaginally), nasally, buccally, ophthalmically, via inhalation spray (e.g., delivered via nebulization, propellant or a dry powder device) or via an implanted reservoir.

Pharmaceutical compositions suitable for parenteral administration comprise one or more polymer-agent conjugate(s), particle(s) or composition(s) in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a nucleic acid agent, it is desirable to slow the absorption of the agent from subcutaneous or intramuscular injection. This may be accomplished by the use of a liquid suspension of crystalline or amorphous material having poor water solubility. The rate of absorption of the conjugate, particle or composition then depends upon its rate of dissolution which, in turn, may depend upon crystal size and crystalline form. Alternatively, delayed absorption of a parenterally administered drug form is accomplished by dissolving or suspending the conjugate, particle or composition in an oil vehicle.

Pharmaceutical compositions suitable for oral administration may be in the form of capsules, cachets, pills, tablets, gums, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouthwashes and the like, each containing a predetermined amount of an agent as an active ingredient. A composition may also be administered as a bolus, electuary or paste.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered peptide or peptidomimetic moistened with an inert liquid diluent.

Tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups and elixirs. In addition to the polymer-agent conjugate, particle or composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the polymer-agent conjugate, particle or composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions suitable for topical administration are useful when the desired treatment involves areas or organs readily accessible by topical application. For application topically to the skin, the pharmaceutical composition should be formulated with a suitable ointment containing the active components suspended or dissolved in a carrier. Carriers for topical administration of the a particle described herein include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active particle suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions described herein may also be topically applied to the lower intestinal tract by rectal suppository formulation or in a suitable enema formulation. Topically-transdermal patches are also included herein.

The pharmaceutical compositions described herein may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

The pharmaceutical compositions described herein may also be administered in the form of suppositories for rectal or vaginal administration. Suppositories may be prepared by mixing one or more polymer-agent conjugate, particle or composition described herein with one or more suitable non-irritating excipients which is solid at room temperature, but liquid at body temperature. The composition will therefore melt in the rectum or vaginal cavity and release the polymer-agent conjugate, particle or composition. Such materials include, for example, cocoa butter, polyethylene glycol, a suppository wax or a salicylate. Compositions of the present invention which are suitable for vaginal administration also include pessaries, tampons, creams, gels, pastes, foams or spray formulations containing such carriers as are known in the art to be appropriate.

Ophthalmic formulations, eye ointments, powders, solutions and the like, are also contemplated as being within the scope of the invention. An ocular tissue (e.g., a deep cortical region, a supranuclear region, or an aqueous humor region of an eye) may be contacted with the ophthalmic formulation, which is allowed to distribute into the lens. Any suitable method(s) of administration or application of the ophthalmic formulations of the invention (e.g., topical, injection, parenteral, airborne, etc.) may be employed. For example, the contacting may occur via topical administration or via injection.

### Dosages and Dosage Regimens

The conjugates, particles, and compositions can be formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular subject, composition, and mode of administration, without being toxic to the subject.

In one embodiment, the conjugate, particle or composition is administered to a subject at a dosage of, e.g., about 0.001 to 300 mg/m², about 0.002 to 200 mg/m², about 0.005 to 100 mg/m², about 0.01 to 100 mg/m², about 0.1 to 100 mg/m², about 5 to 275 mg/m², about 10 to 250 mg/m², e.g., about 0.001, 0.002, 0.005, 0.01, 0.05, 0.1, 15, 20, 25, 30, 35, 40, 45, 50, 55, 60, 65, 70, 80, 90, 100, 110, 120, 130, 140, 150, 160, 170, 180, 190, 200, 210, 220, 230, 240, 250, 260, 270, 280, 290 mg/m². Administration can be at regular intervals, such as every 1, 2, 3, 4, or 5 days, or weekly, or every 2, 3, 4, 5, 6, or 7 or 8 weeks. The administration can be over a period of from about 10 minutes to about 6 hours, e.g., from about 30 minutes to about 2 hours, from about 45 minutes to 90 minutes, e.g., about 30 minutes, 45 minutes, 1 hour, 2 hours, 3 hours, 4 hours, 5 hours or more. In one embodiment, the polymer-agent conjugate, particle or composition is administered as a bolus infusion or intravenous push, e.g., over a period of 15 minutes, 10 minutes, 5 minutes or less. In one embodiment, the conjugate, particle or composition is administered in an amount such the desired dose of the agent is administered. Preferably the dose of the conjugate, particle or composition is a dose described herein.

In one embodiment, the subject receives 1, 2, 3, up to 10, up to 12, up to 15 treatments, or more, or until the disorder or a symptom of the disorder is cured, healed, alleviated, relieved, altered, remedied, ameliorated, palliated, improved or affected. For example, the subject receive an infusion once every 1, 2, 3 or 4 weeks until the disorder or a symptom of the disorder are cured, healed, alleviated, relieved, altered, remedied, ameliorated, palliated, improved or affected. Preferably, the dosing schedule is a dosing schedule described herein.

The conjugate, particle, or composition can be administered as a first line therapy, e.g., alone or in combination with an additional agent or agents. In other embodiments, a conjugate, particle or composition is administered after a subject has developed resistance to, has failed to respond to or has relapsed after a first line therapy. The conjugate, particle or composition may be administered in combination with a second agent. Preferably, the conjugate, particle or composition is administered in combination with a second agent described herein. The second agent may be the same or different as the nucleic acid agent in the particle.

### Kits

A conjugate, particle or composition described herein may be provided in a kit. The kit includes a conjugate, particle or composition described herein and, optionally, a container, a pharmaceutically acceptable carrier and/or informational material. The informational material can be descriptive, instructional, marketing or other material that relates to the methods described herein and/or the use of the particles for the methods described herein.

The informational material of the kits is not limited in its form. In one embodiment, the informational material can include information about production of the conjugate, particle or composition, physical properties of the conjugate, particle or composition, concentration, date of expiration, batch or production site information, and so forth. In one embodiment, the informational material relates to methods for administering the conjugate, particle or composition.

In one embodiment, the informational material can include instructions to administer a conjugate, particle or composition described herein in a suitable manner to perform the methods described herein, e.g., in a suitable dose, dosage form, or mode of administration (e.g., a dose, dosage form, or mode of administration described herein). In another embodiment, the informational material can include instructions to administer a conjugate, particle or composition described herein to a suitable subject, e.g., a human, e.g., a human having or at risk for a disorder described herein. In another embodiment, the informational material can include instructions to reconstitute a conjugate or particle described herein into a pharmaceutically acceptable composition.

In one embodiment, the kit includes instructions to use the conjugate, particle or composition, such as for treatment of a subject. The instructions can include methods for reconstituting or diluting the conjugate, particle or composition for use with a particular subject or in combination with a particular chemotherapeutic agent. The instructions can also include methods for reconstituting or diluting the polymer conjugate composition for use with a particular means of administration, such as by intravenous infusion.

In another embodiment, the kit includes instructions for treating a subject with a particular indication. The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is contact information, e.g., a physical address, email address, website, or telephone number, where a user of the kit can obtain substantive information about a particle described herein and/or its use in the methods described herein. The informational material can also be provided in any combination of formats.

In addition to a conjugate, particle or composition described herein, the composition of the kit can include other ingredients, such as a surfactant, a lyoprotectant or stabilizer, an antioxidant, an antibacterial agent, a bulking agent, a chelating agent, an inert gas, a tonicity agent and/or a viscosity agent, a solvent or buffer, a stabilizer, a preservative, a flavoring agent (e.g., a bitter antagonist or a sweetener), a fragrance, a dye or coloring agent, for example, to tint or color one or more components in the kit, or other cosmetic ingredient, a pharmaceutically acceptable carrier and/or a second agent for treating a condition or disorder described herein. Alternatively, the other ingredients can be included in the kit, but in different compositions or containers than a particle described herein. In such embodiments, the kit can include instructions for admixing a conjugate, particle or composition described herein and the other ingredients, or for using a conjugate, particle or composition described herein together with the other ingredients.

In another embodiment, the kit includes a second therapeutic agent. In one embodiment, the second agent is in lyophilized or in liquid form. In one embodiment, the conjugate, particle or composition and the second therapeutic agent are in separate containers, and in another embodiment, the conjugate, particle or composition and the second therapeutic agent are packaged in the same container.

In some embodiments, a component of the kit is stored in a sealed vial, e.g., with a rubber or silicone enclosure (e.g., a polybutadiene or polyisoprene enclosure). In some embodiments, a component of the kit is stored under inert conditions (e.g., under nitrogen or another inert gas such as argon). In some embodiments, a component of the kit is stored under anhydrous conditions (e.g., with a desiccant). In some embodiments, a component of the kit is stored in a light blocking container such as an amber vial.

A conjugate, particle or composition described herein can be provided in any form, e.g., liquid, frozen, dried or lyophilized form. It is preferred that a conjugate, particle or composition described herein be substantially pure and/or sterile. In some embodiments, the conjugate, particle or composition is sterile. When a conjugate, particle or composition described herein is provided in a liquid solution, the liquid solution preferably is an aqueous solution, with a sterile aqueous solution being preferred. In one embodiment, the conjugate, particle or composition is provided in lyophilized form and, optionally, a diluent solution is provided for reconstituting the lyophilized agent. The diluent can include for example, a salt or saline solution, e.g., a sodium chloride solution having a pH between 6 and 9, lactated Ringer's injection solution, D5W, or PLASMA-LYTE A Injection pH 7.4® (Baxter, Deerfield, Ill.).

The kit can include one or more containers for the composition containing a conjugate, particle or composition described herein. In some embodiments, the kit contains separate containers, dividers or compartments for the composition and informational material. For example, the composition can be contained in a bottle, vial, IV admixture bag, IV infusion set, piggyback set or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of a polymer-agent conjugate, particle or composition described herein. For example, the kit includes a plurality of syringes, ampules, foil packets, or blister packs, each containing a single unit dose of a particle described herein. The containers of the kits can be air tight, waterproof (e.g., impermeable to changes in moisture or evaporation), and/or light-tight.

The kit optionally includes a device suitable for administration of the composition, e.g., a syringe, inhalant, pipette, forceps, measured spoon, dropper (e.g., eye dropper), swab (e.g., a cotton swab or wooden swab), or any such delivery device. In one embodiment, the device is a medical implant device, e.g., packaged for surgical insertion.

### Methods of Using Particles and Compositions

The polymer-agent conjugates, particles and compositions described herein can be administered to cells in culture, e.g. in vitro or ex vivo, or to a subject, e.g., in vivo, to treat or prevent a variety of diseases or disorders (e.g., cancer (for example solid tumors), autoimmune disorders, cardiovascular disorders, inflammatory disorders, metabolic disorders, infectious diseases, etc.).

Thus, in another aspect, the invention features, a method of treating or preventing a disease or disorder in a subject wherein the disease or disorder is cancer (for example a solid tumor), an autoimmune disorder, a cardiovascular disorder, inflammatory disorder, a metabolic disorder, or an infectious disease. The method comprises administering an effective amount of a conjugate, particle, or composition described herein to thereby treat the disease or disorder. In an embodiment the conjugates, particles and compositions can be used as part of a first line, second line, or adjunct therapy, and can also be used alone or in combination with one or more additional treatment regimes.

In an embodiment conjugates (e.g., polymer-nucleic acid agent conjugates), particles, or compositions disclosed herein can be used to treat or prevent a wide variety of diseases or disorders and can be used to deliver nucleic acid agents, for example, to a subject in need thereof, for example, antisense or siRNA; to treat diseases and disorders described herein such as cancer, inflammatory or autoimmune disease, or cardiovascular disease, including those listed in the following tables A, B, or C. In embodiments the polymer-nucleic acid agent conjugates, particles and compositions can be used as part of a first line, second line, or adjunct therapy, and can also be used alone or in combination with one or more additional treatment regimes.

### Cancer

Accordingly, in another aspect, the invention features, a method of treating or preventing a disease or disorder in a subject wherein the disease or disorder is cancer (for example a solid tumor). The method comprises administering an effective amount of a conjugate, particle, or composition described herein to thereby treat the disease or disorder. In an embodiment the conjugates, particles and compositions can be used as part of a first line, second line, or adjunct therapy, and can also be used alone or in combination with one or more additional treatment regimes.

In embodiments the disclosed polymer-agent conjugates, particles and compositions are used to treat or prevent proliferative disorders, e.g., treating a tumor and metastases thereof wherein the tumor or metastases thereof is a cancer described herein. In some embodiments, wherein the agent is a diagnostic agent, the polymer-agent conjugates, particles and compositions described herein can be used to evaluate or diagnose a cancer.

In embodiments, the proliferative disorder is a solid tumor, a soft tissue tumor or a liquid tumor. Exemplary solid tumors include malignancies (e.g., sarcomas and carcinomas (e.g., adenocarcinoma or squamous cell carcinoma)) of the various organ systems, such as those of brain, lung, breast, lymphoid, gastrointestinal (e.g., colon), and genitourinary (e.g., renal, urothelial, or testicular tumors) tracts, pharynx, prostate, and ovary. Exemplary adenocarcinomas include colorectal cancers, renal-cell carcinoma, liver cancer, non-small cell carcinoma of the lung, and cancer of the small intestine. In embodiments the method comprises evaluating or treating soft tissue tumors such as those of the tendons, muscles or fat, and liquid tumors.

In embodiment the cancer is any cancer, for example those described by the National Cancer Institute. The cancer can be a carcinoma, a sarcoma, a myeloma, a leukemia, a lymphoma or a mixed type. Exemplary cancers described by the National Cancer Institute include:

Digestive/gastrointestinal cancers such as anal cancer; bile duct cancer; extrahepatic bile duct cancer; appendix cancer; carcinoid tumor, gastrointestinal cancer; colon cancer; colorectal cancer including childhood colorectal cancer; esophageal cancer including childhood esophageal cancer; gallbladder cancer; gastric (stomach) cancer including childhood gastric (stomach) cancer; hepatocellular (liver) cancer including adult (primary) hepatocellular (liver) cancer and childhood (primary) hepatocellular (liver) cancer; pancreatic cancer including childhood pancreatic cancer; sarcoma, rhabdomyosarcoma; islet cell pancreatic cancer; rectal cancer; and small intestine cancer;

Endocrine cancers such as islet cell carcinoma (endocrine pancreas); adrenocortical carcinoma including childhood adrenocortical carcinoma; gastrointestinal carcinoid tumor; parathyroid cancer; pheochromocytoma; pituitary tumor; thyroid cancer including childhood thyroid cancer; childhood multiple endocrine neoplasia syndrome; and childhood carcinoid tumor;

Eye cancers such as intraocular melanoma; and retinoblastoma;

Musculoskeletal cancers such as Ewing's family of tumors; osteosarcoma/malignant fibrous histiocytoma of the bone; childhood rhabdomyosarcoma; soft tissue sarcoma including adult and childhood soft tissue sarcoma; clear cell sarcoma of tendon sheaths; and uterine sarcoma;

Breast cancer such as breast cancer including childhood and male breast cancer and pregnancy;

Neurologic cancers such as childhood brain stem glioma; brain tumor; childhood cerebellar astrocytoma; childhood cerebral astrocytoma/malignant glioma; childhood ependymoma; childhood medulloblastoma; childhood pineal and supratentorial primitive neuroectodermal tumors; childhood visual pathway and hypothalamic glioma; other childhood brain cancers; adrenocortical carcinoma; central nervous system lymphoma, primary; childhood cerebellar astrocytoma; neuroblastoma; craniopharyngioma; spinal cord tumors; central nervous system atypical teratoid/rhabdoid tumor; central nervous system embryonal tumors; and childhood supratentorial primitive neuroectodermal tumors and pituitary tumor;

Genitourinary cancers such as bladder cancer including childhood bladder cancer; renal cell (kidney) cancer; ovarian cancer including childhood ovarian cancer; ovarian epithelial cancer; ovarian low malignant potential tumor; penile cancer; prostate cancer; renal cell cancer including childhood renal cell cancer; renal pelvis and ureter, transitional cell cancer; testicular cancer; urethral cancer; vaginal cancer; vulvar cancer; cervical cancer; Wilms tumor and other childhood kidney tumors; endometrial cancer; and gestational trophoblastic tumor;

Germ cell cancers such as childhood extracranial germ cell tumor; extragonadal germ cell tumor; ovarian germ cell tumor; and testicular cancer;

Head and neck cancers such as lip and oral cavity cancer; oral cancer including childhood oral cancer; hypopharyngeal cancer; laryngeal cancer including childhood laryngeal cancer; metastatic squamous neck cancer with occult primary; mouth cancer; nasal cavity and paranasal sinus cancer; nasopharyngeal cancer including childhood nasopharyngeal cancer; oropharyngeal cancer; parathyroid cancer; pharyngeal cancer; salivary gland cancer including childhood salivary gland cancer; throat cancer; and thyroid cancer;

Hematologic/blood cell cancers such as a leukemia (e.g., acute lymphoblastic leukemia including adult and childhood acute lymphoblastic leukemia; acute myeloid leukemia including adult and childhood acute myeloid leukemia; chronic lymphocytic leukemia; chronic myelogenous leukemia; and hairy cell leukemia); a lymphoma (e.g., AIDS-related lymphoma; cutaneous T-cell lymphoma; Hodgkin's lymphoma including adult and childhood Hodgkin's lymphoma and Hodgkin's lymphoma during pregnancy; non-Hodgkin's lymphoma including adult and childhood non-Hodgkin's lymphoma and non-Hodgkin's lymphoma during pregnancy; mycosis fungoides; Sézary syndrome; Waldenstrom's macroglobulinemia; and primary central nervous system lymphoma); and other hematologic cancers (e.g., chronic myeloproliferative disorders; multiple myeloma/plasma cell neoplasm; myelodysplastic syndromes; and myelodysplastic/myeloproliferative disorders);

Lung cancer such as non-small cell lung cancer; and small cell lung cancer;

Respiratory cancers such as malignant mesothelioma, adult; malignant mesothelioma, childhood; malignant thymoma; childhood thymoma; thymic carcinoma; bronchial adenomas/carcinoids including childhood bronchial adenomas/carcinoids; pleuropulmonary blastoma; non-small cell lung cancer; and small cell lung cancer;

Skin cancers such as Kaposi's sarcoma; Merkel cell carcinoma; melanoma; and childhood skin cancer;

AIDS-related malignancies;

Other childhood cancers, unusual cancers of childhood and cancers of unknown primary site;

and metastases of the aforementioned cancers can also be treated or prevented in accordance with the methods described herein.

The polymer-agent conjugates, compounds or compositions described herein are particularly suited to treat accelerated or metastatic cancers of the bladder cancer, pancreatic cancer, prostate cancer, renal cancer, non-small cell lung cancer, ovarian cancer, melanoma, colorectal cancer, and breast cancer.

In one embodiment, a method is provided for a combination treatment of a cancer, such as by treatment with a polymer-agent conjugate, compound or composition and a second therapeutic agent. Various combinations are described herein. The combination can reduce the development of tumors, reduces tumor burden, or produce tumor regression in a mammalian host.

In an embodiment, a nucleic acid agent-polymer conjugate, particle or composition, e.g., containing an siRNA that targets a gene listed in Table A, is administered, e.g, to treat or prevent, an associated disease listed in Table A.

TABLE A
The nucleic acid agent, e.g., an siRNA, can target a gene listed in the table,
for example, to treat or prevent the associated disease.
Cancer

| | Disease Associated with siRNA knock |
|---|---|
| Gene | down of gene |
| ICAM-1 | Angiogenesis (associated with cancer: |
| | breast, lung, head and neck, brain, abdominal, |
| | colon, colorectal, esophagus, gastrointestinal, |
| | glioma, liver, tongue, neuroblastoma, |
| | osteosarcoma, ovarian, pancreatic, prostate, |
| | retinoblastoma, Wilm's tumor, multiple myeloma, |
| | skin, lymphoma, blood, tumor metastasis, multiple |
| | myeloma) |
| NPRA | Melanoma, lung, ovarian |
| Akt & p85alpha | Colorectal |
| IL-1, TNFalpha, Fas, FasL | Liver |
| RAS, MYC, FOS, JUN, ERG-2, | Cancer |
| VEGF, FGF, Hcg | |
| KLF5 | Angiogenesis |
| Beta-TrCRP1, Beta-TrCP2, RSK1, | Cancer |
| RSK2 | |
| Notch1 | Cancer |
| HER2 | Breast |
| CD24 | Colorectal |
| ILK | Cancer |
| Nrf2 | Lung |
| Agtr11, Apelin, Stabilin 1, Stabilin | Angiogenesis |
| 2, TNFaip811, TNFaip8, FGD5 | |
| STAT3 | Cancer |
| HIF-1alpha | Cancer |
| STAT5 | Cancer |
| EGR, XIAP | Cancer |
| Akt2 | Cancer |
| TRIM24 | Breast, retinal, prostate, colon, acute |
| | lymphoblastic leukemia |
| PLK1 | Cancer |
| Src-1, Src-2, Src-3, AIB1 | Cancer |
| ANT2 | Cancer |
| EGFR | Breast, lung, colorectal, prostate, brain, |
| | esophageal, stomach, bladder, pancreatic, cervical, |
| | head and neck, kidney, endometrial, ovarian, |
| | meningioma, melanoma, lymphoma, glioblastoma |
| CACNA1E | Breast, lung, liver, colon, prostate, renal, |
| | ovarian, pancreatic, prostate, renal, skin, uterine |
| PAX2 | Breast |
| FZD | Liver |
| ARG2 | Breast, non small cell lung |
| eIFSA1 | Cancer |
| Atg1, Atg2, Atg3, Atg4, Atg5, | Breast, liver, ovarian, gastric, bladder, |
| Beclin1, Atg7, MAP1 LC3B, | colon, prostate, lung, nasopharyngeal carcinoma, |
| Atg9/APG9L1/2, Atg10, Atg12, Atg16, | neuroblastoma, glioma, solid tumor, hematologic |
| mTOR, PIK3C3, VPS34 | malignancy, leukemia, lymphoma |
| SEPT10, LMNB2, HRH1, | Colon, osteosarcoma, liver, melanoma, |
| HOXA10, ERCC3, MIS12, MPHOSPHI1, | head and neck squamous cell carcinoma |
| CDC7, SMARCB1, MAD2L1, DTL, | |
| RACGAP1, MCM10, PIM1, DLG5, BCL2, | |
| CUL5, PRPF38A | |
| Cineurin | Leukemia, lymphoma, melanoma, lung, |
| | bowel, colon, rectal, colorectal, brain, liver, |
| | pancreatic, breast, testicular, retinoblastoma |
| alpha-enolase | Cancer |
| BRAF | Malignant melanoma |
| Androgen receptor | Bladder |
| HOXB13 | Prostate |
| Wnt2 | Breast, ovarian, colorectal, gastric, lung, |
| | kidney, bladder, prostate, uterine, thyroid, |
| | pancreatic, cervical, esophageal, mesothelioma, |
| | head and neck, hepatocellular, melanoma, brain |
| | vulval, testicular, sarcoma, intestine, skin, |
| | leukemia, lymphoma |
| NuMA | Cervical, epidermoid, oral, glioma, |
| | leukemia, brain, esophageal, stomach, bladder, |
| | pancreatic, cervical, head and neck, ovarian, |
| | melanoma, lymphoma |
| Ang-1, Ang-2, Tie2 | Cancer |
| MAGE-B (B1, B2, B3, B4), | Melanoma, lymphoma, T cell leukemia, |
| MAGE-C, MAG-A(A1, A3, A5, A6, A8, | non small cell lung, hepatic carcinoma, gastric, |
| A9, A10, A11, A12), Necdin, MAGE-D, | esophagus, colorectal, gastric, endocrine, ovarian, |
| MAGE-E (E1), MAGE-F, MAGE-G, | pancreatic, ovarian, cervical, salivary, head and |
| MAGE-H | neck squamous cell, spermatocytic seminoma, |
| | sporadic medulalry thyroid carcinoma, bladder, |
| | osteosarcoma, non-proliferating testes cells, |
| | neuroblastoma, glioma, cancers related to |
| | malignant mast cells |
| Galactin-1 | Glioma, pancreatic, non small cell lung, |
| | non-Hodgkin's lymphoma |
| Tpt1 | Cancer |
| c-FLIP | Cancer |
| EBAG9 | Prostate, bladder |
| Nrf2 | Lung |
| E6TMF/ARA160 | Cancer |
| Jun, Erg-2 | Cancer |
| CSN5 | Hepatocellular Carcinoma |
| COP1-1 | Hepatocellular Carcinoma |
| PLK1 | Cancer |
| LMP2, LMP7, MECL1 | Metastatic melanoma |
| M2 subunit ribonucleotide | Solid tumor |
| reductase | |
| AHR | Neuroblastoma |
| B4GALNT3 | Neuroblastoma |
| PKN3 | Colorectal cancer metastasizing to the liver |
| KSP | Liver cancer |
| b-catenin | Familial adenomatous polyposis |

### Inflammation and Autoimmune Disease

In another aspect, the invention features, a method of treating or preventing a disease or disorder in a subject wherein the disease or disorder is inflammation or an autoimmune disease. The method comprises administering an effective amount of a conjugate, particle, or composition described herein to thereby treat the disease or disorder. In an embodiment the conjugates, particles and compositions can be used as part of a first line, second line, or adjunct therapy, and can also be used alone or in combination with one or more additional treatment regimes.

In an embodiment the polymer-agent conjugates, particles, compositions and methods described herein can be used to treat or prevent a disease or disorder associated with inflammation. In embodiments a polymer-agent conjugate, particle or composition described herein may be administered prior to the onset of, at, or after the initiation of inflammation. In embodiments, used prophylactically, the polymer-agent conjugate, particle or composition is provided in advance of any inflammatory response or symptom. In embodiments administration of the polymer-agent conjugate, particle or composition can prevent or attenuate inflammatory responses or symptoms. Exemplary inflammatory conditions include, for example, multiple sclerosis, rheumatoid arthritis, psoriatic arthritis, degenerative joint disease, spondouloarthropathies, gouty arthritis, systemic lupus erythematosus, juvenile arthritis, rheumatoid arthritis, osteoarthritis, osteoporosis, diabetes (e.g., insulin dependent diabetes mellitus or juvenile onset diabetes), menstrual cramps, cystic fibrosis, inflammatory bowel disease, irritable bowel syndrome, Crohn's disease, mucous colitis, ulcerative colitis, gastritis, esophagitis, pancreatitis, peritonitis, Alzheimer's disease, shock, ankylosing spondylitis, gastritis, conjunctivitis, pancreatis (acute or chronic), multiple organ injury syndrome (e.g., secondary to septicemia or trauma), myocardial infarction, atherosclerosis, stroke, reperfusion injury (e.g., due to cardiopulmonary bypass or kidney dialysis), acute glomerulonephritis, vasculitis, thermal injury (i.e., sunburn), necrotizing enterocolitis, granulocyte transfusion associated syndrome, and/or Sjogren's syndrome. Exemplary inflammatory conditions of the skin include, for example, eczema, atopic dermatitis, contact dermatitis, urticaria, scleroderma, psoriasis, and dermatosis with acute inflammatory components.

In another embodiment, a polymer-agent conjugate, particle, composition or method described herein may be used to treat or prevent allergies and respiratory conditions, including asthma, bronchitis, pulmonary fibrosis, allergic rhinitis, oxygen toxicity, emphysema, chronic bronchitis, acute respiratory distress syndrome, and any chronic obstructive pulmonary disease (COPD). The polymer-agent conjugate, particle or composition may be used to treat chronic hepatitis infection, including hepatitis B and hepatitis C.

In embodiments a polymer-agent conjugate, particle, composition or method described herein may be used to treat autoimmune diseases and/or inflammation associated with autoimmune diseases such as organ-tissue autoimmune diseases (e.g., Raynaud's syndrome), scleroderma, myasthenia gravis, transplant rejection, endotoxin shock, sepsis, psoriasis, eczema, dermatitis, multiple sclerosis, autoimmune thyroiditis, uveitis, systemic lupus erythematosis, Addison's disease, autoimmune polyglandular disease (also known as autoimmune polyglandular syndrome), and Grave's disease.

In an embodiment, a nucleic acid agent-polymer conjugate, particle or composition, e.g., containing an siRNA that targets a gene listed in Table B, is administered, e.g, to treat or prevent, an associated disease listed in Table B.

TABLE B
The nucleic acid agent, e.g., an siRNA, can target a gene listed in the table,
for example, to treat or prevent the associated disease.
Inflammatory/Autoimmune Diseases

| Gene | Diseases |
|---|---|
| ICAM-1 | Inflammatory skin diseases (allergic |
| | contact dermatitis, fixed drug eruption, lichen |
| | planus, psoriasis), asthma, allergic rhinitis, |
| | allergic conjunctivitis, immune based nephritis, |
| | contact dermal hypersensitivity, type 1 diabetes, |
| | inflammatory lung diseases, inflammatory bowel |
| | disease, inflammatory skin disorders, allograft |
| | rejection, immune cell interactions, mixed t cell |
| | reaction, meningitis, multiple sclerosis, |
| | rheumatoid arthritis, septic arthritis, uveitis, age |
| | related macular degeneration |
| IL-18 | Chronic Obstructive Pulmonary Disease |
| | (COPD) |
| IFNgamma | COPD |
| PKR | COPD |
| VEGF | Preventing post operative |
| | neovascularization and post operative |
| | inflammation in ophthalmic |
| IL2R | Lupus, nephritis, inflammatory bowel |
| | disease, inflammation associated with |
| | transplanted |
| NPRA | Respiratory allergy, viral infection |
| FIZZ1 | Airway inflammation |
| Akt & p85alpha | Inflammatory bowel disease, chronic |
| | inflammatory state associated with organ |
| | transplants, pancreatitis, arthritis, enterocolitis, |
| | autoimmune disease, chronic inflammatory state |
| | associated with infection, toxin, allergy |
| TREM-1 | Asthma, rheumatoid arthritis |
| BIM, PUMA, BAX, BAK | Sepsis |
| STAT6 | Asthma, non-atopic asthma, rhinitis |
| BLT2 | Asthma |
| FCepsilonR alpha chain, | Allergic rhinitis, asthma |
| FCepsilonRbeta chain, c-Kit, LYN, SYK, | |
| ICOS, OX40L, CD40, CD80, CD86, RELA, | |
| RELB, 4-1BB ligand, TLR1, TLR2, TLR3, | |
| TLR5, TLR6, TLR7, TLR8, TLR9, CD83, | |
| SLAM, common gamma chain, COX2 | |
| IL-1, IL-2, IL-3, IL4, IL-5, IL-6, IL-7, | Allergic rhinitis, asthma, COPD |
| IL-8, IL-9, IL-10, IL-11, IL-12, IL-13, | |
| IL-14, IL-15, IL-16, IL-17, IL-18, IL-19, | |
| IL-20, IL-21, IL-22, IL-23, IL-24, IL-25, | |
| IL-26, IL-27, IL-1R, IL-2R, IL-3R, IL4R, | |
| IL-5R, IL-6R, IL-7R, IL-8R, IL-9R, IL-10R, | |
| IL-11R, IL-12R, IL-13R, IL-14R, IL-15R, | |
| IL-16R, IL-17R, IL-18R, IL-19R, IL-20R, | |
| IL-21R, IL-22R, IL-23R, IL-24R, IL-25R, | |
| IL-26R, IL-27R | |
| Calpain 1 & Calpain 2 | Asthma, asthma exacerbation, chronic |
| | obstructive pulmonary disease, opportunistic |
| | pathogenic infection of cystic fibrosis, respiratory |
| | infection, pneumonia, ventilator associated |
| | pneumonia, obstructive airway disease, bronchial |
| | condition, pulmonary inflammation, eosinophil |
| | related disorder |
| IL-1, TNFalpha, Fas, FasL | Hepatitis, cirrhosis, transplant rejection |
| IL-1, IL-2, IL-4, IL-7, IL-12, IFNs, | Rheumatoid arthritis, chron's disease, |
| GMCSF, TNFalpha | multiple sclerosis, psoriasis |
| ICAM1, VCAM1, IFN gamma, IL-1, | Suppressing rejection of transplanted |
| IL-6, IL-8, TNFalpha, CD8-, CD86, | organ by a recipient of the organ |
| MHC-II, MHC-I, CD28, CTLA4, PV-B19 | |
| TGFB1, COX2 | Wound healing |
| Cyclin D1 | Inflammatory bowel disease, ulcerative |
| | colitis, crohn's disease, celiac disease, |
| | autoimmune hepatitis, chronic rheumatoid |
| | arthritis, psoratic arthritis, insulin dependent |
| | diabetes mellitus, multiple sclerosis, enterogenic |
| | spondyloarthropathies, autoimmune myocarditis, |
| | psoriasis, scleroderma, myasthenia gravis, |
| | multiple myostisis/dermatomyostisis, hashimoto's |
| | disease, autoimmune hypocytosis, pure red cell |
| | apalsia, aplastic anemia, sjogren's syndrome, |
| | vascultis syndrome, systemic lupus |
| | erythematosus, glomerulonephritis, pulmonary |
| | inflammation, septic shock, transplant rejection |

### Cardiovascular Disease

In another aspect, the invention features, a method of treating or preventing a disease or disorder in a subject wherein in the disorder is a cardiovascular disease. The method comprises administering an effective amount of a conjugate, particle, or composition described herein to thereby treat the disease or disorder. In an embodiment the conjugates, particles and compositions can be used as part of a first line, second line, or adjunct therapy, and can also be used alone or in combination with one or more additional treatment regimes.

In embodiments the disclosed methods may be useful in the prevention and treatment of cardiovascular disease. Cardiovascular diseases that can be treated or prevented using polymer-agent conjugates, particles, compositions and methods described herein include cardiomyopathy or myocarditis; such as idiopathic cardiomyopathy, metabolic cardiomyopathy, alcoholic cardiomyopathy, drug-induced cardiomyopathy, ischemic cardiomyopathy, and hypertensive cardiomyopathy. Also treatable or preventable using polymer-agent conjugates, particles, compositions and methods described herein are atheromatous disorders of the major blood vessels (macrovascular disease) such as the aorta, the coronary arteries, the carotid arteries, the cerebrovascular arteries, the renal arteries, the iliac arteries, the femoral arteries, and the popliteal arteries. In embodiments other vascular diseases that can be treated or prevented include those related to platelet aggregation, the retinal arterioles, the glomerular arterioles, the vasa nervorum, cardiac arterioles, and associated capillary beds of the eye, the kidney, the heart, and the central and peripheral nervous systems. The polymer-agent conjugates, particles, compositions and methods described herein may also be used for increasing HDL levels in plasma of an individual.

Yet other disorders that may be treated with polymer-agent conjugates, particles, compositions and methods described herein include restenosis, e.g., following coronary intervention, and disorders relating to an abnormal level of high density and low density cholesterol.

In embodiments the polymer-agent conjugate, particle or composition can be administered to a subject undergoing or who has undergone angioplasty. In one embodiment, the polymer-agent conjugate, particle or composition is administered to a subject undergoing or who has undergone angioplasty with a stent placement. In some embodiments, the polymer-agent conjugate, particle or composition can be used as a coating for a stent.

In embodiments the polymer-agent conjugates, particles or compositions can be used during the implantation of a stent, e.g., as a separate intravenous administration, as a coating for a stent.

In an embodiment, a nucleic acid agent-polymer conjugate, particle or composition, e.g., containing an siRNA that targets a gene listed in Table C, is administered, e.g, to treat or prevent, an associated disease listed in Table C.

TABLE C
The nucleic acid agent, e.g., an siRNA, can target a gene listed in the
table, for example, to treat or prevent the associated disease.
Cardiovascular Diseases

| Gene | Diseases |
|---|---|
| ICAM-1 | Atherosclerosis, myocarditis, pulmonary |
| | fibrosis |
| S1P2 & Caspase 11 | Heart disease, stroke, peripheral vascular |
| | disease, vasculitis |
| ApoB | Hypercholesterolemia, atherosclerosis, |
| | angina pectoris, high blood pressure, diabetes, |
| | hypothyroidism |
| KLF5 | Arteriosclerosis, restenosis occurring after |
| | coronary intervention, cardiac hypertrophy |
| CETP | Cardiovascular disorders |
| PLOD2 | Fibrotic tissue formation occurring in |
| | myocardial infarct related fibrosis, cardiac fibrosis, |
| | valvular stenosis, intimal hyperplasia, diabetic |
| | ulcers, peridural fibrosis, perineural fibrosis |
| Ku | Cardiac hypertrophy, heart failure |
| Agtr11, Apelin, | Cardiovascular disease, atherosclerosis, |
| Stabilin 1, Stabilin | atherosclerotic plaque formation, plaque |
| 2, TNFaip811, | destabilization, vulnerable plaque formation and |
| TNFaip8, FGD5 | rupture |
| ROCK1 | Cardiac failure |
| PCSK9, | Heart disease |
| apolipoprotein B | |
| sNRF | Cardiovascular disease, angina pectoris, |
| | arrhythmia, cardiac fibrosis, congenital |
| | cardiovascular disease, coronary artery disease, |
| | dilated cardiomyopathy, myocardial infarction, |
| | heart failure, hypertrophic cardiomyopathy, |
| | systemic hypertension from any cause, edematous |
| | disorders caused by liver or renal disease, mitral |
| | regurgitation, myocardial tumors, myocarditis, |
| | rheumatic fever, Kawasaki disease, Takaysu |
| | arteritis, cor pulmonale, primary pulmonary |
| | hypertension, amyloidosis, hemachromatosis, toxic |
| | effects on the heart due to poisoning, Chaga's |
| | disease, heart transplantation, cardiac rejection |
| | after heart transplant, cardiomyopathy of |
| | chachexia, arrhythmogenic right ventricular |
| | dysplasia, cardiomyopathy of pregnancy, Marfan |
| | Syndrome, Turner syndrome, Loeys-Dietz |
| | Syndrome, familial bicuspid aortic valve |

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

### Example 1

### Purification and Characterization of 5050 PLGA

Step A: A 3-L round-bottom flask equipped with a mechanical stirrer was charged with 5050PLGA (300 g, Mw: 7.8 kDa; Mn: 2.7 kDa) and acetone (900 mL). The mixture was stirred for 1 h at ambient temperature to form a clear yellowish solution.
Step B: A 22-L jacket reactor with a bottom-outlet valve equipped with a mechanical stirrer was charged with MTBE (9.0 L, 30 vol. to the mass of 5050 PLGA). Celite® (795 g) was added to the solution with overhead stiffing at ˜200 rpm to produce a suspension. To this suspension was slowly added the solution from Step A over 1 h. The mixture was agitated for an additional one hour after addition of the polymer solution and filtered through a polypropylene filter. The filter cake was washed with MTBE (3×300 mL), conditioned for 0.5 h, air-dried at ambient temperature (typically 12 h) until residual MTBE was ≦5 wt % (as determined by ¹H NMR analysis).
Step C: A 12-L jacket reactor with a bottom-outlet valve equipped with a mechanical stirrer was charged with acetone (2.1 L, 7 vol. to the mass of 5050 PLGA). The polymer/Celite® complex from Step B was charged into the reactor with overhead stirring at ˜200 rpm to produce a suspension. The suspension was stirred at ambient temperature for an additional 1 h and filtered through a polypropylene filter. The filter cake was washed with acetone (3×300 mL) and the combined filtrates were clarified through a 0.45 mM in-line filter to produce a clear solution. This solution was concentrated to ˜1000 mL.
Step D: A 22-L jacket reactor with a bottom-outlet valve equipped with a mechanical stirrer was charged with water (9.0 L, 30 vol.) and was cooled down to 0-5° C. using a chiller. The solution from Step C was slowly added over 2 h with overhead stirring at ˜200 rpm. The mixture was stirred for an additional one hour after addition of the solution and filtered through a polypropylene filter. The filter cake was conditioned for 1 h, air-dried for 1 day at ambient temperature, and then vacuum-dried for 3 days to produce the purified 5050 PLGA as a white powder [258 g, 86% yield]. The ¹H NMR analysis was consistent with that of the desired product and Karl Fisher analysis showed 0.52 wt % of water. The product was analyzed by HPLC (AUC, 230 nm) and GPC (AUC, 230 nm). The process produced a narrower polymer polydispersity, i.e. Mw: 8.8 kDa and Mn: 5.8 kDa.

### Example 2

### Purification and Characterization of 5050 PLGA Lauryl Ester

A 12-L round-bottom flask equipped with a mechanical stirrer was charged with MTBE (4 L) and heptanes (0.8 L). The mixture was agitated at ˜300 rpm, to which a solution of 5050 PLGA lauryl ester (65 g) in acetone (300 mL) was added dropwise. Gummy solids were formed over time and finally clumped up on the bottom of the flask. The supernatant was decanted off and the solid was dried under vacuum at 25° C. for 24 h to afford 40 g of purified 5050 PLGA lauryl ester as a white powder [yield: 61.5%]. ¹H NMR (CDCl₃, 300 MHz): δ 5.25-5.16 (m, 53H), 4.86-4.68 (m, 93H), 4.18 (m, 7H), 1.69-1.50 (m, 179H), 1.26 (bs, 37H), 0.88 (t, J=6.9 Hz, 6H). The ¹H NMR analysis was consistent with that of the desired product. GPC (AUC, 230 nm): 6.02-9.9 min, t_{R}=7.91 min.

### Example 3

### Purification and Characterization of 7525 PLGA

A 22-L round-bottom flask equipped with a mechanical stirrer was charged with 12 L of MTBE, to which a solution of 7525 PLGA (150 g, approximately 6.6 kD) in dichloromethane (DCM, 750 mL) was added dropwise over an hour with an agitation of ˜300 rpm, resulting in a gummy solid. The supernatant was decanted off and the gummy solid was dissolved in DCM (3 L). The solution was transferred to a round-bottom flask and concentrated to a residue, which was dried under vacuum at 25° C. for 40 h to afford 94 g of purified 7525 PLGA as a white foam [yield: 62.7%]. ¹H NMR (CDCl₃, 300 MHz): δ 5.24-5.15 (m, 68H), 4.91-4.68 (m, 56H), 3.22 (s, 2.3H, MTBE), 1.60-1.55 (m, 206H), 1.19 (s, 6.6H, MTBE). The ¹H NMR analysis was consistent with that of the desired product. GPC (AUC, 230 nm): 6.02-9.9 min, t_{R}=7.37 min.

### Example 4

### Synthesis, Purification and Characterization of O-acetyl-5050-PLGA

A 2000-mL, round-bottom flask equipped with an overhead stirrer was charged with purified 5050 PLGA [220 g, Mn of 5700] and DCM (660 mL). The mixture was stirred for 10 min to form a clear solution. Ac₂O (11.0 mL, 116 mmol) and pyridine (9.4 mL, 116 mmol) were added to the solution, resulting in a minor exotherm of ˜0.5° C. The reaction was stirred at ambient temperature for 3 h and concentrated to ˜600 mL. The solution was added to a suspension of Celite® (660 g) in MTBE (6.6 L, 30 vol.) over 1 h with overhead stiffing at ˜200 rpm. The suspension was filtered through a polypropylene filter and the filter cake was air-dried at ambient temperature for 1 day. It was suspended in acetone (1.6 L, ˜8 vol) with overhead stiffing for 1 h. The slurry was filtered though a fritted funnel (coarse) and the filter cake was washed with acetone (3×300 mL). The combined filtrates were clarified though a Celite® pad to afford a clear solution. It was concentrated to ˜700 mL and added to cold water (7.0 L, 0-5° C.) with overhead stirring at 200 rpm over 2 h. The suspension was filtered though a polypropylene filter. The filter cake was washed with water (3×500 mL), and conditioned for 1 h to afford 543 g of wet cake. It was transferred to two glass trays and air-dried at ambient temperature overnight to afford 338 g of wet product, which was then vacuum-dried at 25° C. for 2 days to constant weight to afford 201 g of product as a white powder [yield: 91%]. The ¹H NMR analysis was consistent with that of the desired product. The product was analyzed by HPLC (AUC, 230 nm) and GPC (Mw: 9.0 kDa and Mn: 6.3 kDa).

### Example 5

### Synthesis, Purification and Characterization of Folate-PEG-PLGA-Lauryl Ester

The synthesis of folate-PEG-PLGA-lauryl ester involves the direct coupling of folic acid to PEG bisamine (Sigma-Aldrich, n=75, MW 3350 Da). PEG bisamine was purified due to the possibility that small molecular weight amines were present in the product. 4.9 g of PEG bisamine was dissolved in DCM (25 mL, 5 vol) and then transferred into MTBE (250 mL, 50 vol) with vigorous agitation. The polymer precipitated as white powder. The mixture was then filtered and the solid was dried under vacuum to afford 4.5 g of the product [92%]. The ¹H NMR analysis of the solid gave a clean spectrum; however, not all alcohol groups were converted to amines based on the integration of a-methylene to the amine group (63% bisamine, 37% monoamine).

Folate-(γ)CO—NH-PEG-NH₂ was synthesized using the purified PEG bisamine. Folic acid (100 mg, 1.0 equiv.) was dissolved in hot DMSO (4.5 mL, 3 vol to PEG bisamine). The solution was cooled to ambient temperature and (2-(7-Aza-1H-benzotriazole-1-yl)-1,1,3,3-tetramethyluronium hexafluorophosphate) (HATU, 104 mg, 1.2 equiv.) and N,N-Diisopropylethylamine (DIEA, 80 μL, 2.0 equiv.) were added. The resulting yellow solution was stirred for 30 minutes and PEG bisamine (1.5 g, 2 equiv.) in DMSO (3 mL, 2 vol) was added. Excess PEG bisamine was used to avoid the possible formation of di-adduct of PEG bisamine and to improve the conversion of folic acid. The reaction was stirred at 20° C. for 16 h and directly purified by CombiFlash® using a C18 column (RediSep, 43 g, C18). The fractions containing the product were combined and the CH₃CN was removed under vacuum. The remaining water solution (˜200 mL) was extracted with chloroform (200 mL×2). The combined chloroform phases were concentrated to approximately 10 mL and transferred into MTBE to precipitate the product as a yellow powder. In order to completely remove any unreacted PEG bisamine in the material, the yellow powder was washed with acetone (200 mL) three times. The remaining solid was dried under vacuum to afford a yellow semi-solid product (120 mg). HPLC analysis indicated a purity of 97% and the ¹H NMR analysis showed that the product was clean.

Folate-(γ)CO—NH-PEG-NH₂ was reacted with p-nitrophenyl-COO-PLGA-CO₂-lauryl to provide folic acid-PEG-PLGA-lauryl ester. To prepare p-nitrophenyl-COO-PLGA-CO₂-lauryl, PLGA 5050 (lauryl ester) [10.0 g, 1.0 equiv.] and p-nitrophenyl chloroformate (0.79 g, 2.0 equiv.) were dissolved in DCM. To the dissolved polymer solution, one portion of TEA (3.0 equiv.) was added. The resulting solution was stirred at 20° C. for 2 h and the ¹H NMR analysis indicated complete conversion. The reaction solution was then transferred into a solvent mixture of 4:1 MTBE/heptanes (50 vol). The product precipitated and gummed up. The supernatant was decanted off and the solid was dissolved in acetone (20 vol). The resulting acetone suspension was filtered and the filtrate was concentrated to dryness to produce the product as a white foam [7.75 g, 78%, Mn=4648 based on GPC]. The ¹H NMR analysis indicated a clean product with no detectable p-nitrophenol.

Folate-(γ)CO—NH-PEG-NH₂ (120 mg, 1.0 equiv.) was dissolved in DMSO (5 mL) and TEA (3.0 equiv.) was added. The pH of the reaction mixture was 8-9. p-nitrophenyl-COO-PLGA-CO₂-lauryl (158 mg, 1.0 equiv.) in DMSO (1 mL) was added and the reaction was monitored by HPLC. A new peak at 16.1 min (˜40%, AUC, 280 nm) was observed from the HPLC chromatogram in 1 h. A small sample of the reaction mixture was treated with excess 1,8-diazabicyclo[5.4.0]undec-7-ene (DBU) and the color instantly changed to dark yellow. HPLC analysis of this sample indicated complete disappearance of p-nitrophenyl-COO-PLGA-CO₂-lauryl and the 16.1 min peak. Instead, a peak on the right side of folate-(γ)CO—NH-PEG-NH₂ appeared. It can be concluded that the p-nitrophenyl-COO-PLGA-CO₂-lauryl and the possible product were not stable under strong basic conditions. In order to identify the new peak at 16.1 min, ˜⅓ of the reaction mixture was purified by CombiFlash®. The material was finally eluted with a solvent mixture of 1:4 DMSO/CH₃CN. It was observed that this material was yellow which could have indicated folate content. Due to the large amount of DMSO present, this material was not isolated from the solution. The fractions containing unreacted folate-(γ)CO—NH-PEG-NH₂ was combined and concentrated to a residue. A ninhydrin test of this residue gave a negative result, which may imply the lack of amine group at the end of the PEG. This observation can also explain the incomplete conversion of the reaction.

The rest of reaction solution was purified by CombiFlash®. Similarly to the previous purification, the suspected yellow product was retained by the column. MeOH containing 0.5% TFA was used to elute the material. The fractions containing the possible product were combined and concentrated to dryness. The ¹H NMR analysis of this sample indicated the existence of folate, PEG and lauryl-PLGA and the integration of these segments was close to the desired value of 1:1:1 ratio of all three components. High purities were observed from both HPLC and GPC analyses. The Mn based on GPC was 8.7 kDa. The sample in DMSO was recovered by precipitation into MTBE.

### Example 6

### Synthesis of PLGA-PEG-PLGA Nucleic Acid Agent Conjugate

The triblock copolymer PLGA-PEG-PLGA will be synthesized using a method developed by Zentner et al., Journal of Controlled Release, 72, 2001, 203-215. The molecular weight of PLGA obtained using this method will be ˜3 kDa. A similar method reported by Chen et al., International Journal of Pharmaceutics, 288, 2005, 207-218 will be used to synthesize PLGA molecular weights ranging from 1-7 kDa. The LA/GA ratio will typically be, but is not limited to, a ratio of 1:1. The minimum PEG molecular weight will be 2 kDa with an upper limit of 30 kDa. The preferred range of PEG will be 3-12 kDa. The PLGA molecular weight will be a minimum value of 4 kDa and a maximum of 30 kDa. The preferred range of PLGA will be 7-20 kDa. A nucleic acid agent, e.g., an RNA agent, will be conjugated to the PLGA through an appropriate linker (i.e., as listed in the examples) to form a polymer-nucleic acid agent conjugate. In addition, the same nucleic acid agent or a different nucleic acid agent could be attached to the other PLGA to form a dual nucleic acid agent-polymer conjugate with two same nucleic acid agents or two different nucleic acid agents. Particles could be formed from either the PLGA-PEG-PLGA alone or from a single nucleic acid agent or dual nucleic acid agent-polymer conjugate composed of this triblock copolymer.

### Example 7

### Synthesis of polycaprolactone-poly(ethylene glycol)-polycaprolactone (PCL-PEG-PCL) Nucleic Acid Agent Conjugate

The triblock PCL-PEG-PCL will be synthesized using a ring open polymerization method in the presence of a catalyst (i.e., stannous octoate) as reported in Hu et al., Journal of Controlled Release, 118, 2007, 7-17. The molecular weights of PCL obtained from this synthesis range from 2 to 22 kDa. A non-catalyst method shown in the article by Ge et al. Journal of Pharmaceutical Sciences, 91, 2002, 1463-1473 will also be used to synthesize PCL-PEG-PCL. The molecular weights of PCL that could be obtained from this particular synthesis range from 9 to 48 kDa. Similarly, another catalyst free method developed by Cerrai et al., Polymer, 30, 1989, 338-343 will be used to synthesize the triblock copolymer with molecular weights of PCL ranging from 1-9 kDa. The minimum PEG molecular weight will be 2 kDa with an upper limit of 30 kDa. The preferred range of PEG will be 3-12 kDa. The PCL molecular weight will be a minimum value of 4 kDa and a maximum of 30 kDa. The preferred range of PCL will be 7-20 kDa. A nucleic acid agent, e.g., an RNA agent, will be conjugated to the PCL through an appropriate linker (i.e., as listed in the examples) to form a nucleic acid agent-polymer conjugate. In addition, the same nucleic acid agent or a different nucleic acid agent could be attached to the other PCL to form a dual nucleic acid agent-polymer conjugate with two same nucleic acid agents or two different nucleic acid agents. Particles could be formed from either the PCL-PEG-PCL alone or from a single nucleic acid agent- or dual nucleic acid agent-polymer conjugate composed of this triblock copolymer.

### Example 8

### Synthesis of polylactide-poly(ethylene glycol)-polylactide (PLA-PEG-PLA) Nucleic Acid Agent Conjugate

The triblock PLA-PEG-PLA copolymer will be synthesized using a ring opening polymerization using a catalyst (i.e. stannous octoate) reported in Chen et al., Polymers for Advanced Technologies, 14, 2003, 245-253. The molecular weights of PLA that can be formed range from 6 to 46 kDa. A lower molecular weight range (i.e. 1-8 kDa) could be achieved by using the method shown by Zhu et al., Journal of Applied Polymer Science, 39, 1990, 1-9. The minimum PEG molecular weight will be 2 kDa with an upper limit of 30 kDa. The preferred range of PEG will be 3-12 kDa. The PLA molecular weight will be a minimum value of 4 kDa and a maximum of 30 kDa. The preferred range of PLA will be 7-20 kDa. A nucleic acid agent, e.g., an RNA agent, will be conjugated to the PLA through an appropriate linker (i.e., as listed in the examples) to form a nucleic acid agent-polymer conjugate. In addition, the same nucleic acid agent or a different nucleic acid agent could be attached to the other PLA to form a dual nucleic acid agent-polymer conjugate with two same nucleic acid agents or two different nucleic acid agents. Particles could be formed from either the PLA-PEG-PLA alone or from a single nucleic acid agent- or dual nucleic acid agent-polymer conjugate composed of this triblock copolymer.

### Example 9

### Synthesis of p-dioxanone-co-lactide-poly(ethylene glycol)-p-dioxanone-co-lactide (PDO-PEG-PDO) Nucleic Acid Agent Conjugate

The triblock PDO-PEG-PDO will be synthesized in the presence of a catalyst (stannous 2-ethylhexanoate) using a method developed by Bhattari et al., Polymer International, 52, 2003, 6-14. The molecular weight of PDO obtained from this method ranges from 2-19 kDa. The minimum PEG molecular weight will be 2 kDa with an upper limit of 30 kDa. The preferred range of PEG will be 3-12 kDa. The PDO molecular weight will be a minimum value of 4 kDa and a maximum of 30 kDa. The preferred range of PDO will be 7-20 kDa. A nucleic acid agent, e.g., an RNA agent, will be conjugated to the PDO through an appropriate linker (i.e., as listed in the examples) to form a nucleic acid agent-polymer conjugate. In addition, the same nucleic acid agent or a different nucleic acid agent could be attached to the other PDO to form a dual nucleic acid agent-polymer conjugate with two same nucleic acid agents or two different nucleic acid agents. Particles could be formed from either the PDO-PEG-PDO alone or from a single nucleic acid agent- or dual nucleic acid agent-polymer conjugate composed of this triblock copolymer.

### Example 10

### Synthesis of Polyfunctionalized PLGA/PLA Based Polymers

One could synthesize a PLGA/PLA related polymer with functional groups that are dispersed throughout the polymer chain that is readily biodegradable and whose components are all bioacceptable components (i.e. known to be safe in humans). Specifically, PLGA/PLA related polymers derived from 3-S-[benxyloxycarbonyl)methyl]-1,4-dioxane-2,5-dione (BMD) could be synthesized (see structures below). (The structures below are intended to represent random copolymers of the monomeric units shown in brackets.) Exemplary R groups include a negative charge, H, alkyl, and arylalkyl.

1. PLGA/PLA Related Polymer Derived from BMD

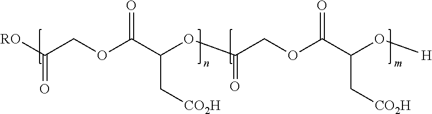

2. PLGA/PLA Related Polymer with BMD and 3,5-dimethyl-1,4-dioxane-2,5-dione (bis-DL-lactic acid cyclic diester)

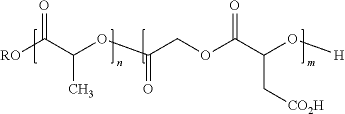

3. PLGA/PLA Related Polymer with BMD and 1,4-dioxane-2,5-dione (bis-glycolic acid cyclic diester

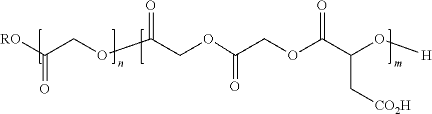

In a preferred embodiment, PLGA/PLA polymers derived from BMD and bis-DL-lactic acid cyclic diester will be prepared with a number of different pendent functional groups by varying the ratio of BMD and lactide. For reference, if it is assumed that each polymer has a number average molecular weight (Mn) of 8 kDa, then a polymer that is 100 wt % derived from BMD has approximately 46 pendant carboxylic acid groups (1 acid group per 0.174 kDa). Similarly, a polymer that is 25 wt % derived from BMD and 75 wt % derived from 3,5-dimethyl-1,4-dioxane-2,5-dione (bis-DL-lactic acid cyclic diester) has approximately 11 pendant carboxylic acid groups (1 acid group per 0.35 kDa). This compares to just 1 acid group for an 8 kDa PLGA polymer that is not functionalized and 1 acid group/2 kDa if there are 4 sites added during functionalization of the terminal groups of a linear PLGA/PLA polymer or 1 acid group/1 kDa if a 4 kDa molecule has four functional groups attached.

Specifically, the PLGA/PLA related polymers derived from BMD will be developed using a method by Kimura et al., Macromolecules, 21, 1988, 3338-3340. This polymer will have repeating units of glycolic and malic acid with a pendant carboxylic acid group on each unit [RO(COCH₂OCOCHR₁O)ₙH where R is H, or alkyl or PEG unit, etc., and R₁ is CO₂H]. There is one pendant carboxylic acid group for each 174 mass units. The molecular weight of the polymer and the polymer polydispersity can vary with different reaction conditions (i.e. type of initiator, temperature, processing condition). The Mn could range from 2 to 21 kDa. Also, there will be a pendant carboxylic acid group for every two monomer components in the polymer. Based on the reference previously sited, NMR analysis showed no detectable amount of the β-malate polymer was produced by ester exchange or other mechanisms.

Another type of PLGA/PLA related polymer derived from BMD and 3,5-dimethyl-1,4-dioxane-2,5-dione (bis-DL-lactic acid cyclic diester) will be synthesized using a method developed by Kimura et al., Polymer, 1993, 34, 1741-1748. They showed that the highest BMD ratio utilized was 15 mol % and this translated into a polymer containing 14 mol % (16.7 wt %) of BMD-derived units. This level of BMD incorporation represents approximately 8 carboxylic acid residues per 8 kDa polymer (1 carboxylic acid residue/kDa of polymer). Similarly to the use of BMD alone, no β-malate derived polymer was detected. Also, Kimura et al. reported that the glass transition temperatures (T_{g}) were in the low 20° C.'s despite the use of high polymer molecular weights (36-67 kDa). The T_{g}'s were in the 20-23° C. for these polymers whether the carboxylic acid was free or still a benzyl group. The inclusion of more rigidifying elements (i.e. carboxylic acids which can form strong hydrogen bonds) should increase the T_{g}. Possible prevention of aggregation of any particles formed from a polymer drug conjugate derived from this specific polymer will have to be evaluated due to possible lower T_{g} values.

Another method for synthesizing a PLA-PEG polymer that contains varying amounts of glycolic acid malic acid benzyl ester involves the polymerization of BMD in the presence of 3,5-dimethyl-1,4-dioxane-2,5-dione (bis-DL-lactic acid cyclic diester), reported by Lee et al., Journal of Controlled Release, 94, 2004, 323-335. They reported that the synthesized polymers contained 1.3-3.7 carboxylic acid units in a PLA chain of approximately 5-8 kDa (total polymer weight was approximately 11-13 kDa with PEG being 5 kDa) depending on the quantity of BMD used in the polymerization. In one polymer there were 3.7 carboxylic acid units/hydrophobic block in which the BMD represents approximately 19 wt % of the weight of the hydrophobic block. The ratio of BMD to lactide was similar to that observed by Kimura et al., Polymer, 1993, 34, 1741-1748 and the acid residues were similar in the resulting polymers (approximately 1 acid unit/kDa of hydrophobic polymer).

Polymers functionalized with BMD that are more readily hydrolysable will be prepared using the method developed by Kimura et al., International Journal of Biological Macromolecules, 25, 1999, 265-271. They reported that the rate of hydrolysis was related to the number of free acid groups present (with polymers with more acid groups hydrolyzing faster). The polymers had approximately 5 or 10 mol % BMD content. Also, in the reference by Lee et al., Journal of Controlled Release, 94, 2004, 323-335, the rate of hydrolysis of the polymer was fastest with the highest concentration of pendent acid groups (6 days for polymer containing 19.5 wt % of BMD and 20 days for polymer containing 0 wt % of BMD).

A nucleic acid agent, e.g., a DNA agent or an RNA agent, could be conjugated to a PLGA/PLA related polymer with BMD (refer to previous examples above). Similarly, a particle could be prepared from such a nucleic acid agent-polymer conjugate.

### Example 11

### Synthesis of Polymers Prepared Using β-Lactone of Malic Acid Benzyl Esters

One could prepare a polymer by polymerizing MePEGOH with RS-β-benzyl malolactonate (a β-lactone) with DL-lactide (cyclic diester of lactic acid) to afford a polymer containing MePEG (lactic acid) (malic acid) Me(OCH₂CH₂O)[OCCCH(CH₃)O]ₘ[COCH₂CH(CO₂H)O] as developed by Wang et al., Colloid Polymer Sci., 2006, 285, 273-281. These polymers will potentially degrade faster because they contain higher levels of acidic groups. It should be noted that the use of β-lactones generate a different polymer from that obtained using 3-[(benzyloxycarbonyl)methyl]-1,4-dioxane-2,5-dione. In these polymers, the carboxylic acid group is directly attached to the polymer chain without a methylene spacer.

Another polymer that could be prepared directly from a β-lactone was reported by Ouhib et al., Ch. Des. Monoeres. Polym, 2005, 1, 25. The resulting polymer (i.e. poly-3,3-dimethylmalic acid) is water soluble as the free acid, has pendant carboxylic acid groups on each unit of the polymer chain and as well it has been reported that 3,3-dimethylmalic acid is a nontoxic molecule.

One could polymerize 4-benzyloxycarbonyl-,3,3-dimethyl-2-oxetanone in the presence of 3,5-dimethyl-1,4-dioxane-2,5-dione (DDD) and β-butyrolactone to generate a block copolymer with pendant carboxylic acid groups as shown by Coulembier et al., Macromolecules, 2006, 39, 4001-4008. This polymerization reaction was carried out with a carbene catalyst in the presence of ethylene glycol. The catalyst used was a triazole carbene catalyst which leads to polymers with narrow polydispersities.

### Example 12

### Synthesis, Purification, and Characterization of 2-(2-(Pyridin-2-yl)disulfanyl)ethylamine

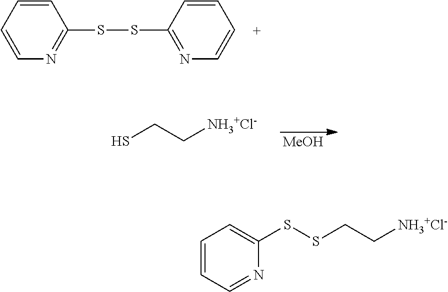

In a 25 mL round bottom flask, 2,2′-dithiodipyridine (2.0 g, 9.1 mmol) was dissolved in methanol (8 mL) with acetic acid (0.3 mL). Cysteamine hydrochloride (520 mg, 4.5 mmol) was dissolved in methanol (5 mL) and added dropwise into the mixture over ½ h. The mixture was stirred overnight. It was then concentrated under vacuum to yield yellow oil. The oil was dissolved back in methanol (5 mL) and then precipitated into diethyl ether (100 mL). The precipitate was filtered off and dried. It was then redissolved in methanol (5 mL) and reprecipitated in diethyl ether (100 mL). This procedure was repeated for two more times. The pale yellow solid was filtered off and dried to yield the final product (0.74 g, 74% yield) which was used without further purification. The ¹H NMR analysis was consistent with that of the desired product.

### Example 13

### Synthesis, Purification, and Characterization of 3-(2-(Pyridin-2-yl)disulfanyl)propionic acid

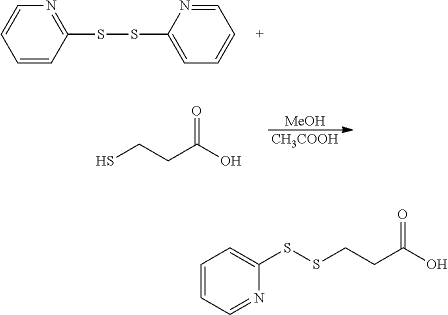

In a 250 mL round bottom flask, 2,2′-dipyridyl disulfide (8.3 g, 38 mmol) was dissolved in methanol (100 mL) with acetic acid (1.5 mL). 3-Mercaptopropionic acid (2.0 g, 19 mmol) was added to the solution and stirred for 18 h at ambient temperature. The solvent was removed under vacuum to yield yellow oil and solid mixtures. The reaction mixture was purified by flash column chromatography with DCM:MeOH (30:1). It was then further purified by recrystallization to yield white crystals (1.2 g, 29%). The ¹H NMR analysis was consistent with that of the desired product.

### Example 14

### Synthesis, Purification, and Characterization of Succinate-5050 PLGA-mPEG₂ₖ

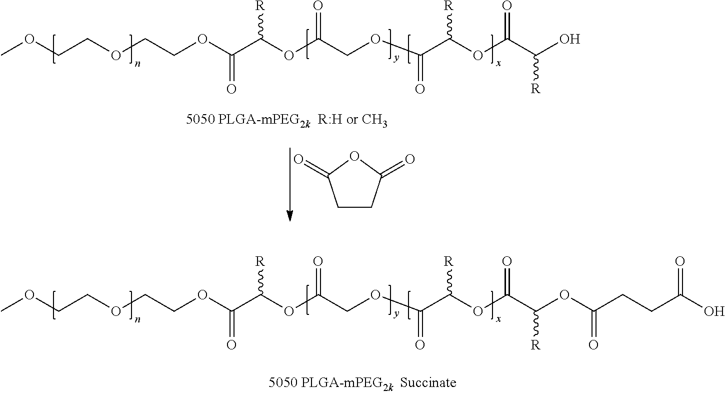

In a 50 mL round bottom flask, mPEG₂ₖ-5050 PLGA₉ₖ (MW=11k, 5.0 g, 0.45 mmol), succinic anhydride (91 mg, 0.91 mmol) and DMAP (56 mg, 0.45 mmol) were dissolved in dichloromethane (15 mL) and was stirred for 18 h at ambient temperature. The polymer was precipitated into suspension of Celite® (15 g) in diethyl ether (100 mL). Celite® was filtered off and dried overnight. Acetone (50 mL) was added to Celite® and stirred for ½ h. It was then filtered, washed with acetone, and concentrated under vacuum to about 5 mL. It was precipitated out in diethyl ether (50 mL) to yield a brown greasy solid with brown gum. The gum was kept in the freezer (−20° C.) until solidified (˜15 min.). It was then dried under vacuum to yield light brown solid (3.2 g, 58% yield). The ¹H NMR analysis was consistent with that of the desired product.

### Example 15

### Synthesis, Purification, and Characterization of N,N-diethyldiethylenetriamine-succinamide-5050 PLGA-mPEG₂ₖ

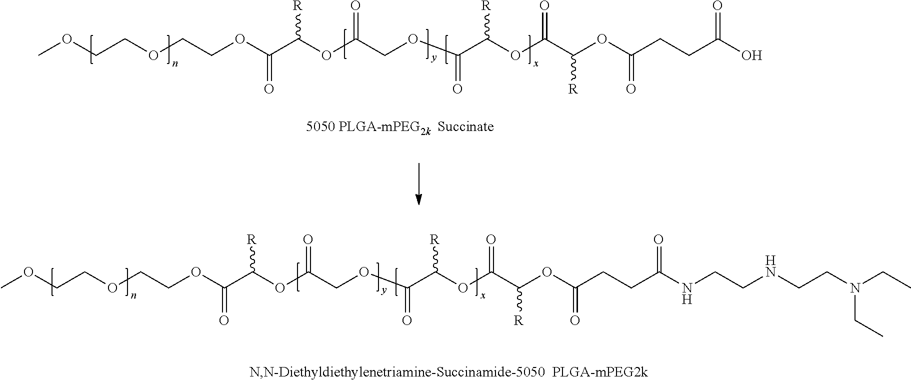

In a 50 mL round bottom flask, mPEG₂ₖ-5050 PLGA₉ₖ-succinate (2.0 g, 0.26 mmol) was dissolved in DCM (10 mL). To the reaction mixture, N,N-diethyldiethylenetriamine (210 mg, 1.3 mmol), NHS (61 mg, 0.53 mmol) and EDC (82 mg, 0.53 mmol) were added. It was then stirred at room temperature for 4 h. The reaction mixture was added Et₂O (100 mL) to precipitate out the polymer. It was then rinsed with Et₂O (20 mL) and dried under vacuum to yield light brown solid (1.9 g, 95% yield). The ¹H NMR analysis was consistent with that of the desired product.

### Example 16

### Synthesis, Purification, and Characterization of 2-(2-(pyridin-2-yl)disulfanyl)ethylamino-5050-PLGA-O-acetyl

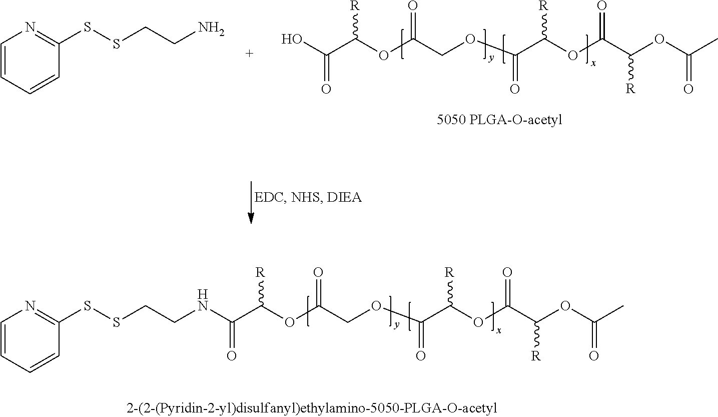

In a 50 mL round bottom flask, 5050 PLGA_{6.3k}-O-acetyl (2.0 g, 0.32 mmol), NHS (66 mg, 0.57 mmol) and EDC (122 mg, 0.63 mmol) was dissolved in DMF (12 mL). To the reaction mixture, 2-(2-(pyridin-2-yl)disulfanyl)ethylamine (127 mg, 0.57 mmol) and diisopropylethylamine (82 mg, 0.63 mmol) in DMF (6 mL) were added. The reaction mixture was then stirred at room temperature for 4 h. Water (40 mL) was added to the reaction mixture to give a gummy solid. The gummy solid was dissolved in DCM (15 mL) and washed twice with 0.1% aqueous HCl solution (50 mL×2) followed by brine (100 mL). The organic layer was dried over sodium sulphate and further purified by precipitation into cold ether (100 mL). Solvent was removed and the material was dried under vacuum to yield white solid (1.4 g, 68% yield). The ¹H NMR analysis was consistent with that of the desired product.

### Example 17

### Synthesis, Purification, and Characterization of N,N-diethyldiethylenetriamine 5050 PLGA-O-acetyl

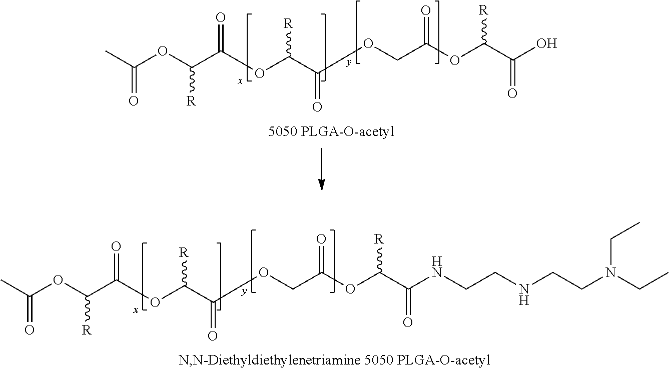

In a 50 mL round bottom flask, 5050 PLGA-O-acetyl (Mw: 16 kDa, 2.0 g, 0.13 mmol) was dissolved in DCM (10 mL). To the reaction mixture, N,N-diethyldiethylenetriamine (100 mg, 0.63 mmol), NHS (29 mg, 0.25 mmol) and EDC (39 mg, 0.25 mmol) were added. It was then stirred at room temperature for 4 h. Cold Et₂O (100 mL) was added to the reaction mixture to precipitate out the polymer. The precipitated polymer was dried under vacuum to yield a white foam. The ¹H NMR analysis was consistent with that of the desired product.

### Example 18

### Synthesis, Purification, and Characterization of succinimidyl-N-hydroxy ester 5050 PLGA-O-acetyl

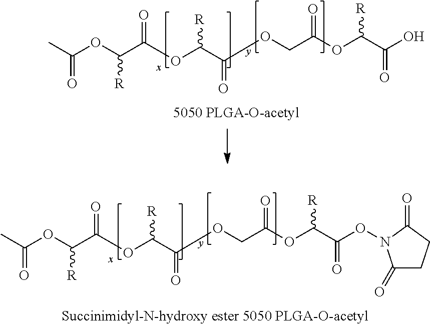

In a 50 mL round bottom flask, 5050-PLGA₉ₖ-O-acetyl (2 g, 0.33 mmol) will be dissolved in DCM (12 mL) followed by the addition of NHS (78 mg, 0.67 mmol) and EDC (100 mg, 0.67 mmol). The reaction mixture will be stirred for 4 hours at room temperature. The polymer will be solvated in DCM and purified by precipitation in cold ether 3 times (50×3 mL). The solid will be dried under vacuum overnight and analyzed by ¹H NMR.

### Example 19

### Synthesis, Purification, and Characterization of 2-(2-(pyridin-2-yl)disulfanyl)ethylamino-mPEG₁₀ₖ

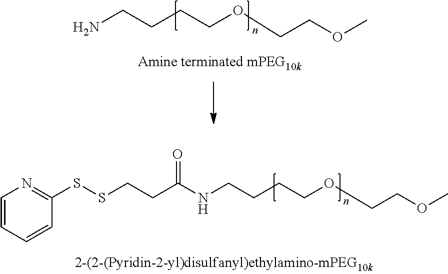

Amine group terminated mPEG₁₀ₖ (5.5 mg, 0.0053 mmol) will be reacted with N-succinimidyl 3-(2-pyridyldithio) propionate (1.12 mg, 0.032 mmol) in PBS buffer (pH 7.2) for 3 hours and purified by dialysis (membrane molecular weight cutoff: 3500). The purified material will be lyophilized and analyzed by ¹H NMR.

### Example 20

### Synthesis, Purification, and Characterization of 2-(2-(pyridin-2-yl)disulfanyl)propanoate-5050-PLGA-mPEG₂ₖ

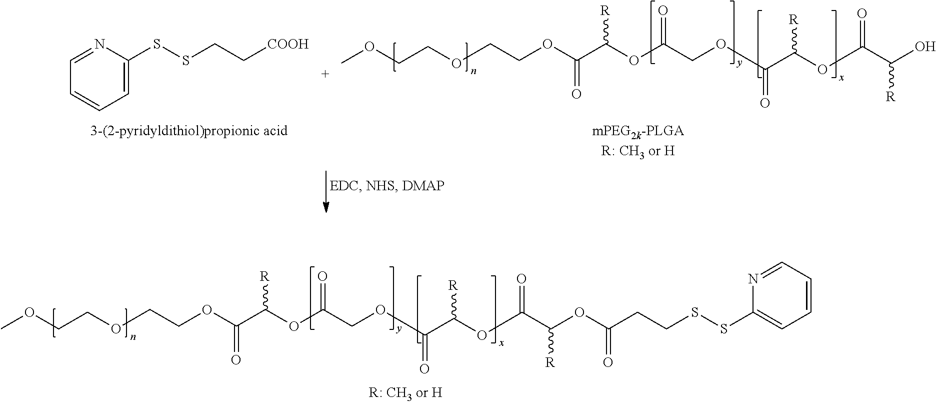

In a 50 mL round bottom flask, 5050 PLGA₉ₖ-O-mPEG₂ₖ (Mw.: 11 kDa, 1.0 g, 0.09 mmol) was dissolved in DCM (8 mL). To the reaction mixture, 3-(2-(pyridin-2-yl)disulfanyl)propionic acid (30 mg, 0.14 mmol), NHS (20 mg, 0.1 mmol) and EDC (22 mg, 0.17 mmol) were added. It was then stirred at room temperature for 4 h. Cold Et₂O (100 mL) was added to the reaction mixture to precipitate out the polymer. The precipitated polymer was dried under vacuum to yield a white foam. The ¹H NMR analysis was consistent with that of the desired product.

### Example 21

### Synthesis, Purification, and Characterization of azide terminated-PEG linker-5050 PLGA-O-acetyl

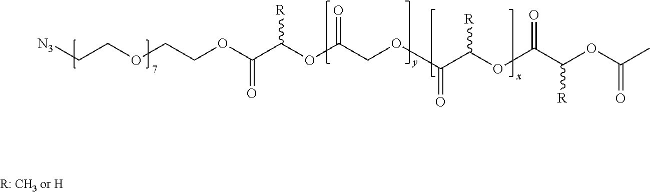

In a 50 mL round bottom flask, 5050 PLGA-O-acetyl (2.0 g, 0.13 mmol) will be dissolved in DCM (10 mL). To the reaction mixture, azide-PEG₈-OH (40 mg, 0.13 mmol), NHS (29 mg, 0.25 mmol) and EDC (39 mg, 0.25 mmol) will be added. It was then stirred at RT for 4 h. Cold Et₂O (100 mL) will then be added to the reaction mixture to precipitate out the polymer. The precipitated polymer will be dried under vacuum to yield a white foam. The ¹H NMR analysis will be carried out to determine the identity of the desired compound.

### Example 21a

### Synthesis, Purification, and Characterization of Glutamic Acid-PLGA5050-O-acetyl

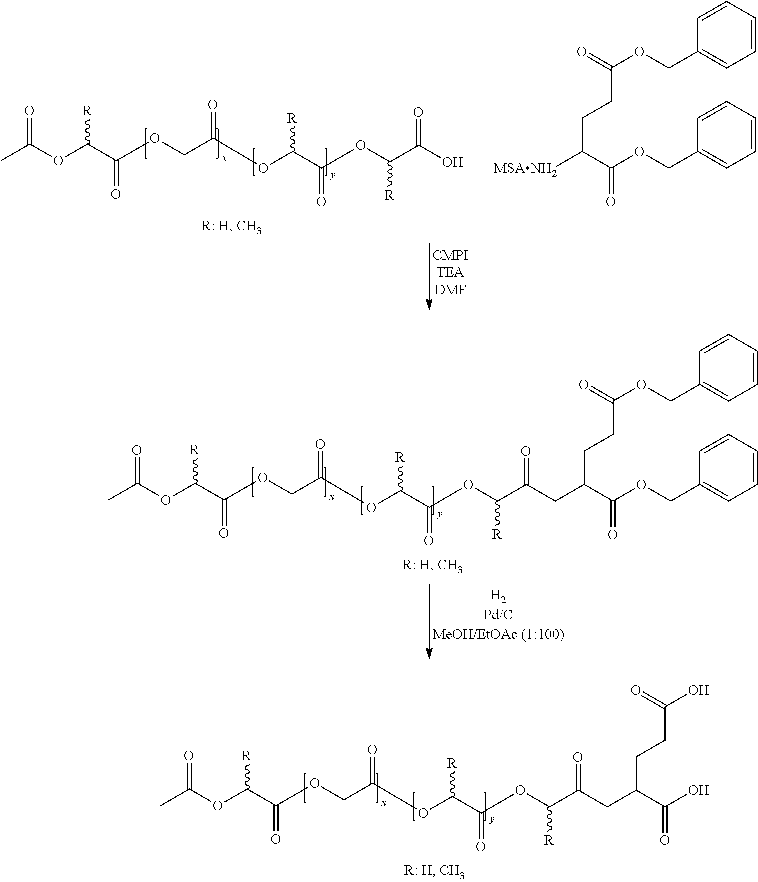

A 500-mL, round-bottom flask was charged with 5050 PLGA-O-Acetyl (40 g, 5.88 mmol), dibenzyl glutamate (3.74 g, 7.35 mmol), and DMF (120 mL, 3 vol.) and allowed to mix for 10 min to afford a clear solution. CMPI (2.1 g, 8.23 mmol) and TEA (2.52 mL) were added and the solution was stirred at ambient temperature for 3 h. The yellowish solution was added to a suspension of Celite® (120 g) in MTBE (2.0 L) over 0.5 h with overhead stiffing. The solid was filtered, washed with MTBE (300 mL), and vacuum dried at 25° C. for 16 h. The solid was then suspended in acetone (400 mL, 10 vol), stirred for 0.5 h, filtered and the filter cake was washed with acetone (3×100 mL). The combined filtrates were concentrated to 150 mL and added to cold water (3.0 L, 0-5° C.) over 0.5 h with overhead stirring. The resulting suspension was stirred for 2 h and filtered through a PP filter. The filter cake was air-dried for 3 h and then vacuum dried at 28° C. for 16 h to afford the product, dibenzylglutamate 5050 PLGA-O-acetyl (40 g, yield: 95%). The ¹H NMR analysis indicated that the ratio of benzyl aromatic protons to methane protons of lactide was 10:46. HPLC analysis indicated 96% purity (AUC, 227 nm) and GPC analysis showed Mw 8.9 kDa and Mn 6.5 kDa.

Dibenzyl glutamate 5050 PLGA-O-acetyl (40 g) was dissolved in ethyl acetate (400 mL) to afford a yellowish solution. Charcoal (10 g) was added to the mixture and stirred for 1 h at ambient temperature. The solution was filtered through a pad of Celite® (60 mL) to afford a colorless filtrate. The filter cake was washed with ethyl acetate (3×50 mL) and the combined filtrates were concentrated to 400 mL. Palladium on activated carbon (Pd/C, 5 wt %, 4.0 g) was added, the mixture was evacuated for 1 min, filled up with H₂ using a balloon and the reaction was stirred at ambient temperature for 3 h. The solution was filtered through a Celite® pad (100 mL) and the filter cake was washed with acetone (3×50 mL). The combined filtrates had a grey color and were concentrated to 200 mL. The solution was added to a suspension of Celite® (120 g) in MTBE (2.0 L) over 0.5 h with overhead stiffing. The suspension was stirred at ambient temperature for 1 h and filtered through a PP filter. The filter cake was dried at ambient temperature for 16 h, suspended in acetone (400 mL), and stirred for 0.5 h. The solution was filtered through a PP filter and the filter cake was washed with acetone (3×50 mL). To remove any residual Pd, macroporous polystyrene-2,4,6-trimercaptotriazine resin (MP-TMT, 2.0 g, Biotage, capacity: 0.68 mmol/g) was added at ambient temperature for 16 h with overhead stirring. The solution was filtered through a Celite® pad to afford a light grey solution. The solution was concentrated to 200 mL and added to cold water (3.0 L, 0-5° C.) over 0.5 h with overhead stirring. The resulting suspension was stirred at <5° C. for 1 h and filtered through a PP filter. The filter cake was air-dried for 12 h and vacuum dried for 2 days to afford a semi-glassy solid (glutamic acid-PLGA5050-O-acetyl, 38 g, yield: 95%). HPLC analysis showed 99.6% purity (AUC, 227 nm) and GPC analysis indicated Mw 8.8 kDa and Mn 6.6 kDa.

### Example 21b

### Synthesis and Purification of bis-(N1-spermine) glutamide-5050 PLGA-O-acetyl

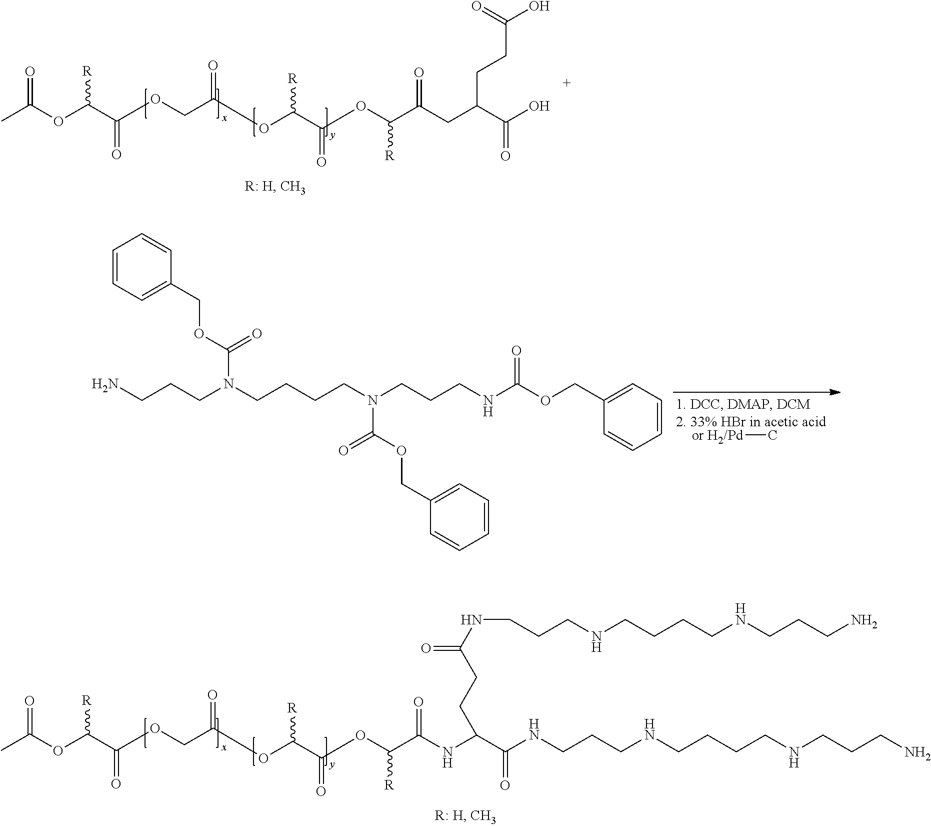

Glutamic acid-PLGA5050-O-acetyl (1.4 g, 0.26 mmol), (N-1-PLGA-N5,N10,N14-tri-Cbz)-spermine (630 mg, 1.0 mmol), DCC (160 mg, 0.77 mmol), NHS (89 mg, 0.77 mmol) and TEA (160 mg, 1.5 mmol) were dissolved in DCM (50 mL) and stirred overnight at rt. DCM was removed under vacuum. DMF solution was added to diethyl ether (50 mL) to isolate the yellow material. It was then washed with MeOH (25 mL) twice and followed by water (25 mL) wash. It was then lyophilized to yield white solid, bis-(N1-PLGA-N5,N10,N14-tri-Cbz)-spermine glutamide-5050 PLGA-O-acetyl (1.3 g, 93% yield).

Bis-(N1-PLGA-N5,N10,N14-tri-Cbz)-spermine glutamide-5050 PLGA-O-acetyl (1.0 g, 0.15 mmol, MW6,600) was dissolved in 33% HBr in acetic acid (5 mL) to yield clear brown solution and the reaction mixture was stirred at room temperature for 2 h. It was then added to diethyl ether (100 mL). The solid was rinsed with MeOH (30 mL). It was decanted and rewashed with water (30 mL). It was then frozen and lyophilized to yield pale yellow solid (0.79 g, 79% yield).

### Example 22

### Synthesis, Purification, and Characterization of Oligonucleotide-C6-SS-5050 PLGA-O-acetyl

C6-thiol modified oligonucleotides (siRNA, 0.2 mg, 14.7 nmol) were conjugated to 2-(2-(pyridin-2-yl)disulfanyl)ethylamino-5050-PLGA-O-acetyl (10 mg, 1.58 μmol) as prepared in Example 16 in a solvent mixture of 95:5 DMSO:TE buffer (1 mL). The reaction mixture was stirred at 65° C. for 2 hours. The oligonucleotide-5050-PLGA-O-acetyl conjugate was analyzed by reverse phase HPLC and gel electrophoresis.

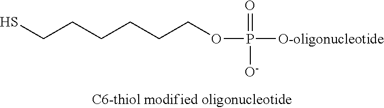

### Example 22a

### Synthesis, Purification, and Characterization of Oligonucleotide-C6-SS-5050 PLGA-O-acetyl

C6-thiol modified oligonucleotides against EGFP (enhanced green fluorescent protein) having a Mw of 13.2 kDa (siRNA, 20 mg, 1.51 μmol) with sense strands having nucleotide sequences substantially identical to a portion of the EGFP sequence, being 19 base pairs in length with a UU overhang, and having complementary antisense strands, were conjugated to 2-(2-(Pyridin-2-yl)disulfanyl)ethylamino-5050-PLGA-O-acetyl (85 mg, 11 μmol) as prepared in Example 16 in a solvent mixture of 95:5 DMSO:TE buffer (10 mL). The reaction mixture was stirred at 65° C. for 3 hours. The oligonucleotide-5050-PLGA-O-acetyl conjugate was analyzed by reverse phase HPLC and gel electrophoresis.

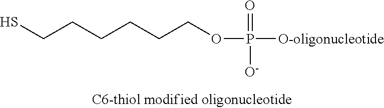

### Example 22b

### Synthesis, Purification, and Characterization of Oligonucleotide-C6-SS-5050 PLGA-O-acetyl

C6-thiol modified oligonucleotides against luciferase (siRNA, 20 mg, 1.51 μmol, Mw of 13.6 kDa) with sense strands having nucleotide sequences substantially identical to a portion of the luciferase sequence, being 19 base pairs in length with a UU overhang, and having complementary antisense strands, were conjugated to 2-(2-(Pyridin-2-yl)disulfanyl)ethylamino-5050-PLGA-O-acetyl (85 mg, 11 μmol) as prepared in Example 16 in a solvent mixture of 95:5 DMSO:TE buffer (10 mL). The reaction mixture was stirred at 65° C. for 3 hours. The oligonucleotide-5050-PLGA-O-acetyl conjugate was analyzed by reverse phase HPLC and gel electrophoresis.

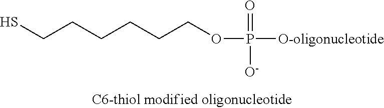

### Example 23

### Synthesis, Purification, and Characterization of Oligonucleotide-C6-SS-5050 PLGA-O-mPEG₂ₖ

C6-Thiol modified oligonucleotides (siRNA or DNA, 2 mg, 0.13 μmol) (as used in Example 22) will be conjugated to 2-(2-(pyridin-2-yl)disulfanyl)ethylamino-5050 PLGA-mPEG₂ₖ (6.9 mg, 0.625 μmol) in a solvent mix (20:80, PBS:ACN, pH 8, 0.6 mL). The reaction mixture will be stirred under argon at room temperature for 48 hours. The oligonucleotide-5050 PLGA-mPEG₂ₖ conjugate will be analyzed and purified by preparative anionic exchange and reverse phase HPLC.

### Example 24

### Synthesis, Purification, and Characterization of Oligonucleotide-C6-SS-5050 PLGA-O-acetyl via particle formation

C6-Thiol modified oligonucleotides (siRNA or DNA, 2 mg, 0.13 μmol) (as used in Example 22) will be conjugated to 2-(2-(pyridin-2-yl)disulfanyl)ethylamino-5050-PLGA-β-acetyl containing, preformed particles (4 mg, 0.625 μmol) in buffer (PBS, pH 8, 0.4 mL). The reaction mixture will be stirred under argon at room temperature for 48 hours. The oligonucleotide-5050 PLGA-mPEG₂ₖ conjugate will be analyzed and purified by preparative anionic exchange and reverse phase HPLC.

### Example 25

### Synthesis, Purification, and Characterization of Oligonucleotide-C12-amide-5050 PLGA-O-acetyl

C12-amino modified oligonucleotides (siRNA or DNA, 2 mg, 0.13 μmol) will be conjugated to succinimidyl-N-hydroxy ester 5050 PLGA-O-acetyl (4 mg, 0.625 μmol) in a solvent mix (20:80, PBS:ACN, pH 8, 0.4 mL). The reaction mixture will be stirred under argon at room temperature for 48 hours. The oligonucleotide-C12 amide 5050 PLGA-O-acetyl conjugate will be analyzed and purified by preparative anionic exchange and reverse phase HPLC.

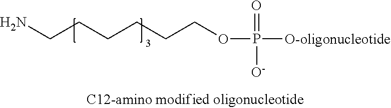

### Example 26

### Synthesis, Purification, and Characterization of Oligonucleotide-PEG-ester-5050 PLGA-O-acetyl

PEG modified oligonucleotides (siRNA or DNA, 2 mg, 0.13 μmol) will be conjugated to succinimidyl-N-hydroxy ester 5050 PLGA-O-acetyl (4 mg, 0.625 μmol) in DMSO (0.4 mL) with DMAP (0.625 mmol). The reaction mixture will be stirred under argon at room temperature for 48 hours. The oligonucleotide-C18 PEG 5050 PLGA-O-acetyl conjugate will be analyzed and purified by preparative anionic exchange and reverse phase HPLC.

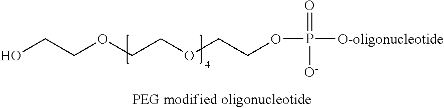

### Example 27

### Synthesis and Purification of Oligonucleotide-SS-mPEG

C6-Thiol modified oligonucleotides (siRNA or DNA, 2 mg, 0.13 μmol) (as used in Example 22) will be conjugated to 2-(2-(pyridin-2-yl)disulfanyl)ethylamino-mPEG₁₀ₖ (6.5 mg, 0.625 μmol) in buffer (PBS, pH 8, 0.4 mL). The reaction mixture will be stirred under argon at room temperature for 48 hours. The reaction mixture will be analyzed and purified by HPLC analysis using Superdex® column.

### Example 28

### Synthesis, Purification, and Characterization of Oligonucleotide-C12-amide-5050 PLGA-mPEG₂ₖ

C12-amino modified oligonucleotides (siRNA or DNA, 2 mg, 0.13 μmol) (as used in Example 25) will be conjugated to mPEG₂ₖ-5050 PLGA-succinate (4 mg, 0.625 μmol) in a solvent mix (50:50, PBS:ACN, pH 8, 0.4 mL). The reaction mixture will be stirred under argon at room temperature for 48 hours. The oligonucleotide-C12 amide 5050 PLGA-mPEG₂ₖ conjugate will be analyzed and purified by preparative anionic exchange and reverse phase HPLC.

### Example 29

### Synthesis, Purification, and Characterization of Oligonucleotide-PEG-ester-5050 PLGA-mPEG₂ₖ

PEG modified oligonucleotides (siRNA or DNA, 2 mg, 0.13 μmol) (as used in Example 26) will be conjugated to mPEG₂ₖ-5050 PLGA-Succinate (4 mg, 0.625 μmol) in a solvent mix (50:50, PBS:ACN, pH 8, 0.4 mL) with DMAP (0.625 mmol). The reaction mixture will be stirred under argon at room temperature for 48 hours. The oligonucleotide-C18 PEG 5050 PLGA-mPEG₂ₖ conjugate will be analyzed and purified by preparative anionic exchange and reverse phase HPLC.

### Example 30

### Synthesis, Purification, and Characterization of Oligonucleotide-C6-triazole-PEG-5050 PLGA-O-acetyl

10 μL precomplexed Cu(I) will be added (10 mM; 1 mg CuBr (99.99%) dissolved in 700 μL of 10 mM TBTA tris(benzyltriazolylmethyl)amine ligand in tert-BuOH:DMSO 1:3) to a reaction mixture of C6-alkyne-modified oligonucleotides (siRNA or DNA) (1 to 4 pmol siRNA or DNA, 10 mM Tris) and azide terminated-PEG-5050 PLGA-O-acetyl solution (10 μL of 5 mM, diluted with 10 mM Tris with 5% tBuOH from a stock of 0.1 N in DMSO) (Example 21). The sample will be stirred at room temperature for 2 hours. The reaction mixture will be analyzed by anionic-exchange and reversed phase HPLC.

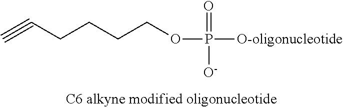

### Example 31

### Synthesis, Purification, and Characterization of Oligonucleotide-PEG-triazole-PEG-5050 PLGA-O-acetyl

10 μL precomplexed Cu(I) will be added (10 mM; 1 mg CuBr (99.99%) dissolved in 700 μL of 10 mM TBTA tris(benzyltriazolylmethyl)amine ligand in tert-BuOH:DMSO 1:3) to a reaction mixture of alkyne-PEG-modified oligonucleotides (siRNA or DNA) (1 to 4 μmol siRNA or DNA, 10 mM Tris) and azide terminated-PEG-5050 PLGA-O-acetyl solution (10 μL of 5 mM, diluted with 10 mM Tris with 5% tBuOH from a stock of 0.1N in DMSO) (Example 21). The sample will be stirred at room temperature for 2 hours. The reaction mixture will be analyzed by anionic-exchange and reversed phase HPLC.

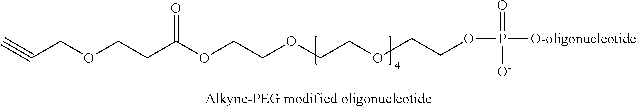

### Example 31a

### Synthesis, Purification, and Characterization of Trimethylpropanaminium PVA (cationic PVA)

PVA (0.056 mmol, 80% hydrolyzed, viscosity 2.5-3.5 cPs, Sigma-Aldrich) was dissolved in DMSO (5 mL) at 65° C. followed by the addition of sodium hydride (12.5 mmol). The reaction mixture was stirred for an hour followed by the addition of glycidyl trimethylammonium chloride (13 mmol). (See scheme below.) The reaction mixture was stirred overnight at 65° C. The reaction mixture was dialyzed for 5 days and lyophilized to give a light brown product. The product was analyzed by H¹ NMR.

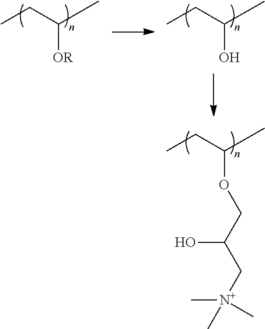

[Cationic PVA can also be purchased from Kuraray, including for example, Cationic PVA CM-318 (Kuraray)(C₁₀H₂₁N₂O.C₄H₆O₂.C₂H₄O.Cl)x1-Propanaminium, N,N,N-trimethyl-s-[(2-methyl-1-oxo-2-propen-1-yl)amino]-chloride (1:1), polymer with ethanol and ethenyl acetate.]

### Example 32

### Formulation and Characterization of siRNA Containing Pegylated Particles, Via Nanoprecipitation, Including Cationic PVA

O-acetyl 5050 PLGA (60 mg, 54.5 wt %) (Example 4), the copolymer mPEG(2k)-PLGA (40 mg, 36.4 wt %, Mw 11 kDa) and siRNA (10 mg, Mw 14,929) were dissolved in a solvent mixture of Tris-EDTA buffer:acetonitrile at a ratio of 1:4. The total concentration of the polymer was 1.0 wt %. In a separate solution, 0.3% w/v PVA (80% hydrolyzed, viscosity 2.5-3.5 cPs) and 0.2% w/v cationic PVA (Kuraray) (see comments in Example 65) (86-91% hydrolyzed, viscosity 17-27 cPs) were dissolved in water. The polymer solution was added using a syringe pump at a rate of 1 mL/min to the aqueous solution (v/v ratio of polymer solution to aqueous phase=1:10), with stirring at 500 rpm. The organic solvent was removed by stiffing the solution for 2-3 hours. The particles were then washed with 10 volumes of buffer and concentrated using a tangential flow filtration system (300 kDa MW cutoff, membrane area=150 cm²). The loading of siRNA was quantitated using a RiboGreen® fluorescence assay. (See Example 70b.) RNA was used as a standard for generating the calibration curve with RiboGreen® reagent. The fluorescence of the siRNA was measured at an excitation wavelength of 480 nm and an emission wavelength of 520 nm.

Particle properties, evaluated by using the resulting plurality of particles made in the method above:
Z_{avg}=119 nm
PDI=0.142
Dᵥ50=94.9 nm
Dᵥ90=191 nm
siRNA loading: 1% w/w

### Example 32a

### Formulation and Characterization of siRNA Containing Pegylated Particles, Via Nanoprecipitation, Including Cationic PVA

The polymer O-acetyl PLGA5050 (120 mg, 57.1 wt %) (Example 4), the copolymer mPEG₂ₖ-PLGA (80 mg, 38.1 wt %, Mw 11 kDa) and siRNA (10 mg, 4.8 wt. %, Mw 13.0 kDa) with a sense strand having a nucleotide sequence substantially identical to a portion of the EGFP sequence, being 19 base pairs in length with a UU overhang, and having a complementary antisense strand, were dissolved in a solvent mixture of Tris-EDTA buffer:acetonitrile at a ratio of 1:4. The total concentration of the polymer was 1.0 wt %. In a separate solution, 0.3% w/v PVA (80% hydrolyzed, viscosity 2.5-3.5 cPs) and 0.2% w/v cationic PVA (86-91% hydrolyzed, viscosity 17-27 cPs) were dissolved in water. The polymer solution was added using a syringe pump at a rate of 1 mL/min to the aqueous solution (v/v ratio of polymer solution to aqueous phase=1:10), with stiffing at 500 rpm. The particles were then washed with 10 volumes of buffer and concentrated using a tangential flow filtration system (300 kDa MW cutoff, membrane area=150 cm²). The loading of siRNA was quantitated using a RiboGreen® fluorescence assay with RNA as a standard. The fluorescence of the siRNA was measured at an excitation wavelength of 480 nm and an emission wavelength of 520 nm.

Particle properties, evaluated by using the resulting plurality of particles made in the method above:
Z_{avg}=131.5 nm
PDI=0.156
Dᵥ50=123 nm
Dᵥ90=202 nm
siRNA loading: 1.1% w/w

### Example 32b

### Formulation and Characterization of siRNA Containing Pegylated Particles, Via Nanoprecipitation, Including Cationic PVA

SiRNA containing pegylated particles were prepared as described in Example 32a. In place of the EGFP siRNA used in Example 32, a luciferase siRNA (Mw of 13617 Da) with a sense strand having a nucleotide sequence substantially identical to a portion of the luciferase sequence, being 19 base pairs in length with a UU overhang, and having a complementary antisense strand, was used.

Particle properties, evaluated by using the resulting plurality of particles made in the method above:
Z_{avg}=114.1 nm
PDI=0.163
Dᵥ50=103 nm
Dᵥ90=182 nm
siRNA loading: 1.4% wt/wt

### Example 33c

### Formation and Characterization of DNA Containing Pegylated Particles without a Cationic Species

O-acetyl PLGA (57 wt. %, Mw 10 kDa) and mPEG₂ₖ-PLGA (38 wt %, Mw 11 kDa) were dissolved to form a total concentration of 1.0% polymer in acetone. In a separate solution, DNA having 21 base pairs (5 wt. %, Mw 12835) was dissolved in a solution of 0.5% w/v PVA (80% hydrolyzed, viscosity 2.5-3.5 cPs, Sigma-Aldrich) in water. The polymer acetone solution was added via nanoprecipitation at a total flow rate of 239 mL/min (v/v ratio of organic to aqueous phase=1:8), with stiffing. Acetone was removed by stirring the solution for 2-3 hours. The particles were then washed with 10 volumes of water and concentrated using a tangential flow filtration system (300 kDa MW cutoff, membrane area=50 cm²).

Particle properties, evaluated by using the resulting plurality of particles made in the method above:
Z_{avg}=217 nm
PDI=0.12
Dᵥ50=233 nm
Dᵥ90=413 nm
Zeta potential=−22 mV
Drug concentration=0.22 mg/mL

### Example 33

### Formation of siRNA Containing Pegylated Particles Including Cationic-PLGA, Via Nanoprecipitation, Using PVA as Surfactant

Cationic-PLGA (60 mg, 54.5%) (Example 17), mPEG₂ₖ-PLGA (40 mg, 36.4 wt %, Mw 11 kDa) and siRNA having 22 base pairs with dTdT overhangs (10 mg, Mw 14929.06) was dissolved to form a total concentration of 1.0% polymer in a solvent mix Tris-EDTA buffer:acetonitrile (2:8). In a separate solution, 0.5% w/v PVA (80% hydrolyzed, viscosity 2.5-3.5 cPs, Sigma-Aldrich) in water was prepared. The polymer solution was added using a syringe pump at a rate of 1 mL/min to the aqueous solution (v/v ratio of polymer solution to aqueous phase=1:10), with stirring at 500 rpm. Organic solvent was removed by stirring the solution for 2-3 hours. The particles were then washed with 10 volumes of TE buffer and concentrated using a tangential flow filtration system (300 kDa MW cutoff, membrane area=150 cm²).

### Example 34

### Formation and Characterization of siRNA Containing Pegylated Particles Including Protamine Sulfate, Via Nanoprecipitation, Using PVA as Surfactant

5050 PLGA-O-acetyl (60 mg, 54.5%), mPEG₂ₖ-5050PLGA₉ₖ (40 mg, 36.4 wt %, Mw 11 kDa) and siRNA (Example 31) (10 mg, Mw 14929.06) were dissolved to form a total concentration of 1.0% polymer in a solvent mix Tris-EDTA buffer:acetonitrile (2:8). In a separate solution, 0.5% w/v PVA (80% hydrolyzed, viscosity 2.5-3.5 cPs, Sigma-Aldrich) and 1% w/v protamine sulfate in water was prepared. The polymer solution was added using a syringe pump at a rate of 1 mL/min to the aqueous solution (v/v ratio of polymer solution to aqueous phase=1:10), with stiffing at 500 rpm. Organic solvent was removed by stirring the solution for 2-3 hours. The particles were washed with 10 volumes of TE buffer and concentrated using a tangential flow filtration system (300 kDa MW cutoff, membrane area=150 cm²).

Particle properties, evaluated by using the resulting plurality of particles made in the method above:
Z_{avg}=116.9 nm
PDI=0.220
Dᵥ50=98.1 nm
Dᵥ90=144 nm

### Example 35

### Formation and Characterization of siRNA Containing Pegylated Particles Including N1-PLGA-N5, N10, N14-Tetramethylated-Spermine, Via Nanoprecipitation, Using PVA as Surfactant

N1-PLGA-N5,N10,N14-tetramethylated-spermine (60 mg, 57.1 wt. %, Mw 5.3 kDa), mPEG₂ₖ-PLGA (40 mg, 38.1 wt %, Mw 11 kDa) and siRNA having 22 base pairs with dTdT overhangs (5 mg, 4.8 wt. %, Mw 14929.06) were dissolved to form a total concentration of 1.0% polymer in a solvent mix Tris-EDTA buffer:acetonitrile (2:8). In a separate solution, 0.5% w/v PVA (80% hydrolyzed, viscosity 2.5-3.5 cPs, Sigma-Aldrich) in water was prepared. The polymer solution was added using a syringe pump at a rate of 1 mL/min to the aqueous solution (v/v ratio of polymer solution to aqueous phase=1:10), with stiffing at 500 rpm. The particles were then washed with 10 volumes of TE buffer and concentrated using a tangential flow filtration system (300 kDa MW cutoff, membrane area=150 cm²).

Particle properties, evaluated by using the resulting plurality of particles made in the method above:
Z_{avg}=118.5 nm
PDI=0.13
Dᵥ50=102 nm
Dᵥ90=162 nm
Zeta potential=−18.4 mV

### Example 36

### Formulation and Characterization of DNA Containing Particles Including N1-PLGA-N5,N10,N14-Tetramethylated-Spermine Using a Two-Step Method

PLGA-O-acetyl (20 wt %, Mw 10 kDa), mPEG₂ₖ-5050PLGA₉ₖ (39 wt %, Mw 11 kDa) and N1-PLGA-N5,N10,N14-tetramethylated-spermine (39 wt %, Mw 8.3 kDa) were dissolved to form a total concentration of 1.0% polymer in acetone. In a separate solution, DNA having 21 base pairs (2 wt. %, Mw 12835) was dissolved in water. The polymer acetone solution was added via nanoprecipitation at a total flow rate of 335 mL/min (v/v ratio of organic to aqueous phase=1:10), with stiffing. Acetone was removed by stirring the solution for 2-3 hours. The particles were then washed with 10 volumes of water and concentrated using a tangential flow filtration system (300 kDa MW cutoff, membrane area=50 cm²). PVA (viscosity 2.5-3.5 cp, Sigma-Aldrich) was added to the particles and allowed to stir for 2-3 hours.

Particle properties, evaluated by using the resulting plurality of particles made in the method above:
Z-average: 108 nm
PDI: 0.24
Dᵥ50: 84 nm
Dᵥ90: 163 nm
Zeta potential: 10.8 mV

### Example 37

### Formation and Characterization of siRNA Containing Pegylated Particles Spermine, Via Nanoprecipitation, Using PVA as Surfactant

SiRNA having 22 base pairs with dTdT overhangs (5 mg, 4.5 wt. %, Mw 14.9 kDa), 5050-O-acetyl-PLGA (60 mg, 54.5 wt. %, Mw 10 kDa), mPEG₂ₖ-PLGA (40 mg, 36.4 wt %, Mw 11 kDa) and spermine tetrahydrochloride (5 mg, 4.5 wt. %, Mw 348 Da) were dissolved to form a total concentration of 1.0% polymer in a solvent mix water:acetonitrile (2:8). In a separate solution, 0.5% w/v PVA (80% hydrolyzed, viscosity 2.5-3.5 cPs, Sigma-Aldrich) in water was prepared. The polymer solution was added using a syringe pump at a rate of 1 mL/min to the aqueous solution (v/v ratio of polymer solution to aqueous phase=1:10), with stirring at 500 rpm. The particles were then washed with 10 volumes of TE buffer and concentrated using a tangential flow filtration system (300 kDa MW cutoff, membrane area=150 cm²).

Particle properties, evaluated by using the resulting plurality of particles made in the method above:
Z_{avg}=210.6 nm
PDI=0.27
Dᵥ50=193 nm
Dᵥ90=323 nm
Zeta potential=−23.3 mV

### Example 38

### Formation and Characterization of siRNA Containing Pegylated Particles Including Spermine, Via Nanoprecipitation

C6-Thiol modified oligonucleotides (as used in Example 22) (siRNA, 5 mg, 0.37 μmol, 2.9 wt. %, Mw 13.6 kDa) were conjugated to 2-(2-(Pyridin-2-yl)disulfanyl)ethylamino-5050-PLGA-O-acetyl (100 mg, 15.8 μmol, 58.1 wt. %, Mw 6.3 kDa) in a solvent mix (95:5, DMSO:TE, 10 mL) with mPEG₂ₖ-5050PLGA₉ₖ (67 mg, 39 wt. %, Mw 11 kDa). In a separate solution, 0.5% w/v PVA (80% hydrolyzed, viscosity 2.5-3.5 cPs, Sigma-Aldrich) and 0.3% w/v of spermine tetrahydrochloride in water was prepared. The polymer solution was added using a syringe pump at a rate of 1 mL/min to the aqueous solution (v/v ratio of polymer solution to aqueous phase=1:10), with stirring at 500 rpm. The particles were then washed with 10 volumes of TE buffer and concentrated using a tangential flow filtration system (300 kDa MW cutoff, membrane area=150 cm²).

Particle properties, evaluated by using the resulting plurality of particles made in the method above:
Z_{avg}=143.2 nm
PDI=0.21
Dᵥ50=119 nm
Dᵥ90=200 nm
Zeta potential=−11.5 mV

### Example 39

### Formation and Characterization of siRNA Containing Pegylated Particles Including N1-PLGA-N5, N10, N14-Tetramethylated-Spermine, Via Nanoprecipitation

C6-Thiol modified oligonucleotides (as used in Example 22) (siRNA, 2 mg, 0.37 μmol, 0.8 wt. %, Mw 13.6 kDa) were conjugated to 2-(2-(Pyridin-2-yl)disulfanyl)ethylamino-5050-PLGA-O-acetyl (50 mg, 15.8 μmol, 19.8 wt. %, Mw 6.3 kDa) in a solvent mix (95:5, DMSO:TE, 10 mL) with mPEG₂ₖ-5050PLGA₉ₖ (100 mg, 39.7 wt. %, Mw 11 kDa) and N1-PLGA-N5,N10,N14-tetramethylated-spermine (100 mg, 39.7 wt. %, Mw 5.3 kDa). In a separate solution, 0.5% w/v PVA (80% hydrolyzed, viscosity 2.5-3.5 cPs, Sigma-Aldrich) in water was prepared. The polymer solution was added using a syringe pump at a rate of 1 mL/min to the aqueous solution (v/v ratio of polymer solution to aqueous phase=1:10), with stirring at 500 rpm. The particles were then washed with 10 volumes of TE buffer and concentrated using a tangential flow filtration system (300 kDa MW cutoff, membrane area=150 cm²).

Particle properties, evaluated by using the resulting plurality of particles made in the method above:
Z_{avg}=135.4 nm
PDI=0.12
Dᵥ50=120 nm
Dᵥ90=208 nm
Zeta potential=−8.39 mV

### Example 39a

### Formulation and Characterization of siRNA Containing Pegylated Particles Including Bis-(N1-Spermine) Glutamide-5050 PLGA-O-Acetyl, Via Nanoprecipitation, Using PVA as Surfactant

Bis-(N1-spermine) glutamide-5050 PLGA-O-acetyl (67 wt. %) and mPEG₂ₖ-PLGA (28 wt %, Mw 11 kDa) were dissolved to form a total concentration of 1.0% polymer in acetone. In a separate solution, siRNA having 22 base pairs with dTdT overhangs (2 wt %, Mw 14929.06) was dissolved in a solution of 0.5% w/v PVA (80% hydrolyzed, viscosity 2.5-3.5 cPs, Sigma-Aldrich) in water. The molar ratio of cation amino groups to siRNA phosphate groups (N/P ratio) was 4.4:1, e.g. ratio of bis-(N1-spermine) glutamide-5050 PLGA-O-acetyl and siRNA respectively. The polymer acetone solution was added via nanoprecipitation at a total flow rate of 335 mL/min (v/v ratio of organic to aqueous phase=1:8), with stiffing. Acetone was removed by stiffing the solution for 2-3 hours. The particles were then washed with 10 volumes of water and concentrated using a tangential flow filtration system (300 kDa MW cutoff, membrane area=50 cm²).

Particle properties, evaluated by using the resulting plurality of particles made in the method above:
Z_{avg}=61 nm
PDI=0.16
Dᵥ50=43 nm
Dᵥ90=72 nm
Zeta potential=−2.6 mV
Drug concentration: 3.1 wt %

### Example 39b

### Formulation and Characterization of siRNA Containing Pegylated Particles Including Bis-(N1-Spermine) Glutamide-5050 PLGA-O-Acetyl, Using a Two-Step Method

Bis-(N1-spermine) glutamide-5050 PLGA-O-acetyl (68 wt %) and mPEG₂ₖ-5050PLGA₉ₖ (29 wt %, Mw 11 kDa) were dissolved to form a total concentration of 1.0% polymer in acetone. In a separate solution, siRNA having 22 base pairs with dTdT overhangs (2 wt %, Mw 14929.06) was dissolved in water. The molar ratio of cation amino groups to siRNA phosphate groups (N/P ratio) was 11:1, e.g. ratio of bis-(N1-spermine) glutamide-5050 PLGA-O-acetyl and siRNA respectively. The polymer acetone solution was added via nanoprecipitation at a total flow rate of 335 mL/min (v/v ratio of organic to aqueous phase=1:8), with stiffing. Acetone was removed by stiffing the solution for 2-3 hours. The particles were then washed with 10 volumes of water and concentrated using a tangential flow filtration system (300 kDa MW cutoff, membrane area=50 cm²). PVA (viscosity 2.5-3.5 cp, Sigma-Aldrich) was added to the particles and allowed to stir for 2-3 hours.

Particle properties, evaluated by using the resulting plurality of particles made in the method above:
Z_{avg}=132 nm
PDI=0.18
Dᵥ50=101 nm
Dᵥ90=226 nm
Zeta potential=−1.6 mV
Drug concentration: 4.6 wt %

### Example 39c

### Formulation and Characterization of siRNA Containing Pegylated Particles Including Bis-(N1-Spermine) Glutamide-5050 PLGA-O-Acetyl, Via Nanoprecipitation, Using PVA as Surfactant

C6-thiol modified oligonucleotide (siRNA, 10 mg, 0.755 μmol, 4.2 wt. %, Mw 13.2 kDa) as shown in Example 22b was conjugated to 2-(2-(pyridin-2-yl)disulfanyl)ethylamino-5050-PLGA-O-acetyl (42.5 mg, 6 μmol, 17.9 wt. %, Mw 6.9 kDa) as shown in Example 16 in a solvent mixture of 95:5 DMSO:TE (10 mL) with mPEG₂ₖ-5050PLGA₉ₖ (100 mg, 42.1 wt. %, Mw 11 kDa) and Bis-(N1-spermine) glutamide-5050 PLGA-O-acetyl (85 mg, 35.8 wt. %). In a separate solution, 0.5% w/v PVA (80% hydrolyzed, viscosity 2.5-3.5 cPs) in water was prepared. The polymer solution was added using a syringe pump at a rate of 1 mL/min to the aqueous solution (v/v ratio of polymer solution to aqueous phase=1:10), with stiffing at 500 rpm. The particles were then washed with 10 volumes of TE buffer and concentrated using a tangential flow filtration system (300 kDa MW cutoff, membrane area=150 cm²). The loading of siRNA was quantitated using a RiboGreen® fluorescence assay with RNA as a standard. The fluorescence of the siRNA was measured at an excitation wavelength of 480 nm and an emission wavelength of 520 nm.

Particle properties, evaluated by using the resulting plurality of particles made in the method above:
Z_{avg}=130.1 nm
PDI=0.205
Dᵥ50=96.5 nm
Dᵥ90=165 nm
Zeta potential=−14.7 mV
siRNA loading: 1.8 wt %

### Example 39d

### Formulation and Characterization of siRNA Containing Pegylated Particles Including Bis-(N1-Spermine) Glutamide-5050 PLGA-O-Acetyl, Via Nanoprecipitation, Using PVA as Surfactant

Bis-(N1-spermine) glutamide-5050 PLGA-O-acetyl (60 mg, 57.1 wt %), mPEG₂ₖ-PLGA (40 mg, 38.1 wt %, Mw 11 kDa), and siRNA (5 mg, 4.8 wt. %, Mw 13029.2) were dissolved in a solvent mixture of Tris-EDTA buffer:acetonitrile at a ratio of 1:4. The total concentration of the polymer was 1.0 wt %. In a separate solution, 0.5% w/v PVA (80% hydrolyzed, viscosity 2.5-3.5 cPs) was dissolved in water. The polymer solution was added using a syringe pump at a rate of 1 mL/min to the aqueous solution (v/v ratio of polymer solution to aqueous phase=1:10), with stirring at 500 rpm. The particles were then washed with 10 volumes of buffer and concentrated using a tangential flow filtration system (300 kDa MW cutoff, membrane area=150 cm²). The loading of siRNA was quantitated using a RiboGreen® fluorescence assay with RNA as a standard. The fluorescence of the siRNA was measured at an excitation wavelength of 480 nm and an emission wavelength of 520 nm.

Particle properties, evaluated by using the resulting plurality of particles made in the method above:
Z_{avg}=67.35 nm
PDI=0.366
Dᵥ50=43.4 nm
Dᵥ90=75.1 nm
Zeta potential=+17.6 mV
siRNA loading: 1.8 wt %

### Example 39e

### Formulation and Characterization of siRNA Containing Pegylated Particles Including bis-(n1-spermine) glutamide-5050 PLGA-o-acetyl, Via Nanoprecipitation, without a Surfactant

Bis-(N1-spermine) glutamide-5050 PLGA-O-acetyl (60 mg, 57.1 wt %), mPEG₂ₖ-PLGA (40 mg, 38.1 wt %, Mw 11 kDa), and siRNA (5 mg, 4.8 wt. %, Mw 13029.2) were dissolved in a solvent mixture of Tris-EDTA buffer:acetonitrile at a ratio of 1:4. The total concentration of the polymer was 1.0 wt %. The polymer solution was added using a syringe pump at a rate of 1 mL/min to water (v/v ratio of polymer solution to aqueous phase=1:10), with stiffing at 500 rpm. The particles were then washed with 10 volumes of buffer and concentrated using a tangential flow filtration system (300 kDa MW cutoff, membrane area=150 cm²). The loading of siRNA was quantitated using a RiboGreen® fluorescence assay with RNA as a standard. The fluorescence of the siRNA was measured at an excitation wavelength of 480 nm and an emission wavelength of 520 nm.

Particle properties, evaluated by using the resulting plurality of particles made in the method above:
Z_{avg}=74.75 nm
PDI=0.233
Dᵥ50=53 nm
Dᵥ90=85.6 nm
Zeta potential=+20 mV
siRNA loading: 2.4 wt %

### Example 40

### Formation of Nucleic Acid Agent Containing Pegylated Particles Including Cationic Polymers, Via Nanoprecipitation, Using PVA as Surfactant

5050-O-acetyl-PLGA (60 mg, 60 wt. %) and nucleic acid-conjugated mPEG₂ₖ-PLGA (Example 23) (40 mg, 40 wt %, Mw ˜25.7 kDa) will be dissolved to form a total concentration of 1.0% polymer in a solvent mix of Tris-EDTA:DMSO (5:95) or alternatively Tris-EDTA:acetonitrile. In a separate solution, 0.3% w/v PVA (80% hydrolyzed, viscosity 2.5-3.5 cPs, Sigma-Aldrich) and 0.2% w/v cationic PVA (86-91% hydrolyzed, viscosity 17-27 cPs, Kuraray) in water will be prepared. The polymer solution will be added using a syringe pump at a rate of 1 mL/min to the aqueous solution (v/v ratio of polymer solution to aqueous phase=1:10), with stirring at 500 rpm. The particles will then be washed with 10 volumes of TE buffer and concentrated using a tangential flow filtration system (300 kDa MW cutoff, membrane area=150 cm²). In some cases, the particles will be lyophilized into powder form.

### Example 41

### Formation of Nucleic Acid Agent Containing Pegylated Particles Including Cationic Moieties, Via Nanoprecipitation, Using PVA as Surfactant

5050 PLGA (60 mg, 54.5%), mPEG₂ₖ-PLGA (40 mg, 36.4 wt %, Mw 11 kDa), and nucleic acid-conjugated mPEG₂ₖ-PLGA (Example 23) (10 mg, Mw ˜25.7 kDa) will be dissolved to form a total concentration of 1.0% polymer in a solvent mix Tris-EDTA buffer:acetonitrile (2:8). In a separate solution, 0.5% w/v PVA (80% hydrolyzed, viscosity 2.5-3.5 cPs, Sigma-Aldrich) in water containing 0.1 mM to 50 mM of cationic moieties (e.g. spermine tetrahydrochloride, hexyldecyltrimethylammonium chloride, hexadimethrine bromide, protamine sulfate, and cationic polymers, e.g., polyhistidine, polylysine, polyarginine, polyethylene imine, and chitosan) could be prepared. The polymer solution will be added using a syringe pump at a rate of 1 mL/min to the aqueous solution (v/v ratio of polymer solution to aqueous phase=1:10), with stiffing at 500 rpm. Organic solvent could be removed by stiffing the solution for 2-3 hours. The particles will then be washed with 10 volumes of TE buffer and concentrated using a tangential flow filtration system (300 kDa MW cutoff, membrane area=150 cm²). In some cases, the particles will be lyophilized into powder form.

### Example 42

### Formation of Nucleic Acid Agent Containing Pegylated Particles Including Cationic-mPEG₂ₖ-PLGA, Via Nanoprecipitation, Using PVA as Surfactant

5050 PLGA (60 mg, 60 wt %), cationic-mPEG₂ₖ-PLGA (Example 15) (30 mg, 30 wt %, Mw 11 kDa) and nucleic acid-conjugated mPEG₂ₖ-PLGA (Example 23) (10 mg, Mw ˜25.7 kDa) will be dissolved to form a total concentration of 1.0% polymer in a solvent mix Tris-EDTA buffer:acetonitrile (2:8). In a separate solution, 0.5% w/v PVA (80% hydrolyzed, viscosity 2.5-3.5 cPs, Sigma-Aldrich) in water will be prepared. The polymer solution will be added using a syringe pump at a rate of 1 mL/min to the aqueous solution (v/v ratio of polymer solution to aqueous phase=1:10), with stirring at 500 rpm. Organic solvent will be removed by stirring the solution for 2-3 hours. The particles will then be washed with 10 volumes of TE buffer and concentrated using a tangential flow filtration system (300 kDa MW cutoff, membrane area=150 cm²). In some cases, the particles will be lyophilized into powder form.

### Example 43

### Formation of Nucleic Acid Agent Containing Pegylated Particles Including Cationic-PLGA, Via Nanoprecipitation, Using PVA as Surfactant

Cationic-PLGA (60 mg, 60%) (Example 68) and nucleic acid-conjugated mPEG₂ₖ-PLGA (Example 23) (40 mg, 40 wt %, Mw ˜25.7 kDa) will be dissolved to form a total concentration of 1.0% polymer in a solvent mix Tris-EDTA buffer:acetonitrile (2:8). In a separate solution, 0.5% w/v PVA (80% hydrolyzed, viscosity 2.5-3.5 cPs, Sigma-Aldrich) in water will be prepared. The polymer solution will be added using a syringe pump at a rate of 1 mL/min to the aqueous solution (v/v ratio of polymer solution to aqueous phase=1:10), with stirring at 500 rpm. Organic solvent will be removed by stirring the solution for 2-3 hours. The particles will then be washed with 10 volumes of TE buffer and concentrated using a tangential flow filtration system (300 kDa MW cutoff, membrane area=150 cm²). In some cases, the particles will be lyophilized into powder form.

### Example 44

### Formation of Nucleic Acid Agent Containing Pegylated Particles, Via Nanoprecipitation, Using PVA as Surfactant

Cationic-PLGA (60 mg, 60%, Mw) (Example 68) and nucleic acid-conjugated mPEG₁₀ₖ (Example 27) (40 mg, 40 wt %, Mw ˜26.7 kDa) will be dissolved to form a total concentration of 1.0% polymer in a solvent mix Tris-EDTA buffer:acetonitrile (2:8). In a separate solution, 0.5% w/v PVA (80% hydrolyzed, viscosity 2.5-3.5 cPs, Sigma-Aldrich) in water will be prepared. The polymer solution will be added using a syringe pump at a rate of 1 mL/min to the aqueous solution (v/v ratio of polymer solution to aqueous phase=1:10), with stirring at 500 rpm. Organic solvent will be removed by stirring the solution for 2-3 hours. The particles will then be washed with 10 volumes of TE buffer and concentrated using a tangential flow filtration system (300 kDa MW cutoff, membrane area=150 cm²). In some cases, the particles will be lyophilized into powder form.

### Example 45

### Formation of Nucleic Acid Agent Containing Pegylated Particles, Via Surface Bioconjugation of Preformulated Intermediate Particles, with Cationic Moieties

2-(2-(pyridin-2-yl)disulfanyl)ethylamino-5050-PLGA-O-acetyl (90 mg, 90 wt. %) and mPEG₂ₖ-PLGA (10 mg, 10 wt %, Mw 11 kDa) will be dissolved to form a total concentration of 1.0% polymer in acetone. The polymer solution will be added using a syringe pump at a rate of 1 mL/min to water (v/v ratio of polymer solution to aqueous phase=1:10), with stiffing at 500 rpm. Organic solvent will be removed by stiffing the solution for 2-3 hours. C6-Thiol modified oligonucleotides (as used in Example 22) (siRNA or DNA, 2 mg, 0.13 μmol) will be conjugated to 2-(2-(pyridin-2-yl)disulfanyl)ethylamino-5050-PLGA-O-acetyl preformed particles (4 mg, 0.625 μmol) in buffer (PBS, pH 8, 0.4 mL), which can be unpegylated or ≦10 wt. % pegylated. The reaction mixture will be stirred under argon at room temperature for 48 hours. The reaction mixture will be analyzed by anionic-exchange and reverse phase HPLC. The particles (60 mg, 60 wt. %) will be lyophilized into powder form. The particles (60 mg, 60 wt. %) and mPEG₂ₖ-PLGA (40 mg, 40 wt. %) will be dissolved in acetone or an appropriate aqueous/organic solvent mix Tris-EDTA buffer:acetonitrile (2:8) to form a 1% polymer concentration. In a separate solution, 0.5% w/v PVA (80% hydrolyzed, viscosity 2.5-3.5 cPs, Sigma-Aldrich) in water containing 0.1 mM to 50 mM of cationic moieties (e.g. spermine tetrahydrochloride, hexyldecyltrimethylammonium chloride, hexadimethrine bromide, protamine sulfate, or cationic polymers, e.g., polyhistidine, polylysine, polyarginine, polyethylene imine, or chitosan) will be prepared. The polymer solution will be added using a syringe pump at a rate of 1 mL/min to the aqueous solution (v/v ratio of polymer solution to aqueous phase=1:10), with stirring at 500 rpm. Organic solvent will be removed by stirring the solution for 2-3 hours. The nucleic acid agent functionalized particles will then be washed with 10 volumes of water and concentrated using a tangential flow filtration system (300 kDa MW cutoff, membrane area=150 cm²). In some cases, the particles will be lyophilized into powder form.

### Example 46

### Formation of Lipid Coated Nucleic Acid Agent Containing Pegylated Particles

Cationic-PLGA (60 mg, 60%) (Example 68) and nucleic acid-conjugated 5050-O-acetyl-PLGA (40 mg, 40 wt %, Mw ˜23.7 kDa) will be dissolved to form a total concentration of 1.0% polymer in acetone or a solvent mix Tris-EDTA buffer:acetonitrile (2:8). The polymer solution will be added using a syringe pump at a rate of 1 mL/min to water (v/v ratio of polymer solution to aqueous phase=1:10), with stirring at 500 rpm to form particle suspension. Organic solvent will be removed by stiffing the solution for 2-3 hours. A lipid mixture of DOTAP, cholesterol and DOPE-PEG₂ₖ in ethanol will be added to the particle suspension via a syringe pump at a rate of 1 mL/min to final concentration of 70% ethanol. The final formulation will be diluted 10 fold with water and washed with 5 volumes of water and concentrated using a tangential flow filtration system (300 kDa MW cutoff, membrane area=150 cm²). In some cases, the particles will be lyophilized into powder form.

### Example 47

### Formation of Nucleic Acid Agent Containing Pegylated Particles

Nucleic acid-conjugated 5050-O-acetyl-PLGA (Mw ˜23.7 kDa) will be dissolved to form a total concentration of 1.0% polymer in acetone or a solvent mix Tris-EDTA buffer:acetonitrile (2:8). The polymer solution will be added using a syringe pump at a rate of 1 mL/min to water (v/v ratio of polymer solution to aqueous phase=1:10), with stirring at 500 rpm to form particle suspension. Organic solvent will be removed by stirring the solution for 2-3 hours. Cationic polymer (e.g., polyhistidine, polylysine, polyarginine, polyethylene imine, or chitosan 60 wt. %) and mPEG₂ₖ-PLGA (40 wt. %) will be dissolved in a water miscible solvent such as acetone to form a 1% polymer solution and will be added to the particle suspension via a syringe pump at a rate of 1 mL/min. The final formulation will be diluted 10 fold with water and washed with 5 volumes of water and concentrated using a tangential flow filtration system (300 kDa MW cutoff, membrane area=150 cm²). In some cases, the particles will be lyophilized into powder form.

### Example 48

### Formulation of siRNA Containing Pegylated Particles Including Cationic Moieties, Via Nanoprecipitation, Using PVA as Surfactant

5050 PLGA (60 mg, 54.5%), mPEG₂ₖ-PLGA₉ₖ (40 mg, 36.4 wt %, Mw 11 kDa), siRNA (10 mg, Mw 14.9 kDa) and cationic moieties (e.g. spermine tetrahydrochloride, hexyldecyltrimethylammonium chloride, hexadimethrine bromide, agamatine, or cationic lipids, e.g., DOTAP) will be dissolved to form a total concentration of 1.0% polymer in a solvent mix Tris-EDTA buffer:acetonitrile (2:8). In a separate solution, 0.5% w/v PVA (80% hydrolyzed, viscosity 2.5-3.5 cPs, Sigma-Aldrich) in water will be prepared. The polymer solution will be added using a syringe pump at a rate of 1 mL/min to the aqueous solution (v/v ratio of polymer solution to aqueous phase=1:10), with stiffing at 500 rpm. Organic solvent will be removed by stirring the solution for 2-3 hours. The particles will then be washed with 10 volumes of TE buffer and concentrated using a tangential flow filtration system (300 kDa MW cutoff, membrane area=150 cm²). In some cases, the particles will be lyophilized into powder form.

### Example 49

### Formulation of Nucleic Acid Agent Containing Pegylated Particles Including Cationic Moieties, Via Nanoprecipitation, Using PVA as Surfactant

5050 PLGA (60 mg, 54.5%), mPEG₂ₖ-PLGA₉ₖ (40 mg, 36.4 wt %, Mw 11 kDa) and nucleic acid conjugated 5050 PLGA (10 mg, Mw ˜23.7 kDa) will be dissolved to form a total concentration of 1.0% polymer in a solvent mix Tris-EDTA buffer:acetonitrile (2:8). In a separate solution, 0.5% w/v PVA (80% hydrolyzed, viscosity 2.5-3.5 cPs, Sigma-Aldrich) in water containing 0.1% w/v of cationic moieties (e.g., spermine tetrahydrochloride, hexyldecyltrimethylammonium chloride, hexadimethrine bromide, agamatine, or cationic polymers, e.g., polyhistidine, polylysine, polyarginine, polyethylene imine, or chitosan) will be prepared. The polymer solution will be added using a syringe pump at a rate of 1 mL/min to the aqueous solution (v/v ratio of polymer solution to aqueous phase=1:10), with stirring at 500 rpm. Organic solvent will be removed by stirring the solution for 2-3 hours. The particles were then be washed with 10 volumes of TE buffer and concentrated using a tangential flow filtration system (300 kDa MW cutoff, membrane area=150 cm²). In some cases, the particles will be lyophilized into powder form.

### Example 50

### Formation of Nucleic Acid Agent Containing Pegylated Particles Including Cationic Moieties, Via Nanoprecipitation, Using PVA as Surfactant

5050 PLGA (60 mg, 54.5%), mPEG₂ₖ-PLGA₉ₖ (40 mg, 36.4 wt %, Mw 11 kDa), nucleic acid conjugated 5050 PLGA (10 mg, Mw ˜23.7 kDa) and cationic moieties (e.g. agamatine, spermine tetrahydrochloride, hexyldecyltrimethylammonium chloride, hexadimethrine bromide, or cationic lipids such as DOTAP) will be dissolved to form a total concentration of 1.0% polymer in a solvent mix Tris-EDTA buffer:acetonitrile (2:8). In a separate solution, 0.5% w/v PVA (80% hydrolyzed, viscosity 2.5-3.5 cPs, Sigma-Aldrich) in water will be prepared. The polymer solution will be added using a syringe pump at a rate of 1 mL/min to the aqueous solution (v/v ratio of polymer solution to aqueous phase=1:10), with stirring at 500 rpm. Organic solvent will be removed by stirring the solution for 2-3 hours. The particles were then be washed with 10 volumes of TE buffer and concentrated using a tangential flow filtration system (300 kDa MW cutoff, membrane area=150 cm²). In some cases, the particles will be lyophilized into powder form.

### Example 51

### Synthesis, Purification, and Characterization of acrylate 5050 PLGA

5050 PLGA (5.0 g, 0.94 mmol, MW 5.3 kDa) and pyridine (200 mg, 2.5 mmol) were dissolved in dichloromethane (DCM, 20 mL). Acryloyl chloride (230 mg, 2.5 mmol) was added dropwise over ½ h and stirred for an additional 3 h. It was then poured into diethyl ether (50 mL) to precipitate out the polymer. The polymer was rinsed with diethyl ether (25 mL) and dried under vacuum to yield a white powder. It was further purified by dissolving the solid in acetone (20 mL) and precipitating into cold water at 5° C. (400 mL) over ½ h. The mixture was then stirred for an additional 2 h. The polymer was removed by filtration and lyophilized to yield a white solid (3.8 g, 76% yield). The product was confirmed by ¹H NMR.

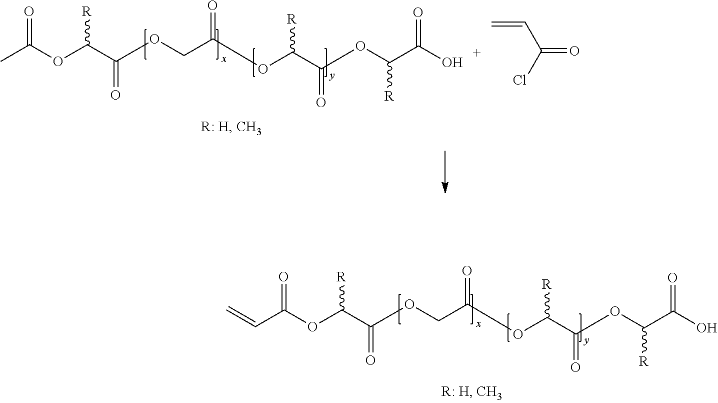

### Example 52

### Synthesis, Purification, and Characterization of 2-(2-aminoethoxy)ethanol acrylate 5050 PLGA-O-acetyl

### Synthesis of Boc-2-(2-aminoethoxy)ethanol

2-(2-aminoethoxy)ethanol (5.0 g, 48 mmol) was dissolved in tetrahydrofuran (THF, 50 mL). To the mixture, 2N sodium hydroxide (24 mL) was added and the entire solution was cooled in an ice bath. Di-tert-butyl dicarbonate (10 g, 48 mmol) was dissolved in THF (50 mL) and it was added to the mixture dropwise over 1 h in an ice bath. The reaction was brought to room temperature and stirred for 2.5 days. THF was removed under vacuum. The aqueous solution was adjusted to pH 3 with concentrated sulfuric acid. It was then extracted with ethyl acetate (EtOAc, 75 mL) twice. The organic layer was washed with water (25 mL) twice and brine (25 mL) once. It was then dried over magnesium sulfate (MgSO₄). EtOAc was removed under vacuum to yield a clear oil (4.1 g, 42% yield). The product was confirmed by ¹H NMR.

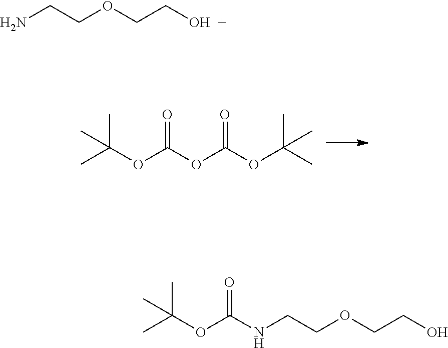

### Synthesis of 2-(2-Aminoethoxy)ethanol acrylate TFA

2-(2-Aminoethoxy)ethanol (1.0 g, 4.9 mmol) and triethanolamine (TEA, 0.54 g, 5.4 mmol) were dissolved in DCM (50 mL). The mixture was cooled in ice bath. Acryloyl chloride (0.49 g, 5.4 mmol) was dissolved in DCM (10 mL) and it was added dropwise over ½ h to the mixture in an ice bath. The reaction was brought to room temperature and stirred overnight. The reaction mixture turned yellow. It was then washed with 0.1N hydrochloric acid (15 mL) twice, brine (15 mL) twice and dried over MgSO₄. It was then pumped down to yield yellow oil (0.54 g, 43% yield). The yellow oil was used without further purification. It was dissolved in a mixture of DCM:TFA (1:1, 10 mL) and stirred for 1 h at room temperature. The solvent was removed under vacuum to yield yellow oil (0.50 g, 94% yield). The product was confirmed by ¹H NMR.

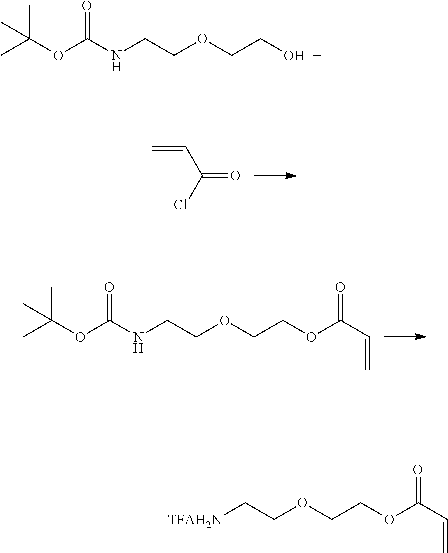

### Synthesis of 2-(2-Aminoethoxy)ethanol acrylate 5050 PLGA-O-Acetyl

5050 PLGA-O-Acetyl (2.0 g, 0.37 mmol, MW 5.3 kDa) and 2-(2-Aminoethoxy)ethanol acrylate TFA (190 mg, 0.75 mmol), EDC (120 mg, 0.75 mmol), NHS (87 mg, 0.75 mmol) and TEA (76 mg, 0.75 mmol) were dissolved in DCM (10 mL) and stirred for 3 h at room temperature. During the process, the solvent, DCM was removed. The polymer was dissolved in acetone (10 mL) and then added to cold water (400 mL) at 5° C. to yield a precipitate. The polymer was lyophilized to yield a white solid (1.2 g, 60% yield). The product was confirmed by ¹H NMR.

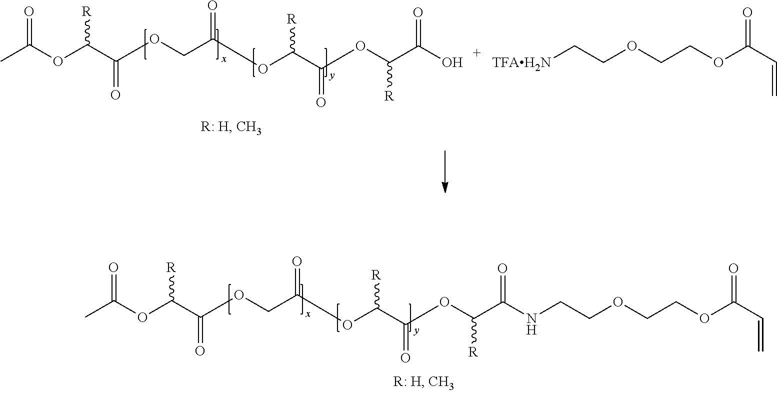

### Example 53

### Synthesis, Purification, and Characterization of N-(2-aminoethyl)maleimide 5050 PLGA-O-acetyl

5050 PLGA-O-acetyl (3.0 g, 0.57 mmol, MW 5.3 kDa), NHS (100 mg, 0.91 mmol) and DCC (190 mg, 0.91 mmol) were added in DCM (15 mL). After 1 h. stirring, N-(2-aminoethyl)maleimide trifluoroacetate (230 mg, 0.91 mmol) and TEA (180 mg, 1.8 mmol) were added and stirred for an additional 3 h. The precipitate was removed by filtration and DCM was removed under vacuum. It was then re-dissolved in acetone (30 mL) and precipitated out in water (400 mL) at 5° C. The precipitate was lyophilized to yield a white solid (2.3 g, 77% yield). The product was confirmed by ¹H NMR.

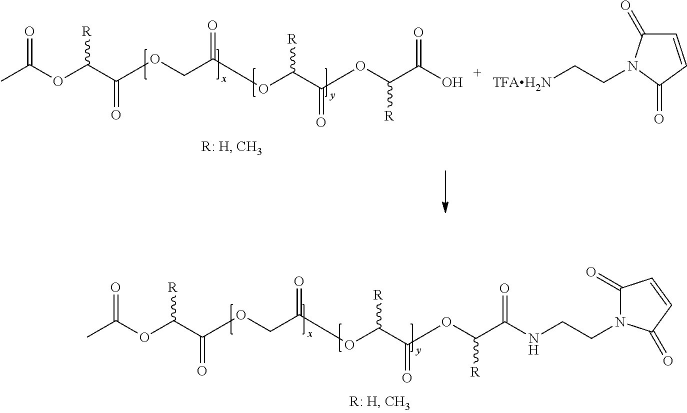

### Example 54

### Synthesis of oligonucleotide-C6-S—N-(2-aminoethyl)maleimide 5050 PLGA-β-acetyl

C6-Thiol modified oligonucleotides (as used in Example 22) (siRNA, 5.0 mg, 0.37 μmol, 3 wt. %, Mw 13.6 kDa) with sense strands having nucleotide sequences substantially identical to a portion of the luciferase sequence, being 19 base pairs in length with a UU overhang, and having a complementary antisense strands, were conjugated to N-(2-Aminoethyl)maleimide 5050 PLGA-O-Acetyl (100 mg, 18.9 μmol, 57 wt. %, Mw 5.3 kDa) in a solvent mixture of DMSO:TE buffer (95:5, 10 mL). The reaction mixture was stirred under argon at 65° C. for 3 h. This mixture was allowed to cool to room temperature.

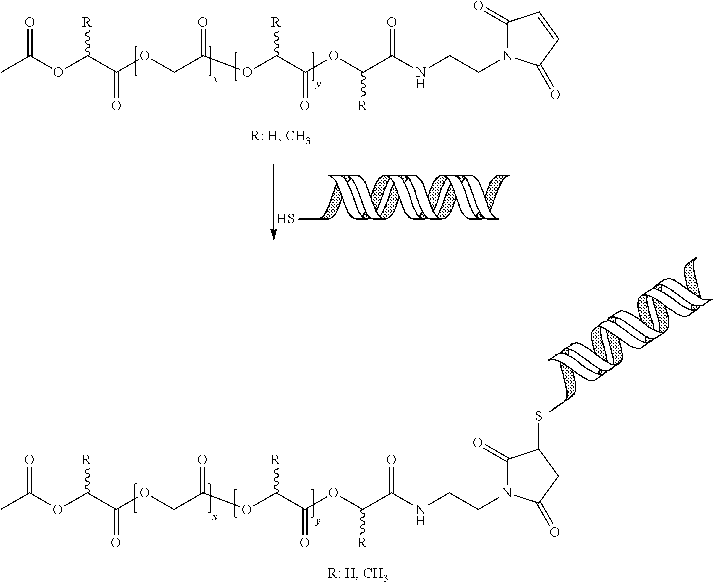

### Example 54a

### Synthesis of oligonucleotide-C6-S—N-(2-Aminoethyl)maleimide 5050 PLGA-O-Acetyl

C6-Thiol modified oligonucleotides (siRNA, 20 mg, 1.51 μmol, Mw 13.2 kDa) with sense strands having nucleotide sequences that are at least 90% identical to a portion of the EGFP sequence, being 19 base pairs in length with a UU overhang, and having a complementary antisense strands, were conjugated to N-(2-Aminoethyl)maleimide 5050 PLGA-O-Acetyl (85 mg, 16.1 μmol, Mw 5.3 kDa) in a solvent mixture of DMSO:TE buffer (95:5, 10 mL). The reaction mixture was stirred under argon at 65° C. for 3 h. This mixture was allowed to cool to room temperature

### Example 55

### Formulation and Characterization of siRNA Containing Pegylated Particles Using a Blend of PVA and Cationic PVA as Surfactant, Via Nanoprecipitation

Si-RNA-C6-S—N-(2-Aminoethyl)maleimide 5050 PLGA-O-acetyl (Example 54) was mixed with mPEG₂ₖ-5050PLGA₉ₖ (67 mg, 40 wt %, Mw 11 kDa) in DMSO (6.7 mL). In a separate solution, 0.3% w/v PVA (80% hydrolyzed, viscosity 2.5-3.5 cPs, Sigma-Aldrich) and 0.2% w/v cationic PVA CM-318 (86-91% hydrolyzed, viscosity 17-27 cPs, Kuraray) in water (167 mL) was prepared. The polymer solution was added to the PVA/cationic PVA solution using a syringe pump at a rate of 1 mL/min to the aqueous solution (v/v ratio of polymer solution to aqueous phase=1:10), with stiffing at 500 rpm. The particles were then washed with 10 volumes of TE buffer and concentrated using a tangential flow filtration system (300 kDa MW cutoff, membrane area=150 cm²). The loading was determined to be 0.92% siRNA w/w. Particle properties, evaluated by using the resulting plurality of particles made in the method above:
Z_{avg}: 103.3 nm
PDI: 0.229
Dᵥ50: 83.3 nm
Dᵥ90: 157 nm
Zeta potential: +16.6 mV

### Example 55a

### Formulation and Characterization of siRNA Containing Pegylated Particles Using a Blend of PVA and Cationic PVA as Surfactant, Via Nanoprecipitation

C6-Thiol modified oligonucleotides (siRNA, 20 mg, 1.51 μmol, Mw 13.2 kDa) conjugated to N-(2-Aminoethyl)maleimide 5050 PLGA-O-Acetyl (as in Example 54a) were mixed with mPEG₂ₖ-5050 PLGA₉ₖ (67 mg, 40 wt %, Mw 11 kDa) in DMSO (6.7 mL). In a separate solution, 0.3% w/v PVA (80% hydrolyzed, viscosity 2.5-3.5 cPs, Sigma-Aldrich) and 0.2% w/v cationic PVA CM-318 (86-91% hydrolyzed, viscosity 17-27 cPs, Kuraray) in water (167 mL) was prepared. The polymer solution was added to a solution of C6-S—N-(2-aminoethyl)maleimide 5050 PLGA-O-acetyl (Example 54) using a syringe pump at a rate of 1 mL/min to the aqueous solution (v/v ratio of polymer solution to aqueous phase=1:10), with stiffing at 500 rpm. The particles were then washed with 10 volumes of TE buffer and concentrated using a tangential flow filtration system (300 kDa MW cutoff, membrane area=150 cm²). The loading was determined to be 3% siRNA w/w. Particle properties, evaluated by using the resulting plurality of particles made in the method above:
Z_{avg}=127 nm
PDI=0.244
Dᵥ50=76.5 nm
Dᵥ90=222 nm
Zeta potential=10.7 mV

### Example 56

### Synthesis, Purification, and Characterization of Oligonucleotide-C6-SS-DSPE-PEG₂ₖ

C6-thiol modified oligonucleotides (as used in Example 22) (siRNA, 0.2 mg, 14.7 nmol) were conjugated to 1,2-distearoyl-sn-glycero-3-phosphoethanolamine-N-[PDP(polyethylene glycol)-2k] (4 mg, 1.36 μmol) in TE buffer (1 mL). The reaction mixture was stirred at 65° C. for 2 hours. The oligonucleotide-C6-SS-DSPE-PEG₂ₖ conjugate was analyzed by reverse phase HPLC and gel electrophoresis.

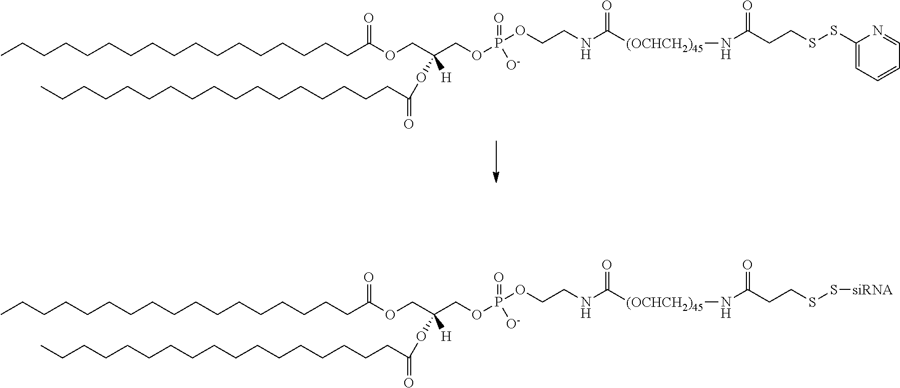

### Example 57

### Synthesis, Purification, and Characterization of Oligonucleotide-C6-thioether-DSPE-PEG₂ₖ

C6-thiol modified oligonucleotides (as used in Example 22) (siRNA, 0.2 mg, 14.7 nmol) with sense strands having nucleotide sequences substantially identical to a portion of the luciferase sequence, being 19 base pairs in length with a UU overhang, and having a complementary antisense strands, were conjugated to 1,2-distearoyl-sn-glycero-3-phosphoethanolamine-N-[maleimide(polyethylene glycol)-2k] (4 mg, 1.36 μmol) in PBS buffer (1 mL). The reaction mixture was stirred at 65° C. for 2 hours. The oligonucleotide-C6-thioether-DSPE-PEG (2000) conjugate was analyzed by reverse phase HPLC and gel electrophoresis.

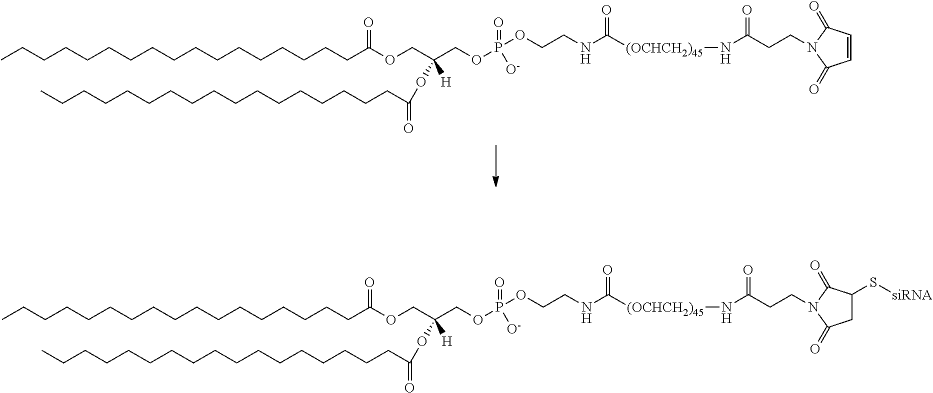

### Example 58

### Viability of Cells Treated with siRNA in Pegylated Particles Including Cationic PVA

To determine if siRNA in pegylated particles including cationic PVA (see Example 32) caused cell death, the CellTiter-Glo® luminescent cell viability assay (CTG) was used. The assay is based on quantization of the ATP present, which signals the presence of metabolically active cells. MDA-MB-231 EGFP cells were grown to 85-90% confluency in 75 cm² flasks (passage <20) in complete media (DMEM, high glucose, 10% HI-FBS, 0.1 mM MEM non-essential amino acids, 2 mM L-glutamine and 1% antibiotic/antimycotic solution) at 37° C. with 5% CO₂. The MDA-MB-231 EGFP cells were added to 96-well opaque-clear bottom plates at a concentration of 1500 cells/well in 200 μL/well. The cells were incubated at 37° C. with 5% CO₂ for 24 hours. The following day, serial dilutions of 2× concentrated siRNA in pegylated particles including cationic PVA were made in 12-well reservoirs with complete media to final concentrations between 5000 nM and 0.05 nM siRNA. The media in the plates was replaced with 100 μL of fresh complete media and 100 μL of respective serially diluted treatment, in duplicate. Three sets of plates were prepared with duplicate treatments. Following 24, 48 and 72 hours of incubation at 37° C. with 5% CO₂, the media in the plates was replaced with 100 μL of fresh complete media and 100 μL of CTG solution, and then incubated for 5 minutes on a plate shaker at room temperature set to 450 rpm and allowed to rest for 15 minutes. Viable cells were measured in a microtiter plate reader set to luminescence. The data was plotted as % viability versus concentration and standardized to untreated cells as shown below.

| siRNA Concentration | T₂₄% | T₄₈% | T₇₂% |
|---|---|---|---|
| (nM) | Viability | Viability | Viability |
| 5000 | 104 | 90 | 106 |
| 500 | 104 | 88 | 113 |
| 50 | 103 | 97 | 112 |
| 5 | 108 | 93 | 118 |
| 0.5 | 99 | 94 | 109 |
| 0.05 | 89 | 88 | 101 |
| 0 | 100 | 100 | 100 |

### Example 59

### Knockdown Activity of siRNA in Pegylated PVA Particles Including Cationic PVA

To measure knockdown activity of siRNA in pegylated particles including cationic PVA (Example 32), MDA-MB-231 EGFP cells were grown to 85-90% confluency in 75 cm² flasks (passage <20) in complete media (DMEM, high glucose, 10% HI-FBS, 0.1 mM MEM non-essential amino acids, 2 mM L-glutamine and 1% antibiotic/antimycotic solution) at 37° C. with 5% CO₂. Three thousand cell per well in 100 μL/well were added to 96-well opaque-clear bottom plates and grown for 24 hours at 37° C. with 5% CO₂. The following day, the media was replaced with 100 μL, in duplicate, of serially diluted siRNA in particles including cationic PVA using concentrations between 1000 and 0.1 nM siRNA. The treated cells were incubated for 48 hours at 37° C. with 5% CO₂. The cells were then washed once with PBS and lysed with 60 μL/well of M-Per Mammalian Protein Extraction Reagent supplemented with Complete Protease Inhibitor Cocktail on ice for 20 minutes. The cell lysates were pipetted up and down 4-5 times prior to measurement on a fluorimeter set to an excitation of 488 nm and an emission of 535 nm. The percent EGFP knockdown of treated cells was compared to an untreated control as shown below.

| siRNA Concentration | % EGFP |
|---|---|
| (nM) | Knockdown |
| 1000 | 44.33 |
| 100 | 15.45 |
| 10 | 3.53 |
| 1 | 2.02 |
| 0.1 | 5.34 |
| 0 | 0 |

### Example 60

### Knockdown of Luciferase Activity with siRNA Containing Pegylated Particles

B16F10-luc2 cells expressing luciferase were grown in complete media (RPMI 1640, 10% HI-FBS and 1% antibiotic/antimycotic solution) at 37° C. with 5% CO₂. Five thousand cells per well in 100 μL/well were added to 96-well plate and grown for 24 hours at 37° C. with 5% CO₂. In separate reactions, the cells were treated with siRNA embedded in pegylated particles, or with siRNA-PLGA (0.01 μM-7.5 μM) conjugate pegylated particles, each for 48 hours. Cells were analyzed for luciferase activity using Bright-Glo® luciferase assay system (Promega). The percentage of cells with luciferase knockdown activity was compared to the luciferase activity of untreated cells. The luciferase knockdown activity was adjusted to the viability of the cells.

The particles used in Example 60 are as follows:

| Particles | Cationic moiety | siRNA Configuration |
|---|---|---|
| A¹. | Cationic PVA | Embedded |
| B². | Cationic PVA | siRNA-disulfide-PLGA |
| | | conjugates |
| C³. | Cationic PVA | siRNA- thioether-PLGA |
| | | conjugates |
| D⁴. | N1-PLGA-N5,N10,N14- | Embedded |
| | tetramethylated-spermine | |
| E⁵. | ⁶N1-PLGA-N5,N10,N14- | Embedded |
| | tetramethylated-spermine | |

¹These particles were prepared essentially as described in Example 32, except the nucleic acid agent targets luciferase (not EGFP) (particle properties measured as described herein: Z_{avg} = 131, Dᵥ90 = 232, Zeta = +15.1).
²These particles were prepared essentially as described in Example 33 (particle properties measured as described herein: Z_{avg} = 130, Dᵥ90 = 231, Zeta = +15.9).
³These particles were prepared as described in Example 55.
⁴These particles were prepared as described in Example 62 (corresponding to a 1:1 N/P ratio).
⁵These particles were prepared as described in Example 62 (corresponding to a 1.5:1 N/P ratio).
⁶As described in Example 68.

The results of the knockdown experiments for the particles described herein are provided below.

| siRNA | % knockdown | % knockdown | % knockdown |
|---|---|---|---|
| Concentration | Treatment A, | Treatment B, | Treatment C, |
| (μM) | Particle A | Particle B | Particle C |
| 0.01 | 1.2 | 11.3 | 29.0 |
| 0.1 | 9.6 | 5.6 | 27.1 |
| 1 | 18.5 | 17.5 | 15.0 |
| 3.75 | 32.0 | 23.0 | 36.0 |

| | % knockdown | % knockdown |
|---|---|---|
| Concentration | Treatment D, | Treatment E, |
| (μM) | Particle D | Particle E |
| 0.01 | 16.9 | 8.6 |
| 0.1 | 5.2 | 4.1 |
| 1 | 10.6 | 4.9 |
| 3.75 | 18.1 | 18.7 |
| 7.5 | 28.1 | 28.0 |

### Example 61

### Formulation and Characterization of siRNA Containing Pegylated Particles Including a Blend of PVA and Cationic PVA as Surfactant, Via Nanoprecipitation

C6-thiol modified oligonucleotides (as used in Example 22) (siRNA, 5 mg, 0.37 μmol, 3 wt. %, Mw 13.6 kDa) were conjugated to 2-(2-(pyridin-2-yl)disulfanyl)ethylamino-5050-PLGA-O-acetyl (100 mg, 15.8 μmol, 57 wt. %, Mw 6.3 kDa) (Example 16) in a solvent mixture of 95:5 DMSO:TE (10 mL) with mPEG₂ₖ-5050PLGA₉ₖ (70 mg, 40 wt %, Mw 11 kDa). In a separate solution, 0.3% w/v PVA (80% hydrolyzed, viscosity 2.5-3.5 cPs) and 0.2% w/v cationic PVA (86-91% hydrolyzed, viscosity 17-27 cPs) in water was prepared. The polymer solution was added using a syringe pump at a rate of 1 mL/min to the aqueous solution (v/v ratio of polymer solution to aqueous phase=1:10), with stiffing at 500 rpm. The particles were then washed with 10 volumes of TE buffer and concentrated using a tangential flow filtration system (300 kDa MW cutoff, membrane area=150 cm²).

Particle properties, evaluated by using the resulting plurality of particles made in the method above:
Z_{avg}=94.0 nm
PDI=0.17
Dᵥ50=79.8 nm
Dᵥ90=139 nm
Zeta potential=+9 mV

### Example 61a

### Formulation and Characterization of siRNA Containing Pegylated Particles Including a Blend of PVA and Cationic PVA as Surfactant, Via Nanoprecipitation

C6-thiol modified oligonucleotides (siRNA, 20 mg, 1.51 μmol, 11.6 wt. %, Mw 13.2 kDa) (as used in Example 22a) were conjugated to 2-(2-(pyridin-2-yl)disulfanyl)ethylamino-5050-PLGA-O-acetyl (85 mg, 12 μmol, 49.4 wt. %, Mw 6.9 kDa) (Example 16) in a solvent mixture of 95:5 DMSO:TE (10 mL) with mPEG₂ₖ-5050PLGA₉ₖ (67 mg, 39 wt %, Mw 11 kDa). In a separate solution, 0.3% w/v PVA (80% hydrolyzed, viscosity 2.5-3.5 cPs) and 0.2% w/v cationic PVA (86-91% hydrolyzed, viscosity 17-27 cPs) in water was prepared. The polymer solution was added using a syringe pump at a rate of 1 mL/min to the aqueous solution (v/v ratio of polymer solution to aqueous phase=1:10), with stiffing at 500 rpm. The particles were then washed with 10 volumes of TE buffer and concentrated using a tangential flow filtration system (300 kDa MW cutoff, membrane area=150 cm²). The loading of siRNA was quantitated using a RiboGreen® fluorescence assay with RNA as a standard. The fluorescence of the siRNA was measured at an excitation wavelength of 480 nm and an emission wavelength of 520 nm.

Particle properties, evaluated by using the resulting plurality of particles made in the method above:
Z_{avg}=84.09 nm
PDI=0.23
Dᵥ50=64.3 nm
Dᵥ90=96.8 nm
Zeta potential=+7.78 mV
siRNA loading: 4.2 wt. %

### Example 61b

### Formulation and Characterization of siRNA Containing Pegylated Particles Including a Blend of PVA and Cationic PVA as Surfactant, Via Nanoprecipitation

C6-thiol modified oligonucleotides (siRNA, 20 mg, 1.51 μmol, 11.6 wt. %, Mw 13617 Da) (as used in Example 22b) were conjugated to 2-(2-(pyridin-2-yl)disulfanyl)ethylamino-5050-PLGA-O-acetyl (85 mg, 12 μmol, 49.4 wt. %, Mw 6.9 kDa) (Example 16) in a solvent mixture of 95:5 DMSO:TE (10 mL) with mPEG₂ₖ-5050 PLGA₉ₖ (67 mg, 39 wt %, Mw 11 kDa). In a separate solution, 0.3% w/v PVA (80% hydrolyzed, viscosity 2.5-3.5 cPs) and 0.2% w/v cationic PVA (86-91% hydrolyzed, viscosity 17-27 cPs) in water was prepared. The polymer solution was added using a syringe pump at a rate of 1 mL/min to the aqueous solution (v/v ratio of polymer solution to aqueous phase=1:10), with stiffing at 500 rpm. The particles were then washed with 10 volumes of TE buffer and concentrated using a tangential flow filtration system (300 kDa MW cutoff, membrane area=150 cm²). The loading of siRNA was quantitated using a RiboGreen® fluorescence assay with RNA as a standard. The fluorescence of the siRNA was measured at an excitation wavelength of 480 nm and an emission wavelength of 520 nm.

Particle properties, evaluated by using the resulting plurality of particles made in the method above:
Z_{avg}=82.42 nm
PDI=0.167
Dᵥ50=62.8 nm
Dᵥ90=112 nm
Zeta potential=+10.5 mV
siRNA loading: 2.97 wt. %

### Example 62

### Formulation and Characterization of siRNA Containing Pegylated Particles Including N1-PLGA-N5,N10,N14-Tetramethylated-Spermine, Using a Two-Step Method

PLGA-O-acetyl (11-19 wt %, Mw 10 kDa), mPEG₂ₖ-5050PLGA₉ₖ (38-48 wt %, Mw 11 kDa) and N1-PLGA-N5,N10,N14-tetramethylated-spermine (37-38 wt %, Mw 8.3 kDa) (described in Example 68) were dissolved to form a total concentration of 1.0% polymer in acetone. In a separate solution, siRNA having 22 base pairs with dTdT overhangs (5-6 wt. %, Mw 14929.06) was dissolved in water. The molar ratio of cation amino groups to siRNA phosphate groups (N/P ratio) was adjusted from 1:1 to 1.5 to 1 by varying the amount of N1-PLGA-N5,N10,N14-tetramethylated-spermine and siRNA used. The polymer acetone solution was added via nanoprecipitation at a total flow rate of 335 mL/min (v/v ratio of organic to aqueous phase=1:10), with stiffing. Acetone was removed by stirring the solution for 2-3 hours. The particles were then washed with 10 volumes of water and concentrated using a tangential flow filtration system (300 kDa MW cutoff, membrane area=50 cm²). PVA (viscosity 2.5-3.5 cp, Sigma-Aldrich) was added to the particles and allowed to stir for 2-3 hours.

Particle properties, evaluated by using the resulting plurality of particles made in the method above:

| | | | | | Zeta | siRNA |
|---|---|---|---|---|---|---|
| N/P | Z_{avg} | | Dᵥ50 | Dᵥ90 | potential | concentration |
| Ratio | (nm) | PDI | (nm) | (nm) | (mV) | (mg/mL) |
| 1:1 | 94 | 0.23 | 55 | 121 | −12.5 | 0.29 |
| 1.5:1 | 108 | 0.22 | 70 | 163 | −9.5 | 0.30 |

### Example 62a

### Viability of Cells Treated with siRNA in Pegylated Particles Including N1-PLGA-N5,N10,N14-Tetramethylated-Spermine

To measure cell viability of siRNA containing pegylated particles including N1-PLGA-N5,N10,N14-tetramethylated-spermine, MDA-MB-231/GFP cells were plated in (2) 96-well white opaque-clear bottom plates at a density of 10,000 cells per well. Prior to treatment with particles, cells were cultured overnight in modified complete culture media; DMEM, 10% fetal bovine serum, 0.1 mM MEM non-essential amino acids, 2 mM L-glutamine and 1% penicillin streptomycin (all from Life Technologies) at 37° C. with 5% CO₂. Cells were then treated with 5 to 0.01 μM of entrapped siRNA containing pegylated particles including N1-PLGA-N5,N10,N14-tetramethylated-spermine in triplicate and incubated for 24 and 48 hours at 37° C., 5% CO₂, respectively. Following incubation, 20 μL of CellTiter96® AQueous One™ viability reagent (Promega) was added to each well containing 100 μL of media±entrapped CPX1310/PLGA/PEG. The plate was then incubated at 37° C. for 2 hours. Viability was determined by measuring the absorbance at 490 nm using a SpectraMax® M5 (Molecular Devices) plate reader. The percent of viable cells of which were treated were compared directly to those of which were not treated at similar time points, as shown below.

| siRNA Concentration | | |
|---|---|---|
| (μM) | % Viable - 24 hrs | % Viable - 48 hrs |
| 5 | 88.21 ± 0.81 | 96.48 ± 5.1 |
| 1 | 93.77 ± 1.04 | 91.67 ± 6.78 |
| 0.1 | 95.74 ± 2.45 | 99.94 ± 4.82 |
| 0.01 | 97.95 ± 1.56 | 104.79 ± 1.35 |

### Example 62b

### Knockdown Activity of siRNA by siRNA in Pegylated Particles Including N1-PLGA-N5,N10,N14-Tetramethylated-Spermine

To measure EGFP knockdown activity of siRNA by siRNA containing pegylated particles including N1-PLGA-N5,N10,N14-tetramethylated-spermine, MDA-MB-231 EGFP cells were plated in (2) 96-well white opaque-clear bottom plates at a density of 10,000 cells per well. MDA-MB-231 EGFP cells were grown overnight in modified complete culture media; DMEM, 10% fetal bovine serum, 0.1 mM MEM non-essential amino acids, 2 mM L-glutamine and 1% penicillin streptomycin (all from Life Technologies) at 37° C. with 5% CO₂. The following day, the volume of media corresponding to the volume of formulation was removed from each well. Cells were then treated with 5 to 0.01 μM of siRNA containing pegylated particles including N1-PLGA-N5,N10,N14-tetramethylated-spermine in triplicate. The treated cells were incubated for 24 and 48 hours at 37° C., 5% CO₂, respectively. At designated time points (24 hours and 48 hours); cells were washed once with PBS and lysed with M-PER (mammalian protein extraction reagent, Thermo Fisher) supplemented with HALT® protease inhibitor cocktail (Thermo Fisher) on ice for 15 minutes followed by incubation for 10 minutes at room temperature on the orbital plate shaker (200 rpm). EGFP measurements were completed using a SpectraMax® M5 (Molecular Devices) fluorescent plate reader set with an excitation of 488 nm and emission of 535 nm, with a cutoff designated at 535 nm. The percent knockdown of treated cells was generated from the decrease of EGFP signal when compared to untreated control wells from similar time points as shown below.

| siRNA Concentration | % EGFP Knockdown | % EGFP Knockdown |
|---|---|---|
| (μM) | (24 hrs) | (48 hrs) |
| 5 | 31.98 ± 2.4 | 68.05 ± 0.28 |
| 1 | 18.39 ± 0.47 | 52.88 ± 2.07 |
| 0.1 | 20.91 ± 0.74 | 26.15 ± 1.80 |
| 0.01 | 12.65 ± 3.05 | 18.56 ± 2.19 |

### Example 62c

### Viability of Cells Treated with siRNA in Pegylated Particles Including N1-PLGA-N5,N10,N14-Tetramethylated-Spermine and O-acetyl PLGA

To measure cell viability of siRNA by siRNA containing pegylated particles including N1-PLGA-N5,N10,N14-tetramethylated-spermine and O-acetyl PLGA, MDA-MB-231 EGFP cells were plated in (2) 96-well white opaque-clear bottom plates at a density of 10,000 cells per well. Prior to treatment with particles, cells were cultured overnight in modified complete culture media; DMEM, 10% fetal bovine serum, 0.1 mM MEM non-essential amino acids, 2 mM L-glutamine and 1% penicillin streptomycin (all from Life Technologies) at 37° C. with 5% CO₂. Cells were then treated with 5 to 0.01 μM of siRNA containing pegylated particles including N1-PLGA-N5,N10,N14-tetramethylated-spermine and O-acetyl PLGA in triplicate and incubated for 24 and 48 hours at 37° C., 5% CO₂, respectively. Following incubation, 20 μL of CellTiter96® AQueous One™ viability reagent (Promega) was added to each well containing 100 μL of media±entrapped CPX1310/CPX1025/PLGA-mPEG. The plate was then incubated at 37° C. for 2 hours. Viability was determined by measuring the absorbance at 490 nm using a SpectraMax® M5 (Molecular Devices) plate reader. The percent of viable cells of which were treated were compared directly to those of which were not treated at similar time points, as shown below.

| siRNA Concentration | | |
|---|---|---|
| (μM) | % Viable - 24 hrs | % Viable - 48 hrs |
| 5 | 95.57 ± 2.78 | 91.57 ± 6.30 |
| 1 | 98.08 ± 2.22 | 96.8 ± 2.80 |
| 0.1 | 96.76 ± 0.74 | 98.11 ± 2.40 |
| 0.01 | 101.14 ± 0.92 | 99.34 ± 0.41 |

### Example 62d

### Knockdown Activity of siRNA by siRNA in Pegylated Particles Including N1-PLGA-N5,N10,N14-Tetramethylated-Spermine and O-acetyl PLGA

To measure EGFP knockdown activity of siRNA in pegylated particles including N1-PLGA-N5,N10,N14-tetramethylated-spermine and O-acetyl PLGA, MDA-MB-231 EGFP cells were plated in (2) 96-well white opaque-clear bottom plates at a density of 10,000 cells per well. MDA-MB-231 EGFP cells were grown overnight in modified complete culture media; DMEM, 10% fetal bovine serum, 0.1 mM MEM non-essential amino acids, 2 mM L-glutamine and 1% penicillin streptomycin (all from Life Technologies) at 37° C. with 5% CO₂. The following day, the volume of media corresponding to the volume of formulation was removed from each well. Cells were then treated with 5 to 0.01 μM of siRNA in pegylated particles including N1-PLGA-N5,N10,N14-tetramethylated-spermine and O-acetyl PLGA. in triplicate. The treated cells were incubated for 24 and 48 hours at 37° C., 5% CO₂, respectively. At designated time points (24 hours and 48 hours); cells were washed once with PBS and lysed with M-PER (mammalian protein extraction reagent, Thermo Fisher) supplemented with HALT® protease inhibitor cocktail (Thermo Fisher) on ice for 15 minutes followed by incubation for 10 minutes at room temperature on the orbital plate shaker (200 rpm). EGFP measurements were completed using a SpectraMax® M5 (Molecular Devices) fluorescent plate reader set with an excitation of 488 nm and emission of 535 nm, with a cutoff designated at 535 nm. The percent knockdown of treated cells was generated from the decrease of EGFP signal when compared to untreated control wells from similar time points as shown below.

| siRNA Concentration | % EGFP Knockdown | % EGFP Knockdown |
|---|---|---|
| (μM) | (24 hrs) | (48 hrs) |
| 5 | 30.54 ± 1.55 | 34.85 ± 6.72 |
| 1 | 19.69 ± 2.24 | 15.53 ± 3.38 |
| 0.1 | 11.79 ± 2.34 | 29.24 ± 0.44 |
| 0.01 | 7.28 ± 0.51 | 18.94 ± 9.8 |

### Example 63

### Formulation and Characterization of siRNA Containing Pegylated Particles Including a Blend of PVA and Cationic PVA as Surfactant, Via Nanoprecipitation

C6-thiol modified oligonucleotides (as used in Example 22) (siRNA, 5 mg, 0.37 μmol, 3 wt. %, Mw 13.6 kDa) were conjugated to 1,2-distearoyl-sn-glycero-3-phosphoethanolamine-N—[PDP(polyethylene glycol)-2k] (40 mg, 13.4 μmol, 28 wt. %, Mw 2.98 kDa) (as done Example 56) in Tris-EDTA buffer with addition of mPEG₂ₖ-5050PLGA₉ₖ (60 mg, 28 wt %, Mw 11 kDa) and 5050 PLGA-O-acetyl (40 mg, 41 wt. %) in a solvent mixture of 8:2 acetonitrile:TE (14 mL). In a separate solution, 0.3% w/v PVA (80% hydrolyzed, viscosity 2.5-3.5 cPs) and 0.2% w/v cationic PVA CM-318 (86-91% hydrolyzed, viscosity 17-27 cPs) in water was prepared. The polymer solution was added using a syringe pump at a rate of 1 mL/min to the aqueous solution (v/v ratio of polymer solution to aqueous phase=1:10), with stirring at 500 rpm. Organic solvent was removed by stirring the solution for 2-3 hours. The particles were then washed with 10 volumes of TE buffer and concentrated using a tangential flow filtration system (300 kDa MW cutoff, membrane area=150 cm²).

Particle properties, evaluated by using the resulting plurality of particles made in the method above:
Z_{avg}=124.9 nm
PDI=0.118
Dᵥ50=112 nm
Dᵥ90=196 nm
Zeta potential=+8 mV

### Example 64

### Formation of Nucleic Acid Agent Containing Pegylated Particles Including Cationic Polymers, Via Nanoprecipitation, Using PVA as Surfactant

5050-O-acetyl-PLGA (60 mg, 60 wt. %) and nucleic acid-conjugated mPEG₂ₖPLGA (Example 23) (40 mg, 40 wt %, Mw ˜25.7 kDa) will be dissolved to form a total concentration of 1.0% polymer in a solvent mix of Tris-EDTA:DMSO (5:95) or alternatively Tris-EDTA:acetonitrile. In a separate solution, 0.3% w/v PVA (80% hydrolyzed, viscosity 2.5-3.5 cPs, Sigma-Aldrich) and 0.2% w/v cationic PVA (86-91% hydrolyzed, viscosity 17-27 cPs, Kuraray) in water will be prepared. The polymer solution will be added using a syringe pump at a rate of 1 mL/min to the aqueous solution (v/v ratio of polymer solution to aqueous phase=1:10), with stirring at 500 rpm. The particles will then be washed with 10 volumes of TE buffer and concentrated using a tangential flow filtration system (300 kDa MW cutoff, membrane area=150 cm²).

### Example 65

### Formulation of siRNA Containing Pegylated Particles Including 1-hexyltriethyl-ammonium phosphate (Q6) and PVA as a Surfactant, Via Nanoprecipitation

PLGA-O-acetyl (11-19 wt %, Mw 10 kDa), mPEG₂ₖ-5050 PLGA₉ₖ (38-48 wt %, Mw 11 kDa) and 1-hexyltriethyl-ammonium phosphate (37-38 wt %, Mw 8.3 kDa) were dissolved to form a total concentration of 1.0% polymer in acetone. In a separate solution, siRNA having 22 base pairs with dTdT overhangs (5-6 wt. %, Mw 14929.06) was dissolved in water. The molar ratio of cation amino groups to siRNA phosphate groups (N/P ratio) was 15:1, specifically the amount of 1-hexyltriethyl-ammonium phosphate and siRNA used. The polymer acetone solution was added via nanoprecipitation at a total flow rate of 335 mL/min (v/v ratio of organic to aqueous phase=1:10), with stiffing. Acetone was removed by stirring the solution for 2-3 hours. The particles were then washed with 10 volumes of water and concentrated using a tangential flow filtration system (300 kDa MW cutoff, membrane area=50 cm²). PVA (viscosity 2.5-3.5 cp, Sigma-Aldrich) was added to the particles and allowed to stir for 2-3 hours.

Particle properties, evaluated by using the resulting plurality of particles made in the method above:
Z_{avg}=98 nm
PDI=0.41
Dᵥ50=34 nm
Dᵥ90=68 nm
Zeta potential=−11.5 mV
siRNA drug loading=1.51 wt %

### Example 66

### Characterization of siRNA Embedded in Pegylated Particles (Non-Conjugated) with Cationic PVA Using Enzymatic Digestion Assay

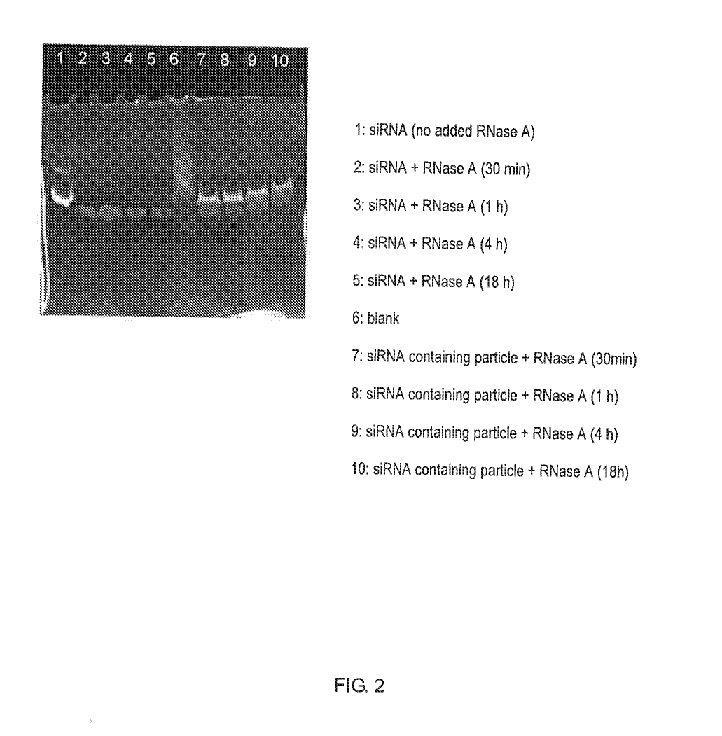

Aliquots of pegylated particles containing 0.5 μg siRNA (Example 32) were incubated at 37° C. with RNase A (1 μg) for each of four time periods (30 min, 1 h, 4 h and 18 h). Each reaction was quenched with proteinase K (0.07 mg) and SDS (0.2 mg) with further incubation at 37° C. for 30 mins. Samples were then frozen and analyzed by 20% PAGE with ethidium bromide staining. The same protocol was repeated with free siRNA. The results are provided in FIG. 2.

In lanes 2-5, faint bands of material were observed due to the digestion of siRNA by RNase to shorter length products. Complete digestion of siRNA to shorter species was observed after 30 mins of incubation of siRNA with RNase, see lane 2.

In lanes 7-10, bands of stronger intensities, having migration similar to that of undigested siRNA in lane 1, were observed above faint, diffuse bands, having migration similar to that of the digestion products in lanes 2-5. High molecular weight bands, having migrations similar to that of undigested siRNA, lane 1, were observed at all time periods for siRNA contained in particles, lanes 7-10. A high molecular weight band was still observed after 18 hours of digestion with RNase A, lanes 7-10.

### Example 67

### Characterization of siRNA-Polymer Conjugate Particles with Cationic PVA Using Enzymatic Digestion Assay

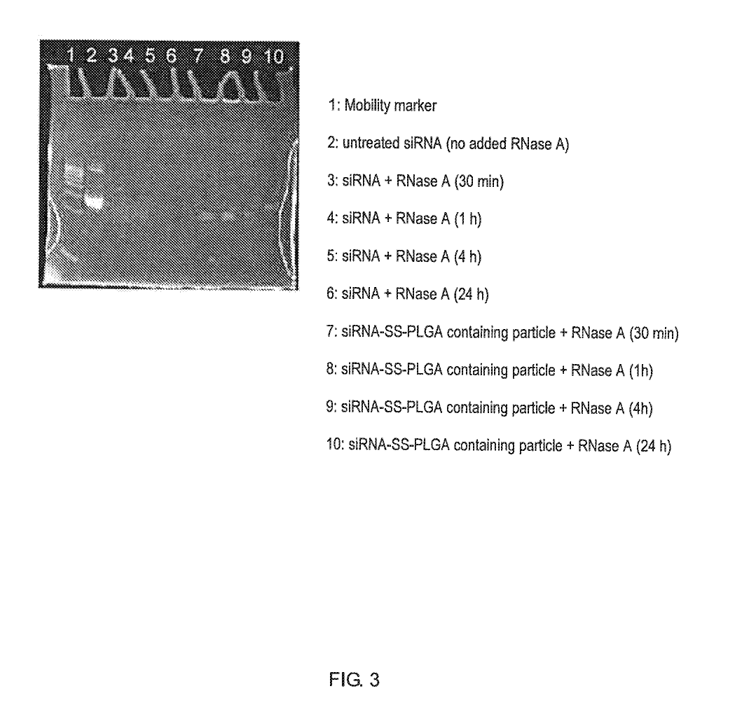

Aliquots of pegylated particles containing 26 μg of siRNA-S—S-PLGA (Example 58) were incubated at 37° C. with RNase A (50 μg) for each of four time periods (30 min, 1 h, 4 h and 18 h). Each reaction was quenched with proteinase K (0.28 mg) and SDS (0.8 mg) with further incubation at 37° C. for 30 mins. Samples were then frozen and analyzed by 20% PAGE with ethidium bromide staining. The same protocol was repeated with free siRNA. The results are provided in FIG. 3.

Incubation of the free siRNA with RNase A, lanes 3-6, showed that all the siRNA is digested at each incubation time. With siRNA-SS-PLGA particles, lines 7-10, faint bands corresponding to siRNA were still visible, showing that the particles slowed down digestion of the siRNA by RNase A.

### Example 68

### Synthesis, Purification, and Characterization of N1-PLGA-N5,N10,N14-tetramethylated-spermine

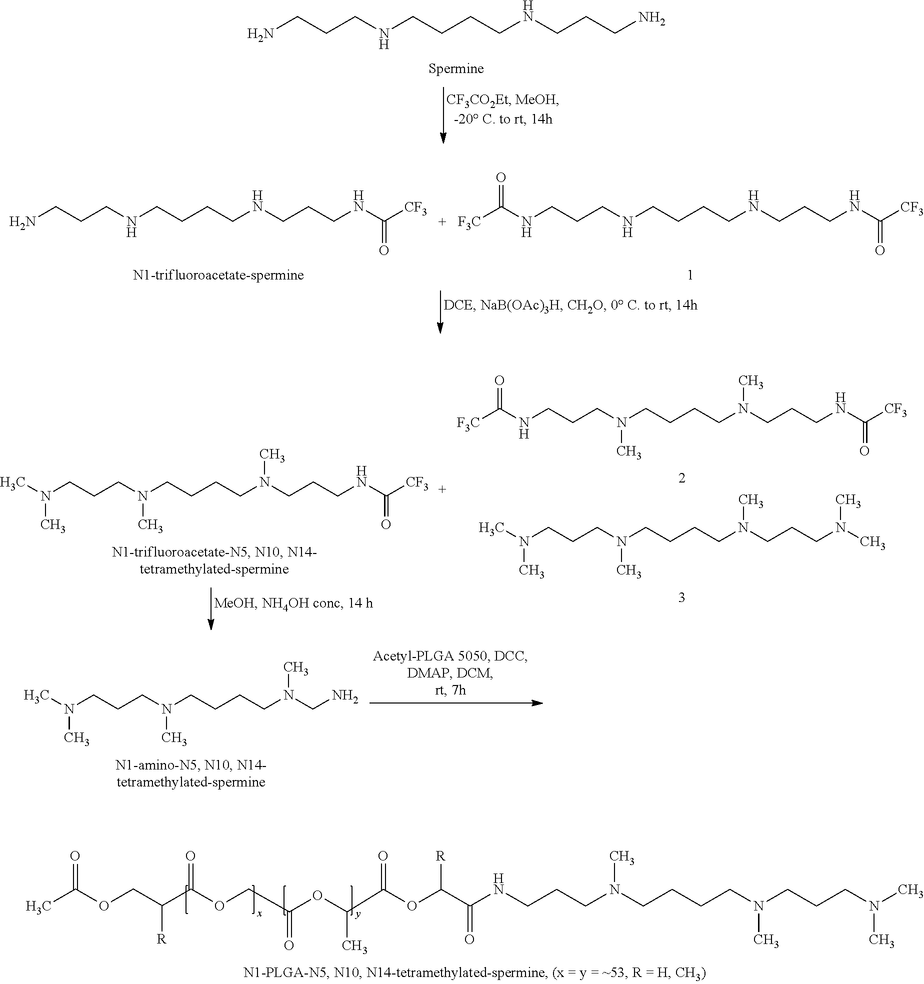

A 3-L three-neck round bottom flask equipped with an internal temperature probe, mechanical stirrer and addition funnel was flushed with nitrogen and charged with spermine (9.60 g, 47.44 mmol) and MeOH (670 mL). The solution was cooled to −20° C. After that, a solution of ethyl trifluoroacetate (6.74 g, 47.44 mmol) in MeOH (100 mL) was added dropwise for 90 min via addition funnel. The mixture was stirred for 14 h, allowing the temperature to rise to room temperature. The progress of the reaction was monitored by MS [direct injection ESI(+)]. The mixture contained spermine, N1-trifluoroacetate-spermine and compound 1. N1-trifluoroacetate-spermine was a major peak.

After that time, the organic solvents were removed under vacuum to afford an oil residue, which was added as a solution in 1,2-dichloroethane (DCE, 950 mL) into 3-L three-neck round bottom flask equipped with an internal temperature probe, mechanical stirrer and addition funnel. The mixture was cooled to 0° C. and 37% wt aqueous solution of formamide (19.2 g, 237.3 mmol) was added in 15 min. The mixture was stirred for 30 min at 0° C. and then sodium triacetoxyborohydride (60.3 g, 281.5 mmol) was added in 3 portions over 15 min as a solid. The mixture was stirred for 14 h, allowing the temperature to rise to room temperature. The progress of the reaction was monitored by MS [direct injection ESI(+)]. The mixture contained N1-trifluoroacetate-N5,N10,N14-tetramethylated-spermine, compounds 2, 3 and no material from the previous step.

The reaction mixture was transferred into a 1-L separatory funnel and washed with saturated sodium bicarbonate (150 mL). The layers were separated, the aqueous layer was extracted with methylene chloride (3×100 mL). The combined organic layers were dried over sodium sulfate, filtered and concentrated in vacuum. The aqueous layer was charged into 500-mL round bottom flask and freeze-dried overnight. The residue was diluted with a solution of methylene chloride (250 mL) and triethyl amine (25 mL), and it stirred with a mechanical stirrer for 30 min. After that, the mixture was filtered and the filter cake was transferred back into a flask and diluted with a solution of methylene chloride (250 mL) and triethyl amine (25 mL). The described process was repeated two times.

The methylene chloride/TEA extracts were combined with methylene chloride extracts from separation and dried over sodium sulfate, filtered and concentrated in vacuum into a residue ˜15 g. The residue was purified by column chromatography on silica (350 g), using a mixture of DCM/MeOH/TEA (6/3/1 (v/v/v)) as an eluent (total solvent used 2 L). The fractions were visualized by phosphomolybdic acid stain. The fractions containing the product [R_{f}=0.41] were pulled out and concentrated in vacuum to afford N1-trifluoroacetate-N5,N10,N14-tetramethylated-spermine [˜7 g]. A 500-mL single-neck round bottom flask equipped with a magnetic stirrer was charged with N1-trifluoroacetate-N5,N10,N14-tetramethylated-spermine (7.00 g, 19.7 mmol), MeOH (70 mL) and NH₄OH (conc. 210 mL). The mixture was stirred for 14 h at room temperature. After that time, [direct injection ESI(+)] showed completion of the reaction. The mixture was concentrated in vacuum and dry-loaded on silica column (350 g silica).

The column was eluted with THF/MeOH/conc. NH₄OH in ratios 7/2/1 (1.5 L). The fractions were visualized with 2% ninhydrin in ethanol stain. The fractions containing the product [R_{f}=0.6] were pulled out and concentrated in vacuum to afford N1-amino-N5,N10,N14-tetramethylated-spermine [740 mg], the structure of which was confirmed by ¹H NMR and MS (ESI+). The combined mixed fractions were concentrated and loaded and dry-loaded on silica column (350 g silica). The column was eluted with THF/MeOH/conc.NH₄OH in ratios 3/1/1 (1.5 L).

A 500-mL round bottom flask was charged with acetyl-PLGA 5050-7K (15.00 g, 2.83 mmol based on a Mn of 5300 Da), DCM (40 mL) and toluene (100 mL). The content was concentrated under vacuum to remove residual water. After that, the same flask was charged with DCC (877 mg, 4.25 mmol, 1.5 equiv.), DMAP (69 mg, 0.57 mmol, 0.2 equiv.), N1-amino-N5,N10,N14-tetramethylated-spermine (1.10 g, 4.25 mmol, 1.5 equiv.), and DCM (125 mL). The mixture slowly turned cloudy. After stirred for 7 h, the mixture was diluted with DCM (100 mL) and filtered. The filter cake was washed with fresh DCM (30 mL). The DCM solutions were combined, transferred into a 500-mL separatory funnel and gently washed with 0.0001 N NaOH solution (100 mL, pH=10). Some emulsion formation was observed. The emulsion was rested for 30 min and the layers separated. The organic layer was separated, and the aqueous layer was extracted with DCM (2×50 mL). The organic layers were combined, dried over Na₂SO₄, filtered through a Celite® pad and concentrated under vacuum.

The residue was dissolved in acetone (100 mL) and concentrated under vacuum. The residue was re-dissolved in acetone (100 mL), filtered through 0.2 μm PTFE filter and precipitated into MTBE. using a 2-L three neck round bottom flask equipped with a mechanical stirrer, and cooled to 0° C. A solution of crude N1-PLGA-N5,N10,N14-tetramethylated-spermine in acetone was added dropwise into the flask with a constant stirring. The polymer started to precipitate right away as a sticky material. The resulted suspension was stirred for 30 min at 0° C. and then at room temperature for 30 min. The liquid was decanted off and the residue was re-dissolved in acetone to allow the transfer of solid material and then was concentrated in vacuum to afford the desired product [12.0 g, 80%]. ¹H NMR analysis showed conjugation of N1-amino-N5,N10,N14-tetramethylated-spermine to the polymer and absence of DMAP. The loading of N1-PLGA-N5,N10,N14-tetramethylated-spermine was 4.3 wt % (92% of theoretical loading based on a MW of 5.3 kDa) as estimated by ¹H NMR analysis. HPLC analysis showed 96.9% purity (AUC, 230 nm).

### Example 69

### Synthesis, Purification, and Characterization of N1-PLGA-N5,N10,N14-tri-Cbz-spermine

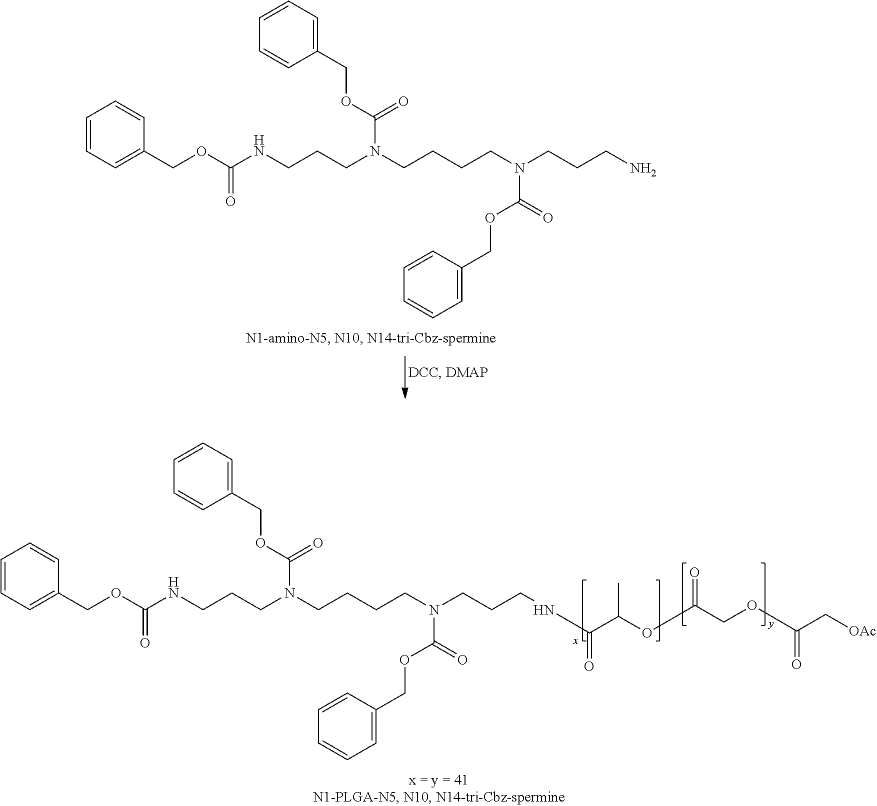

Acetyl-PLGA 5050-7K (8.7 g, 1.65 mmol) was dissolved in DCM (22 mL, 2.5 vol) and diluted with toluene (61 mL, 7.0 vol). The viscous mixture was concentrated to dryness using a rotary evaporator at bath temperature of 40° C. to give white solid material. The solid was dissolved in DCM (70 mL, 8.0 vol) and DCC (0.51 g, 2.48 mmol) followed by DMAP (40 mg, 0.33) were added. N1-amino-N5,N10,N14-tri-Cbz-spermine (1.5 g, 2.48 mmol) in DCM (9 mL) was then added at which time formation of precipitate was observed. The batch was stirred at 20-25° C. for 16.5 h. The heterogeneous reaction mixture was monitored by HPLC which was similar to that of previous batches prepared. The batch was diluted with DCM (61 mL) and filtered through a 0.3 μm in-line filter to remove DCU. The filter was rinsed with DCM (15 mL). The filtrate was washed with cooled 2 M HCl solution (0-5° C., 2×61.0 mL). (HPLC analysis of the aqueous waste streams indicated that N1-amino-N5,N10,N14-tri-Cbz-spermine wasn't purged.) The mixture was diluted with DCM (61 mL) and stirred with activated Dowex™ 50WX8 (20 g wet) for 3 h. The batch was filtered and analyzed by HPLC which showed that the concentration of N1-amino-N5,N10,N14-tri-Cbz-spermine was significantly reduced. N1-amino-N5,N10,N14-tri-Cbz-spermine was present in 35.8% AUC while the product was present in 64.1% AUC at 205 nm.

The filtrate was concentrated to dryness to give the crude as off white foam (10.0 g). The crude was dissolved in acetone (150 mL). Celite® (40 g, 4 vol) was added to the batch. MTBE (400 mL) was then added while agitating the batch with an overhead stirrer. The slurry was stirred for 2 h and filtered. The filtrate was set aside and the product that was mixed with Celite® was rinsed with DCM (350 mL). The filtrate was analyzed by HPLC which showed traces amount of N1-amino-N5,N10,N14-tri-Cbz-spermine. The filtrate was concentrated to dryness and N1-PLGA-N5,N10,N14-tri-Cbz-spermine was submitted to a second purification by precipitation in acetone/MTBE mixture in the presence of Celite®. The second precipitation removed N1-amino-N5,N10,N14-tri-Cbz-spermine completely. The batch was filtered and the filtrate was discarded. The Celite® was rinsed with DCM (350 mL) and the filtrate was then concentrated to dryness and dried under high vacuum overnight to give the product as white foam (5.4 g). HPLC analysis of the batch showed that N1-amino-N5,N10,N14-tri-Cbz-spermine was purged completely. Based on ¹H NMR, the loading was 84% (8.5% wt loading). GPC analysis of the batch which showed an MP (molecular weight peak) of 11.9 Da.

### Example 70

### Synthesis, Purification, and Characterization of N14-acetyl PLGA-spermine

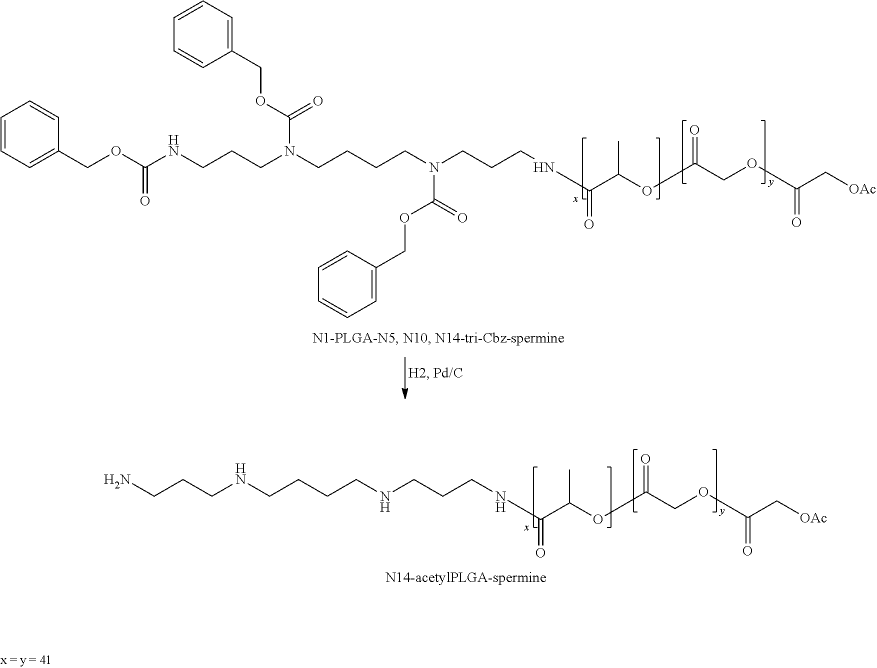

In a 250 mL autoclave, N1-PLGA-N5,N10,N14-tri-Cbz-spermine (5.1 g), DCM (76.5 mL, 15 vol), MeOH (38 mL, 2 M HCl (1.7 mL) and 10% Pd/C (1.0 g) were added. The reaction mixture was purged with N₂ (3×15 psig) followed by H₂ (25 psig). Hydrogenation then began at 20-25° C. and 25 psig H₂ pressure. The reaction was monitored after 4 and 6.5 h, but there was small amount of starting material remaining. After 8.5 h, there were only trace amounts of starting material remaining. The mixture was then filtered through a bed of Celite® and rinsed with DCM (2×20 mL). The filtrate was concentrated to dryness to give the crude product as off white foam (5.08 g). GPC analysis of the crude showed that the MP (molecular weight peak) was 10.6 Da. N14-acetylPLGA-spermine was purified by precipitation in DCM/MTBE.

### Example 70a

### Formation and Characterization of siRNA Containing Pegylated Particles Including N14-Acetyl PLGA-Spermine, Via Nanoprecipitation, Using PVA as Surfactant

N14-acetyl PLGA-spermine (68 wt. %, Mw 10.7 kDa) and mPEG₂ₖ-PLGA (29 wt %, Mw 11 kDa) were dissolved to form a total concentration of 1.0% polymer in acetone. In a separate solution, siRNA having 22 base pairs with dTdT overhangs (3 wt %, Mw 14929.06) was dissolved in a solution of 0.5% w/v PVA (80% hydrolyzed, viscosity 2.5-3.5 cPs, Sigma-Aldrich) in water. The molar ratio of cation amino groups to siRNA phosphate groups (N/P ratio) was 1.8:1, e.g. ratio of N14-acetyl PLGA-spermine and siRNA respectively. The polymer acetone solution was added via nanoprecipitation at a total flow rate of 335 mL/min (v/v ratio of organic to aqueous phase=1:8), with stirring. Acetone was removed by stirring the solution for 2-3 hours. The particles were then washed with 10 volumes of water and concentrated using a tangential flow filtration system (300 kDa MW cutoff, membrane area=50 cm²).

Particle properties, evaluated by using the resulting plurality of particles made in the method above:
Z_{avg}=69 nm
PDI=0.24
Dᵥ50=43 nm
Dᵥ90=78 nm
Zeta potential=−7.8 mV
Drug loading=1.27 wt %

### Example 70b

### Methods to Characterize siRNA Loading in Mpeg-PLGA/PVA Particles

mPEG-PLGA analysis: mPEG2k-PLGA as standard and lyophilized samples were digested with sodium hydroxide (1N) for 2.5 hr at 90° C., then they were neutralized with Formic acid (1N) for the HPLC analysis. ELSD detector was used for all analysis. Based on this method of analysis, the range of mPEG-PLGA in siRNA particles is in the range of 8-15 wt. %.

PVA Assay:

Particle formulation and PVA standards were analyzed with the colormetric assay (iodine assay). Samples were digested with 2 ml sodium hydroxide (0.5N) at 60° C. for 20 min. Then they were neutralized with 0.9 ml hydrochloric acid (1N). 3 ml of Boric acid (0.65M) and 0.5 ml of Iodine/potassium iodide (0.05M/0.15M) were added to the neutralized samples. Analytes were diluted with water then measured at 690 nm with UV spectrophotometer. Based on this method of analysis, the range of PVA in siRNA particles is in the range of 35-55 wt. %.

RiboGreen® Assay for siRNA Loading:

RiboGreen® assay was used to quantify the RNA content of the RNA-cationic PVA particle with RNA as a standard. RNA standard was diluted in TE buffer in different concentration (2 ug/ml to 0.01 ug/ml). The samples were excited at 480 nm and the fluorescent emission intensity was measured at 520 nm. The particle sample was diluted with buffer for fluorescent analysis.

Wt. % of components in particles of Examples 71-75.

| Components in siRNA particles | Wt % |
|---|---|
| siRNA | 1-6 |
| mPEG-PLGA | 8-15 |
| Derivatised PLGA | 24-56 |
| PVA blend | 35-55 |
| (cationic and non-cationic) | |

### Example 71

### In Vivo siRNA Knockdown of EGFP

Cultured MDA-MB-231 breast cancer cells genetically engineered to express EGFP were implanted into the mammary fat pad of nude mice. On Day 8 post-implantation, mice in each of six groups (Groups 1-6, nine mice per group) were administered control or siEGFP formulations, as described in Table AA. Mice in Group 1 were administered a formulation of vehicle (10% sucrose), which provided a “positive” control of no knockdown. Mice in Group 2 were administered particles prepared according to example 32b, which provided a control siRNA particle against a non-targeted luciferase gene. Mice in Group 6 were administered a formulation of lipopolysaccharide (LPS 0111:B4, Sigma-Aldrich), which stimulated cytokine release as an additional control group. Mice in Groups 3, 4, and 5, were administered formulations of siEGFP particles as a 10 mL/kg bolus into the tail vein.

The formulations were administered intravenously every other day (a total of two administrations for each mouse). The dosages, in mg/kg, and volume of formulation administered, are given in Table AA. Tumor samples were collected from 3 mice at each of 24 hours, 72 hours, and 168 hours, after the 2^{nd} (final) administration, in each of Groups 1-6. Collected tumor material was sectioned into 3 individual pieces for analysis. Sections were directly placed onto dry ice, placed into RNAlater® (Life Technologies) or dissociated into single cells with phosphate buffer saline supplemented with 5% fetal bovine serum and 0.1% sodium azide.

TABLE AA
Dosage schedule.

| | | Dose | | | |
|---|---|---|---|---|---|
| Group | Formulation | mg/kg | Volume | Schedule | N |
| 1 | Vehicle 10% sucrose | — | | q2d x 2 | 9 |
| 2 | Particles prepared | 3 | | q2d x 2 | 9 |
| | according to Example | | | | |
| | 32b | | | | |
| 3 | Particles prepared | 3 | | q2d x 2 | 9 |
| | according to Example | | | | |
| | 55a | | | | |
| 4 | Particles prepared | 3 | | q2d x 2 | 9 |
| | according to Example | | | | |
| | 61a | | | | |
| 5 | Particles prepared | 3 | | q2d x 2 | 9 |
| | according to Example | | | | |
| | 32a | | | | |
| 6 | LPS | 0.1 | | q2d x 2 | 9 |

The effect of the treatment on EGFP knockdown in tumor cells was analyzed by FACS analysis, EGFP fluorescence and EGFP RNA levels.

The FACScan™ cytometry was used to measure the fluorescence in individual cells isolated from collected tumor samples. The FACScan™ flow cytometer utilized CellQuest™ as the acquisition software, with the desired number of events set at 10,000. To consider specific population of cells within the collected data, two gates were created. The non-fluorescent gate was determined using a non-EGFP cell line, in parallel, the fluorescent gate was selected using the vehicle controlled isolated cells. The nature of the gates scored the cells as either no longer fluorescent following treatment or unaffected by the treatment.

In the EGFP fluorescence analysis, the level of EGFP fluorescence in samples of collected tumors was first normalized for total protein. Total protein was determined using a BCA assay kit (ThermoFisher). Following the calculations of total protein, 50 μg of protein was measured for fluorescence by a fluorescent plate reader (Excitation wavelength=488, Emission wavelength=535). The % knockdown value for EGFP fluorescence was calculated by determining the percent decrease in the fluorescent output when compared to the vehicle control.

In the RNA analysis, the level of EGFP mRNA, in samples of extracted RNA from collected tumors was determined by hybridization to an EGFP specific probe and detection with sandwich nucleic acid hybridization to branched probes. The % knockdown value for EGFP mRNA was calculated by determining the % decrease of luminescence created by the hybridization to the label probe. Prior to this, the samples were normalized against human GAPDH (glyceraldehyde 3-phosphate dehydrogenase) which was completed in parallel to the EGFP hybridization steps. The human gene allowed for comparison of the injected tumor cells and prevented any contamination from the mouse. Results are shown in Table BB.

TABLE BB
In vivo knockdown results.

| | % Knockdown | | | | | | | | |
|---|---|---|---|---|---|---|---|---|---|
| | | | | EGFP | | | QuantiGene ® | | |
| | FACS | | | Fluorescence | | | 2.0 (RNA levels) | | |
| | 24 Hr | 72 Hr | 168 Hr | 24 Hr | 72 Hr | 168 Hr | 24 Hr | 72 Hr | 168 Hr |
| Particles prepared | ** | 4.77 | ** | 8.41 ± 4.97 | 1.69 ± 10.4 | ** | 3.49 | ** | ** |
| as in Example 32b | | | | | | | | | |
| Particles prepared | ** | 2.49 | ** | 28.6 ± 6.45 | 20.19 ± 14.75 | 9.22 ± 1.98 | 21.45 | 15.89 | ** |
| according to | | | | | | | | | |
| Example 55a | | | | | | | | | |
| Particles prepared | 30.88 | 49.89 | ** | 54.45 ± 2.2 | 37.88 ± 3.1 | 23.31 ± 10.89 | 42.65 | 20.56 | ** |
| according to | | | | | | | | | |
| Example 61a | | | | | | | | | |
| Particles prepared | ** | 18.79 | ** | 21.28 ± 1.58 | 26.56 ± 7.31 | 16.43 ± 4.24 | 11.76 | 11.05 | ** |
| according to | | | | | | | | | |
| Example 32a | | | | | | | | | |
| LPS | ** | 8.9 | ** | 10.11 ± 1.64 | 6.45 ± 3.83 | 3.85 ± 7.86 | ** | ** | ** |

** No Knockdown was observed.

5′-RLM-RACE PCR was used to confirm that reduction in EGFP mRNA was due to site-specific siRNA-directed cleavage. siRNA-directed cleavage by the siEGFP results in the specific cleavage between nucleotides 414 and 415 of the gene by a multiprotein complex that activates RNase and cleaves the RNA. Purified RNA extracted from tumor samples (24 hour time point) was then used in the GeneRacer™ Advanced RACE kit (Invitrogen, L1502-01). Using a gene specific primer (5′ TCAGGTTCAGGG GGAGGTGTGG-3′), the sample was reverse transcribed allowing for PCR amplification to occur using a forward GeneRacer™ 5′ primer (designed for the specific ligated RNA oligo) (5′ CGACTGGAGCACGAGGACACTGA-3′) and a reverse gene specific primer (5′ CGCCGATGGGGGTGTTCTGC-3′). Standard PCR conditions were used and 25 cycles of amplification were completed.

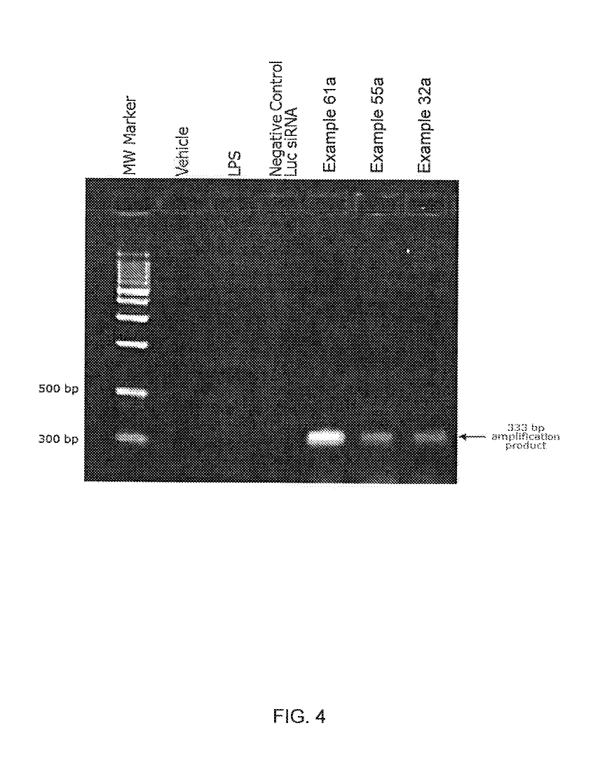

The amplified product is shown in FIG. 4, which depicts a 4% agarose gel of the PCE products, and shows confirmation of knockdown by 5′ RLM RACE-PCR for 24 hour time period samples. The predicted primer length was 333 base pairs. The lanes are as follows: 1 marker (100 bp DNA ladder; Promega); 2, vehicle; 3,LPS; 4, siLUC; 5, particles prepared according to Example 61a; 6, particles prepared according to Example 55a; and 7, particles prepared according to Example 32a. Lanes 5, 6 and 7 show prominent bands having the same mobility as the 300 base pairs band in lane 1, the Marker lane. Thus, alignment of a major band in lanes 5, 6, and 7 with the band of the predicted length for 300 bp confirms the presence of the RNAi cut site in the particle configurations as described in Examples 61a, 55a, and 32a respectively.

### Methods Used in Example 71

### MDA-MB-231/GFP Cells

A MDA-MB-231/GFP human breast cancer cell line (Cell Biolabs, Inc.) was stably transfected into the genome with the enhanced EGFP gene using a lentivirus vector (not on a plasmid).

### Cell Culture

MDA-MB-231/EGFP cells were grown in complete media (DMEM, 10% FBS, pen/strep solution, 0.1 mM MEM non-essential amino acids, 2 mM L-glutamine) at 37° C., 5% CO₂. The seventh Passage, i.e., the seventh trypsinization of the cells to remove them from cell culture flasks to put into new flasks as the flasks become confluent, was implanted into mammary fat pad on nude mice.

### Flow Cytometry

Following sectioning of the tumor, tissue dissociation was completed utilizing a dissociation buffer consisting of phosphate buffered saline (PBS), 5% fetal bovine serum (FBS) and 0.1% sodium azide. Tissues were dissociated using a hand held pestle and mortar with sufficient clearance for intact cells to pass. Equal volumes of ice cold 2% paraformaldehyde solution were added for fixation of isolated cells and stored at 4° C. until FACS analysis. 2×10⁶ cells were analyzed utilizing a Becton Dickinson FACScan™ flow cytometer. MDA-MB-231 parent cells (non-EGFP) were used to determine the proper gating of non-EGFP cells compared to the EGFP cells.

### Fluorescent Protein Analysis

Tumor samples that were immediately frozen on dry ice were allowed to thaw in the presence of T-PER (Thermo Fisher) and supplemented with HALT® protease inhibitors (Thermo Fisher). Samples were then homogenized utilizing a Tissuemiser (Thermo Fisher) in 2 mL of T-PER. Total protein concentrations were measured using a BCA protein assay (Thermo Fisher) as described by the manufacturer to be completed using the microplate procedure. Protein concentrations were determined by preparing an albumin standard curve from a stock concentration of 2 mg/mL. EGFP fluorescence was detected using a SpectraMax® M5 (Molecular Devices) with the addition of 50 μg of total protein per well.

### RNA Extraction and Quantification

Tumor samples were stored at −20° C. in 1.5 mL of RNAlater® (Life Technologies) until processing. Tissues were homogenized in lysis buffer using hand-held micro centrifuge tube pestles, followed by centrifugation at 12,000 g for 1 min to remove any debris. The supernatant was then transferred into a micro-centrifuge tube. RNA was then extracted from the lysate utilizing the PureLink™ RNA Mini-Kit (Life Technologies) as described by manufactures suggested protocol. Purified RNA samples were then stored at −80° C. until further quantification and downstream analysis.

Quantification was completed using a RiboGreen® RNA quantization kit (Life Technologies), which is a 96-well plate fluorescence-based RNA quantification assay. The RNA determination is based on the provided RNA standards to generate a standard curve. The fluorescence signals were plotted against the RNA concentration with a background subtraction.

All samples were completed in triplicate. Modifications to the suggested protocol were limited to reduced total volumes, and the high-range standard curved was prepared as described by the manufacturer. Fluorescence was measured utilizing a SpectraMax® M5.

### QuantiGene® 2.0

The QuantiGene® 2.0 reagent system and assay kit (Affymetrix) was used to quantify target specific RNA, in particular, EGFP and GAPDH. Signals from the housekeeping gene will then be used to normalize gene expression across all data samples collected. A ratio of EGFP to GAPDH was used to normalize each sample, respectively. The percent knockdown was calculated from percent change of each sample when compared to the time point.

### 5′ RLM RACE-PCR

5′ RNA-ligand-mediated rapid amplification of cDNA ends polymerase chain reaction (5′ RLM RACE-PCR) was performed as described by the Invitrogen GeneRacer™ manual with slight modifications. Briefly, 100 ng of total isolated RNA was ligated directly to the GeneRacer™ RNA adaptor (5′-CGACUGGAGCACGAGGACACUGACAUGGACUGAAGGAGUAGAAA-3′) using T4 RNA ligase (5U) for 1 h at 37° C. The dephosphorlyation of RNA by calf intestinal phosphatase was omitted as well as the removal of the mRNA cap structure. After phenol extraction and precipitation, samples were reverse-transcribed using the SuperScript™ III module of the GeneRacer™ kit and the EGFP gene-specific reverse primer (5′-TCAGGTTCAGGGGGAGGTGTGG-3′). To detect cleavage products, PCR was performed using primers complementary to the RNA adaptor (GR5′:5′-CTCTAGAGCGACTGGAGCACG-3′) and with EGFP primers (EGFP #1: 5′-AGCCCCTCTAGAGTCGCGGC-3′) (EGFP#2:5′-CGCCGATGGGGGTGTTCTGC-3′) (EGFP#3: 5′-CGGTTCACCAGGGTGTCGCC-3′). Amplification products were resolved by 4% E-Gel® EX (Life Technologies) electrophoresis and visualized with E-Gel® sample loading buffer (Life Technologies).

### Example 72

### In Vivo siRNA Knockdown of EGFP

Cultured MDA-MB-231 breast cancer cells genetically engineered to express EGFP (MDA-MB-231/GFP, Cell Biolabs, Inc.) were implanted into the mammary fat pad of nude mice. Mice in each of 13 groups (nine mice per group) were administered control or siEGFP formulations as described in Table WWW. Mice in Group 1 were administered a formulation of vehicle (10% sucrose) which provided a control of no knockdown. Mice in Group 2 were administered particles prepared according to Example 61b, which provided a control siRNA particle against a non-targeted luciferase gene. Mice in Groups 3-13 were administered formulations of siEGFP particles prepared as described in the examples referenced in Table WWW as a 10 mL/kg bolus into the tail vein.

The properties of the particles are shown below in Table VVV. Two batches of particles according to Example 61a were prepared using identical components and methods except that the siRNA (against EGFP) was obtained from two different batches from the manufacturer.

TABLE VVV
Particle properties for knockdown and tolerability studies.

| | | | | | | siRNA |
|---|---|---|---|---|---|---|
| | | | | | Zeta | wt. % |
| Formulations | Z_{avg} | PDI | Dv50 | Dv90 | potential | loading |
| Particles prepared | 82.42 | 0.167 | 62.8 | 112 | +10.5 | 2.97 |
| according to | | | | | | |
| example 61b. | | | | | | |
| Particles prepared | 84.57 | 0.186 | 62.7 | 114 | +10.6 | 4.08 |
| according to | | | | | | |
| example 55a. | | | | | | |
| Particles prepared | 85.99 | 0.181 | 55.3 | 112 | +9.28 | 5.27 |
| according to | | | | | | |
| example 61a. | | | | | | |
| (Batch 1) | | | | | | |
| Particles prepared | 81.7 | 0.133 | 63.5 | 109 | +9.86 | 4.42 |
| according to | | | | | | |
| example 61a. | | | | | | |
| (Batch 2) | | | | | | |
| Particles prepared | 85.32 | 0.14 | 65.6 | 115 | +9.13 | 2.21 |
| according to | | | | | | |
| example 32a. | | | | | | |

Tumor samples were collected from 3 mice at each of 24 hours, 72 hours and 120 hours after the administration. Collected tumor material was then sectioned into 3 individual pieces for analysis. Sections were either placed into 1.5 mL of RNAlater® (Life Technologies), or immediately frozen on dry ice or dissociated into cells with phosphate buffered saline supplemented with 5% fetal bovine serum and 0.1% sodium azide.

TABLE WWW
Groups, dosing, and schedule.

| | | Dose | | |
|---|---|---|---|---|
| Group | Formulation | (mg/kg) | Schedule | N |
| 1 | Vehicle 10% | n/a | 1x | 9 |
| | Sucrose | | | |
| 2 | Particles prepared | 3 | 1x | 9 |
| | according to 61b. | | | |
| 3 | Particles prepared | 0.3 | 1x | 9 |
| | according to | | | |
| | example 55a. | | | |
| 4 | Particles prepared | 1.0 | 1x | 9 |
| | according to | | | |
| | example 55a. | | | |
| 5 | Particles prepared | 3.0 | 1x | 9 |
| | according to | | | |
| | example 55a. | | | |
| 6 | Particles prepared | 0.3 | 1x | 9 |
| | according to | | | |
| | example 61a. | | | |
| | (Batch 1) | | | |
| 7 | Particles prepared | 1.0 | 1x | 9 |
| | according to | | | |
| | example 61a. | | | |
| | (Batch 1) | | | |
| 8 | Particles prepared | 3.0 | 1x | 9 |
| | according to | | | |
| | example 61a. | | | |
| | (Batch 1) | | | |
| 9 | Particles prepared | 3.0 | q2d x2 | 9 |
| | according to | | | |
| | example 61a. | | | |
| | (Batch 1) | | | |
| 10 | Particles prepared | 3.0 | q2d x2 | 9 |
| | according to | | | |
| | example 61a. | | | |
| | (Batch 2) | | | |
| 11 | Particles prepared | 0.3 | 1x | 9 |
| | according to | | | |
| | example 32a. | | | |
| 12 | Particles prepared | 1.0 | 1x | 9 |
| | according to | | | |
| | example 32a. | | | |
| 13 | Particles prepared | 3.0 | 1x | 9 |
| | according to | | | |
| | example 32a. | | | |

The effect of the treatment on EGFP knockdown was determined by analysis of EGFP fluorescence. In the EGFP fluorescence analysis, total protein was extracted from the tumor samples utilizing T-PER (tissue protein extraction reagent, ThermoFisher) supplemented with HALT® protease inhibitor cocktail (ThermoFisher). Frozen tumors were thawed in the presence of 1.5 mL of T-PER prior to homogenization. Total protein was determined using a BCA assay kit (ThermoFisher). Following the calculations of total protein, 50 μg of total protein was measured for EGFP fluorescence using a SpectraMax® M5 (Molecular Devices) fluorescent plate reader with a filter set with an excitation of 488 nm and emission of 535 nm, with a designated cutoff at 535 nm. The percent knockdown of tumor protein was generated from the decrease of EGFP signal when directly compared to the untreated (vehicle) protein samples from identical time points, as shown below in Table XXX.

TABLE XXX
In vivo knockdown data.

| | | Dose | % Knockdown | % Knockdown | % Knockdown |
|---|---|---|---|---|---|
| Group | Formulation | (mg/kg) | (24 Hrs) | (72 Hrs) | (120 Hrs) |
| 1 | Vehicle 10% Sucrose | n/a | n/a | n/a | n/a |
| 2 | Particles prepared | 3 | 6.81 ± 10.18 | 0.40 ± 10.69 | 2.3 ± 9.64 |
| | according to 61b. | | | | |
| 3 | Particles prepared | 0.3 | ** | 12.91 ± 4.53 | 3.59 ± 6.24 |
| | according to | | | | |
| | example 55a. | | | | |
| 4 | Particles prepared | 1.0 | 7.52 ± 4.94 | 32.29 ± 4.93 | 11.19 ± 6.02 |
| | according to | | | | |
| | example 55a. | | | | |
| 5 | Particles prepared | 3.0 | 22.42 ± 14.04 | 27.29 ± 0.83 | 10.86 ± 2.01 |
| | according to | | | | |
| | Example 55a. | | | | |
| 6 | Particles prepared | 0.3 | 8.87 ± 9.27 | 16.36 ± 6.53 | 1.22 + 9.27 |
| | according to | | | | |
| | example 61a. | | | | |
| | (Batch 1) | | | | |
| 7 | Particles prepared | 1.0 | 20.52 ± 8.51 | 30.29 ± 3.71 | 24.12 ± 1.00 |
| | according to | | | | |
| | example 61a. | | | | |
| | (Batch 1) | | | | |
| 8 | Particles prepared | 3.0 | 29.11 ± 6.41 | 42.03 ± 8.15 | 34.68 ± 2.63 |
| | according to | | | | |
| | example 61a. | | | | |
| | (Batch 1) | | | | |
| 9 | Particles prepared | 3.0 | n/a | 29.76 ± 4.29 | n/a |
| | according to | | | | |
| | example 61a. | | | | |
| | (Batch 1) | | | | |
| 10 | Particles prepared | 3.0 | n/a | 39.39 ± 2.50 | n/a |
| | according to | | | | |
| | example 61a. | | | | |
| | (Batch 2) | | | | |
| 11 | Particles prepared | 0.3 | −2.34 ± 17.22 | 12.22 ± 1.89 | 1.72 ± 0.52 |
| | according to | | | | |
| | example 32a. | | | | |
| 12 | Particles prepared | 1.0 | 8.80 ± 4.57 | 19.86 ± 3.87 | 11.60 ± 1.46 |
| | according to | | | | |
| | example 32a. | | | | |
| 13 | Particles prepared | 3.0 | 25.86 ± 2.90 | 27.74 ± 4.90 | 15.95 ± 1.66 |
| | according to | | | | |
| | example 32a. | | | | |

** Indicates no knockdown observed.
n/a = data points not obtained.

As compared to the mice of group 2 that were treated with a control particle, all of the particles of groups 3-13 demonstrated an increase in knockdown, e.g., at 72 hours, as compared to the vehicle control group and the control particle of group 2. The particles prepared according to example 61a showed the greatest percentage of knockdown.

### Example 72a

### In Vivo siRNA Knockdown of EGFP

MDA-MB-468/GFP cells (Cell Biolabs, Inc.) were grown in RPMI-1640/10% FBS/1% Penn/Strep antibiotics (all from Invitrogen) until Passage 10. The MDA-MB-468/GFP model is a slow-growing tumor model, i.e., as compared to MDA-MB-231/GFP, the faster-growing tumor model used in Example 72.

The following in vivo study was performed on homozygous female NCR nu/nu nude mice (Taconic Farms): On Day 1, 5×10⁶ cells (MDA-MB-468/GFP-Passage 10, see above) were mixed into 100 μL of 50% RPMI-1640/50% Matrigel (BD Biosciences, Inc.) and implanted into the mammary fat pad of each mouse. On Day 13 mice weighing 20.4-26.4 g and having a mean tumor volume 57-69 mm³ were put into 2 groups (vehicle and particle), each group having mice for each of three time points measured, i.e., 24, 72, and 120 hours. Each tumor-bearing mouse received a single treatment of vehicle (10% sucrose in Tris EDTA buffer) or particles prepared according to Example 61a (2.2 mg/kg), administered intravenously into the tail vein at a dose volume of 10 mL/kg. At the 24 hour (Day 14), 72 hour (Day 16), and 120 hour (Day 18) time points, tumors were removed from each treatment group. Collected tumor material was then sectioned into 3 individual pieces for analysis. Sections were either directly placed into 1.5 mL of RNAlater® (Life Technologies) or immediately frozen on dry ice or dissociated into cells with phosphate buffered saline supplemented with 5% fetal bovine serum and 0.1% sodium azide. The frozen samples were stored at −80° C. until processed for protein and EGFP determination.

The effect of the treatment with particles prepared according to Example 61a was determined by analyzing EGFP fluorescence. Each tumor sample was thawed in the presence of T-PER (ThermoFisher) and supplemented with HALT® protease inhibitor cocktail (ThermoFisher). Samples were then homogenized using a hand-held mortar and pestle in 400 μL of supplemented T-PER. Total protein was determined using a BCA protein assay kit (ThermoFisher), where protein concentrations were determined by preparing an albumin standard curve from a stock of 2 mg/mL. Following the calculations of total protein, 50 μg of protein was diluted into 100 μL of PBS and measured for fluorescence using a SpectraMax® M5 (Molecular Devices) fluorescent plate reader (excitation wavelength=488 nm, emission wavelength=535 nm). The percent knockdown value for EGFP fluorescence was calculated by determining the percent decrease in EGFP fluorescent signal when compared to the Vehicle control from identical time points, as shown in Table YYY below.

TABLE YYY
In vivo EGFP knockdown data in MDA-MB-468/GFP tumors

| | | Dose, | % Knockdown | % Knockdown | % Knockdown |
|---|---|---|---|---|---|
| Group | Formulation | mg/kg | 24 hrs | 72 hrs | 120 hrs |
| 1 | Vehicle 10% Sucrose in TE | n/a | n/a | n/a | n/a |
| 2 | Particles prepared according | 2.2 | 12.1 ± 7.3 | 35.4 ± 15.1 | 69.9 ± 0.8 |
| | to Example 61a | | | | |

As seen in Table YYY, MDA-MB-468/GFP mice treated with particles prepared according to Example 61a demonstrated extended EGFP knockdown, e.g., up to 120 hours after administration, at levels much greater than the knockdown levels seen in MDA-MB-231/GFP tumors (see, in contrast, Example 72) at the same time point.

This result likely has a physiological basis because there was no measurable variation between the MDA-MB-231/GFP and MDA-MB-468/GFP cell lines in in vitro viability studies of the cells after exposure to particles prepared according to Example 61a. Additionally, the overall tumor volume in the MDA-MB-468/GFP model appeared to be independent of treatment, i.e., both the vehicle-treated and particle treated tumors increased in volume between 0 and 72 hours, and then decreased in volume by the 120 hour time point. The vehicle and particle groups were expected to have similar tumor growth characteristics because the EGFP knockdown is not relevant to tumor growth.

### Example 73

### Tolerability of siRNA Particles in Mice

Male C57BL/6 mice were administered free siEGFP solution, siLUC disulfide particles as described in Example 61b, siEGFP particles as described in Example 32a, siEGFP particles as described in Example 55a, or siEGFP particles as described in Example 61a (see, also, Table VVV). The administrations were intravenous at a dose of 3 mg/kg on a schedule of q2d×2, (ie., treated on the 1^{st} study day and the 3^{rd} study day, i.e., on Day 1 and on Day 3, i.e., 2 treatments 2 days apart).

Blood was collected 48 hrs after the 2^{nd} (final) treatment. Blood was analyzed for white blood cell number, red blood cell number, hemoglobin, hematocrit, mean corpuscular volume, mean corpuscular hemoglobin concentration, percent neutrophil (of WBC number), percent lymphocyte, percent monocyte, percent eosinophil, percent basophil, platelet estimate, polychromasia, anisocytosis, absolute neutrophil number, absolute lymphocyte number, absolute monocyte number, absolute eosinophil number, absolute basophil number. There were no significant changes in these parameters in mice receiving free siEGFP solution or any of the siEGFP particle formulations.

Serum was separated from the blood and analyzed for alkaline phosphatase, SGPT, SGOT, CPK, albumin, total protein, globulin, total bilirubin, direct bilirubin, indirect bilirubin, BUN, creatinine, cholesterol, glucose, calcium, phosphorus and bicarbonate. There were no significant changes in these parameters in mice receiving free siEGFP solution or any of the siEGFP particle formulations. Additional parameters that are normally analyzed in the serum of treated animals are chloride, potassium and sodium, but there was not enough serum collected from the mice for these parameters to be analyzed. In light of the lack of changes in the serum chemistry, it is not thought that there were any effects on chloride, potassium, and sodium by the siEGFP particle formulations.

### Example 74

### Circulating Cytokine Concentrations in Mice

Male C57BL/6 mice were administered siEGFP particles as described in Example 32a; siEGFP particles as described in Example 55a; or siEGFP particles as described in Example 61a (see, also, Table VVV). The treatment was a single intravenous administration, at a dose of 3 mg/kg.

A positive control, lipopolysaccharide (LPS 0111:B4, Sigma-Aldrich), was administered at a dose of 0.1 mg/kg intravenously. Particle controls were free (non-polymer-bound) siEGFP solution and siLUC particles as described in Example 61b, each administered at a dose of 3 mg/kg intravenously.

Blood was collected 2 hours and 6 hours after treatment.

Serum from the 2 hour time point was analyzed for tumor necrosis factor-alpha, interleukin-1alpha, interleukin-1beta, interleukin-6, interleukin-10, interleukin-12, keratinocyte-derived cytokine and interferon-gamma. The results of this study are shown in Table EEE. The positive control lipopolysaccharide treatment was accompanied by significant increases in all the cytokines measured. The particle controls, (free (non-polymer-bound) siEGFP solution and siLUC particles as described in 61b, and the particle formulations, i.e., siEGFP particles as described in Example 32a, siEGFP particles as described in Example 55a, or siEGFP particles as described in 61a, did not stimulate an increase in any of the cytokines measured.

Serum from the 6 hour time point was analyzed for the same cytokines. The positive control lipopolysaccharide treatment was accompanied by significant increases in all the cytokines measured at the 6 hour time point, but the concentrations were lower than at the 2 hour time point. The free (non-particle-bound) siEGFP solution, siEGFP particles as described in Example 32a, siEGFP particles as described in Example 55a, or siEGFP particles as described in Example 61a did not stimulate an increase in any of the cytokines measured. The siLUC particles as described in Example 61b stimulated a significant increase only in interferon-gamma at the 6 hour time point, not in any of the other cytokines measured. The increase in circulating interferon-gamma stimulated by the siLUC particles, as described in Example 61b, may be an off-target effect of the siLUC.

TABLE EEE
Mouse serum cytokine concentrations at 2 and 6 hours post-injection.

| | mIFNg | mIL-10 | mIL-1a | mIL-1b | mIL-6 | mKC | mTNFa | mIL-12p70 |
|---|---|---|---|---|---|---|---|---|
| Group | pg/ml | pg/ml | pg/ml | pg/ml | pg/ml | pg/ml | pg/ml | pg/ml |
| Vehicle: 2 h | 0 | 0 | 6 | 0 | 0 | 0 | 11 | 0 |
| LPS: 2h | 14 | 28 | 10 | 32 | 328553 | 389 | 222 | 1.9 |
| particle-free | 0 | 0 | 0 | 0 | 0 | 0 | 0 | 0 |
| siEGFP: 2 h | | | | | | | | |
| Particles prepared | 0 | 0 | 0 | 0 | 0 | 157 | 0 | 0 |
| according to | | | | | | | | |
| Example 61b: 2 h | | | | | | | | |
| Particles prepared | 0 | 0 | 0 | 0 | 2 | 0 | 0 | 0.1 |
| according to | | | | | | | | |
| Example 32a: 2 h | | | | | | | | |
| Particles prepared | 0 | 0 | 0 | 0 | 19 | 2 | 0 | 0 |
| according to | | | | | | | | |
| Example 61a: 2 h | | | | | | | | |
| Particles prepared | 0 | 0 | 0 | 0 | 0 | 0 | 0 | 0.1 |
| according to | | | | | | | | |
| Example 55a.: 2 h | | | | | | | | |
| Vehicle: 6 h | 0 | 0 | 0 | 0 | 0 | 0 | 0 | 0.1 |
| LPS: 6 h | 250 | 1.3 | 2 | 0 | 5887 | 146 | 28 | 1.4 |
| particle-free | 12 | 0 | 0 | 0 | 0 | 0 | 0 | 0 |
| siEGFP: 6 h | | | | | | | | |
| Particles prepared | 0 | 0 | 0 | 0 | 0 | 0 | 0 | 0 |
| according to | | | | | | | | |
| Example 61b: 6 h | | | | | | | | |
| Particles prepared | 0 | 0 | 0 | 0 | 0 | 0 | 0 | 0.02 |
| according to | | | | | | | | |
| Example 32a: 6 h | | | | | | | | |
| Particles prepared | 0 | 0 | 0 | 0 | 65 | 0 | 0 | 0 |
| according to | | | | | | | | |
| Example 61a: 6 h | | | | | | | | |
| Particles prepared | 0 | 0 | 0 | 0 | 0 | 0 | 0 | 0.4 |
| according to | | | | | | | | |
| Example 55a.: 6 h | | | | | | | | |

### Example 75

### Tolerability of siRNA Particle Formulations in Mice

Non-tumor bearing, male C57BL/6 mice with body weights in the range of 22.5-26.5 g/mouse were injected intravenously via tail vein with the formulations in Table FFF. The mice were assessed for the changes in body weights at day 1, day 3 and day 5 post-injection. Table FFF describes the groups, formulation administered, dose, regimen and number of mice per group.

TABLE FFF
Groups, dosing, and schedule.

| | | Dose | | |
|---|---|---|---|---|
| Group | Formulation | (mg/kg) | Schedule | N |
| 1 | Vehicle 10% | n/a | q2d x 2 | 5 |
| | Sucrose | | | |
| 2 | Particle-free | 3 | q2d x 2 | 5 |
| | siEGFP | | | |
| 3 | Particles prepared | 3 | q2d x 2 | 5 |
| | according to | | | |
| | Example 55a. | | | |
| 4 | Particles prepared | 3 | q2d x 2 | 5 |
| | according to | | | |
| | Example 61a. | | | |
| 5 | Particles prepared | 3 | q2d x 2 | 5 |
| | according to | | | |
| | Example 32a. | | | |

As shown in Table GGG, administration of the siEGFP particle formulations at a dose of 3 mg/kg and at a schedule of q2d×2 (administered on Day 1, and Day 3) did not cause body weight loss in the mice.

TABLE GGG
Post-injection body weight change.
Percent of Initial Body weights of mice administered SiEGFP particles

| Formulations | Day 1 | Day 3 | Day 5 |
|---|---|---|---|
| Group 1 | | | |
| Vehicle 10% sucrose | | | |
| n | 5 | 5 | 5 |
| mean | 100.0 | 99.4 | 101.4 |
| SD | 0.0 | 1.2 | 1.2 |
| SEM | 0.0 | 0.6 | 0.5 |
| Group 2 | | | |
| Particle-free siEGFP | | | |
| n | 5 | 5 | 5 |
| mean | 100.0 | 99.5 | 100.5 |
| SD | 0.0 | 0.9 | 1.3 |
| SEM | 0.0 | 0.4 | 0.6 |
| Group 3 | | | |
| Particles prepared according | | | |
| to Example 55a. | | | |
| n | 5 | 5 | 5 |
| mean | 100.0 | 100.2 | 102.7 |
| SD | 0.0 | 0.8 | 1.2 |
| SEM | 0.0 | 0.4 | 0.5 |
| Group 4 | | | |
| Particles prepared according | | | |
| to Example 61a. | | | |
| n | 5 | 5 | 5 |
| mean | 100.0 | 99.6 | 101.4 |
| SD | 0.0 | 1.1 | 1.3 |
| SEM | 0.0 | 0.5 | 0.6 |
| Group 5 | | | |
| Particles prepared according | | | |
| to example 32a. | | | |
| n | 5 | 5 | 5 |
| mean | 100.0 | 102.0 | 102.8 |
| SD | 0.0 | 1.4 | 3.0 |
| SEM | 0.0 | 0.6 | 1.3 |

### Example 76

### Assay for Complement Activation in Human Blood by siRNA Particle Formulations

Human whole blood was exposed to particles prepared according to Example 61a and Example 32a to determine if the particles activated complement (C3a or Bb) in the blood. Three samples of heparinized human whole blood were obtained from Bioreclamation LLC (Westbury, N.Y.) and were analyzed approximately 1 day after draw. The subjects were male, aged 36, 49 and 52 years. The blood was placed into wells on a 12 well cell culture plate, one plate for each individual's blood. Two mLs of blood were put into each of 8 wells per plate (i.e., not all the plate wells were used). Each 2 mL blood aliquot was treated according to Table HHH below, so that each treatment group had n=3. Lipopolysaccharide (LPS) was used as a positive control.

TABLE HHH
Treatment schedule for human blood.

| Group | Treatment | Dose | Schedule | n |
|---|---|---|---|---|
| 1 | Vehicle 10% sucrose TE | — | 1 hr | 3 |
| 2 | LPS | 70 μg/ml | 1 hr | 3 |
| 3 | Particle free siEGFP | 2.4 μM/0.032 mg/ml | 1 hr | 3 |
| 4 | Particles prepared | 2.4 μM/0.032 mg/ml | 1 hr | 3 |
| | according to | | | |
| | Example 61 a | | | |
| 5 | Particles prepared | 2.4 μM/0.032 mg/ml | 1 hr | 3 |
| | according to | | | |
| | Example 32 a | | | |

After the treatments were added to each corresponding well, the plates were covered and put in a desktop incubator/shaker and shaken moderately slowly at 37° C. (150 rpm). After 1 hour, 1 mL of blood from each well was transferred into a 1.5 mL Eppendorf tube and centrifuged at 10,000 rpm for 10 minutes. The plasma was immediately analyzed with MicroVue™ complement EIA kits (Quidel Corp., San Diego, Calif.) for C3a as a marker of classical and alternate pathways of complement activation, and for Bb as a marker of the alternate pathway of complement activation. C3a and Bb were measured according to the instructions included with the respective MicroVue™ complement EIA kits.

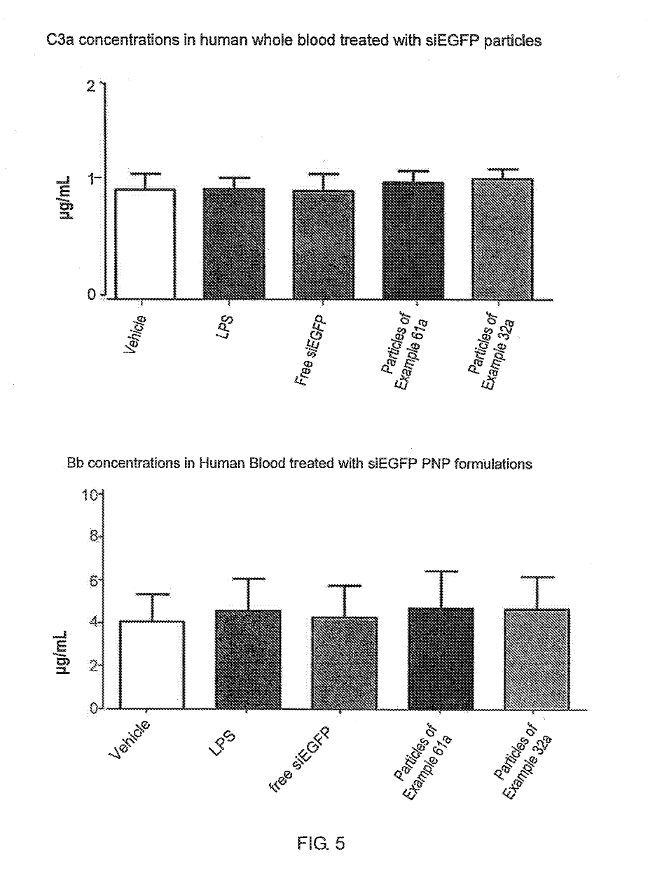

As shown in FIG. 5, the levels of C3a and Bb did not change, and remained within normal physiological ranges. Neither particle formulation activated complement (C3a or Bb), suggesting that siEGFP particles do not activate complement in human whole blood.

Other embodiments are in the claims.

1. A particle comprising:
a) a plurality of hydrophobic moieties;
b) a plurality of hydrophilic-hydrophobic polymers;
c) optionally, a plurality of cationic moieties; and
d) a plurality of nucleic acid agents, wherein at least a portion of the plurality of nucleic acid agents are
(i) covalently attached to either of
a hydrophobic moiety of a) or
a hydrophilic-hydrophobic polymer of b), or
(ii) form a duplex with a nucleic acid which is covalently attached to either of a hydrophobic moiety of a) or the hydrophilic-hydrophobic polymer b).

2. The particle of claim 1, wherein the particle comprises a plurality of cationic moieties.

3. The particle of claim 1, wherein the hydrophobic moieties are hydrophobic polymers.

4. The particle of claim 1, wherein the hydrophobic moieties are hydrophobic polymers, and at least a portion of the hydrophobic polymers of a) are each covalently attached to a nucleic acid agent of d).

5. The particle of claim 4, wherein at least a portion of the nucleic acid agents of d) are covalently attached to the hydrophobic polymer via a linker.

6. The particle of claim 4, wherein at least a portion of the hydrophobic polymers of a) are each covalently attached to a nucleic acid agent of d) through the 5′ position of the nucleic acid agent.

7. A particle comprising:
a) a plurality of nucleic acid agent-polymer conjugates, each of which comprises a nucleic acid agent which
(i) is attached to a hydrophobic polymer or
(ii) forms a duplex with a nucleic acid which is covalently attached to a hydrophobic polymer;
b) a plurality of hydrophilic-hydrophobic polymers; and
c) optionally, a plurality of cationic moieties.

8. The particle of claim 7, wherein the particle comprises a plurality of cationic moieties.

9. The particle of claim 7, wherein the particle further comprises a hydrophobic polymer, wherein the hydrophobic polymer is not attached to a nucleic acid agent.

10. The particle of claim 7, wherein the nucleic acid agent is covalently attached to a hydrophobic polymer via a linker.

11. A particle comprising:
a) a plurality of hydrophobic moieties;
b) a plurality of nucleic acid agent-hydrophilic-hydrophobic polymer conjugates wherein the nucleic acid agent of each nucleic acid agent-hydrophilic-hydrophobic polymer conjugate of the plurality
(i) is covalently attached to the hydrophilic-hydrophobic polymer or
(ii) forms a duplex with a nucleic acid which is covalently attached the hydrophilic-hydrophobic polymer; and
c) optionally, a plurality of cationic moieties.

12. The particle of claim 11, wherein the particle comprises a plurality of cationic moieties.

13. A particle comprising:
a) a plurality of hydrophobic polymers;
b) a plurality of hydrophilic-hydrophobic polymers;
c) a plurality of cationic moieties, wherein at least a portion of the plurality of cationic moieties is attached to either a hydrophobic polymer of a) or a hydrophilic-hydrophobic polymer of b); and
d) a plurality of nucleic acid agents.

14. The particle of claim 13, wherein at least a portion of the plurality of hydrophobic polymers of a) is not covalently attached to a cationic moiety of c) or a nucleic acid agent of d).

15. The particle of claim 13, wherein at least a portion of the plurality of hydrophobic polymers of a) are each covalently attached to a cationic moiety of c).

16. The particle of claim 13, wherein the cationic moiety is spermine or tetramethylated spermine.

17. A particle comprising:
a) a plurality of hydrophobic polymers;
b) optionally a plurality of hydrophilic-hydrophobic polymers;
c) a plurality of cationic moieties; and
d) a plurality of nucleic acid agents, wherein at least a portion of the plurality of nucleic acid agents are covalently attached to a hydrophilic polymer or form a duplex with a nucleic acid that is covalently attached to a hydrophilic polymer.

18. The particle of claim 17, wherein the nucleic acid agent is covalently attached to a hydrophilic polymer.

19. The particle of claim 17, wherein the hydrophilic polymer is polyethylene glycol (PEG).

20. The particle of claim 17, wherein the hydrophilic polymer is covalently attached to a lipid.

21. The particle of claim 17, wherein the particle is substantially free of a hydrophobic-hydrophilic polymer.

22. A particle comprising:
a) a plurality of hydrophobic polymers;
b) a plurality of hydrophilic-hydrophobic polymers; and
c) a plurality of nucleic acid agent-cationic polymer conjugates.

23. The particle of claim 22, wherein the cationic polymer is PVA.

24. The particle of claim 22, wherein the nucleic acid agent-cationic polymer conjugate is an siRNA-cationic PVA conjugate.

25. A particle comprising:
a) a plurality of hydrophobic polymers;
b) a plurality of hydrophilic-hydrophobic polymers;
c) optionally, a plurality of cationic moieties; and
d) a plurality of nucleic acid agents;
wherein a substantial portion of the cationic moieties of c) and a substantial portion of the nucleic acid agents of d) is not covalently attached to a hydrophobic polymer or a hydrophilic-hydrophobic polymer.

26. The particle of claim 25, wherein the nucleic acid agents or cationic moieties are embedded in the particle.

27. The particle of claim 25, wherein the particle comprises a plurality of cationic moieties.

28. The particle of claim 27, wherein the cationic moieties are cationic polymers.

29. The particle of claim 1, wherein the hydrophobic moiety or hydrophobic polymer is PLGA.

30. The particle of claim 29, wherein the PLGA is modified on a terminal end with a reactive group.

31. The particle of claim 1, wherein the hydrophilic-hydrophobic polymer is PEG-PLGA.

32. The particle of claim 1, wherein the cationic moiety includes at least one primary, secondary, tertiary or quaternary amine.

33. The particle of claim 1, wherein the nucleic acid agent is an siRNA.

34. The particle of claim 1, wherein the particle further comprises a surfactant.

35. The particle of claim 1, wherein the particle has a zeta potential of from about −20 mV to about +20 mV

36. A composition comprising a plurality of particles of claim 1.

37. The composition of claim 36, wherein the composition is a pharmaceutical composition.

38. The composition of claim 36, wherein the particles have a Dv90 of less than 200 nm.

39. A kit comprising a plurality of particles of claim 1.

40. A kit comprising a composition of claim 36.

41. A single dosage unit comprising a plurality of particles of claim 1.

42. A single dosage unit comprising a composition of claim 36.

43. A method of treating a subject having a disorder comprising administering to the subject an effective amount of a composition of claim 36, to thereby treat a subject.

44. A nucleic acid agent-hydrophobic polymer conjugate comprising a nucleic acid agent covalently attached to a hydrophobic polymer, wherein the nucleic acid agent is an siRNA which is attached to the polymer via the 5′ end of the sense strand.

45. The conjugate of claim 44, wherein the nucleic acid agent is covalently attached to the hydrophobic polymer via a linker.

46. A nucleic acid agent-hydrophilic-hydrophobic polymer conjugate comprising a nucleic acid agent covalently attached to a hydrophilic-hydrophobic polymer.

47. The conjugate of claim 46, wherein the nucleic acid agent is attached to the hydrophilic portion of the hydrophilic-hydrophobic polymer.

48. The conjugate of claim 46, wherein the nucleic acid agent is attached to the hydrophobic portion of the hydrophilic-hydrophobic polymer.

49. A method of making a particle comprising a nucleic acid agent, the method comprising, combining, in a polar solvent under conditions that allow formation of a particle:
(a) nucleic acid agent-hydrophobic polymer conjugates, each nucleic acid agent-hydrophobic polymer conjugate comprising a nucleic acid agent covalently attached to a hydrophobic polymer, wherein the nucleic acid agent-hydrophobic polymer conjugates are associated with a cationic moiety;
(b) a plurality of hydrophilic-hydrophobic polymers; and
(c) a plurality of hydrophobic polymers
to thereby form a particle.

50. A composition of claim 36, which, when administered to a subject, results in a reduction in the expression of a target gene that is at least 10, 20, 50, 75, 80, 90, 100, 200, or 500%, greater than:
(a) the reduction in the expression of the target gene seen with the nucleic acid agent administered in a formulation other than a particle or a conjugate to the subject; or
(b) the expression of the target gene in the absence of administration of the nucleic acid agent.

51. A method of storing a composition of claim 36, the method comprising:
(a) providing said composition disposed in a container;
(b) storing said composition; and
(c) moving said container to a second location or removing all or an aliquot of said composition, from said container.

52. The method of claim 51, wherein the composition stored is a re-constituted formulation.


SEQUENCE LISTING (8 sequences)

SEQ ID NO: 1 - DNA, 22 bp, Artificial Sequence
  Description of Artificial Sequence Synthetic primer
tcaggttcagggggaggtgtgg

SEQ ID NO: 2 - DNA, 23 bp, Artificial Sequence
  Description of Artificial Sequence Synthetic primer
cgactggagcacgaggacactga

SEQ ID NO: 3 - DNA, 20 bp, Artificial Sequence
  Description of Artificial Sequence Synthetic primer
cgccgatgggggtgttctgc

SEQ ID NO: 4 - RNA, 44 nt, Artificial Sequence
  Description of Artificial Sequence Synthetic oligonucleotide
cgacuggagcacgaggacacugacauggacugaaggaguagaaa

SEQ ID NO: 5 - DNA, 22 bp, Artificial Sequence
  Description of Artificial Sequence Synthetic primer
tcaggttcagggggaggtgtgg

SEQ ID NO: 6 - DNA, 21 bp, Artificial Sequence
  Description of Artificial Sequence Synthetic primer
ctctagagcgactggagcacg

SEQ ID NO: 7 - DNA, 20 bp, Artificial Sequence
  Description of Artificial Sequence Synthetic primer
agcccctctagagtcgcggc

SEQ ID NO: 8 - DNA, 20 bp, Artificial Sequence
  Description of Artificial Sequence Synthetic primer
cggttcaccagggtgtcgcc